(19) United States
(12) Patent Application Publication
(10) Pub. No.: US 2004/0267017 A1
(43) Pub. Date: Dec. 30, 2004

(54) 3-pyridyl or 4-isoquinolinyl thiazoles as c17, 20 lyase inhibitors

(76) Inventors: Donald E Bierer, Bethany, CT (US); Jeffrey Johnson, Branford, CT (US); Gaetan Ladouceur, Guilford, CT (US); William J Scott, Guilford, CT (US); Andrea C McClure, West Haven, CT (US); Wenlang Fu, Hamden, CT (US); Furahi Achebe, West Haven, CT (US); Michael Burke, New Haven, CT (US); Chen Bi, West Haven, CT (US); Barry P Hart, Palo Alto, CA (US)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/490,822

(22) PCT Filed: Sep. 26, 2002
(86) PCT No.: PCT/US02/30483

Related U.S. Application Data
(60) Provisional application No. 60/324,993, filed on Sep. 26, 2001.

(51) Int. Cl. C 07D 4 3/02; C07D 233/70

(52) U.S. Cl. 544/370; 548/322.5

(57) ABSTRACT

The invention provides novel thiazoles bearing 3-pyridyl or 4-isoquinilinyl substituents, and pharmaceutical compositions thereof. The invention also provides methods of using compounds of the invention and pharmaceutical compositions thereof as inhibitors of lyases, e.g., the 17a-hydroxylase-C17,20 enzyme. The invention further provides methods for treating cancer in a subject, comprising administering to the subject a compound of the invention or a pharmaceutical composition thereof. The cancer can be, e.g., prostate cancer or breast cancer.

### BACKGROUND OF THE INVENTION

Steroid biosynthesis begins in cells of the adrenal gland where the initial product in sterol biosynthesis, cholesterol, is converted into the adrenal steroid hormones aldosterone, hydrocortisone, and corticosterone by a series of P₄₅₀-mediated hydroxylation steps. The cholesterol side-chain cleavage activity that represents the first step in steroid hormone biosynthesis is a P₄₅₀-mediated oxidation and cleavage of a pair of adjacent methylene groups to two carbonyl fragments, pregnenolone and isocaprylaldehyde (see Walsh (1979) Enzymatic Reaction Mechanisms; W.H. Freeman and Company, pp. 474-77). Another critical set of enzymatic conversions in steroid metabolism is facilitated by 17-alpha-hydroxylase-17,20-lyase (CYP17, P₄₅₀ 17). CYP17 is a bifunctional enzyme which possesses both a C17,20-lyase activity and a C17-hydroxylase activity. Significantly, these two alternative enzymatic activities of CYP 17 result in the formation of critically different intermediates in steroid biosynthesis and each activity appear to be differentially and developmentally regulated (see e.g. I'Allemand et al. (2000) Eur. J. Clin. Invest 30: 28-33).

The C17,20-lyase activity of CYP17 catalyzes the conversion of 17α-hydroxy-pregnenolone and 17α-hydroxy progesterone to dehydroepiandrosterone (DHEA) and delta4-androstenedione (androstenedione) respectively. Both DHEA and androstenedione lyase products are key intermediates in the synthesis of not only the androgens testosterone and dihydrotestosterone (DHT), but also the estrogens 17-beta-estradiol and estrone. Indeed, adrenal and ovarian estrogens are the main sources of estrogens in postmenopausal women (see e.g. Harris et al. (1988) Br. J. Cancer 58: 493-6). In contrast, the C17-hydroxylase activity of CYP 17 catalyzes the conversion of the common intermediate progesterone to 17-hydroxyprogesterone, a precursor of cortisol. Therefore the first activity of CYP 17, the C17-hydroxylase activity, promotes the formation of glucocorticoids while the second activity of CYP17, the C17,20-lyase activity, promotes the formation of sex hormones—particularly androgens including testosterone as well as estrogens.

Prostate cancer is currently one of the most frequently diagnosed forms of cancer in men in the U.S. and Europe. Prostate cancer is typically androgen-dependent and, accordingly, the reduction in androgen production via surgical or pharmacological castration remains the major treatment option for this indication. However, complete rather than partial withdrawal of androgens may be more effective in treating prostate cancer (Labrie, F. et al., Prostate, 1983, 4, 579 and Crawford, E. D. et al., N. Engl. J Med., 1989, 321, 419). Pharmacological inhibition of CYP 17 may be a promising alternative treatment to antiandrogens and LHRH agonists in that testicular, adrenal, and peripheral androgen biosynthesis would be reduced rather than only testicular androgen production (Njar V, et al., J. Med. Chem., 1998, 41, 902). One such CYP17 inhibitor, the fungicide ketoconazole, has been used previously for prostate cancer treatment (Trachtenberg, J., J. Urol., 1984, 132, 61 and Williams, G. et al., Br. J. Urol., 1986, 58, 45). However, this drug is a relatively non-selective inhibitor of cytochrome P450 (CYP) enzymes, has weak CYP 17 activity, and has a number of notable side effects associated with it including liver damage (De Coster, R. et al., J. Steroid Biochem. Mol. Biol., 1996, 56, 133 and Lake-Bakaar, G. et al., Br. Med. J, 1987, 294, 419).

The importance of potent and selective inhibitors of CYP 17 as potential prostate cancer treatments has been the subject of numerous studies and reviews (Njar, V. et al., Curr. Pharm. Design, 1999, 5, 163; Barrie, S. E. et al., Endocr. Relat. Cancer, 1996, 3, 25 and Jarman, M. et al., Nat. Prod. Rep., 1998, 495). Finasteride, a 5α-reductase inhibitor, is an approved treatment for benign prostatic hyperplasia (BPH), although it is only effective with patients exhibiting minimal disease. While finasteride reduces serum DHT levels, it increases testosterone levels, and may therefore be insufficient for prostate cancer treatment (Peters, D. H. et al., Drugs, 1993, 46, 177). Certain anti-androgenic steroids, for example, cyproterone acetate (17α-acetoxy-6-chloro-1α, 2α-methylene-4,6-pregnadiene-3,20-dione), have been tested as adjuvant treatments for prostate cancer. Many other steroids have been tested as hydroxylase/lyase inhibitors. See, for example, PCT Specification WO 92/00992 (Schering A G) which describes anti-androgenic steroids having a pyrazole or triazole ring fused to the A ring at the 2,3-position, or European specifications EP-A288053 and EP-A413270 (Merrell Dow) which propose 17β-cyclopropylamino-androst-5-en-3β-ol or -4-en-3-one and their derivatives.

In addition to the use of CYP17 inhibitors in the treatment of prostate cancer, a second potential indication would be for estrogen-dependent breast cancer. In postmenopausal patients with advanced breast cancer, treatment with high doses of ketoconazole resulted in suppression of both testosterone and estradiol levels, implicating CYP17 as a potential target for hormone therapy (Harris, A. L. et al., Br. J Cancer, 1988, 58,493).

Chemotherapy is usually not highly effective, and is not a practical option for most patients with prostate cancer because of the adverse side effects which are particularly detrimental in older patients. However, the majority of patients initially respond to hormone ablative therapy although they eventually relapse, as is typical with all cancer treatments (McGuire, in: Hormones and Cancer, Iacobelli et al. Eds.; Raven Press, New York, 1980, Vol. 15, 337-344). Current treatment by orchidectomy or administration of gonadotropin-releasing hormone (GnRH) agonists results in reduced androgen production by the testis, but does not interfere with androgen synthesis by the adrenals. Following three months of treatment with a GnRH agonist, testosterone and DHT concentrations in the prostate remained at 25% and 10%, respectively, of pretreatment levels (Forti et al., J. Clin. Endocrinol. Metab., 1989, 68, 461). Similarly, about 20% of castrated patients in relapse had significant levels of DHT in their prostatic tissue (Geller etal., J. Urol., 1984, 132, 693). These findings suggest that the adrenals contribute precursor androgens to the prostate. This is supported by clinical studies of patients receiving combined treatment with either GnRH or orchidectomy and an anti-androgen, such as flutamide, to block the actions of androgens, including adrenal androgens. Such patients have increased progression-free survival time compared to patients treated with GnRH agonist or orchidectomy alone (Crawford et al., N. Engl. J Med., 1989, 321, 419 and Labrie et al., Cancer Suppl., 1993, 71, 1059).

Although patients initially respond to endocrine therapy, they frequently relapse. It was reported recently that in 30% of recurring tumors of patients treated with endocrine therapy, high-level androgen receptor (AR) amplification was found (Visakorpi, et al., Nature Genetics, 1995, 9, 401). Also, flutamide tends to interact with mutant ARs, and stimulate prostatic cell growth. This suggests that AR amplification may facilitate tumor cell growth in low androgen concentrations. Thus, total androgen blockade as first line therapy may be more effective than conventional androgen deprivation by achieving maximum suppression of androgen concentrations which may also prevent AR amplification. It is presently unclear whether sequential treatment with different agents can prolong the benefits of the initial therapy. This strategy has been found effective in breast cancer treatment. New agents which act by different mechanisms could produce second responses in a portion of relapsed patients. Although the percentage of patients who respond to second-line hormonal therapy may be relatively low, a substantial number of patients may benefit because of the high incidence of prostate cancer. Furthermore, there is the potential for developing more potent agents than current therapies, none of which are completely effective in blocking androgen effects.

The need exists for C17,20 lyase inhibitors that overcome the above-mentioned deficiencies.

### SUMMARY OF THE INVENTION

The invention provides substituted 3-pyridyl heterocyclic compounds which inhibit the lyase activity of enzymes, e.g., 17α-hydroxylase-C17,20 lyase. The compounds of the invention have the formula (I)

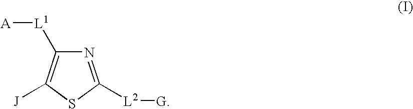

In formula (I),

L¹ represents

a chemical bond;

a carbonyl group;

—(CH₂)ₐ— in which a is 1, 2, or 3;

—CH₂O—;

—OCH₂—;

—O—;

—N(R¹)— in which R¹ represents H or C₁₋₄ alkyl;

—NHC(O)—;

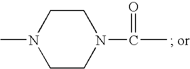

—CH₂NHC(O)—.

L² represents

a chemical bond;

—(CH₂)ₐ—;

—CH₂O—;

—N(R¹)—; or

—NH(CH₂)ₙ—.

J represents

H;

C₁₋₄ alkyl; or

halogen.

Furthermore,

1) when L¹ is a chemical bond, A represents

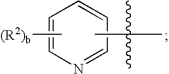

in which

b is 0, 1, or 2; and

R² is selected from

C₁₋₆ alkyl;

C₁₋₄ haloalkyl;

OR¹;

C₃₋₆ cycloalkyl;

halogen;

phenyl optionally substituted by halogen;

NO₂;

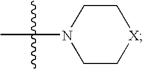

in which X represents CH₂, O, S, or N(R¹);

—N(³)₂ ; in which R³ represents H, C₁₋₄ alkyl, C₄₋₆ cycloalkyl, or phenyl optionally substituted by halogen;

—(CH₂)ₐN(R¹)(R⁴) in which R⁴ represents —(CH₂)ₐOR¹ or —(CH₂)ₐN(R¹)₂; and

—(CH₂)ₐR⁵ ; in which

R⁵ represents

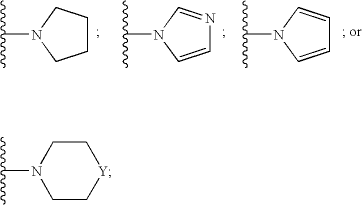

in which

Y represents N(R¹), O, S, or

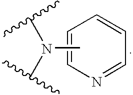

When L¹ is a bond, A may also be

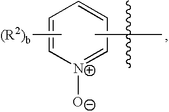

provided that G is other than a pyridyl or an N-oxide-containing group.

When L¹ is a bond, A may also be

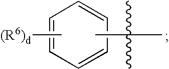

in which

d is 0, 1, or 2; and

R⁶ is selected from

C₁₋₆ alkyl;

C₁₋₄ haloalkyl;

OR⁷; in which

R⁷ represents H, C₁₋₄ alkyl, C₁₋₄ haloalkyl, phenyl, benzyl, or pyridyl optionally substituted by C₁₋₃ haloalkyl;

halogen;

NO₂;

CN;

CO₂R¹;

C₁₋₄ acyl;

phenyl optionally substituted by halogen;

benzyl;

N(R₁)²;

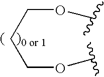

in which the O atoms are bonded to the phenyl ring at adjacent carbons;

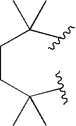

in which the terminal carbons are bonded to the phenyl ring at adjacent carbons;

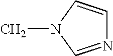

optionally substituted by halogen;

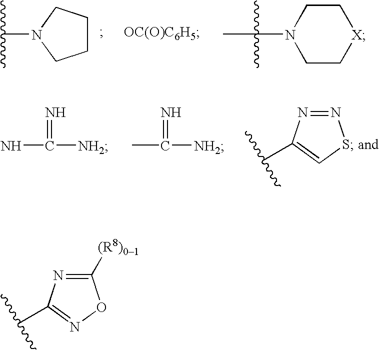

wherein R⁸ represents C₁₋₄ alkyl or phenyl optionally substituted by halogen.

When L¹ is a bond, A may also be

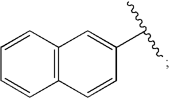

C₃₋₈ cycloalkyl;

C₅₋₆ cycloalkenyl;

adamantyl;

norbomyl;

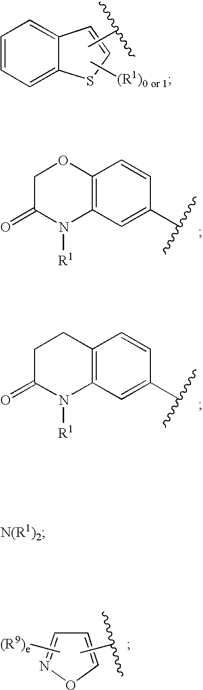

in which

e is 0, 1, or 2;and

R⁹ represents C₁₋₄ alkyl or phenyl optionally substituted by halogen;

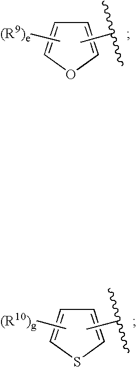

in which

g is 0 1, or 2;and

R¹⁰ represents CN, NO₂, or halogen.

Furthermore,

2) when L² is a bond, G represents

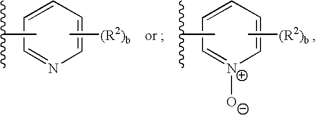

provided that A is other than a pyridyl or an N-oxide-containing group;

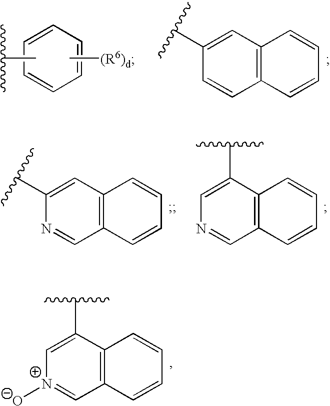

provided that A is other than a pyridyl or an N-oxide-containing group;

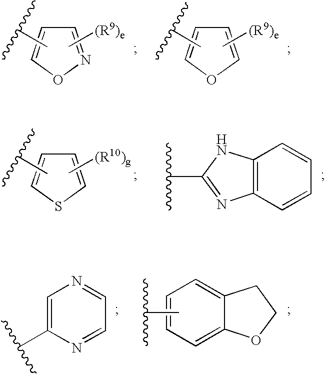

a diazole selected from

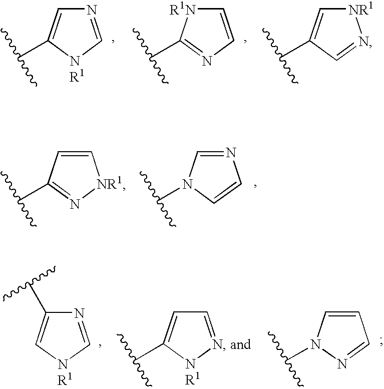

or

a triazole.

Furthermore,

3) when L¹ is carbonyl, A represents

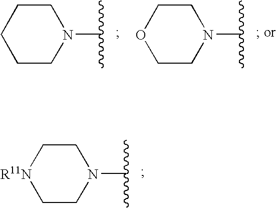

in which

R¹¹ represents H, C₁₋₄ alkyl, or phenyl optionally substituted by halogen;

Furthermore,

4) when L¹ is —(CH₂)ₐ—, A represents

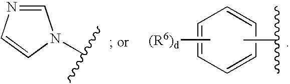

Furthermore,

5) when L² is —(CH₂)ₐ—, G represents

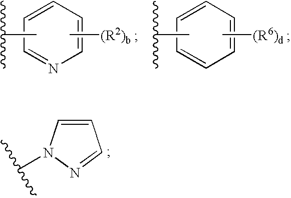

or

a triazole.

Furthermore,

6) when L¹ is —CH₂O, —OCH₂— or O, A represents

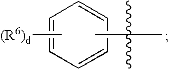

C₁₋₄ alkyl;

C₃₋₈ cycloalkyl; or

C₆₋₇ bicycloalkyl.

Furthermore,

7) when L² is —CH₂O—, G represents

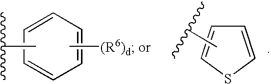

Furthermore,

8) when L¹ is —N(R¹)—, A represents

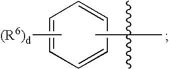

or

C₅₋₆ cycloalkyl.

Furthermore,

9) when L² is —N(R¹)— or —NH(CH₂)ₐ—, G represents

C₁₋₆ alkyl;

C₃₋₆ cycloalkyl;

N(R¹)₂;

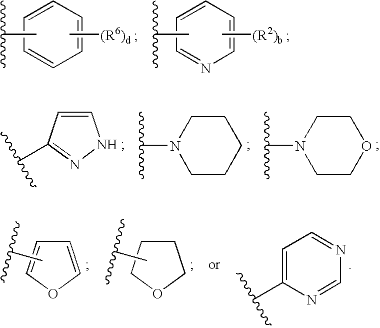

Furthermore,

10) when L¹ is —NHC(O)—,

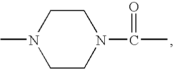

or —CH₂NHC(O)—, A represents

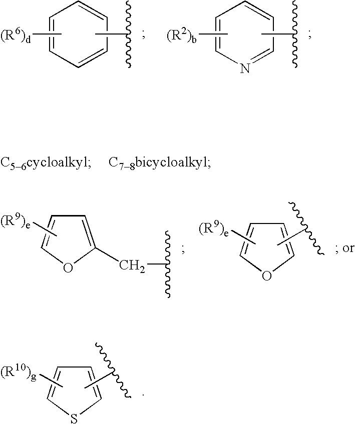

Furthermore,

11) one of A and G is a 3-pyridyl moiety of formula (II) or (IIA), or a 4-isoquinolyl moiety of formula (IIB) or (IIC)

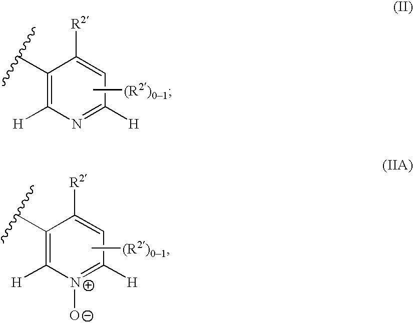

provided that the other of A and G is other than a pyridyl or an N-oxide-containing group;

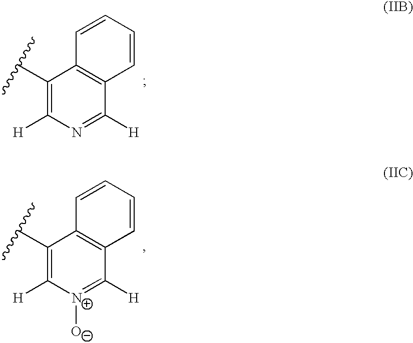

provided that the other of A and G is other than a pyridyl or an N-oxide-containing group;

and this 3-pyridyl or 4-isoquinolyl moiety is joined to the thiazole ring via a chemical bond L¹ or L² respectively; and the other of A and G is as defined above.

In addition, when the other of A and G is joined to the thiazole ring via linker L¹ or L² respectively where L¹ or L² is not a chemical bond, then R²′ of formulae (II) and (IIA) is R²; but when each of A and G is joined to the thiazole ring via a chemical bond L¹ and L² respectively, then R²′ of formulae (II) and (IIA) is selected from the group consisting of

C₂₋₆ alkyl, provided that when said 3-pyridyl moiety of formula (II) constitutes A, then G is other than phenyl substituted with an amide or sulfonamide group; and when said 3-pyridyl moiety of formula (II) constitutes G, then A is other than phenyl substituted with F;

C₂₋₄ haloalkyl;

C₂₋₄ alkoxy;

C₃₋₆ cycloalkyl;

phenyl optionally substituted by halogen;

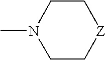

in which

Z represents CH₂, S, or N(R¹)

—N(R³′)₂ in which

R₃′ represents H, C₃₋₄ alkyl, C₄₋₆ cycloalkyl, or phenyl optionally substituted with halogen;

—(CH₂)ₐN(R¹)(R⁴);

—(CH₂)ₐR⁵;

Alternatively,

12) A-L¹ and J may be joined and together with the carbon atoms to which they are connected form a ring moiety selected from the group consisting of

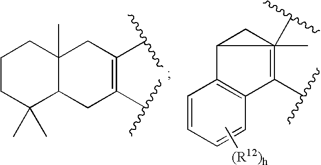

in which

h is 0, 1, or 2; and

R¹² represents C₁₋₄ alkyl or C₁₋₄ alkoxy;

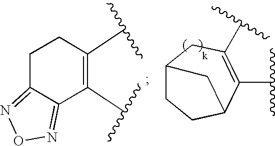

in which

k is 0 or 1; or

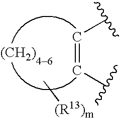

in which

m is 0, 1, or 2;

R¹³ represents C₁₋₄ alkyl or phenyl,

said ring moiety being joined to the thiazole at the positions indicated by the truncated valences shown in the partial structures above to form a fused ring thiazole; and for these fused ring thiazoles, L² is a bond and G is a 3-pyridyl moiety of formula (III) or (IIIA)

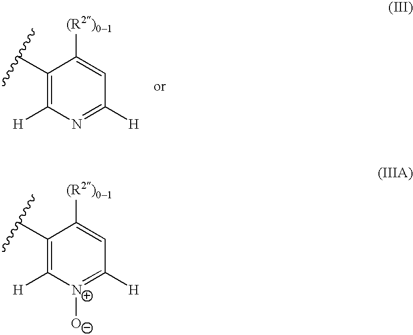

in which R²″ is C₁₋₄ alkyl.

Pharmaceutically acceptable salts of these compounds are also within the scope of the invention.

The invention also provides pharmaceutical compositions for inhibiting lyase activity, comprising a compound of the invention plus a pharmaceutically acceptable carrier.

The invention also provides methods for inhibiting lyases, comprising contacting the lyase with a compound of the invention. In particular, the invention provides a method of inhibiting a 17α-hydroxylase-C17,20 lyase, comprising contacting a 17α-hydroxylase-C17,20 lyase with a compound of the invention.

The invention further provides methods for treating diseases which can benefit from an inhibition of a lyase enzyme. Exemplary diseases are lyase-associated diseases, e.g., diseases resulting from an excess of androgens or estrogens. For example, the invention provides a method for treating cancer in a subject, comprising administering to the subject a pharmaceutically effective amount of a compound of the invention, such that the cancer is treated.

The method of treatment may be applied where the subject is equine, canine, feline, or a primate, in particular, a human.

The cancer may, for example, be prostate or breast cancer. Accordingly, a method for treating prostate cancer in a subject, comprises administering to the subject a therapeutically effective amount of a compound of the invention, such that the prostate cancer in the subject is treated. Similarly, a method for treating breast cancer in a subject comprises administering to the subject a therapeutically effective amount of a compound of the invention, such that the breast cancer in the subject is treated.

### DETAILED DESCRIPTION OF THE INVENTION

The invention is based at least in part on the discovery that substituted 3-pyridyl heterocyclic compounds inhibit the enzyme 17α-hydroxylase-C17,20 lyase.

In the broadest embodiment, the compounds of the invention have the formula (I) in which the several substituent moieties are as described in claim 1 and in the above summary of the invention.

In a preferred embodiment the compounds of the invention have the formula (I)

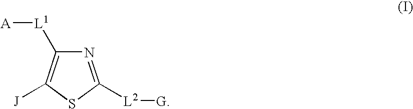

In formula (I),

L¹ preferebly represents

a chemical bond;

a carbonyl group;

—(CH₂)ₐ— in which a is 1, 2, or 3; or

—OCH₂—;

L² preferably represents

a chemical bond;

—(CH₂)ₐ; or

—N(R¹)— in which R¹ represents H or C₁₋₄ alkyl;

J preferably represents

H; or

C₁₋₄ alkyl.

Furthermore, in this preferred embodiment

1) when L¹ is a chemical bond, A represents

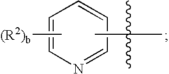

in which

b is 0, 1, or 2; and

R² is selected from

C₁₋₆ alkyl;

C₁₋₄ haloalkyl;

C₃₋₆ cycloalkyl;

halogen;

phenyl optionally substituted by halogen; and

—(CH₂)ₐR⁵; in which

R⁵ represents

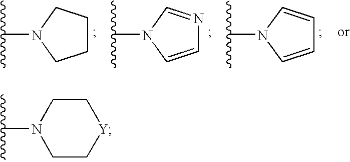

in which

Y represents N(R¹), O, S, or

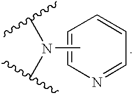

When L¹ is a bond, A may also be

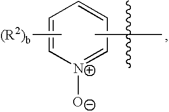

provided that G is other than a pyridyl or an N-oxide-containing group.

When L¹ is a bond, A may also be

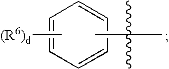

in which

d is 0, 1, or 2; and

R⁶ is selected from

C₁₋₆ alkyl;

C₁₋₄ haloalkyl;

OR⁷; in which

R⁷ represents C₁₋₄ alkyl or C₁₋₄ haloalkyl;

halogen;

NO₂;

CN;

CO₂R¹;

C₁₋₄ acyl;

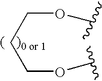

in which the O atoms are bonded to the phenyl ring at adjacent carbons;

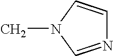

optionally substituted by halogen;

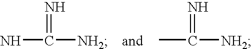

When L¹ is a bond, A may also be

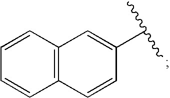

C₃₋₈ cycloalkyl;

C₅₋₆ cycloalkyl;

adamantyl;

norbomyl;

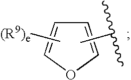

in which

e is 0, 1, or 2; and

R⁹ represents C₁₋₄ alkyl or phenyl optionally substituted by halogen;

or

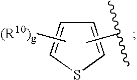

in which

g is 0, 1, or 2; and

R¹⁰ represents CN, NO₂, or halogen.

Furthermore,

2) when L² is a bond, G preferably represents

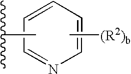

wherein

R² is selected from

C₁₋₆ alkyl;

C₁₋₄ haloalkyl;

C₃₋₆ cycloalkyl;

halogen;

phenyl optionally substituted by halogen; and

—(CH₂)ₐ—R⁵—, in which

R⁵ represents

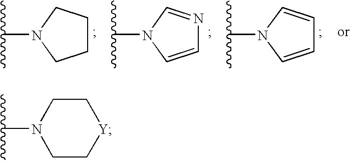

in which

Y represents N(R¹) , O, S, or

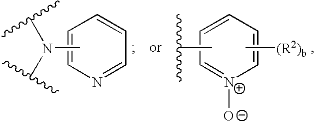

provided that A is other than a pyridyl or an N-oxide-containing group;

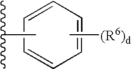

wherein

R⁶ is selected from

C₁₋₆ allyl;

C₁₋₄ haloalkyl;

OR⁷; in which

R⁷ represents C₁₋₄ alkyl or C₁₋₄ haloalkyl;

halogen;

NO₂;

CN;

CO₂R¹;

C₁₋₄ acyl;

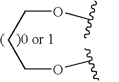

in which the O atoms are bonded to the phenyl ring at adjacent carbons;

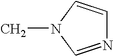

optionally substituted by halogen;

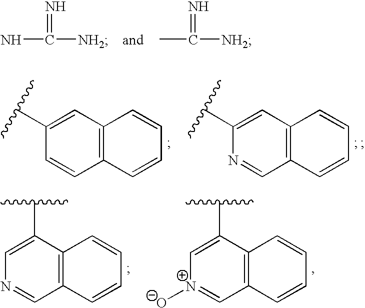

provided that A is other than a pyridyl or an N-oxide-containing group;

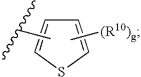

a diazole selected from

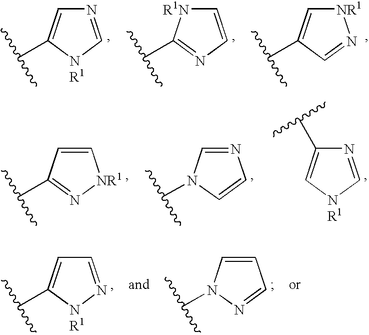

a triazole.

Furthermore,

11) one of A and G is a 3-pyridyl moiety of formula (II) or (IIA), or a 4-isoquinolyl moiety of formula (IIB) or (IIC)

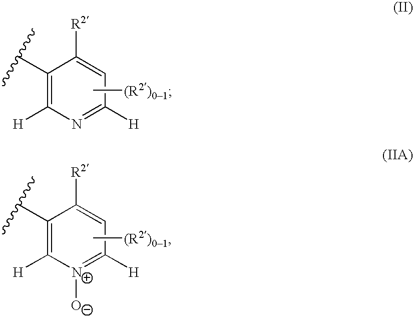

provided that the other of A and G is other than a pyridyl or an N-oxide-containing group;

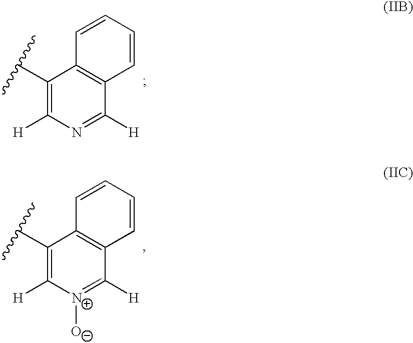

provided that the other of A and G is other than a pyridyl or an N-oxide-containing group;

and this 3-pyridyl or 4-isoquinolyl moiety is joined to the thiazole ring via a chemical bond L¹ or L² respectively, and the other of A and G is as defined above.

In addition, when the other of A and G is joined to the thiazole ring via linker L¹ or L² respectively where L¹ or L² is not a chemical bond, then R²′ of formulae (II) and (IIA) is R²; but when each of A and G is joined to the thiazole ring via a chemical bond L¹ and L² respectively, then R²′ of formulae (II) and (IIA) is selected from the group consisting of

C₂₋₆ alkyl, provided that when said 3-pyridyl moiety of formula (II) constitutes A, then G is other than phenyl substituted with an amide or sulfonamide group; and when said 3-pyridyl moiety of formula (II) constitutes G, then A is other than phenyl substituted with F;

C₃₋₆ cycloalkyl;

phenyl optionally substituted by halogen;

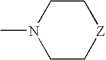

in which

Z represents CH₂, S, or N(R¹); and

—(CH₂)ₐR⁵.

Alternatively,

12) A-L¹ and J may be joined and together with the carbon atoms to which they are connected form a ring moiety selected from the group consisting of

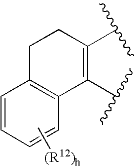

in which

h is 0, 1, or 2; and

R¹² represents C₁₋₄ alkyl or C₁₋₄ alkoxy, and

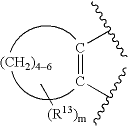

in which

m is 0, 1, or 2;

R¹³ represents C₁₋₄ alkyl or phenyl;

said ring moiety being joined to the thiazole at the positions indicated by the truncated valences shown in the partial structures above to form a fused ring thiazole; and for these fused ring thiazoles, L² is a bond and G is a 3-pyridyl moiety of formula (III) or (IIIA)

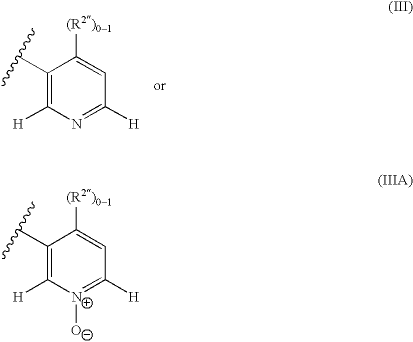

in which R²″ is C₁₋₄ alkyl.

In a more preferred embodiment the compounds of the invention have the formula (I)

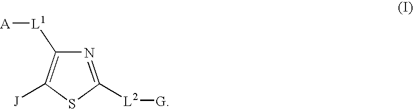

In formula (I),

L¹ more preterebly represents

a chemical bond;

—(CH₂)ₐ— in which a is 1, 2, or 3; or

—OCH₂—;

L² more preferably represents

a chemical bond;

—(CH₂)ₐ—; or

—N(R¹)— in which R¹ represents H or C₁₋₄ alkyl; and

J more preferably represents H.

Furthermore, in this more preferred embodiment

1) when L¹ is a chemical bond, A represents

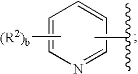

in which

b is 0, 1, or 2; and

R² is selected from

C₁₋₆ alkyl;

C₁₋₄ haloalkyl;

C₃₋₆ cycloalkyl; and

phenyl optionally substituted by halogen.

When L¹ is a bond, A may also be

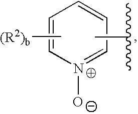

provided that G is other than a pyridyl or an N-oxide-containing group.

When L¹ is a bond, A may also be

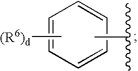

in which

d is 0, 1,or 2; and

R⁶ is selected from

C₁₋₆ alkyl;

C₁₋₄ haloalkyl;

OR⁷; in which

R⁷ represents C₁₋₄ alkyl or C₁₋₄ haloalkyl;

halogen;

NO₂;

CN;

CO₂R¹; and

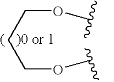

in which the O atoms are bonded to the phenyl ring at adjacent carbons.

When L¹ is a bond, A may also be

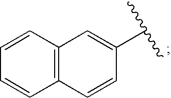

C₃₋₈ cycloalkyl;

C₅₋₆ cycloalkenyl;

adamantyl; or

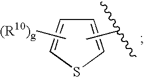

in which

g is 0, 1, or 2; and

R¹⁰ represents CN, NO₂, or halogen.

Furthermore,

2) when L² is a bond, G more preferably represents

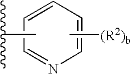

wherein

R² is selected from

C₁₋₆ alkyl;

C₁₋₄ haloalkyl;

C₃₋₆ cycloalkyl; and

phenyl optionally substituted by halogen;

or

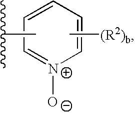

provided that A is other than a pyridyl or an N-oxide-containing group;

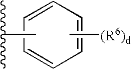

in which

R⁶ is selected from

C₁₋₆ alkyl;

C₁₋₄ haloalkyl;

OR⁷; in which

R⁷ represents C₁₋₄ alkyl or C₁₋₄ haloalkyl;

halogen;

NO₂;

CN;

CO₂R¹; and

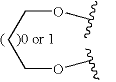

in which the O atoms are bonded to the phenyl ring at adjacent carbons;

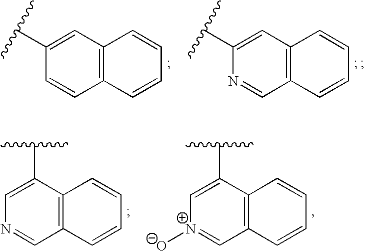

provided that A is other than a pyridyl or an N-oxide-containing group; or

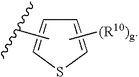

Furthermore,

11) one of A and G is a 3-pyridyl moiety of formula (II) or (IIA), or a 4-isoquinolyl moiety of formula (IIB) or (IIC)

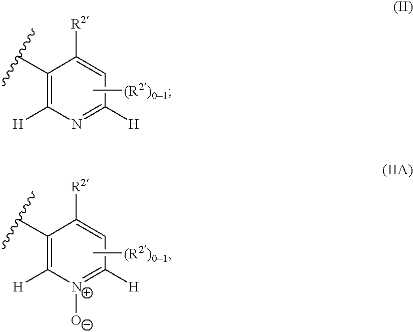

provided that the other of A and G is other than a pyridyl or an N-oxide-containing group;

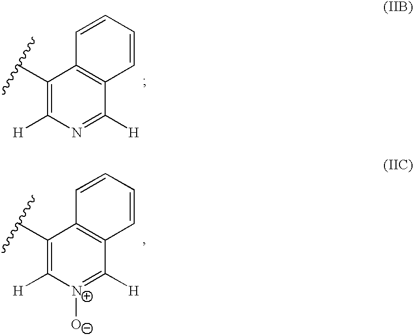

provided that the other of A and G is other than a pyridyl or an N-oxide-containing group;

and this 3-pyridyl or 4-isoquinolyl moiety is joined to the thiazole ring via a chemical bond L¹ or L² respectively; and the other of A and G is as defined above.

In addition, when the other of A and G is joined to the thiazole ring via linker L¹ or L² respectively where L¹ or L² is not a chemical bond, then R²′ of formulae (II) and (IIA) is R²; but when each of A and G is joined to the thiazole ring via a chemical bond L¹ and L² respectively, then R²′ of formulae (II) and (IIA) is selected from the group consisting of

C₂₋₆ alkyl, provided that when said 3-pyridyl moiety of formula (II) constitutes A, then G is other than phenyl substituted with an amide or sulfonamide group; and when said 3-pyridyl moiety of formula (II) constitutes G, then A is other than phenyl substituted with F;

C₃₋₆ cycloalkyl; and

phenyl optionally substituted by halogen.

In a most preferred embodiment the compounds of the invention have the formula (I)

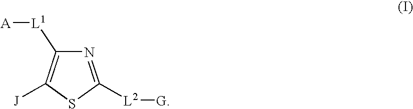

In formula (I),

L¹ most preferebly represents a chemical bond;

L² most preferably represents a chemical bond; and

J most preferably represents H.

Furthermore, in this most preferred embodiment

1) when L¹ is a chemical bond, A represents

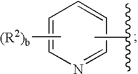

in which

b is 0, 1, or 2; and

R² is selected from

C₁₋₆ alkyl; and

phenyl optionally substituted by halogen.

When L¹ is a bond, A may also be

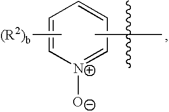

provided that G is other than a pyridyl or an N-oxide-containing group.

When L¹ is a bond, A may also be

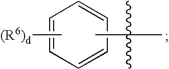

in which

d is 0, 1, or 2; and

R⁶ is selected from

C₁₋₆ alkyl;

C₁₋₄ haloalkyl;

OR⁷; in which

R⁷ represents C₁₋₄ alkyl or C₁₋₄ haloalkyl;

halogen;

NO₂ ; and

CN;

or

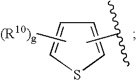

in which

g is 0, 1, or 2; and

R¹⁰ represents CN, NO₂, or halogen.

Furthermore,

2) G most preferably represents

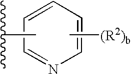

wherein

R² is selected from

C₁₋₆ alkyl;

C₃₋₆ cycloalkyl; and

phenyl optionally substituted by halogen;

or

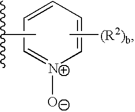

provided that A is other than a pyridyl or an N-oxide-containing group;

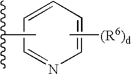

in which

R⁶ is selected from

C₁₋₆ alkyl;

C₁₋₄ haloalkyl;

OR⁷ ; in which

R⁷ represents C₁₋₄ alkyl or C₁₋₄ haloalkyl;

halogen;

NO₂;

CN;

or

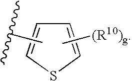

Furthermore,

11) one of A and G is a 3-pyridyl moiety of formula (II) or (IIA), or a 4-isoquinolyl moiety of formula (IIB) or (IIC)

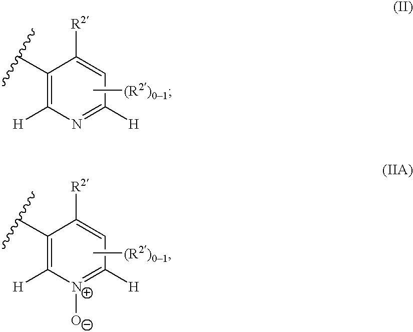

provided that the other of A and G is other than a pyridyl or an N-oxide-containing group;

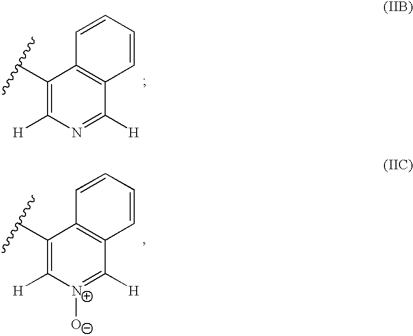

provided that the other of A and G is other than a pyridyl or an N-oxide-containing group;

and this 3-pyridyl or 4-isoquinolyl moiety is joined to the thiazole ring via a chemical bond L¹ or L² respectively, and the other of A and G is as defined above.

In addition, when the other of A and G is joined to the thiazole ring via linker L¹ or L² respectively where L¹ or L² is not a chemical bond, then R²′ of formulae (II) and (IIA) is R²; but when each of A and G is joined to the thiazole ring via a chemical bond L¹ and L² respectively, then R²′ of formulae (II) and (IIA) is selected from the group consisting of

C₂₋₆ alkyl, provided that when said 3-pyridyl moiety of formula (II) constitutes A, then G is other than phenyl substituted with an amide or sulfonamide group; and when said 3-pyridyl moiety of formula (II) constitutes G, then A is other than phenyl substituted with F; and

C₃₋₆ cycloalkyl.

### Definitions

For convenience, certain terms employed in the specification, examples, and appended claims are collected here.

The term “agonist” of an enzyme refers to a compound that binds to the enzyme and stimulates the action of the naturally occurring enzyme, or a compound which mimics the activity of the naturally occurring enzyme.

The term “antagonist” of an enzyme refers to a compound that binds to the enzyme and inhibits the action of the naturally occurring enzyme.

The term “analog” of a compound refers to a compound having a some structural similarity to a particular compound and having essentially the same type of biological activity as the compound.

The term “CYP17 substrate” includes any of the various steroid hormones acted upon by a CYP17 or a CYP17-like P₄₅₀ enzyme. Examples include pregnenolone, progesterone and their 17α-hydroxylated forms. Pregnenolone is converted to DHEA via a CYP17 C17,20-lyase reaction, but is also subject to C17a-hydroxylation via the C17,20-lyase activity. Progesterone is converted to delta 4- androstenedione via a CYP17 C17,20-lyase reaction, but is also subject to C17 alpha-hydroxylation via the C17-hydroxylase activity to form 17-hydroxyl-progesterone, a precursor to hydrocortisone (i.e. cortisol).

The term “CYP 17 metabolite” refers to any of the steroid hormones that are synthesized from a cholesterol precursor via a CYP17-mediated reaction, such as a C17-hydroxylase reaction or a C17,20-lyase reaction. Examples of CYP17 metabolites include the androgens, such as testosterone, which are synthesized via a CYP17 C17,20-lyase reaction from CYP17 substrate precursors such as pregnenolone (converted to DHEA by the CYP17 C17,20-lyase activity), and progesterone (converted to delta 4 androstenedione by the CYP17 Cl7,20-lyase activity). Progestagens such as progesterone are primarily synthesized in the corpus luteum. The androgens are responsible for, among other things, development of male secondary sex characteristics and are primarily synthesized in the testis. Other examples include the estrogens, which are also synthesized from a cholesterol precursor via a CYP17-mediated reaction. The estrogens are responsible for, among other things, the development of female secondary sex characteristics and they also participate in the ovarian cycle and are primarily synthesized in the ovary. Another group of CYP17 metabolites are the glucocorticoids, such as hydrocortisone (i.e. cortisol), which is synthesized from progesterone via a CYP17-mediated reaction. The glucocorticoids, among other functions, promote gluconeogenesis and the formation of glycogen and also enhance the degradation of fat. The glucocorticoids are primarily synthesized in the adrenal cortex.

The term “CYP 17 metabolite” is further meant to include other steroid hormones which, although not necessarily synthesized by a CYP17-mediated reaction, may nonetheless be understood by the skilled artisan to be readily affected by an alteration in a CYP17-mediated activity. For example, the mineralocorticoids, such as aldosterone, are derived from cholesterol via a progesterone intermediate. Since progesterone is also converted to the glucocorticoids and sex steroids via CYP 17-mediated reactions, an alteration of a CYP17 activity can alter the amount of progesterone available for conversion to aldosterone. For example, inhibition of CYP17 activity can increase the amount of progesterone available for conversion into aldosterone. Therefore, inhibition of CYP17 can lead to an increase in the level of aldosterone. The mineralocorticoids function, among other things, to increase reabsorption of sodium ions, chloride ions, and bicarbonate ions by the kidney, which leads to an increase in blood volume and blood pressure. The mineralocorticoids are primarily synthesized in the adrenal cortex.

The term “CYP17 metabolite-associated disease or disorder” refers to a disease or disorder which may be treated by alteration of the level of one or more CYP17 metabolites. Examples include a hormone dependent cancer, such as an androgen-dependent prostate cancer, which may be treated by inhibiting CYP17-mediated androgen synthesis, and an estrogen-dependent breast cancer or ovarian cancer, which may be treated by inhibiting CYP17-mediated estrogen synthesis. Other examples of “CYP17 metabolite-associated diseases or disorders” are Cushing's disease, hypertension, prostatic hyperplasia, and glucocorticoid deficiency. Patients with Cushing's syndrome are relatively insensitive to glucocorticoid feedback and exhibit an oversecretion of cortisol devoid of a circadian cycle (see e.g. Newell-Price & Grossman (2001) Ann. Endocrinol. 62:173-9). Another CYP17 metabolite-associated disease or disorder is hypertension. Mineralocorticoid excess causes hypertension by facilitating the sodium retention at renal tubules.

The term “derivative” of a compound refers to another compound which can be derived, e.g., by chemical synthesis, from the original compound. Thus a derivative of a compound has certain structural similarities with the original compound.

“Disease associated with an abnormal activity or level of a lyase” refers to diseases in which an abnormal activity or protein level of a lyase is present in certain cells, and in which the abnormal activity or protein level of the lyase is at least partly responsible for the disease.

A “disease associated with a lyase” refers to a disease that can be treated with a lyase inhibitor, such as the compounds disclosed herein.

A “lyase” refers to an enzyme having a lyase activity.

“Lyase activity” refers to the activity of an enzyme to catalyze the cleavage of the bond C17-C20 in 17α-hydroxy-pregnenolone and 17α-hydroxy-progesterone to form dehydroepiandrosterone (DHEA) and delta4-androstenedione, respectively. Lyase activity also refers to the cleavage of a similar bond in related compounds.

A “lyase inhibitor” is a compound which inhibits at least part of the activity of a lyase in a cell. The inhibition can be at least about 20%, preferably at least about 40%, even more preferably at least about 50%, 70%, 80%, 90%, 95%, and most preferably at least about 98% of the activity of the lyase.

A “patient” or “subject” to be treated by the subject method can mean either a human or non-human animal.

“Treating” a disease refers to preventing, curing or improving at least one symptom of a disease.

The following definitions pertain to the chemical structure of compounds:

The term “heteroatom” as used herein means an atom of nitrogen, oxygen, or sulfur.

The term “alkyl” refers to the radicals of saturated aliphatic groups, including straight-chain alkyl groups and branched-chain alkyl groups.

The term “cycloalkyl” (alicyclic) refers to radicals of cycloalkyl compounds, examples being cyclopropyl, cyclobutyl, cyclopentyl, cyclohexyl, etc.

The term “aralkyl”, as used herein, refers to an alkyl group substituted with an aryl group (e.g., an aromatic or heteroaromatic group).

The terms “alkenyl” and “alkynyl” refer to unsaturated aliphatic groups that contain at least one double or triple bond respectively.

Unless the number of carbons is otherwise specified, “lower alkyl” as used herein means an alkyl group but having from one to six carbons, preferably from one to four carbon atoms in its backbone structure. Likewise, “lower alkenyl” and “lower alkynyl” have similar chain lengths. Preferred alkyl groups are lower alkyls.

The term “aryl” as used herein means an aromatic group of 6 to 14 carbon atoms in the ring(s), for example, phenyl and naphthyl. As indicated, the term “aryl” includes polycyclic ring systems having two or more rings in which two or more carbons are common to two adjoining rings (the rings are “fused rings”) wherein at least one of the rings is aromatic.

The term “heteroaryl” as used herein means an aromatic group which contains at least one heteroatom in at least one ring. Typical examples include 5-, 6- and 7-membered single-ring aromatic groups that may include from one to four heteroatoms. Examples include pyrrole, furan, thiophene, imidazole, oxazole, thiazole, triazole, tetrazole, pyrazole, pyridine, pyrazine, pyridazine and pyrimidine, and the like. These aryl groups may also be referred to as “aryl heterocycles” or “heteroaromatics.”

The terms orthzo, meta and para apply to 1,2-, 1,3- and 1,4-disubstituted benzenes, respectively. For example, the names 1,2-dimethylbenzene and ortho-dimethylbenzene are synonymous.

The terms “alkoxyl” or “alkoxy” as used herein refer to moiety in which an alkyl group is bonded to an oxygen atom, which is in turn bonded to the rest of the molecule. Examples are methoxy, ethoxy, propyloxy, tert-butoxy, etc.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry;(i.e., J. Org. Chem. 2002, 67(1), 24A. The abbreviations contained in said list, and all abbreviations utilized by organic chemists of ordinary skill in the art are hereby incorporated by reference.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 3^{rd} ed.; Wiley: New York, 1999).

### Abbreviations and Acronyms

When the following abbreviations are used throughout the disclosure, they have the follow meaning:

| | |
|---|---|
| A | angstroms |
| AcOH | acetic acid |
| amu | atomic mass units |
| Anal. Calcd | analysis calculated |
| Ar | argon |
| BSA | bovine serum albumin |
| n-BuLi | butyllithium |
| CDCl₃ | chloroform-d |
| CD₃OD | methanol-d₄ |
| CHCl₃ | chloroform |
| CH₂Cl₂ | methylene chloride |
| CH₃CN | acetonitrile |
| CI | chemical ionization (in mass spectrometry) |
| CuI | copper iodide |
| Cs₂CO₃ | cesium carbonate |
| CPM | counts per minute |
| DMF | dimethylformamide |
| DMSO | dimethylsulfoxide |
| DMSO-d₆ | dimethylsulfoxide-d₆ |
| EDCI | |
| EI | electron impact (in mass spectrometry) |
| EPA | Environmental Protection Agency (as in EPA vial) |
| ES | electrospray ionization (in mass spectrometry) |
| Et₃N | triethylamine |
| EtOAc | ethyl acetate |
| Et₂O | diethyl ether |
| EtOH | ethanol |
| ETPB | ethyltriphenylphosphonium bromide |
| g | gram |
| GCEI | gas chromatography - electron impact mass spectrometry |
| GCMS | gas chromatography/mass spectrometry |
| h | hour(s) |
| H₂ | hydrogen gas |
| HBr | hydrogen bromide |
| HCl | hydrochloric acid |
| ¹H NMR | proton nuclear magnetic resonance |
| HEPES | 4-(2-Hydroxyethyl)piperazine-1-ethanesulfonic acid |
| HOAc | acetic acid |
| HOAt | n-hydroxyazatriazole |
| HPLC | high performance liquid chromatography |
| H₂S | hydrogen sulfide |
| Hz | hertz |
| KHMDS | potassium bis(trimethylsilyl)amide |
| KOH | potassium hydroxide |
| L | liter(s) |
| LCMS | liquid chromatography/mass spectroscopy |
| LDA | lithium diisopropyl amide |
| M | molar |
| MCPBA | m-chloroperbenzoic acid |
| MeCN | acetonitrile |
| MeOH | methanol |
| min | minute |
| μg | microgram |
| mg | milligram |
| MgSO₄ | magnesium sulfate |
| mL | microliter |
| μm | micrometer |
| μM | micromolar |
| mm | millimeter |
| mmol | millimol |
| mL | milliliter |
| mm | millimeter |
| mol | mole |
| mp | melting point |
| MS | mass spectrometry |
| m/z | mass to charge ratio |
| MTBE | methyl tert-butyl ether |
| N | normal |
| NaHCO₃ | sodium bicarbonate |
| NaHMDS | sodium bis(trimethylsilyl)amide |
| NaOH | sodium hydroxide |
| Na₂SO₄ | sodium sulfate |
| NCS | n-chlorosuccinimide |
| NH₄Cl | ammonium chloride |
| NH₄OH | ammonium hydroxide |
| NMR | nuclear magnetic resonance |
| nM | nanomolar |
| PCC | pyridinium chlorochromate |
| Pd/C | palladium on carbon |
| POCl₃ | Phosphorous oxychloride |
| P₂O₅ | phosphorous pentoxide |
| psi | pounds per square inch |
| Rf | TLC retention factor |
| rt | room temperature |
| SPA | Scintillation Proximity Assay |
| THF | tetrahydrofuran |
| TFA | trifluoroacetic acid |
| TMS | tetramethylsilane |
| TLC | thin layer chromatography |
| t_{R} | HPLC retention time |

### Compounds of the Invention

The present invention is directed to compounds which inhibit 17α-hydroxylase-C17,20-lyase.

Exemplary compounds of the invention are set forth in Table 1 below. The exemplary compounds of Table 1 are producible from known compounds (or from starting materials which, in turn, are producible from known compounds), through the general preparative methods described in the Examples. The compounds are grouped in the Tables according to the method used for their synthesis, as described in the Examples.

TABLE 1
Exemplary Compounds of the Invention

| Example# | Compound Name |
|---|---|
| 1 | 2-(2-(3-pyridyl)-1,3-thiazol-4-yl)phenyl-benzoate |
| 2 | 6-(2-(3-pyridyl)-1,3-thiazol-4-yl)benzo[b]morpholin-3-one |
| 3 | 3-(2-(3-pyridyl)-1,3-thiazol-4-yl)phenyl benzoate |
| 4 | 5-methyl-4-phenyl-2-(3-pyridyl)-1,3-thiazole |
| 5 | 4-[(4-chlorophenyl)methyl]-2-(3-pyridyl)-1,3-thiazole |
| 6 | 2-(4-methyl(3-pyridyl))-4-phenyl-1,3-thiazole |
| 7 | 4-(4-chlorophenyl)-2-(4-methyl(3-pyridyl))-1,3-thiazole |
| 8 | 4-methoxy-1-[2-(4-methyl(3-pyridyl))(1,3-thiazol-4-yl)]benzene |
| 9 | 4-[(3,4-dichlorophenyl)methyl]-2-(3-pyridyl)-1,3-thiazole |
| 10 | 4-[(4-methylphenyl)methyl]-2-(3-pyridyl)-1,3-thiazole |
| 11 | 4-[(3-methylphenyl)methyl]-2-(3-pyridyl)-1,3-thiazole |
| 12 | 4-[(3-chlorophenyl)methyl]-2-(3-pyridyl)-1,3-thiazole |
| 13 | 4-[(3-nitrophenyl)methyl]-2-(3-pyridyl)-1,3-thiazole |
| 14 | 4-[(4-bromophenyl)methyl]-2-(3-pyridyl)-1,3-thiazole |
| 15 | 4-[(4-fluorophenyl)methyl]-2-(3-pyridyl)-1,3-thiazole |
| 16 | 4-(2,4-dichlorophenyl)-2-(4-methyl(3-pyridyl))-1,3-thiazole |
| 17 | 4-(4-chlorophenyl)-5-methyl-2-(3-pyridyl)-1,3-thiazole |
| 18 | 4-(4-chlorophenyl)-5-methyl-2-(4-methyl(3-pyridyl))-1,3-thiazole |
| 19 | 4-adamantanyl-2-(3-pyridyl)-1,3-thiazole |
| 20 | 4-(tert-butyl)-2-(3-pyridyl)-1,3-thiazole |
| 21 | 4-cyclobutyl-2-(3-pyridyl)-1,3-thiazole |
| 22 | 4-cyclopentyl-2-(3-pyridyl)-1,3-thiazole |
| 23 | (5-methyl-2-(3-pyridyl)(1,3-thiazol-4-yl))phenylamine |
| 24 | 3-methoxy-1-[2-(4-methyl(3-pyridyl))(1,3-thiazol-4-yl)]benzene |
| 25 | 4-(4-fluorophenyl)-2-(4-methyl(3-pyridyl))-1,3-thiazole |
| 26 | 2-(4-methyl(3-pyridyl))-4-(3-nitrophenyl)-1,3-thiazole |
| 27 | 2-(4-methyl(3-pyridyl))-4-(2-nitrophenyl)-1,3-thiazole |
| 28 | 4-(3,4-difluorophenyl)-2-(4-methyl(3-pyridyl))-1,3-thiazole |
| 29 | 4-(5-chloro(2-thienyl))-2-(4-methyl(3-pyridyl))-1,3-thiazole |
| 30 | ethyl 3-methyl-3-(2-(3-pyridyl)(1,3-thiazol-4-yl))butanoate |
| 31 | 2-(4-methyl(3-pyridyl))-4-(2-naphthyl)-1,3-thiazole |
| 32 | 4-[(4-chlorophenyl)methyl]-2-(4-methyl(3-pyridyl))-1,3-thiazole |
| 33 | 2-(4-methyl(3-pyridyl))-4-[(4-methylphenyl)methyl]-1,3-thiazole |
| 34 | 4-(4-chlorophenyl)-2-(4-methyl(3-pyridyl))-1,3-thiazole, hydrogen |
| | chloride |
| 35 | 2-[4-(methylethyl)(3-pyridyl)]-4-phenyl-1,3-thiazole |
| 36 | 4-(4-chlorophenyl)-2-[4-(methylethyl)(3-pyridyl)]-1,3-thiazole |
| 37 | 4-methoxy-1-{2-[4-(methylethyl)(3-pyridyl)](1,3-thiazol-4-yl)}benzene |
| 38 | 4-(4-chlorophenyl)-2-(4-cyclopropyl(3-pyridyl))-1,3-thiazole |
| 39 | 1-[2-(4-cyclopropyl(3-pyridyl))(1,3-thiazol-4-yl)]-4-methoxybenzene |
| 40 | 4-(2,4-dichlorophenyl)-2-[4-(methylethyl)(3-pyridyl)]-1,3-thiazole |
| 41 | diethyl(2-(3-pyridyl)(1,3-thiazol-4-yl))amine |
| 42 | 4-cyclohexyl-2-(4-methyl(3-pyridyl))-1,3-thiazole |
| 43 | 4-adamantanyl-2-(4-methyl(3-pyridyl))-1,3-thiazole |
| 44 | 4-(tert-butyl)-2-(4-methyl(3-pyridyl))-1,3-thiazole |
| 45 | 3-[4-(4-chlorophenyl)-1,3-thiazol-2-yl]pyridin-4-ol |
| 46 | 4-(2,4-dichlorophenyl)-2-(4-methyl(3-pyridyl))-1,3-thiazole, hydrogen |
| | chloride |
| 47 | 4-(4-chlorophenyl)-2-(4-phenyl(3-pyridyl))-1,3-thiazole |
| 48 | 4-phenyl-2-(4-phenyl(3-pyridyl))-1,3-thiazole |
| 49 | 4-(4-chlorophenyl)-2-(4-methyl(3-pyridyl))-1,3-thiazole, bromide |
| 50 | 2-(4-methyl(3-pyridyl))-4-(3-pyridyl)-1,3-thiazole |
| 51 | 4-methoxy-1-[2-(4-phenyl(3-pyridyl))(1,3-thiazol-4-yl)]benzene |
| 52 | 4-(2,4-dichlorophenyl)-2-(4-phenyl(3-pyridyl))-1,3-thiazole |
| 53 | 4-(4-chlorophenyl)-5-methyl-2-[4-(methylethyl)(3-pyridyl)]-1,3-thiazole |
| 54 | cyclohexylmethyl[2-(4-methyl(3-pyridyl))(1,3-thiazol-4-yl)]amine |
| 55 | 4-cyclopent-1-enyl-2-(3-pyridyl)-1,3-thiazole |
| 56 | 4-cyclopent-1-enyl-2-(4-methyl(3-pyridyl))-1,3-thiazole |
| 57 | 4-cyclohexyl-2-[4-(methylethyl)(3-pyridyl)]-1,3-thiazole |
| 58 | 4-adamantanyl-2-[4-(methylethyl)(3-pyridyl)]-1,3-thiazole |
| 59 | 4-(tert-butyl)-2-[4-(methylethyl)(3-pyridyl)]-1,3-thiazole |
| 60 | 4-cycloheptyl-2-(4-methyl(3-pyridyl))-1,3-thiazole |
| 61 | 2-(3-chloro-4-fluorophenyl)-4-(3-pyridyl)-1,3-thiazole |
| 62 | 4-(3-pyridyl)-2-(2-thienyl)-1,3-thiazole |
| 63 | 1,3-dimethoxy-2-{2-[4-(methylethyl)(3-pyridyl)](1,3-thiazol-4- |
| | yl)}benzene |
| 64 | 4-(4-fluorophenyl)-2-[4-(methylethyl)(3-pyridyl)]-1,3-thiazole |
| 65 | 2-[4-(methylethyl)(3-pyridyl)]-4-(4-methylphenyl)-1,3-thiazole |
| 66 | 4-[(4-chlorophenyl)methyl]-2-(4-methyl(3-pyridyl))-1,3-thiazole, |
| | hydrogen chloride |
| 67 | 4,5-dimethyl-2-(4-methyl(3-pyridyl))-1,3-thiazole |
| 68 | 4-ethyl-2-(4-methyl(3-pyridyl))-1,3-thiazole |
| 69 | 4-ethoxy-2-(4-methyl(3-pyridyl))-1,3-thiazole |
| 70 | 2-(4-methyl(3-pyridyl))-4-(methylethoxy)-1,3-thiazole |
| 71 | (3,5-dichlorophenyl)[2-(4-methyl(3-pyridyl))(1,3-thiazol-4-yl)]amine |
| 72 | 2-[4-(methylethyl)(3-pyridyl)]-4-(4-nitrophenyl)-1,3-thiazole |
| 73 | 4-(3,4-dichlorophenyl)-2-[4-(methylethyl)(3-pyridyl)]-1,3-thiazole |
| 74 | 4-(4-chloro-3-nitrophenyl)-2-[4-(methylethyl)(3-pyridyl)]-1,3-thiazole |
| 75 | 2-methoxy-1-{2-[4-(methylethyl)(3-pyridyl)](1,3-thiazol-4-yl)}benzene |
| 76 | l,4-dimethoxy-2-.{2-[4-(methylethyl)(3-pyridyl)](1,3-thiazol-4- |
| | yl)}benzene |
| 77 | 4-(3-bromophenyl)-2-[4-(methylethyl)(3-pyridyl)]-1,3-thiazole |
| 78 | 4-(4-bromophenyl)-5-methyl-2-[4-(methylethyl)(3-pyridyl)]-1,3-thiazole |
| 79 | 4-(2,4-dimethylphenyl)-2-[4-(methylethyl)(3-pyridyl)]-1,3-thiazole |
| 80 | 4-(3-fluorophenyl)-2-[4-(methylethyl)(3-pyridyl)]-1,3-thiazole |
| 81 | 4-(3,4-difluorophenyl)-2-[4-(methylethyl)(3-pyridyl)]-1,3-thiazole |
| 82 | 4-(3-chlorophenyl)-2-[4-(methylethyl)(3-pyridyl)]-1,3-thiazole |
| 83 | 4-(2-chlorophenyl)-2-[4-(methylethyl)(3-pyridyl)]-1,3-thiazole |
| 84 | 2-[4-(methylethyl)(3-pyridyl)]-4-(2-naphthyl)-1,3-thiazole |
| 85 | 2-(4-methyl(3-pyridyl))-4-(2-pyridyl)-1,3-thiazole |
| 86 | 2,4-di(3-pyridyl)-1,3-thiazole |
| 87 | 6-[2-(4-methyl-3-pyridyl)-1,3-thiazol-4-yl]-1,3,4-trihydroquinolin-2-one |
| 88 | ethyl 2-(4-methyl-3-pyridyl)-1,3-thiazole-4-carboxylate |
| 89 | 4-(4-bromophenyl)-2-[4-(methylethyl)(3-pyridyl)]-1,3-thiazole |
| 90 | 2-[4-(methylethyl)(3-pyridyl)]-4-(3-nitrophenyl)-1,3-thiazole |
| 91 | 2-[4-(methylethyl)(3-pyridyl)]-4-(2-nitrophenyl)-1,3-thiazole |
| 92 | 2-(4-cyclopropyl(3-pyridyl))-4-(4-fluorophenyl)-1,3-thiazole |
| 93 | 4-(4-fluorophenyl)-2-(4-phenyl(3-pyridyl))-1,3-thiazole |
| 94 | 2-(4-methyl(3-pyridyl))-4-(4-phenylphenyl)-1,3-thiazole |
| 95 | 4-(4-bromophenyl)-2-(4-methyl(3-pyridyl))-1,3-thiazole |
| 96 | 4-[3,5-bis(trifluoromethyl)phenyl]-2-(4-methyl(3-pyridyl))-1,3-thiazole |
| 97 | 4-(4a,9b-dihydrobenzo[b]benzo[1,2-d]furan-8-yl)-2-(4-methyl(3- |
| | pyridyl))-1,3-thiazole |
| 98 | 4-cycloheptyl-2-[4-(methylethyl)(3-pyridyl)]-1,3-thiazole |
| 99 | 2-[4-(methylethyl)(3-pyridyl)]-4-(4-phenylphenyl)-1,3-thiazole |
| 100 | 4-{2-[4-(methylethyl)-3-pyridyl]-1,3-thiazol-4-yl}benzenecarbonitrile |
| 101 | 3-{2-[4-(methylethyl)-3-pyridyl]-1,3-thiazol-4-yl}benzenecarbonitrile |
| 102 | trifluoro(4-{2-[4-(methylethyl)(3-pyridyl)](1,3-thiazol-4- |
| | yl)}phenoxy)methane |
| 103 | difluoro(4-{2-[4-(methylethyl)(3-pyridyl)](1,3-thiazol-4- |
| | yl)}phenoxy)methane |
| 104 | 4-(2-fluorophenyl)-2-[4-(methylethyl)(3-pyridyl)]-1,3-thiazole |
| 105 | 2-[4-(methylethyl)(3-pyridyl)]-4-(4-pyrrolidinylphenyl)-1,3-thiazole |
| 106 | 3-{2-[4-(methylethyl)-3-pyridyl]-1,3-thiazol-4-yl}phenyl benzoate |
| 107 | 4-[(4-chlorophenyl)methyl]-2-[4-(methylethyl)(3-pyridyl)]-1,3-thiazole |
| 108 | 4-cyclopentyloxy-2-(3-pyridyl)-1,3-thiazole |
| 109 | 4-(methylethoxy)-2-[4-(methylethyl)(3-pyridyl)]-1,3-thiazole |
| 110 | (3,5-dichlorophenyl){2-[4-(methylethyl)(3-pyridyl)](1,3-thiazol-4- |
| | yl)}amine |
| 111 | 4-(3-chloro(2-thienyl))-2-(4-methyl(3-pyridyl))-1,3-thiazole, 2,2,2- |
| | trifluoroacetic acid |
| 112 | 4-(3-bromo(2-thienyl))-2-(4-methyl(3-pyridyl))-1,3-thiazole, 2,2,2- |
| | trifluoroacetic acid |
| 113 | 2-(4-methyl(3-pyridyl))-4-(4-nitrophenyl)-1,3-thiazole, 2,2,2- |
| | trifluoroacetic acid |
| 114 | 2-methoxy-1-[2-(4-methyl(3-pyridyl))(1,3-thiazol-4-yl)]benzene, 2,2,2- |
| | trifluoroacetic acid |
| 115 | 2,4-dimethoxy-1-[2-(4-methyl(3-pyridyl))(1,3-thiazol-4-yl)]benzene, |
| | 2,2,2-trifluoroacetic acid |
| 116 | 4-[2-(4-methyl-3-pyridyl)-1,3-thiazol-4-yl]benzenecarbonitrile |
| 117 | 4-cyclohexyloxy-2-(3-pyridyl)-1,3-thiazole |
| 118 | 4-cyclopent-1-enyl-2-[4-(methylethyl)(3-pyridyl)]-1,3-thiazole |
| 119 | 4-cyclopentyl-2-(4-methyl(3-pyridyl))-1,3-thiazole |
| 120 | 4-cyclopentyl-2-[4-(methylethyl)(3-pyridyl)]-1,3-thiazole |
| 121 | 4-cyclopentyloxy-2-(4-methyl(3-pyridyl))-1,3-thiazole |
| 122 | 4-cyclohexyloxy-2-(4-methyl(3-pyridyl))-1,3-thiazole |
| 123 | 4-adamantanyl-2-(5-methyl(3-pyridyl))-1,3-thiazole |
| 124 | 2-(4-cyclopentyl(3-pyridyl))-4-(4-fluorophenyl)-1,3-thiazole |
| 125 | 4-(4-chlorophenyl)-2-(4-cyclopentyl(3-pyridyl))-1,3-thiazole |
| 126 | 4-(4-bromophenyl)-2-(4-cyclopentyl(3-pyridyl))-1,3-thiazole |
| 127 | 2-(4-cyclopentyl(3-pyridyl))-4-(4-methylphenyl)-1,3-thiazole |
| 128 | 1-[2-(4-cyclopentyl(3-pyridyl))(1,3-thiazol-4-yl)]-4-methoxybenzene |
| 129 | 2-(4-cyclopentyl(3-pyridyl))-4-[4-(trifluoromethyl)phenyl]-1,3-thiazole |
| 130 | 4-(3-chlorophenyl)-2-(4-cyclopentyl(3-pyridyl))-1,3-thiazole |
| 131 | 4-(3-bromophenyl)-2-(4-cyclopentyl(3-pyridyl))-1,3-thiazole |
| 132 | 3-[2-(4-cyclopentyl-3-pyridyl)-1,3-thiazol-4-yl]benzenecarbonitrile |
| 133 | 2-(4-cyclopentyl(3-pyridyl))-4-(3-nitrophenyl)-1,3-thiazole |
| 134 | 2-(4-cyclopentyl(3-pyridyl))-4-(4-phenylphenyl)-1,3-thiazole |
| 135 | 2-(4-cyclopentyl(3-pyridyl))-4-(3-fluorophenyl)-1,3-thiazole |
| 136 | 4-(4-chloro-3-nitrophenyl)-2-(4-methyl(3-pyridyl))-1,3-thiazole |
| 137 | 4-(3-chlorophenyl)-2-(4-methyl(3-pyridyl))-1,3-thiazole |
| 138 | 3-[2-(4-methyl-3-pyridyl)-1,3-thiazol-4-yl]benzenecarbonitrile |
| 139 | 4-(2-chlorophenyl)-2-(4-methyl(3-pyridyl))-1,3-thiazole |
| 140 | 4-(3,4-dichlorophenyl)-2-(4-methyl(3-pyridyl))-1,3-thiazole |
| 141 | 4-(2-fluorophenyl)-2-(4-methyl(3-pyridyl))-1,3-thiazole |
| 142 | 4-(3-fluorophenyl)-2-(4-methyl(3-pyridyl))-1,3-thiazole |
| 143 | 4-(3-bromophenyl)-2-(4-methyl(3-pyridyl))-1,3-thiazole |
| 144 | difluoro{4-[2-(4-methyl(3-pyridyl))(1,3-thiazol-4-yl)]phenoxy}methane |
| 145 | trifluoro{4-[2-(4-methyl(3-pyridyl))(1,3-thiazol-4-yl)]phenoxy}methane |
| 146 | 2-[4-(methylethyl)(3-pyridyl)]-4-(2-pyridyl)-1,3-thiazole, 2,2,2- |
| | trifluoroacetic acid, 2,2,2-trifluoroacetic acid |
| 147 | 2-(3-pyridyl)-4-(4-pyridyl)-1,3-thiazole, 2,2,2-trifluoroacetic acid, 2,2,2- |
| | trifluoroacetic acid |
| 148 | 2-(4-methyl(3-pyridyl))-4-(4-pyridyl)-1,3-thiazole, 2,2,2-trifluoroacetic |
| | acid, 2,2,2-trifluoroacetic acid |
| 149 | 2-[4-(methylethyl)(3-pyridyl)]-4-(4-pyridyl)-1,3-thiazole, 2,2,2- |
| | trifluoroacetic acid, 2,2,2-trifluoroacetic acid |
| 150 | 2-(4-methyl(3-pyridyl))-4-(4-pyridyl)-1,3-thiazole |
| 151 | 4-cyclohexyl-2-(4-ethyl(3-pyridyl))-1,3-thiazole |
| 152 | 2-(4-ethyl(3-pyridyl))-4-phenyl-1,3-thiazole |
| 153 | 2-(4-ethyl(3-pyridyl))-4-(4-fluorophenyl)-1,3-thiazole |
| 154 | 4-(4-chlorophenyl)-2-(4-ethyl(3-pyridyl))-1,3-thiazole |
| 155 | 4-[2-(4-ethyl-3-pyridyl)-1,3-thiazol-4-yl]benzenecarbonitrile |
| 156 | 2-(4-ethyl(3-pyridyl))-4-[4-(trifluoromethyl)phenyl]-1,3-thiazole |
| 157 | 2-(4-ethyl(3-pyridyl))-4-(4-phenylphenyl)-1,3-thiazole |
| 158 | 1-[2-(4-ethyl(3-pyridyl))(1,3-thiazol-4-yl)]-4-methoxybenzene |
| 159 | {4-[2-(4-ethyl(3-pyridyl))(1,3-thiazol-4-yl)]phenoxy}difluoromethane |
| 160 | {4-[2-(4-ethyl(3-pyridyl))(1,3-thiazol-4-yl)]phenoxy}trifluoromethane |
| 161 | 2-(4-ethyl(3-pyridyl))-4-(3-fluorophenyl)-1,3-thiazole |
| 162 | 4-(3-chlorophenyl)-2-(4-ethyl(3-pyridyl))-1,3-thiazole |
| 163 | 4-(3-bromophenyl)-2-(4-ethyl(3-pyridyl))-1,3-thiazole |
| 164 | 3-[2-(4-ethyl-3-pyridyl)-1,3-thiazol-4-yl]benzenecarbonitrile |
| 165 | 2-(4-ethyl(3-pyridyl))-4-(3-nitrophenyl)-1,3-thiazole |
| 166 | 4-cyclobutoxy-2-(3-pyridyl)-1,3-thiazole |
| 167 | 4-cyclobutoxy-2-(4-methyl(3-pyridyl))-1,3-thiazole |
| 168 | 4-cycloheptyloxy-2-(4-methyl(3-pyridyl))-1,3-thiazole |
| 169 | 4-cycloheptyloxy-2-(3-pyridyl)-1,3-thiazole |
| 170 | 4-((2S)bicyclo[2.2.1]hept-2-yloxy)-2-(3-pyridyl)-1,3-thiazole |
| 171 | 4-((2S)bicyclo[2.2.1]hept-2-yloxy)-2-(4-methyl(3-pyridyl))-1,3-thiazole |
| 172 | 2-(4-methyl(3-pyridyl))-4-(phenylmethoxy)-1,3-thiazole |
| 173 | 4-(phenylmethoxy)-2-(3-pyridyl)-1,3-thiazole |
| 174 | 4-(bicyclo[2.2.1]hept-2-ylmethoxy)-2-(3-pyridyl)-1,3-thiazole |
| 175 | 2-(4-ethyl(3-pyridyl))-4-(2-fluorophenyl)-1,3-thiazole |
| 176 | 4-(2-chlorophenyl)-2-(4-ethyl(3-pyridyl))-1,3-thiazole |
| 177 | 2-(4-ethyl(3-pyridyl))-4-(2-nitrophenyl)-1,3-thiazole |
| 178 | 1-[2-(4-ethyl(3-pyridyl))(1,3-thiazol-4-yl)]-2-methoxybenzene |
| 179 | 2-(4-ethyl(3-pyridyl))-4-(2-naphthyl)-1,3-thiazole |
| 180 | 2-(4-ethyl(3-pyridyl))-5-methyl-4-phenyl-1,3-thiazole |
| 181 | 4-(4-chlorophenyl)-2-(4-ethyl(3-pyridyl))-5-methyl-1,3-thiazole |
| 182 | 4-(4-bromophenyl)-2-(4-ethyl(3-pyridyl))-5-methyl-1,3-thiazole |
| 183 | 4-(3,4-dichlorophenyl)-2-(4-ethyl(3-pyridyl))-1,3-thiazole |
| 184 | 1-[2-(4-ethyl(3-pyridyl))-5-methyl(1,3-thiazol-4-yl)]-4-methoxybenzene |
| 185 | 3-[4-(4-chlorophenyl)(1,3-thiazol-2-yl)]-4-methoxypyridine |
| 186 | 2-(4-chloro(3-pyridyl))-4-(4-chlorophenyl)-1,3-thiazole, hydrogen |
| | chloride |
| 187 | 4-(4-chloro-3-nitrophenyl)-2-(4-ethyl(3-pyridyl))-1,3-thiazole |
| 188 | 2-[2-(4-ethyl(3-pyridyl))(1,3-thiazol-4-yl)]-1,4-dimethoxybenzene |
| 189 | 4-(2,4-dichlorophenyl)-2-(4-ethyl(3-pyridyl))-1,3-thiazole |
| 190 | 2-(4-cyclopropyl(3-pyridyl))-4-(4-phenylphenyl)-1,3-thiazole |
| 191 | {4-[2-(4-cyclopropyl(3-pyridyl))(1,3-thiazol-4- |
| | yl)]phenoxy}difluoromethane |
| 192 | 2-(4-cyclopropyl(3-pyridyl))-4-[4-(trifluoromethyl)phenyl]-1,3-thiazole |
| 193 | {4-[2-(4-cyclopropyl(3-pyridyl))(1,3-thiazol-4- |
| | yl)]phenoxy}trifluoromethane |
| 194 | 4-(4-chlorophenyl)-2-(4-cyclopropyl(3-pyridyl))-5-methyl-1,3-thiazole |
| 195 | 4-(4-bromophenyl)-2-(4-cyclopropyl(3-pyridyl))-5-methyl-1,3-thiazole |
| 196 | 2-(4-cyclopropyl(3-pyridyl))-5-methyl-4-phenyl-1,3-thiazole |
| 197 | 1-[2-(4-cyclopropyl(3-pyridyl))-5-methyl(1,3-thiazol-4-yl)]-4- |
| | methoxybenzene |
| 198 | 4-[2-(4-cyclopropyl-3-pyridyl)-1,3-thiazol-4-yl]benzenecarbonitrile |
| 199 | 4-(2,4-dimethylphenyl)-2-(4-ethyl(3-pyridyl))-1,3-thiazole |
| 200 | 4-cyclohexyl-2-(4-cyclopropyl(3-pyridyl))-1,3-thiazole |
| 201 | 4-cyclohexyl-5-methyl-2-(4-methyl(3-pyridyl))-1,3-thiazole |
| 202 | 4-cyclohexyl-5-iodo-2-(4-methyl(3-pyridyl))-1,3-thiazole |
| 203 | 4-cyclohexyl-2-[4-(2-methylpropyl)(3-pyridyl)]-1,3-thiazole |
| 204 | 4-(bicyclo[2.2.1]hept-2-ylmethoxy)-2-(4-methyl(3-pyridyl))-1,3-thiazole |
| 205 | 4-(4-chlorophenyl)-5-iodo-2-(4-methyl(3-pyridyl))-1,3-thiazole |
| 206 | 4-(3,4-difluorophenyl)-5-iodo-2-(4-methyl(3-pyridyl))-1,3-thiazole |
| 207 | 4-(4-chlorophenyl)-5-ethyl-2-(4-propyl(3-pyridyl))-1,3-thiazole |
| 208 | 4-(4-chlorophenyl)-2-[4-(2-methylpropyl)(3-pyridyl)]-1,3-thiazole |
| 209 | 2-(4-butyl(3-pyridyl))-4-(4-chlorophenyl)-5-propyl-1,3-thiazole |
| 210 | 4-adamantanyl-2-(4-cyclopropyl(3-pyridyl))-1,3-thiazole |
| 211 | 4-(3-bromophenyl)-2-(4-cyclopropyl(3-pyridyl))-1,3-thiazole |
| 212 | 3-[2-(4-cyclopropyl-3-pyridyl)-1,3-thiazol-4-yl]benzenecarbonitrile |
| 213 | 2-(4-cyclopropyl(3-pyridyl))-4-(2-nitrophenyl)-1,3-thiazole |
| 214 | 4-(3,4-dichlorophenyl)-2-(4-cyclopropyl(3-pyridyl))-1,3-thiazole |
| 215 | 2-(4-cyclopropyl(3-pyridyl))-4-(2-naphthyl)-1,3-thiazole |
| 216 | 1-[2-(4-cyclopropyl(3-pyridyl))(1,3-thiazol-4-yl)]-2-methoxybenzene |
| 217 | 4-(2,4-dimethylphenyl)-2-(4-cyclopropyl(3-pyridyl))-1,3-thiazole |
| 218 | 2-[2-(4-cyclopropyl(3-pyridyl))(1,3-thiazol-4-yl)]-1,4-dimethoxybenzene |
| 219 | 4-(4-chloro-3-nitrophenyl)-2-(4-cyclopropyl(3-pyridyl))-1,3-thiazole |
| 220 | 2-(4-cyclopropyl(3-pyridyl))-4-(2-fluorophenyl)1,3-thiazole |
| 221 | 2-(4-methyl(3-pyridyl))-4-(4-nitrophenyl)-1,3-thiazole |
| 222 | 4-(5-chloro(2-thienyl))-2-(4-methyl(3-pyridyl))-1,3-thiazole, hydrogen |
| | chloride |
| 223 | 4-(3-chlorophenyl)-2-(4-cyclopropyl(3-pyridyl))-1,3-thiazole |
| 224 | 4-(2-chlorophenyl)-2-(4-cyclopropyl(3-pyridyl))-1,3-thiazole |
| 225 | 2-(4-cyclopropyl(3-pyridyl))-4-phenyl-1,3-thiazole |
| 226 | 2-(4-cyclopropyl(3-pyridyl))-4-(3-fluorophenyl)-1,3-thiazole |
| 227 | 2-(4-cyclopropyl(3-pyridyl))-4-(4-methylphenyl)-1,3-thiazole |
| 228 | 2-(4-cyclopropyl(3-pyridyl))-4-(3-nitrophenyl)-1,3-thiazole |
| 229 | 2-(4-cyclopropyl(3-pyridyl))-4-(4-nitrophenyl)-1,3-thiazole |
| 230 | 4-(4-bromophenyl)-2-(4-cyclopropyl(3-pyridyl))-1,3-thiazole |
| 231 | 2-(4-cyclopropyl(3-pyridyl))-4-(3-pyridyl)-1,3-thiazole |
| 232 | 2-(4-cyclopropyl(3-pyridyl))-4-(4-methyl(3-pyridyl))-1,3-thiazole |
| 233 | 2-(4-cyclopropyl(3-pyridyl))-4-(2-pyridyl)-1,3-thiazole |
| 234 | 2-(4-cyclopropyl(3-pyridyl))-4-(4-pyridyl)-1,3-thiazole |
| 235 | 2-(4-methyl(3-pyridyl))-4-(2-pyridyl)-1,3-thiazole, 2,2,2-trifluoroacetic |
| | acid |
| 236 | 4-(2,4-dimethylphenyl)-2-(4-methyl(3-pyridyl))-1,3-thiazole, bromide |
| 237 | 2-(4-ethyl(3-pyridyl))-4-(3-pyridyl)-1,3-thiazole |
| 238 | 2-(4-ethyl(3-pyridyl))-4-(4-methylphenyl)-1,3-thiazole |
| 239 | 4-(4-bromophenyl)-2-(4-ethyl(3-pyridyl))-1,3-thiazole |
| 240 | 2-(4-ethyl(3-pyridyl))-4-(4-nitrophenyl)-1,3-thiazole |
| 241 | 4-(2H,3H-benzo[3,4-e]1,4-dioxan-6-yl)-2-(4-ethyl(3-pyridyl))-1,3-thiazole |
| 242 | 2-(4-ethyl(3-pyridyl))-5-methyl-4-[4-(2-methylpropyl)phenyl]-1,3- |
| | thiazole |
| 243 | 4-adamantanyl-2-(4-ethyl(3-pyridyl))-1,3-thiazole |
| 244 | 4-(4-chlorophenyl)-2-(4-piperidyl(3-pyridyl))-1,3-thiazole |
| 245 | 4-(4-chlorophenyl)-2-(4-propyl(3-pyridyl))-1,3-thiazole |
| 246 | 2-(4-butyl(3-pyridyl))-4-(4-chlorophenyl)-1,3-thiazole |
| 247 | 4-(4-chlorophenyl)-5-ethyl-2-(4-methyl(3-pyridyl))-1,3-thiazole |
| 248 | 4-(4-chlorophenyl)-2-(4-methyl(3-pyridyl))-5-propyl-1,3-thiazole |
| 249 | 2-[4-(methylethyl)(3-pyridyl)]-4-(3-pyridyl)-1,3-thiazole |
| 250 | [4-(4-methyl(3-pyridyl))(1,3-thiazol-2-yl)](2-morpholin-4-ylethyl)amine, |
| | 2,2,2-trifluoroacetic acid, 2,2,2-trifluoroacetic acid |
| 251 | 4-(4-chlorophenyl)-2-(4-piperazinyl(3-pyridyl))-1,3-thiazole |
| 252 | {3-[4-(4-chlorophenyl)(1,3-thiazol-2-yl)](4-pyridyl)}cyclobutylamine |
| 253 | {3-[4-(4-chlorophenyl)(1,3-thiazol-2-yl)](4-pyridyl)}propylamine |
| 254 | {3-[4-(4-chlorophenyl)(1,3-thiazol-2-yl)](4- |
| | pyridyl)}(methylpropyl)amine |
| 255 | 4-(4-chlorophenyl)-2-(4-morpholin-4-yl(3-pyridyl))-1,3-thiazole |
| 256 | {3-[4-(4-chlorophenyl)(1,3-thiazol-2-yl)](4-pyridyl)}(4- |
| | fluorophenyl)amine |
| 257 | 4-{3-[4-(4-chlorophenyl)-1,3-thiazol-2-yl]-4-pyridyl}-1,4- |
| | thiazaperhydroine |
| 258 | {3-[4-(4-chlorophenyl)(1,3-thiazol-2-yl)](4-pyridyl)}phenylamine |
| 259 | 2-(4-cyclopentyl(3-pyridyl))-4-phenyl-1,3-thiazole |
| 260 | 4-(2-chlorophenyl)-2-(4-cyclopentyl(3-pyridyl))-1,3-thiazole |
| 261 | 4-(4-chlorophenyl)-2-(4-cyclopentyl(3-pyridyl))-5-methyl-1,3-thiazole |
| 262 | 4-(4-bromophenyl)-2-(4-cyclopentyl(3-pyridyl))-5-methyl-1,3-thiazole |
| 263 | 2-(4-cyclopentyl(3-pyridyl))-4-(2-nitrophenyl)-1,3-thiazole |
| 264 | 4-(2,4-dimethylphenyl)-2-(4-cyclopentyl(3-pyridyl))-1,3-thiazole |
| 265 | 2-[2-(4-cyclopentyl(3-pyridyl))(1,3-thiazol-4-yl)]-1,4-dimethoxybenzene |
| 266 | 4-(2H,3H,4H-benzo[b]1,4-dioxepan-7-yl)-2-(4-cyclopentyl(3-pyridyl))- |
| | 1,3-thiazole |
| 267 | 4-[3,5-bis(trifluoromethyl)phenyl]-2-(4-cyclopentyl(3-pyridyl))-1,3- |
| | thiazole, C |
| 268 | 4-[3,5-bis(trifluoromethyl)phenyl]-2-[4-(methylethyl)(3-pyridyl)]-1,3- |
| | thiazole, C |
| 269 | 4-(2H,3H,4H-benzo[b]1,4-dioxepin-7-yl)-2-[4-(methylethyl)(3-pyridyl)]- |
| | 1,3-thiazole |
| 270 | 2-[4-(methylethyl)(3-pyridyl)]-4-[4-(trifluoromethyl)phenyl]-1,3-thiazole |
| 271 | 1-[2-(4-cyclopentyl(3-pyridyl))(1,3-thiazol-4-yl)]-2-methoxybenzene |
| 272 | (4-[2-(4-cyclopentyl(3-pyridyl))(1,3-thiazol-4- |
| | yl)]phenoxy}difluoromethane |
| 273 | {4-[2-(4-cyclopentyl(3-pyridyl))(1,3-thiazol-4- |
| | yl)]phenoxy}trifluoromethane |
| 274 | 2-(4-cyclopentyl(3-pyridyl))-4-(4-nitrophenyl)-1,3-thiazole |
| 275 | 4-(4-chloro-3-nitrophenyl)-2-(4-cyclopentyl(3-pyridyl))-1,3-thiazole |
| 276 | 2-[4-(methylethyl)(3-pyridyl)]-4-(5-methyl-3-phenylisoxazol-4-yl)-1,3- |
| | thiazole |
| 277 | 4-methoxy-1-{5-methyl-2-[4-(methylethyl)(3-pyridyl)](1,3-thiazol-4- |
| | yl)}benzene |
| 278 | 1-[2-(4-cyclopentyl(3-pyridyl))-5-methyl(1,3-thiazol-4-yl)]-4- |
| | methoxybenzene |
| 279 | 4-(3,4-dichlorophenyl)-2-(4-cyclopentyl(3-pyridyl))-1,3-thiazole |
| 280 | 4-(2,4-dichlorophenyl)-2-(4-cyclopentyl(3-pyridyl))-1,3-thiazole |
| 281 | 2-(4-cyclopentyl(3-pyridyl))-4-(2-naphthyl)-1,3-thiazole |
| 282 | 4-(4-chlorophenyl)-2-{4-[2,2,2-trifluoro-1-(trifluoromethyl)ethyl](3- |
| | pyridyl)}-1,3-thiazole |
| 283 | [2-(4-methyl(3-pyridyl))(1,3-thiazol-4-yl)]-N-(4- |
| | methylphenyl)carboxamide |
| 284 | N-cyclohexyl[2-(4-methyl(3-pyridyl))(1,3-thiazol-4-yl)]carboxamide |
| 285 | 2-(4-methyl(3-pyridyl))(1,3-thiazol-4-yl)morpholin-4-yl ketone |
| 286 | [2-(4-methyl(3-pyridyl))(1,3-thiazol-4-yl)]-N-benzamide |
| 287 | N-(4-methoxyphenyl)[2-(4-methyl(3-pyridyl))(1,3-thiazol-4- |
| | yl)]carboxamide |
| 288 | [2-(4-methyl(3-pyridyl))(1,3-thiazol-4-yl)]-N-(4-pyridyl)carboxamide |
| 289 | N-bicyclo[2.2.1]hept-2-yl[2-(4-methyl(3-pyridyl))(1,3-thiazol-4- |
| | yl)]carboxamide |
| 290 | N-(3,4-difluorophenyl)[2-(4-methyl(3-pyridyl))(1,3-thiazol-4- |
| | yl)]carboxamide, 2,2,2-trifluoroacetic acid |
| 291 | N-(3-chloro-4-fluorophenyl)[2-(4-methyl(3-pyridyl))(1,3-thiazol-4- |
| | yl)]carboxamide, 2,2,2-trifluoroacetic acid |
| 292 | 4-(4-{[2-(4-methyl-3-pyridyl)-1,3-thiazol-4- |
| | yl]carbonyl}piperazinyl)benzenecarbonitrile, 2,2,2-trifluoroacetic acid |
| 293 | 4-(4-chlorophenyl)piperazinyl 2-(4-methyl(3-pyridyl))(1,3-thiazol-4-yl) |
| | ketone, 2,2,2-trifluoroacetic acid |
| 294 | N-(3-cyanophenyl)[2-(4-methyl(3-pyridyl))(1,3-thiazol-4- |
| | yl)]carboxamide, 2,2,2-trifluoroacetic acid |
| 295 | N-(2-furylmethyl)[2-(4-methyl(3-pyridyl))(1,3-thiazol-4- |
| | yl)]carboxamide, 2,2,2-trifluoroacetic acid |
| 296 | 2-(4-methyl(3-pyridyl))(1,3-thiazol-4-yl)piperidyl ketone |
| 297 | N-(4-fluorophenyl)[2-(4-methyl(3-pyridyl))(1,3-thiazol-4- |
| | yl)]carboxamide |
| 298 | [2-(4-methyl(3-pyridyl))(1,3-thiazol-4-yl)]-N-(3-pyridyl)carboxamide |
| 299 | N-(4-chlorophenyl)[2-(4-methyl(3-pyridyl))(1,3-thiazol-4- |
| | yl)]carboxamide |
| 300 | 4-[2-(4-phenyl-3-pyridyl)-1,3-thiazol-4-yl]benzenecarbonitrile |
| 301 | difluoro{4-[2-(4-phenyl(3-pyridyl))(1,3-thiazol-4-yl)]phenoxy}methane |
| 302 | 4-(4-nitrophenyl)-2-(4-phenyl(3-pyridyl))-1,3-thiazole |
| 303 | 4-(4-methylphenyl)-2-(4-phenyl(3-pyridyl))-1,3-thiazole |
| 304 | 2-(4-phenyl(3-pyridyl))-4-(4-pyrrolidinylphenyl)-1,3-thiazole |
| 305 | 4-(3-chlorophenyl)-2-(4-phenyl(3-pyridyl))-1,3-thiazole |
| 306 | 4-(2-chlorophenyl)-2-(4-phenyl(3-pyridyl))-1,3-thiazole |
| 307 | 3-[2-(4-phenyl-3-pyridyl)-1,3-thiazol-4-yl]benzenecarbonitrile |
| 308 | 4-(3-fluorophenyl)-2-(4-phenyl(3-pyridyl))-1,3-thiazole |
| 309 | 4-(2-fluorophenyl)-2-(4-phenyl(3-pyridyl))-1,3-thiazole |
| 310 | 4-(3-nitrophenyl)-2-(4-phenyl(3-pyridyl))-1,3-thiazole |
| 311 | 4-(2-nitrophenyl)-2-(4-phenyl(3-pyridyl))-1,3-thiazole |
| 312 | 4-(3-bromophenyl)-2-(4-phenyl(3-pyridyl))-1,3-thiazole |
| 313 | 2-(4-phenyl(3-pyridyl))-4-[4-(trifluoromethyl)phenyl]-1,3-thiazole |
| 314 | 4-[4-(methylethyl)(3-pyridyl)]-2-(4-phenyl(3-pyridyl))-1,3-thiazole |
| 315 | 2-(4-phenyl(3-pyridyl))-4-(3-pyridyl)-1,3-thiazole |
| 316 | 4-(4-methyl(3-pyridyl))-2-(4-phenyl(3-pyridyl))-1,3-thiazole |
| 317 | 2-(4-phenyl(3-pyridyl))-4-(2-pyridyl)-1,3-thiazole |
| 318 | 2-(4-phenyl(3-pyridyl))-4-(4-pyridyl)-1,3-thiazole |
| 319 | 4-{2-[4-(4-fluorophenyl)-3-pyridyl]-1,3-thiazol-4-yl}benzenecarbonitrile |
| 320 | 4-(4-fluorophenyl)-2-[4-(4-fluorophenyl)(3-pyridyl)]-1,3-thiazole |
| 321 | 4-(4-chlorophenyl)-2-[4-(4-fluorophenyl)(3-pyridyl)]-1,3-thiazole |
| 322 | 2-[4-(4-fluorophenyl)(3-pyridyl)]-4-(4-nitrophenyl)-1,3-thiazole |
| 323 | difluoro(4-{2-[4-(4-fluorophenyl)(3-pyridyl)](1,3-thiazol-4- |
| | yl)}phenoxy)methane |
| 324 | 2-[4-(4-fluorophenyl)(3-pyridyl)]-4-[4-(trifluoromethyl)phenyl]-1,3- |
| | thiazole |
| 325 | 4-(3-fluorophenyl)-2-[4-(4-fluorophenyl)(3-pyridyl)]-1,3-thiazole |
| 326 | 4-(3-chlorophenyl)-2-[4-(4-fluorophenyl)(3-pyridyl)]-1,3-thiazole |
| 327 | 3-{2-[4-(4-fluorophenyl)-3-pyridyl]-1,3-thiazol-4-yl}benzenecarbonitrile |
| 328 | 4-(3,4-dichlorophenyl)-2-[4-(4-fluorophenyl)(3-pyridyl)]-1,3-thiazole |
| 329 | 4-[2-(4-cyclopropyl-3-pyridyl)-1,3-thiazol-4-yl]benzenecarbonitrile, |
| | hydrogen chloride |
| 330 | 4-(tert-butyl)-2-(4-propyl(3-pyridyl))-1,3-thiazole |
| 331 | 2-(4-propyl(3-pyridyl))-4-[4-(trifluoromethyl)phenyl]-1,3-thiazole |
| 332 | 4-(2-naphthyl)-2-(4-propyl(3-pyridyl))-1,3-thiazole |
| 333 | 4-(3-chlorophenyl)-2-(4-propyl(3-pyridyl))-1,3-thiazole |
| 334 | 4-(2-chlorophenyl)-2-(4-propyl(3-pyridyl))-1,3-thiazole |
| 335 | 4-(4-bromophenyl)-2-(4-propyl(3-pyridyl))-1,3-thiazole |
| 336 | 4-(3-bromophenyl)-2-(4-propyl(3-pyridyl))-1,3-thiazole |
| 337 | 4-phenyl-2-(4-propyl(3-pyridyl))-1,3-thiazole |
| 338 | 4-(4-bromophenyl)-5-methyl-2-(4-propyl(3-pyridyl))-1,3-thiazole |
| 339 | trifluoro{4-[2-(4-propyl(3-pyridyl))(1,3-thiazol-4-yl)]phenoxy}methane |
| 340 | 4-(2,4-dimethylphenyl)-2-(4-propyl(3-pyridyl))-1,3-thiazole |
| 341 | 4-(4-phenylphenyl)-2-(4-propyl(3-pyridyl))-1,3-thiazole |
| 342 | 3-[2-(4-propyl-3-pyridyl)-1,3-thiazol-4-yl]benzenecarbonitrile |
| 343 | 4-methoxy-1-[2-(4-propyl(3-pyridyl))(1,3-thiazol-4-yl)]benzene |
| 344 | 4-(2-fluorophenyl)-2-(4-propyl(3-pyridyl))-1,3-thiazole |
| 345 | 4-(4-fluorophenyl)-2-(4-propyl(3-pyridyl))-1,3-thiazole |
| 346 | 4-(4-methylphenyl)-2-(4-propyl(3-pyridyl))-1,3-thiazole |
| 347 | difluoro{4-[2-(4-propyl(3-pyridyl))(1,3-thiazol-4-yl)]phenoxy}methane |
| 348 | 2-methoxy-1-[2-(4-propyl(3-pyridyl))(1,3-thiazol-4-yl)]benzene |
| 349 | 4-(3-nitrophenyl)-2-(4-propyl(3-pyridyl))-1,3-thiazole |
| 350 | 4-(2-nitrophenyl)-2-(4-propyl(3-pyridyl))-1,3-thiazole |
| 351 | 4-(5-methyl-3-phenylisoxazol-4-yl)-2-(4-propyl(3-pyridyl))-1,3-thiazole |
| 352 | 4-[2-(4-methyl-3-pyridyl)-1,3-thiazol-4-yl]benzoic acid, N, hydrogen |
| | chloride |
| 353 | 4-[2-(4-methyl-3-pyridyl)-1,3-thiazol-4-yl]benzenecarbonitrile, 4- |
| | methylbenzenesulfonic acid |
| 354 | 4-[2-(4-methyl-3-pyridyl)-1,3-thiazol-4-yl]benzenecarbonitrile, |
| | methanesulfonic acid |
| 355 | 4-[2-(4-methyl-3-pyridyl)-1,3-thiazol-4-yl]benzenecarbonitrile, |
| | (1Z)ethene-1,2-dicarboxylic acid |
| 356 | 4-[2-(4-methyl-3-pyridyl)-1,3-thiazol-4-yl]benzenecarbonitrile, hydrogen |
| | chloride |
| 357 | 4-[2-(4-methyl-3-pyridyl)-1,3-thiazol-4-yl]benzenecarboxamidine |
| 358 | 5-[2-(4-methyl-3-pyridyl)-1,3-thiazol-4-yl]thiophene-2-carbonitrile, |
| | 2,2,2-trifluoroacetic acid |
| 359 | 2-[4-(4-fluorophenyl)(3-pyridyl)]-4-(3-nitrophenyl)-1,3-thiazole |
| 360 | 4-(2,4-dichlorophenyl)-2-[4-(4-fluorophenyl)(3-pyridyl)]-1,3-thiazole |
| 361 | 3-[2-(4-propyl-3-pyridyl)-1,3-thiazol-4-yl]phenyl benzoate |
| 362 | 4-[2-(4-propyl-3-pyridyl)-1,3-thiazol-4-yl]benzenecarbonitrile |
| 363 | 4-(4-nitrophenyl)-2-(4-propyl(3-pyridyl))-1,3-thiazole |
| 364 | 4-(4-chlorophenyl)-5-(4-methylphenyl)-2-(4-propyl(3-pyridyl))-1,3- |
| | thiazole |
| 365 | 4-{2-[4-(tert-butyl)-3-pyridyl]-1,3-thiazol-4-yl}benzenecarbonitrile |
| 366 | 3-{2-[4-(tert-butyl)-3-pyridyl]-1,3-thiazol-4-yl}benzenecarbonitrile |
| 367 | 2-[4-(tert-butyl)(3-pyridyl)]-4-(4-fluorophenyl)-1,3-thiazole |
| 368 | 2-[4-(tert-butyl)(3-pyridyl)]-4-(3-fluorophenyl)-1,3-thiazole |
| 369 | 2-[4-(tert-butyl)(3-pyridyl)]-4-(4-nitrophenyl)-1,3-thiazole |
| 370 | 2-t4-(tert-butyl)(3-pyridyl)]-4-(4-chlorophenyl)-1,3-thiazole |
| 371 | 2-[4-(tert-butyl)(3-pyridyl)]-4-(2-chlorophenyl)-1,3-thiazole |
| 372 | 2-[4-(tert-butyl)(3-pyridyl)]-4-(3-chlorophenyl)-1,3-thiazole |
| 373 | 4-(2,4-dichlorophenyl)-2-[4-(tert-butyl)(3-pyridyl)]-1,3-thiazole |
| 374 | 2-[4-(tert-butyl)(3-pyridyl)]-4-(4-bromophenyl)-1,3-thiazole |
| 375 | 2-[4-(tert-butyl)(3-pyridyl)]-4-(3-bromophenyl)-1,3-thiazole |
| 376 | 2-[4-(tert-butyl)(3-pyridyl)]-4-(4-bromophenyl)-5-methyl-1,3-thiazole |
| 377 | 2-[4-(tert-butyl)(3-pyridyl)]-4-(4-methylphenyl)-1,3-thiazole |
| 378 | 2-[4-(tert-butyl)(3-pyridyl)]-4-(2,4-dimethylphenyl)-1,3-thiazole |
| 379 | 2-[4-(tert-butyl)(3-pyridyl)]-4-[4-(trifluoromethyl)phenyl]-1,3-thiazole |
| 380 | (4-{2-[4-(tert-butyl)(3-pyridyl)](1,3-thiazol-4- |
| | yl)}phenoxy)difluoromethane |
| 381 | (4-{2-[4-(tert-butyl)(3-pyridyl)](1,3-thiazol-4- |
| | yl)}phenoxy)trifluoromethane |
| 382 | 1-{2-[4-(tert-butyl)(3-pyridyl)](1,3-thiazol-4-yl)}-4-methoxybenzene |
| 383 | 1-{2-[4-(tert-butyl)(3-pyridyl)](1,3-thiazol-4-yl)}-2-methoxybenzene |
| 384 | 2-[4-(tert-butyl)(3-pyridyl)]-4-(4-pyrrolidinylphenyl)-1,3-thiazole |
| 385 | 2-[4-(tert-butyl)(3-pyridyl)]-4-phenyl-1,3-thiazole |
| 386 | 2-[4-(tert-butyl)(3-pyridyl)]-4-(4-chloro-3-nitrophenyl)-1,3-thiazole |
| 387 | 2-[4-(tert-butyl)(3-pyridyl)]-4-(4-methyl(3-pyridyl))-1,3-thiazole |
| 388 | [2-(4-methyl(3-pyridyl))(1,3-thiazol-4-yl)]-N-piperidylcarboxamide, |
| | 2,2,2-trifluoroacetic acid |
| 389 | 2-[4-(methylethyl)(3-pyridyl)]-4-(3-thienyl)-1,3-thiazole, bromide |
| 390 | 2-(4-cyclopropyl(3-pyridyl))-4-(3-thienyl)-1,3-thiazole, bromide |
| 391 | 4-(4-methyl(3-pyridyl))-2-[4-(methylethyl)(3-pyridyl)]-1,3-thiazole, |
| | 2,2,2-trifluoroacetic acid, 2,2,2-trifluoroacetic acid |
| 392 | 2-[4-(methylethyl)(3-pyridyl)]-4-(5,5,8,8-tetramethyl(2-5,6,7,8- |
| | tetrahydronaphthyl))-1,3-thiazole, 2,2,2-trifluoroacetic acid |
| 393 | 4-[3-(3,4-dichlorophenyl)isoxazol-5-yl]-2-(4-cyclopropyl(3-pyridyl))-1,3- |
| | thiazole, 2,2,2-trifluoroacetic acid |
| 394 | 2-(4-ethyl(3-pyridyl))-4-(3-thienyl)-1,3-thiazole, bromide |
| 395 | 4-(3-furyl)-2-[4-(methylethyl)(3-pyridyl)]-1,3-thiazole, 2,2,2- |
| | trifluoroacetic acid |
| 396 | 2-(5-bromo(3-pyridyl))-4-(3-fluorophenyl)-1,3-thiazole |
| 397 | 2-(5-bromo(3-pyridyl))-4-(3-chlorophenyl)-1,3-thiazole |
| 398 | 4-(2,4-dimethylphenyl)-2-(5-bromo(3-pyridyl))-1,3-thiazole |
| 399 | 2-[4-(tert-butyl)(3-pyridyl)]-4-(2-bromophenyl)-1,3-thiazole |
| 400 | 2-[4-(tert-butyl)(3-pyridyl)]-4-(2-nitrophenyl)-1,3-thiazole |
| 401 | 2-[4-(tert-butyl)(3-pyridyl)]-4-(3-nitrophenyl)-1,3-thiazole |
| 402 | 4-(3,4-dichlorophenyl)-2-[4-(tert-butyl)(3-pyridyl)]-1,3-thiazole |
| 403 | l-{2-[4-(tert-butyl)(3-pyridyl)]-5-methyl(1,3-thiazol-4-yl)}-4-methoxyhenzene |
| 404 | 2-[4-(tert-butyl)(3-pyridyl)]-5-methyl-4-[4-(2-methylpropyl)phenyl]-1,3- |
| | thiazole |
| 405 | 2-[4-(tert-butyl)(3-pyridyl)]-4-(3-chloro-4-methylphenyl)-5-methyl-1,3- |
| | thiazole |
| 406 | 4-(tert-butyl)-2-(5-bromo(3-pyridyl))-1,3-thiazole |
| 407 | 2-(5-bromo(3-pyridyl))-4-(3-pyridyl)-1,3-thiazole |
| 408 | 2-[4-(tert-butyl)(3-pyridyl)]-5-methyl-4-phenyl-1,3-thiazole |
| 409 | 4-(2-{4-[(dimethylamino)methyl]-3-pyridyl}-1,3-thiazol-4- |
| | yl)benzenecarbonitrile |
| 410 | 4-(2-{4-[(4-methylpiperazinyl)methyl]-3-pyridyl}-1,3-thiazol-4- |
| | yl)benzenecarbonitrile, 2,2,2-trifluoroacetic acid, 2,2,2-trifluoroacetic |
| | acid, 2,2,2-trifluoroacetic acid |
| 411 | 4-[2-(4-{[4-(methylethyl)piperazinyl]methyl}-3-pyridyl)-1,3-thiazol-4- |
| | yl]benzenecarbonitrile, 2,2,2-trifluoroacetic acid, 2,2,2-trifluoroacetic |
| | acid, 2,2,2-trifluoroacetic acid |
| 412 | 4-[2-(4-ethyl-3-pyridyl)-1,3-thiazol-4-yl]benzenecarbonitrile, |
| | methanesulfonic acid |
| 413 | 4-[2-(4-cyclopropyl-3-pyridyl)-1,3-thiazol-4-yl]benzenecarbonitrile, |
| | methanesulfonic acid |
| 414 | diethyl{3-[2-(4-methyl(3-pyridyl))(1,3-thiazol-4-yl)]phenyl}amine |
| 415 | 4-{2-[4-(pyrrolidinylmethyl)-3-pyridyl]-1,3-thiazol-4- |
| | yl}benzenecarbonitrile, 2,2,2-trifluoroacetic acid, 2,2,2-trifluoroacetic |
| | acid |
| 416 | 4-{2-[4-(imidazolylmethyl)-3-pyridyl]-1,3-thiazol-4- |
| | yl}benzenecarbonitrile, 2,2,2-trifluoroacetic acid, 2,2,2-trifluoroacetic |
| | acid |
| 417 | 4-{2-[4-(morpholin-4-ylmethyl)-3-pyridyl]-1,3-thiazol-4- |
| | yl}benzenecarbonitrile, 2,2,2-trifluoroacetic acid, 2,2,2-trifluoroacetic |
| | acid |
| 418 | 4-(2-{4-[(4-(4-pyridyl)piperazinyl)methyl]-3-pyridyl}-1,3-thiazol-4- |
| | yl)benzenecarbonitrile, 2,2,2-trifluoroacetic acid, 2,2,2-trifluoroacetic |
| | acid, 2,2,2-trifluoroacetic acid, 2,2,2-trifluoroacetic acid |
| 419 | 4-[2-(4-{[(2-methoxyethyl)amino]methyl}-3-pyridyl)-1,3-thiazol-4- |
| | yl]benzenecarbonitrile |
| 420 | 4-{2-[4-({[2-(dimethylamino)ethyl]amino}methyl)-3-pyridyl]-1,3- |
| | thiazol-4-yl}benzenecarbonitrile |
| 421 | 4-(2-{4-[(methylamino)methyl]-3-pyridyl}-1,3-thiazol-4- |
| | yl)benzenecarbonitrile |
| 422 | 4-(2-{4-[(ethylamino)methyl]-3-pyridyl}-1,3-thiazol-4- |
| | yl)benzenecarbonitrile |
| 423 | 4-[2-(4-{[(methylethyl)amino]methyl}-3-pyridyl)-1,3-thiazol-4- |
| | yl]benzenecarbonitrile |
| 424 | 2-(2-fluorophenyl)-4-(4-methyl(3-pyridyl))-1,3-thiazole, hydrogen |
| | chloride |
| 425 | 2-(3-fluorophenyl)-4-(4-methyl(3-pyridyl))-1,3-thiazole, hydrogen |
| | chloride |
| 426 | 3-methoxy-1-[4-(4-methyl(3-pyridyl))(1,3-thiazol-2-yl)]benzene, |
| | hydrogen chloride |
| 427 | 2-(2,4-dichlorophenyl)-4-(4-methyl(3-pyridyl))-1,3-thiazole, hydrogen |
| | chloride |
| 428 | 4-(4-methyl(3-pyridyl))-2-[4-(trifluoromethyl)phenyl]-1,3-thiazole, |
| | hydrogen chloride |
| 429 | 2-(2-chlorophenyl)-4-(4-methyl(3-pyridyl))-1,3-thiazole, hydrogen |
| | chloride |
| 430 | 2-(3-chlorophenyl)-4-(4-methyl(3-pyridyl))-1,3-thiazole, hydrogen |
| | chloride |
| 431 | 2-(3-chloro-4-fluorophenyl)-4-(4-methyl(3-pyridyl))-1,3-thiazole, |
| | hydrogen chloride |
| 432 | 2-(2,3-dihydrobenzo[b]furan-5-yl)-4-(4-methyl(3-pyridyl))-1,3-thiazole, |
| | hydrogen chloride |
| 433 | 2-(2,3-dichlorophenyl)-4-(4-methyl(3-pyridyl))-1,3-thiazole, hydrogen |
| | chloride |
| 434 | 4-(4-methyl(3-pyridyl))-2-(4-methylphenyl)-1,3-thiazole, hydrogen |
| | chloride |
| 435 | 2-(3-fluoro-4-methylphenyl)-4-(4-methyl(3-pyridyl))-1,3-thiazole, |
| | hydrogen chloride |
| 436 | 2-[4-(tert-butyl)phenyl]-4-(4-methyl(3-pyridyl))-1,3-thiazole |
| 437 | 2-methoxy-1-[4-(4-methyl(3-pyridyl))(1,3-thiazol-2-yl)]benzene, |
| | hydrogen chloride |
| 438 | 4-(4-methyl(3-pyridyl))-2-(2-naphthyl)-1,3-thiazole, hydrogen chloride |
| 439 | (4-fluorophenyl)[4-(4-methyl(3-pyridyl))(1,3-thiazol-2-yl)]amine, |
| | hydrogen chloride |
| 440 | (4-chlorophenyl)[4-(4-methyl(3-pyridyl))(1,3-thiazol-2-yl)]amine, |
| | hydrogen chloride |
| 441 | (4-methoxyphenyl)[4-(4-methyl(3-pyridyl))(1,3-thiazol-2-yl)]amine, |
| | hydrogen chloride |
| 442 | [4-(4-methyl(3-pyridyl))(1,3-thiazol-2-yl)]-3-pyridylamine, hydrogen |
| | chloride |
| 443 | (2-fluorophenyl)[4-(4-methyl(3-pyridyl))(1,3-thiazol-2-yl)]amine, |
| | hydrogen chloride |
| 444 | (2-chlorophenyl)[4-(4-methyl(3-pyridyl))(1,3-thiazol-2-yl)]amine, |
| | hydrogen chloride |
| 445 | (3-chlorophenyl)[4-(4-methyl(3-pyridyl))(1,3-thiazol-2-yl)]amine, |
| | hydrogen chloride |
| 446 | ethyl 4-{[4-(4-methyl-3-pyridyl)-1,3-thiazol-2-yl]amino}benzoate, |
| | hydrogen chloride |
| 447 | 4-(4-methyl(3-pyridyl))-2-(2-nitrophenyl)-1,3-thiazole, hydrogen chloride |
| 448 | 4-(4-methyl(3-pyridyl))-2-(3-nitrophenyl)-1,3-thiazole, hydrogen chloride |
| 449 | 4-(4-methyl(3-pyridyl))-2-(4-nitrophenyl)-1,3-thiazole, hydrogen chloride |
| 450 | (3-fluorophenyl)[4-(4-methyl(3-pyridyl))(1,3-thiazol-2-yl)]amine, |
| | hydrogen chloride |
| 451 | (2-methoxyphenyl)[4-(4-methyl(3-pyridyl))(1,3-thiazol-2-yl)]amine, |
| | hydrogen chloride |
| 452 | methyl[4-(4-methyl(3-pyridyl))(1,3-thiazol-2-yl)]phenylamine, hydrogen |
| | chloride |
| 453 | dimethyl(4-{[4-(4-methyl(3-pyridyl))(1,3-thiazol-2- |
| | yl)]amino}phenyl)amine, hydrogen chloride |
| 454 | [4-(4-methyl(3-pyridyl))(1,3-thiazol-2-yl)](4-methylphenyl)amine, |
| | hydrogen chloride |
| 455 | 4-chloro-1-{[4-(4-methyl(3-pyridyl))(1,3-thiazol-2-yl)]methoxy}benzene, |
| | hydrogen chloride |
| 456 | 4-{[4-(4-methyl-3-pyridyl)-1,3-thiazol-2-yl]amino}benzenecarbonitrile, |
| | hydrogen chloride |
| 457 | [4-(4-methyl(3-pyridyl))(1,3-thiazol-2-yl)]phenylamine, hydrogen |
| | chloride |
| 458 | (3,5-dichlorophenyl)[4-(4-methyl(3-pyridyl))(1,3-thiazol-2-yl)]amine, |
| | hydrogen chloride |
| 459 | 4-{[4-(4-methyl-3-pyridyl)-1,3-thiazol-2-yl]amino}benzoic acid, 2,2,2- |
| | trifluoroacetic acid |
| 460 | 2-chloro-1-{[4-(4-methyl(3-pyridyl))(1,3-thiazol-2-yl)]methoxy}benzene, |
| | 2,2,2-trifluoroacetic acid |
| 461 | 1-methoxy-4-[4-(4-methyl(3-pyridyl))(1,3-thiazol-2-yl)]-2-nitrobenzene, |
| | hydrogen chloride |
| 462 | 2-[2,6-dichloro-4-(trifluoromethyl)phenyl]-4-(4-methyl(3-pyridyl))-1,3- |
| | thiazole, hydrogen chloride |
| 463 | 4-(4-methyl(3-pyridyl))-2-[3-(trifluoromethyl)phenyl]-1,3-thiazole, |
| | hydrogen chloride |
| 464 | 2-[3,5-bis(trifluoromethyl)phenyl]-4-(4-methyl(3-pyridyl))-1,3-thiazole, |
| | hydrogen chloride |
| 465 | 2-isoxazol-5-yl-4-(4-methyl(3-pyridyl))-1,3-thiazole, hydrogen chloride |
| 466 | 4-(4-methyl(3-pyridyl))-2-(4-phenylphenyl)-1,3-thiazole, hydrogen |
| | chloride |
| 467 | (2,4-dimethoxyphenyl)[4-(4-methyl(3-pyridyl))(1,3-thiazol-2-yl)]amine, |
| | hydrogen chloride |
| 468 | 2,5-dimethoxyphenyl)[4-(4-methyl(3-pyridyl))(1,3-thiazol-2-yl)]amine, |
| | hydrogen chloride |
| 469 | (3-methoxyphenyl)[4-(4-methyl(3-pyridyl))(1,3-thiazol-2-yl)]amine, |
| | hydrogen chloride |
| 470 | [4-(4-methyl(3-pyridyl))(1,3-thiazol-2-yl)](3-methylthiophenyl)amine, |
| | hydrogen chloride |
| 471 | ethyl 3-{[4-(4-methyl-3-pyridyl)-1,3-thiazol-2-yl]amino}benzoate, |
| | hydrogen chloride |
| 472 | 3-{[4-(4-methyl-3-pyridyl)-1,3-thiazol-2-yl]amino}benzenecarbonitrile, |
| | hydrogen chloride |
| 473 | [4-(4-methyl(3-pyridyl))(1,3-thiazol-2-yl)][4- |
| | (phenylmethoxy)phenyl]amine,hydrogen chloride |
| 474 | 2-(2-chlorophenyl)-5-ethyl-1-[4-(methylethyl)phenyl]imidazole-4- |
| | carboxylic acid |
| 475 | 4-methoxy-1-[4-(4-methyl(3-pyridyl))(1,3-thiazol-2-yl)]benzene, |
| | hydrogen chloride |
| 476 | 2-{4-[4-(4-methyl(3-pyridyl))(1,3-thiazol-2-yl)]phenoxy}-5- |
| | (trifluoromethyl)pyridine, 2,2,2-trifluoroacetic acid, 2,2,2-trifluoroacetic |
| | acid |
| 477 | 2-(4-chlorophenyl)-4-(4-methyl(3-pyridyl))-1,3-thiazole, hydrogen |
| | chloride |
| 478 | 4-{4-[4-(4-methyl-3-pyridyl)-1,3-thiazol-2-yl]phenyl}-1,2,3-thiadiazole, |
| | hydrogen chloride |
| 479 | 2-{4-[(4,5-dichloroimidazolyl)methyl]phenyl}-4-(4-methyl(3-pyridyl))- |
| | 1,3-thiazole, hydrogen chloride |
| 480 | 2,4-bis(4-methyl-3-pyridyl)-1,3-thiazole, hydrogen chloride, hydrogen |
| | chloride |
| 481 | (4-chloro-2-methoxyphenyl)[4-(4-methyl(3-pyridyl))(1,3-thiazol-2- |
| | yl)]amine, hydrogen chloride |
| 482 | (5-fluoro-2-methylphenyl)[4-(4-methyl(3-pyridyl))(1,3-thiazol-2- |
| | yl)]amine, hydrogen chloride |
| 483 | (2,4-dichlorophenyl)[4-(4-methyl(3-pyridyl))(1,3-thiazol-2-yl)]amine, |
| | hydrogen chloride |
| 484 | (2,4-difluorophenyl)[4-(4-methyl(3-pyridyl))(1,3-thiazol-2-yl)]amine, |
| | hydrogen chloride |
| 485 | [4-(4-methyl(3-pyridyl))(1,3-thiazol-2-yl)][3- |
| | (trifluoromethyl)phenyl]amine, hydrogen chloride |
| 486 | [4-(4-methyl(3-pyridyl))(1,3-thiazol-2-yl)][4- |
| | (trifluoromethyl)phenyl]amine, hydrogen chloride |
| 487 | [4-(4-methyl(3-pyridyl))(1,3-thiazol-2-yl)][2- |
| | (trifluoromethyl)phenyl]amine, hydrogen chloride |
| 488 | 1-(4-{[4-(4-methyl-3-pyridyl)-1,3-thiazol-2-yl]amino}phenyl)ethan-1- |
| | one, hydrogen chloride |
| 489 | 4-[4-(4-methyl-3-pyridyl)-1,3-thiazol-2-yl]benzenecarbonitrile, hydrogen |
| | chloride |
| 490 | 4-[4-(4-methyl-3-pyridyl)-1,3-thiazol-2-yl]benzenecarbonitrile |
| 491 | 2-(4-cyclopropyl(3-pyridyl))-4-(3-nitrophenyl)-1,3-thiazole |
| 492 | [4-(4-methyl(3-pyridyl))(1,3-thiazol-2-yl)](4-nitrophenyl)amine, |
| | hydrogen chloride |
| 493 | (2-fluorophenyl)[4-(4-methyl(3-pyridyl))(1,3-thiazol-2-yl)]amine |
| 494 | [4-(4-methyl(3-pyridyl))(1,3-thiazol-2-yl)]phenylamine |
| 495 | methyl[4-(4-methyl(3-pyridyl))(1,3-thiazol-2-yl)]phenylamine |
| 496 | (4-fluorophenyl)[4-(4-methyl(3-pyridyl))(1,3-thiazol-2-yl)]amine |
| 497 | 2-(2-fluorophenyl)-4-(4-methyl(3-pyridyl))-1,3-thiazole |
| 498 | 4-(4-methyl(3-pyridyl))-2-[4-(trifluoromethyl)phenyl]-1,3-thiazole |
| 499 | 2-(2,4-dichlorophenyl)-4-(4-methyl(3-pyridyl))-1,3-thiazole |
| 500 | 2-methoxy-1-[4-(4-methyl(3-pyridyl))(1,3-thiazol-2-yl)]benzene |
| 501 | 4-(4-methyl(3-pyridyl))-2-(2-naphthyl)-1,3-thiazole |
| 502 | (4-chlorophenyl)[4-(4-methyl(3-pyridyl))(1,3-thiazol-2-yl)]amine |
| 503 | (4-methoxyphenyl)[4-(4-methyl(3-pyridyl))(1,3-thiazol-2-yl)]amine |
| 504 | [4-(4-methyl(3-pyridyl))(1,3-thiazol-2-yl)]-3-pyridylamine |
| 505 | [4-(4-methyl(3-pyridyl))(1,3-thiazol-2-yl)]benzylamine |
| 506 | [(4-methoxyphenyl)methyl][4-(4-methyl(3-pyridyl))(1,3-thiazol-2- |
| | yl)]amine |
| 507 | [4-(4-methyl(3-pyridyl))(1,3-thiazol-2-yl)][(4- |
| | methylphenyl)methyl]amine |
| 508 | [(4-chlorophenyl)methyl][4-(4-methyl(3-pyridyl))(1,3-thiazol-2-yl)]amine |
| 509 | (diphenylmethyl)[4-(4-methyl(3-pyridyl))(1,3-thiazol-2-yl)]amine |
| 510 | [4-(4-methyl(3-pyridyl))(1,3-thiazol-2-yl)](2-phenylethyl)amine |
| 511 | cyclohexyl[4-(4-methyl(3-pyridyl))(1,3-thiazol-2-yl)]amine |
| 512 | [4-(4-methyl(3-pyridyl))(1,3-thiazol-2-yl)](3-morpholin-4-ylpropyl)amine |
| 513 | [4-(4-methyl(3-pyridyl))(1,3-thiazol-2-yl)](2-piperidylethyl)amine |
| 514 | butyl[4-(4-methyl(3-pyridyl))(1,3-thiazol-2-yl)]amine,2,2,2- |
| | trifluoroacetic acid |
| 515 | (2-furylmethyl)[4-(4-methyl(3-pyridyl))(1,3-thiazol-2-yl)]amine, 2,2,2- |
| | trifluoroacetic acid |
| 516 | [4-(4-methyl(3-pyridyl))(1,3-thiazol-2-yl)](oxolan-2-ylmethyl)amine, |
| | 2,2,2-trifluoroacetic acid |
| 517 | [4-(4-methyl(3-pyridyl))(1,3-thiazol-2-yl)](2-morpholin-4-ylethyl)amine, |
| | 2,2,2-trifluoroacetic acid, 2,2,2-trifluoroacetic acid |
| 518 | dimethyl(3-{[4-(4-methyl(3-pyridyl))(1,3-thiazol-2- |
| | yl)]amino}propyl)amine, 2,2,2-trifluoroacetic acid, 2,2,2-trifluoroacetic |
| | acid |
| 519 | 2-(4-chlorophenyl)-5-ethyl-4-(4-propyl(3-pyridyl))-1,3-thiazole |
| 520 | 2-(4-chlorophenyl)-5-[4-(2-methylpropyl)(3-pyridyl)]-1,3-thiazole |
| 521 | 5-chloro-2-(4-chlorophenyl)-4-(4-methyl(3-pyridyl))-1,3-thiazole |
| 522 | [6-(2,6-difluorophenyl)(3a-hydroimidazolo[1,2-e]pyrimidin-4-yl)](3- |
| | methoxyphenyl)amine |
| 523 | 4-(4-propyl(3-pyridyl))-2-(4-pyridyl)-1,3-thiazole |
| 524 | 2-(4-nitrophenyl)-4-(4-propyl(3-pyridyl))-1,3-thiazole |
| 525 | 2-(3-nitrophenyl)-4-(4-propyl(3-pyridyl))-1,3-thiazole |
| 526 | 4-[4-(4-propyl-3-pyridyl)-1,3-thiazol-2-yl]benzenecarbonitrile |
| 527 | 2-phenyl-4-(4-propyl(3-pyridyl))-1,3-thiazole |
| 528 | 2-(4-chlorophenyl)-4-(4-propyl(3-pyridyl))-1,3-thiazole |
| 529 | 4-(4-propyl(3-pyridyl))-2-(3-thienyl)-1,3-thiazole |
| 530 | 4-(4-propyl(3-pyridyl))-2-(2-thienyl)-1,3-thiazole |
| 531 | 2-(5-nitro(3-thienyl))-4-(4-propyl(3-pyridyl))-1,3-thiazole |
| 532 | 4-(4-propyl(3-pyridyl))-2-pyrazin-2-yl-1,3-thiazole |
| 533 | 4-(4-propyl(3-pyridyl))-2-[4-(trifluoromethyl)(3-pyridyl)]-1,3-thiazole |
| 534 | 3-[4-(4-methyl-3-pyridyl)-1,3-thiazol-2-yl]benzenecarbonitrile, 2,2,2- |
| | trifluoroacetic acid |
| 535 | 4-[4-(4-methyl-3-pyridyl)-1,3-thiazol-2-yl]benzenecarboxamidine |
| 536 | 4-(4-methyl(3-pyridyl))-2-pyrazin-2-yl-1,3-thiazole |
| 537 | 4-(4-methyl(3-pyridyl))-2-(2-thienyl)-1,3-thiazole |
| 538 | 4-(5,5,8,8-tetramethyl(2-5,6,7,8-tetrahydronaphthyl))-2-[4- |
| | (trifluoromethyl)(3-pyridyl)]-1,3-thiazole, 2,2,2-trifluoroacetic acid |
| 539 | 4-(4-methyl-3-pyridyl)-1,3-thiazole-2-ylamine, hydrogen chloride |
| 540 | 4-[4-(4-methyl-3-pyridyl)-1,3-thiazol-2-yl]benzenecarbonitrile, |
| | methanesulfonic acid |
| 541 | 4-{4-[4-(trifluoromethyl)-3-pyridyl]-1,3-thiazol-2-yl}benzenecarbonitrile |
| 542 | 2-isoquinolyl-4-[4-(methylethyl)(3-pyridyl)]-1,3-thiazole, 2,2,2- |
| | trifluoroacetic acid |
| 543 | 2-(2,6-dichloro(4-pyridyl))-4-[4-(methylethyl)(3-pyridyl)]-1,3-thiazole, |
| | 2,2,2-trifluoroacetic acid |
| 544 | 2-(3-chlorophenyl)-4-(4-ethyl(3-pyridyl))-1,3-thiazole, 2,2,2- |
| | trifluoroacetic acid |
| 545 | 2-(3-chlorophenyl)-4-(4-cyclopropyl(3-pyridyl))-1,3-thiazole, 2,2,2- |
| | trifluoroacetic acid |
| 546 | 2-(3-chlorophenyl)-4-[4-(methylethyl)(3-pyridyl)]-1,3-thiazole, 2,2,2- |
| | trifluoroacetic acid |
| 547 | 4-(4-cyclopropyl(3-pyridyl))-2-phenyl-1,3-thiazole, 2,2,2-trifluoroacetic |
| | acid |
| 548 | 4-[4-(methylethyl)(3-pyridyl)]-2-phenyl-1,3-thiazole, 2,2,2-trifluoroacetic |
| | acid |
| 549 | 2-(4-chlorophenyl)-4-(4-ethyl(3-pyridyl))-1,3-thiazole, 2,2,2- |
| | trifluoroacetic acid |
| 550 | 2-(4-chlorophenyl)-4-[4-(methylethyl)(3-pyridyl)]-1,3-thiazole, 2,2,2- |
| | trifluoroacetic acid |
| 551 | 4-(4-ethyl(3-pyridyl))-2-(3-nitrophenyl)-1,3-thiazole, 2,2,2-trifluoroacetic |
| | acid |
| 552 | 2-(4-chlorophenyl)-4-(4-cyclopropyl(3-pyridyl))-1,3-thiazole, 2,2,2- |
| | trifluoroacetic acid |
| 553 | 4-(4-cyclopropyl(3-pyridyl))-2-(3-nitrophenyl)-1,3-thiazole, 2,2,2- |
| | trifluoroacetic acid |
| 554 | 4-[4-(methylethyl)(3-pyridyl)]-2-(3-nitrophenyl)-1,3-thiazole, 2,2,2- |
| | trifluoroacetic acid |
| 555 | 3-[4-(4-ethyl-3-pyridyl)-1,3-thiazol-2-yl]benzenecarbonitrile, 2,2,2- |
| | trifluoroacetic acid |
| 556 | 3-{4-[4-(methylethyl)-3-pyridyl]-1,3-thiazol-2-yl}benzenecarbonitrile, |
| | 2,2,2-trifluoroacetic acid |
| 557 | 3-[4-(4-cyclopropyl-3-pyridyl)-1,3-thiazol-2-yl]benzenecarbonitrile, |
| | 2,2,2-trifluoroacetic acid |
| 558 | 4-(4-ethyl(3-pyridyl))-2-(4-nitrophenyl)-1,3-thiazole, 2,2,2-trifluoroacetic |
| | acid |
| 559 | 4-(4-cyclopropyl(3-pyridyl))-2-(4-nitrophenyl)-1,3-thiazole, 2,2,2- |
| | trifluoroacetic acid |
| 560 | 4-[4-(methylethyl)(3-pyridyl)]-2-(4-nitrophenyl)-1,3-thiazole,2,2,2- |
| | trifluoroacetic acid |
| 561 | 3-[4-(4-cyclopropyl(3-pyridyl))(1,3-thiazol-2-yl)]-6-methylpyridin-2-ol, |
| | 2,2,2-trifluoroacetic acid |
| 562 | 6-methyl-3-{4-[4-(methylethyl)(3-pyridyl)](1,3-thiazol-2-yl)}pyridin-2- |
| | ol, 2,2,2-trifluoroacetic acid |
| 563 | 4-(4-ethyl(3-pyridyl))-2-(6-methyl(3-pyridyl))-1,3-thiazole, 2,2,2- |
| | trifluoroacetic acid |
| 564 | 4-(4-cyclopropyl(3-pyridyl))-2-(6-methyl(3-pyridyl))-1,3-thiazole, 2,2,2- |
| | trifluoroacetic acid |
| 565 | 2-(6-methyl(3-pyridyl))-4-[4-(methylethyl)(3-pyridyl)]-1,3-thiazole, |
| | 2,2,2-trifluoroacetic acid |
| 566 | 4-(4-cyclopropyl(3-pyridyl))-2-(4-methylphenyl)-1,3-thiazole, 2,2,2- |
| | trifluoroacetic acid |
| 567 | 4-[4-(methylethyl)(3-pyridyl)]-2-(4-methylphenyl)-1,3-thiazole, 2,2,2- |
| | trifluoroacetic acid |
| 568 | l-[4-(4-cyclopropyl(3-pyridyl))(1,3-thiazol-2-yl)]-4-methoxybenzene, |
| | 2,2,2-trifluoroacetic acid |
| 569 | 4-methoxy-1-{4-[4-(methylethyl)(3-pyridyl)](1,3-thiazol-2-yl)}benzene, |
| | 2,2,2-trifluoroacetic acid |
| 570 | 4-(4-ethyl(3-pyridyl))-2-[4-(trifluoromethyl)phenyl]-1,3-thiazole, 2,2,2- |
| | trifluoroacetic acid |
| 571 | 4-(4-cyclopropyl(3-pyridyl))-2-[4-(trifluoromethyl)phenyl]-1,3-thiazole, |
| | 2,2,2-trifluoroacetic acid |
| 572 | 2-(3-pyridyl)-4,5-dihydrobenzo[e]benzothiazole |
| 573 | 7-methoxy-2-(3-pyridyl)-4,5-dihydrobenzo[e]benzothiazole |
| 574 | 8-methoxy-2-(3-pyridyl)-4,5-dihydrobenzo[e]benzothiazole |
| 575 | (1R)-6-aza-1,10,10-trimethyl-5-(3-pyridyl)-4- |
| | thiatricyclo[7.4.0.0<3,7>]trideca-3(7),5-diene |
| 576 | 5-[(4-methylphenyl)sulfonyl]-2-(3-pyridyl)-4,5,6,7-tetrahydro-1,3- |
| | thiazolo[5,4-c]pyridine |
| 577 | 2-(4-methyl-3-pyridyl)-4,5-dihydrobenzo[e]benzothiazole |
| 578 | 2-(3-pyridyl)-4,6,7-trihydro-1,3-thiazolo[4,5-d]pyrimidin-5-one |
| 579 | 6-methyl-2-(3-pyridyl)-4,6,7-trihydro-1,3-thiazolo[4,5-d]pyrimidin-5-one |
| 580 | 4-phenyl-2-(3-pyridyl)-2-pyrrolino[2,3-d]1,3-thiazole |
| 581 | 3-[4-(4-chlorophenyl)-1,3-thiazol-2-yl]pyridin-1-ol |
| 582 | 3-[4-(4-chlorophenyl)(1,3-thiazol-2-yl)]-4-methylpyridin-1-ol |
| 583 | 4-(4-chlorophenyl)-2-(5-methyl(3-pyridyl))-1,3-thiazole |
| 584 | 2-(3-pyridyl)-4,5,6,7-tetrahydrobenzothiazole |
| 585 | 2-(4-methyl-3-pyridyl)-4,5,6,7,8-pentahydrocyclohepta[1,2-d]1,3-thiazole |
| 586 | 2-(3-pyridyl)-4,5,6,7,8-pentahydrocyclohepta[1,2-d]1,3-thiazole |
| 587 | 1,3-dimethoxy-2-[2-(5-methyl(3-pyridyl))(1,3-thiazol-4-yl)]benzene |
| 588 | 2-(3-pyridyl)-4,5,6-trihydrocyclopenta[1,2-d]1,3-thiazole |
| 589 | 2-(4-isoquinolyl)-4-phenyl-1,3-thiazole |
| 590 | 4-(4-chlorophenyl)-2-(4-isoquinolyl)-1,3-thiazole |
| 591 | 2-(4-methyl-3-pyridyl)-4,5,6,7-tetrahydrobenzothiazole |
| 592 | 2-(4-methyl-3-pyridyl)-4,5,6-trihydrocyclopenta[1,2-d]1,3-thiazole |
| 593 | 2-(4-methyl(3-pyridyl))-4-phenyl-4,5,6,7,8-pentahydrocyclohepta[1,2- |
| | d]1,3-thiazole |
| 594 | 2-[4-(methylethyl)(3-pyridyl)]-4-phenyl-4,5,6,7,8- |
| | pentahydrocyclohepta[1,2-d]1,3-thiazole |
| 595 | 4-phenyl-2-(3-pyridyl)-4,5,6,7,8-pentahydrocyclohepta[1,2-d]1,3-thiazole |
| 596 | 7-(4-methyl-3-pyridyl)-4,5-dihydro-1,2,5-oxadiazolo[3,4-e]benzothiazole |
| 597 | 7-(3-pyridyl)-4,5-dihydro-1,2,5-oxadiazolo[3,4-e]benzothiazole |
| 598 | 2-(4-methyl-3-pyridyl)-6,7-dihydrobenzothiazole-4,5-diimine |
| 599 | 4-(4-fluorophenyl)-2-(4-isoquinolyl)-1,3-thiazole |
| 600 | 4-(3-chlorophenyl)-2-(4-isoquinolyl)-1,3-thiazole |
| 601 | 3-(2-(4-isoquinolyl)-1,3-thiazol-4-yl)benzenecarbonitrile |
| 602 | 2-(4-isoquinolyl)-4-(3-nitrophenyl)-1,3-thiazole |
| 603 | 4-(3-fluorophenyl)-2-(4-isoquinolyl)-1,3-thiazole |
| 604 | 4-(3-bromophenyl)-2-(4-isoquinolyl)-1,3-thiazole |
| 605 | 4-(4-bromophenyl)-2-(4-isoquinolyl)-1,3-thiazole |
| 606 | 2-(4-isoquinolyl)-4-(4-nitrophenyl)-1,3-thiazole |
| 607 | 2-(4-isoquinolyl)-4-(4-methylphenyl)-1,3-thiazole |
| 608 | 1-(2-(4-isoquinolyl)(1,3-thiazol-4-yl))-4-methoxybenzene |
| 609 | difluoro[4-(2-(4-isoquinolyl)(1,3-thiazol-4-yl))phenoxy]methane |
| 610 | trifluoro[4-(2-(4-isoquinolyl)(1,3-thiazol-4-yl))phenoxy]methane |
| 611 | 4-(4-bromophenyl)-2-(4-isoquinolyl)-5-methyl-1,3-thiazole |
| 612 | 3-aza-4-(3-pyridyl)-5-thiatricyclo[6.2.1.0<2,6>]undeca-2(6),3-diene |
| 613 | 3-aza-4-(4-methyl(3-pyridyl))-5-thiatricyclo[6.2.1.0<2,6>]undeca-2(6),3- |
| | diene |
| 614 | 3-aza-4-[4-(methylethyl)(3-pyridyl)]-5-thiatricyclo[6.2.1.0<2,6>]undeca- |
| | 2(6),3-diene |
| 615 | 4-(2-fluorophenyl)-2-(4-isoquinolyl)-1,3-thiazole |
| 616 | 4-(2-chlorophenyl)-2-(4-isoquinolyl)-1,3-thiazole |
| 617 | 1-(2-(4-isoquinolyl)(1,3-thiazol-4-yl))-2-methoxybenzene |
| 618 | 2-(4-isoquinolyl)-4-(4-phenylphenyl)-1,3-thiazole |
| 619 | 4-(3,4-dichlorophenyl)-2-(4-isoquinolyl)-1,3-thiazole |
| 620 | 4-(2,4-dimethylphenyl)-2-(4-isoquinolyl)-1,3-thiazole |
| 621 | 1-(2-(4-isoquinolyl)(1,3-thiazol-4-yl))-2,4-dimethoxybenzene |
| 622 | 4-(4-chloro-3-nitrophenyl)-2-(4-isoquinolyl)-1,3-thiazole |
| 623 | 2-(4-isoquinolyl)-4-(2-naphthyl)-1,3-thiazole |
| 624 | 4-cyclohexyl-2-(4-isoquinolyl)-1,3-thiazole |
| 625 | 2-(4-isoquinolyl)-4-(2-nitrophenyl)-1,3-thiazole |
| 626 | 4-adamantanyl-2-(4-isoquinolyl)-1,3-thiazole |
| 627 | 4-(3,5-dimethylphenyl)-2-(3-pyridylmethyl)-1,3-thiazole |
| 628 | 4-phenyl-2-(3-pyridylmethyl)-1,3-thiazole |
| 629 | 3-methoxy-1-[2-(3-pyridylmethyl)(1,3-thiazol-4-yl)]benzene |
| 630 | 4-(2-nitrophenyl)-2-(3-pyridylmethyl)-1,3-thiazole |
| 631 | 4-(3-fluorophenyl)-2-(3-pyridylmethyl)-1,3-thiazole |
| 632 | 2-pyrazin-2-yl-4-(4-pyridyl)-1,3-thiazole, 2,2-difluoropropanoic acid, 2,2- |
| | difluoropropanoic acid, 2,2,2-trifluoroacetic acid, fluoride, fluoride |
| 633 | 4-(4-fluorophenyl)-2-(1-methylimidazol-5-yl)-1,3-thiazole |
| 634 | 2-(4-chloro(3-pyridyl))-4-(4-chlorophenyl)-1,3-thiazole, hydrogen |
| | chloride |
| 635 | 4-(4-chlorophenyl)-2-(imidazol-2-ylmethyl)-1,3-thiazole |
| 636 | 2-(5-bromo(3-pyridyl))-4-(4-fluorophenyl)-1,3-thiazole |
| 637 | 2-(5-bromo(3-pyridyl))-4-(2-fluorophenyl)-1,3-thiazole |
| 638 | 2-(5-bromo(3-pyridyl))-4-(3-nitrophenyl)-1,3-thiazole |
| 639 | 2-(5-bromo(3-pyridyl))-4-(4-chlorophenyl)-1,3-thiazole |
| 640 | 2-(5-bromo(3-pyridyl))-4-(2-chlorophenyl)-1,3-thiazole |
| 641 | 4-(3,4-dichlorophenyl)-2-(5-bromo(3-pyridyl))-1,3-thiazole |
| 642 | 2-(5-bromo(3-pyridyl))-4-(4-bromophenyl)-1,3-thiazole |
| 643 | 2-(5-bromo(3-pyridyl))-4-(3-bromophenyl)-1,3-thiazole |
| 644 | 2-(5-bromo(3-pyridyl))-4-(4-bromophenyl)-5-methyl-1,3-thiazole |
| 645 | 2-(5-bromo(3-pyridyl))-4-(4-methylphenyl)-1,3-thiazole |
| 646 | {4-[2-(5-bromo(3-pyridyl))(1,3-thiazol-4-yl)]phenoxy}difluoromethane |
| 647 | {4-[2-(5-bromo(3-pyridyl))(1,3-thiazol-4-yl)]phenoxy}trifluoromethane |
| 648 | 1-[2-(5-bromo(3-pyridyl))(1,3-thiazol-4-yl)]-4-methoxybenzene |
| 649 | 4-[2-(5-bromo-3-pyridyl)-1,3-thiazol-4-yl]benzenecarbonitrile |
| 650 | 3-[2-(5-bromo-3-pyridyl)-1,3-thiazol-4-yl]benzenecarbonitrile |
| 651 | 2-(5-bromo(3-pyridyl))-4-[4-(trifluoromethyl)phenyl]-1,3-thiazole |
| 652 | 1-[2-(5-bromo(3-pyridyl))(1,3-thiazol-4-yl)]-2-methoxybenzene |
| 653 | 2-[2-(5-bromo(3-pyridyl))(1,3-thiazol-4-yl)]-1,4-dimethoxybenzene |
| 654 | 2-(5-bromp(3-pyridyl))-4-(4-phenylphenyl)-1,3-thiazole |
| 655 | 2-(5-bromo(3-pyridyl))-5-methyl-4-phenyl-1,3-thiazole |
| 656 | l-[2-(5-bromo(3-pyridyl))-5-methyl(1,3-thiazol-4-yl)]-4-methoxybenzene |
| 657 | 2-(5-bromo(3-pyridyl))-4-(4-chloro-3-nitrophenyl)-1,3-thiazole |
| 658 | 4-(2H,3H,4H-benzo[b]1,4-dioxepan-7-yl)-2-(5-bromo(3-pyridyl))-1,3- |
| | thiazole |
| 659 | 4-[2-(l-hydroxy-4-methyl-3-pyridyl)-1,3-thiazol-4-yl]benzenecarbonitrile |
| 660 | 4-[4-(1-hydroxy-4-methyl-3-pyridyl)-1,3-thiazol-2-yl]benzenecarbonitrile |
| 661 | 2-(5-bromo(3-pyridyl))-4-(4-ethyl(3-pyridyl))-1,3-thiazole, 2,2,2- |
| | trifluoroacetic acid |
| 662 | 2-(5-bromo(3-pyridyl))-4-[4-(methylethyl)(3-pyridyl)]-1,3-thiazole, 2,2,2- |
| | trifluoroacetic acid |
| 663 | 2-(5-bromo(3-pyridyl))-4-(5-methyl-3-phenylisoxazol-4-yl)-1,3-thiazole, |
| | 2,2,2-trifluoroacetic acid |

Certain compounds of the present invention may exist in particular geometric or stereoisomeric forms. The present invention contemplates all such compounds, including cis- and trans-isomers, R— and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivatizaton with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Compounds may contain a basic functional group, such as amino or alkylamino, and are, thus, capable of forming pharmaceutically acceptable salts with pharmaceutically acceptable acids. The term “pharmaceutically acceptable salts” in this respect, refers to the relatively nontoxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

Pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloric, hydrobromic, sulfuric, suflamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. These salts can be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Contemplated equivalents of the compounds described above include compounds which otherwise correspond thereto, and which have the same general properties thereof (e.g., functioning as 17α-hydroxylase-C17,20-lyase inhibitors), wherein one or more simple variations of substituents are made which do not adversely affect the efficacy of the compound in binding to 17α-hydroxylase-C17,20-lyase receptors. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

### Diseases that can be Treated with the Compounds of the Invention

The present invention provides a method of inhibiting a lyase, e.g., 17α-hydroxylase-C17,20 lyase, comprising contacting a lyase with a compound of the invention. The activity can be inhibited by at least 20%, preferably at least about 50%, more preferably at least about 60%, 70%, 80%, 90%, 95%, and most preferably at least about 98%. In one embodiment, the invention provides a method for inhibiting a lyase in vitro. In a preferred embodiment, the lyase is in vivo or ex vivo. For example, the invention provides methods for inhibiting a lyase in a cell, comprising contacting the cell with a compound of the invention, such that the activity of the lyase is inhibited. The cell may further be contacted with a composition stimulating the uptake of the compound into the cell, e.g., liposomes. In one embodiment, the invention provides a method for inhibiting a lyase in a cell of a subject, comprising administering to the subject a therapeutically effective amount of a compound of the present invention, or a formulation comprising a compound of the present invention, such that the lyase is inhibited in a cell of the subject. The subject can be one having a disease associated with a lyase, e.g., cancer. Preferred types of cancer that can be treated according to the invention include prostate cancer and breast cancer. Other diseases that can be treated include diseases in which it is desired to prevent or inhibit the formation of a hormone selected from the group consisting of the androgens testosterone and dihydrotestosterone (DHT) and the estrogens 17β-estradiol and estrone. Generally, any disease that can be treated by inhibiting the activity of a lyase, e.g., 17α-hydroxylase-C17,20-lyase, can be treated with the compounds of the invention.

In general, the invention provides methods and compositions for the treatment of CYP17 metabolite-associated diseases and disorders. Examples include particularly sex steroid hormone dependent cancers, such as androgen-dependent prostate cancer, which may be treated by inhibiting CYP17-mediated androgen synthesis, and estrogen-dependent breast cancer or ovarian cancer, which may be treated by inhibiting CYP17-mediated estrogen synthesis.

For example, adenocarcinoma of the prostate is a common disease that causes significant morbidity and mortality in the adult male population (see Han and Nelson (2000) Expert Opin. Pharmacother. 1: 443-9). Hormonal therapy for prostate cancer is considered when a patient fails with initial curative therapy, such as radical prostatectomy or definitive radiation therapy, or if he is found with an advanced disease. Hormonal agents have been developed to exploit the fact that prostate cancer growth is dependent on androgen. Non-steroidal anti-androgens (NSAAs) block androgen at the cellular level. Castration is another, albeit drastic means of decreasing androgens levels in order to treat or prevent prostate cancer. The methods and compositions of the invention are useful in inhibiting the C17,20-lyase activity of CYP 17 and thereby decreasing levels of androgen production and the associated growth of androgen-dependent cancers such as prostate cancer.

In another example, breast cancer, particularly breast cancer in postmenopausal women, can be treated by administration of a C17,20-lyase inhibitor of the invention because adrenal and ovarian androgens are the main precursors of the etrogens which stimulate the growth of hormone dependent breast cancer. In addition, breast cancer can be treated with inhibitors of aromatase that prevent interconversion of estrogens and adrenal and ovarian androgens (see Harris et al. (1983) Eur. J. Cancer Clin. Oncol. 19: 11). Patients failing to respond to aromatase inhibitors show elevated levels of androgens in response to aromatase inhibitor treatment (see Harris et al. (1988) Br. J. Cancer 58: 493-6). Accordingly sequential blockade to inhibit androgen production as well as inhibit aromatase may produce greater estrogen suppression and enhanced therapeutic effects in treating breast and other estrogen hormone-dependent forms of cancer. Therefore the inhibitors of the invention may be used alone or in combination with other drugs to treat or prevent hormone-dependent cancers such as breast and prostate cancer.

Furthermore, susceptibility to prostate cancer and breast cancer has been associated with particular polymorphic alleles of the CYP17 gene (see e.g. McKean-Cowdin (2001) Cancer Res. 61: 848-9; Haiman et al. (2001) Cancer Epidmeiol. Biomarkers 10: 743-8; Huang et al. (2001) Cancer Res. 59: 4870-5). Accordingly, the compositions of the invention are particularly suited to treating or preventing hormone-dependent cancers in individuals genetically predisposed to such cancers, particularly those predisposed due to an alteration in the CYP17 gene.

Another group of CYP17 metabolite-associated diseases or disorders amenable to treatment with the compositions and methods of the invention include those associated with mineralocorticoid excess such as hypertension caused by sodium retention at renal tubules. Such a mechanism operates in hypertension such as primary hyperaldosteronism and some forms of congenital adrenal hyperplasia. Recently, deficient cortisol metabolism in the aldosterone target organ has been recognized as a novel form of hypertension known as apparent mineralocorticoid excess. Disorders associated with mineralocorticoid synthesis include abnormalities of mineralocorticoid synthesis and/or metabolism which profoundly affect the regulation of electrolyte and water balance and of blood pressure (see e.g. Connell et al. (2001) Baillieres Best Pract. Res. Clin. Endocrinol. Metab. 15:43-60). Characteristic changes in extracellular potassium, sodium and hydrogen ion concentrations are usually diagnostic of such disorders. Serious deficiency may be acquired, for example, in Addison's disease, or inherited. In most of the inherited syndromes, the precise molecular changes in specific steroidogenic enzymes have been identified. Mineralocorticoid excess may be caused by aldosterone or 11-deoxycorticosterone by inadequate conversion of cortisol to cortisone by 11β-hydroxysteroid dehydrogenase type 2 in target tissues, by glucocorticoid receptor deficiency or by constitutive activation of renal sodium channels. Changes in electrolyte balance and renin as well as the abnormal pattern of corticosteroid metabolism are usually diagnostic. Where these abnormalities are inherited (e.g. 11beta- or 17alpha-hydroxylase deficiencies, glucocorticoid remediable hyperaldosteronism (GRA), receptor defects, Liddle's syndrome), the molecular basis is again usually known and, in some cases, may provide the simplest diagnostic tests. Primary aldosteronism, although readily identifiable, presents problems of differential diagnosis, important because optimal treatment is different for each variant. Finally, a significant proportion of patients with essential hypertension show characteristics of mild mineralocorticoid excess, for example low renin levels. As described above, a decrease in CYP17 activity can result in an alteration in mineralorticoid (e.g. aldosterone) biosynthesis. Accordingly, the “CYP17 metabolite-associated diseases or disorders” of the invention would include those associated with altered levels of aldosterone production (e.g. hypertension, primary adrenal hyperplasia).

Still other examples of CYP17 metabolite-associated diseases or disorders” are Cushing's disease, prostatic hyperplasia, glucocorticoid deficiency, and endometrial cancer.

The subject that can be treated according to the invention can be a mammal, e.g., a primate, equine, canine, bovine, ovine, porcine, or feline. In preferred embodiments of this method, the mammal is a human. In other embodiments, the invention provides methods for inhibiting the lyase activity of enzymes that are present in organisms other than mammals, e.g., yeast and fungus, e.g., mildew. Certain compounds of the invention may function as antifungal compounds.

### Methods of Administering the Compounds of the Invention

The therapeutic methods of the invention generally comprise administering to a subject in need thereof, a pharmaceutically effective amount of a compound of the invention, or a salt, prodrug or composition thereof The compounds of the invention can be administered in an amount effective to inhibit the activity of a 17α-hydroxylase-C17,20-lyase. The compounds of this invention may be administered to mammals, preferably humans, either alone or, preferably, in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

Toxicity and therapeutic efficacy of the compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such reagents to the site of affected tissue in order to minimize potential damage to normal cells and, thereby, reduce side effects.

Data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of activity) as determined in cell culture. Such information can be used to more accurately determine useful doses in humans. The compounds of the invention have an IC₅₀ less than 10 μM as determined by the biochemical or cellular assay described herein. Some compounds of the invention are effective at concentrations of 10 nM, 100 nM, or 1 μM. Based on these numbers, it is possible to derive an appropriate dosage for administration to subjects.

Formation of prodrugs is well known in the art in order to enhance the properties of the parent compound. Such properties include solubility, absorption, biostability and release time (see “Pharmaceutical Dosage Form and Drug Delivery Systems” (Sixth Edition), edited by Ansel et al., publ. by Williams & Wilkins, pgs. 27-29, (1995)). Commonly used prodrugs of the disclosed compounds can be designed to take advantage of the major drug biotransformation reactions and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics (Ninth Edition), editor Molinoff et al., publ. by McGraw-Hill, pages 11-13, (1996)).

The pharmaceutical compositions can be prepared so that they may be administered orally, dermally, parenterally, nasally, ophthalmically, otically, sublingually, rectally or vaginally. Dermal administration includes topical application or transdermal administration. Parenteral administration includes intravenous, intraarticular, intramuscular, intraperitoneal, and subcutaneous injections, as well as use of infusion techniques. One or more compounds of the invention may be present in association with one or more non-toxic pharmaceutically acceptable ingredients and optionally, other active anti-proliferative agents, to form the pharmaceutical composition. These compositions can be prepared by applying known techniques in the art such as those taught in Remington's Pharmaceutical Sciences (Fourteenth Edition), Managing Editor, John E. Hoover, Mack Publishing Co., (1970) or Pharmaceutical Dosage Form and Drug Delivery Systenis (Sixth Edition), edited by Ansel et al., publ. by Williams & Willins, (1995).

As indicated above, pharmaceutical compositions containing a compound of the invention may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically acceptable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia; and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally occurring phosphatide, for example lecithin; or condensation products of an alkylene oxide with fatty acids, for example polyoxyethylene stearate; or condensation products of ethylene oxide with long chain aliphatic alcohols, for example heptadecaethylene-oxycetanol; or condensation products of ethylene oxide with partial esters derived from fatty acids and a hexitol such as polyoxyethylene sorbitol monooleate; or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the compound of the invention in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

Pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Pharmaceutical compositions may be in the form of a sterile injectable aqueous solutions. Among the acceptable vehicles and solvents that may be employed are water, Ringer's solution and isotonic sodium chloride solution.

Sterile injectable preparation may also be a sterile injectable oil-in-water microemulsion where the compound of the invention is dissolved in the oily phase. For example, the active ingredient may be first dissolved in a mixture of soybean oil and lecithin. The oil solution is then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the active compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a nontoxic parenterally acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of the invention may also be administered in the form of a suppository for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of the invention can be employed. For purposes of this application, topical application shall include mouth washes and gargles.

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will preferably be continuous rather than intermittent throughout the dosage regimen.

The compounds of the invention may also be co-administered with other well known therapeutic agents that are selected for their particular usefulness against the condition that is being treated. The compounds may be administered simultaneously or sequentially. For example, the active compounds may be useful in combination with known anti-cancer and cytotoxic agents. Similarly, the active compounds may be useful in combination with agents that are effective in the treatment and prevention of osteoporosis, inflammation, neurofibromatosis, restinosis, and viral infections. The active compounds may also be useful in combination with inhibitors of other components of signaling pathways of cell surface growth factor receptors.

Drugs that can be co-administered to a subject being treated with a compound of the invention include antineoplastic agents selected from vinca alkaloids, epipodophyllotoxins, anthracycline antibiotics, actinomycin D, plicamycin, puromycin, gramicidin D, taxol, colchicine, cytochalasin B, emetine, maytansine, or amsacrine. Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), 1996 edition (Medical Economics Company, Montvale, N.J. 07645-1742, USA).

Radiation therapy, including x-rays or gamma rays which are delivered from either an externally applied beam or-by implantation of tiny radioactive sources, may also be used in combination with a compound of the invention to treat a disease, e.g., cancer.

When a composition according to this invention is administered into a human subject, the daily dosage will normally be determined by the prescribing physician with the dosage generally varying according to the age, weight, and response of the individual patient, as well as the severity of the patient's symptoms.

### Kits of the Invention

In one embodiment, a compound of the invention, materials and/or reagents required for administering the compounds of the invention may be assembled together in a kit. When the components of the kit are provided in one or more liquid solutions, the liquid solution preferably is an aqueous solution, with a sterile aqueous solution being particularly preferred.

The kit may further comprise one or more other drugs, e.g., a chemo- or radiotherapeutic agent. These normally will be a separate formulation, but may be formulated into a single pharmaceutically acceptable composition. The container means may itself be geared for administration, such as an inhalant, syringe, pipette, eye dropper, or other such like apparatus, from which the formulation may be applied to an infected are of the body, such as the lungs, or injected into an animal, or even applied to and mixed with the other components of the kit.

The compositions of these kits also may be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the agent. Kits may also comprise a compound of the invention, labeled for detecting lyases.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with a separate instrument for assisting with the injection/administration or placement of the ultimate complex composition within the body of an animal. Such an instrument may be an inhalant, syringe, pipette, forceps, measured spoon, eye dropper or any such medically approved delivery vehicle. Other instrumentation includes devices that permit the reading or monitoring of reactions or amounts of compounds or polypeptides.

The present invention is further illustrated by the following examples which should not be construed as limiting in any way. The contents of all cited references (including literature references, issued patents, published patent applications as cited throughout this application) are hereby expressly incorporated by reference.

### General Method for the Preparation of Compounds of Formula I

3-Pyridyl thiazoles of Formula I, wherein A, L¹, J, L² and G are as described in claim 1, are

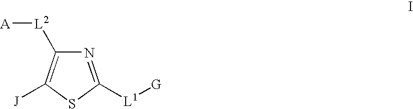

prepared by the general method described below, according to methods described below, or according to methods commonly employed in the art. Compounds of Formula I are prepared according to Scheme 1, whereby halo ketone III, wherein X is Cl, Br, I, or other leaving group commonly employed in the art, is treated with thioamide VI in a polar solvent, such as an alcoholic solvent, at a temperature between 40-120° C. Preferably the polar solvent is an alcohol such as ethanol, 1-propanol, or 2-propanol. Most preferably, compounds of Formula 1 are prepared according to General Methods M, N, O, T, U, and V. Alternatively and preferably, compounds of Formula I can be prepared according to Methods G, H, I, J, K, L, P, Q, R, S, and W. Halo ketones m are commercially available or may be prepared using an electophilic halogen reagent such as bromine, N-chlorosuccinimide, N-bromosuccinimide, or phenyltrimethylammonium tribromide using the general methods or specific examples described below or other methods commonly employed in the art.

Alternatively, the corresponding alphahydroxy ketone can be converted into III using standard conditions employed in the art to convert an alcohol functionality into a halogen or other leaving group commonly employed in the art. Ketones II are commercially available, are prepared prepared according to methods specifically described below, or are prepared according methods described in the following references: Comins, D. L., Smith, R., Stroud, E., Heterocycles, Vol. 22, No. 2, 1984, 339; Leete, E.; Leete, S. A. S., J. Org. Chem. Vol. 43, No. 11, 1978, 2122; Kim, J. G.; Yu, D. S.; Moon, S. H.; Park, J.; Park, W. W. J. Korean Chem. Soc. Vol. 37, No. 9, 1993, 826. Alternatively, the required ketones II can be prepared from the corresponding carboxylic acids using standard conditions employed in the art to convert a carboxylic acid functionality into a ketone. Thioamide VI can be prepared from nitrile V upon treatment with hydrogen sulphide using procedures described below. Alternatively, VI can be prepared from amide IV upon treatment with Lawessons reagent or P₄S₁₀. Nitriles V are commercially available or can be prepared according to the methods described below for Intermediates A-H, or they can be prepared according the methods described in the following references: Comins, D. L., Smith, R., Stroud, E., Heterocycles, Vol. 22, No.2, 1984,339; Leete, E.; Leete, S. A. S., J. Org. Chem. Vol. 43, No. 11, 1978, 2122; Kim, J. G.; Yu, D. S.; Moon, S. H.; Park, J.; Park, W. W. J. Korean Chem. Soc. Vol. 37, No. 9, 1993, 826. Other methods commonly employed in the art may also be used to prepare V. Amides IV are commercially available or they can be prepared by methods commonly employed in the art to prepare amide functionality from carboxylic acid functionality, whereby the requisite carboxylic acid is commercially available or can be prepared according to the following reference: Comins, D. L., Smith, R., Stroud, E., Heterocycles, Vol. 22, NO. 1984;339.

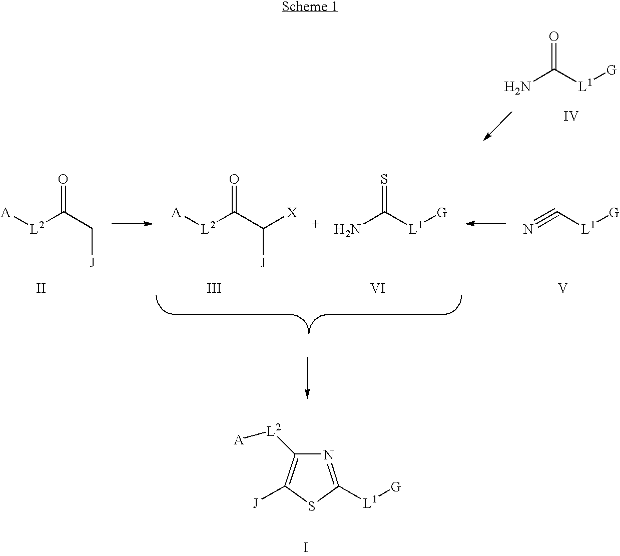

Compounds of Formula I, when A or G is pyridyl, can be converted to an N-oxide upon treatment with a peroxide, such as hydrogen peroxide or MCPBA, in an acidic solvent such as acetic acid, as shown in Scheme 2.

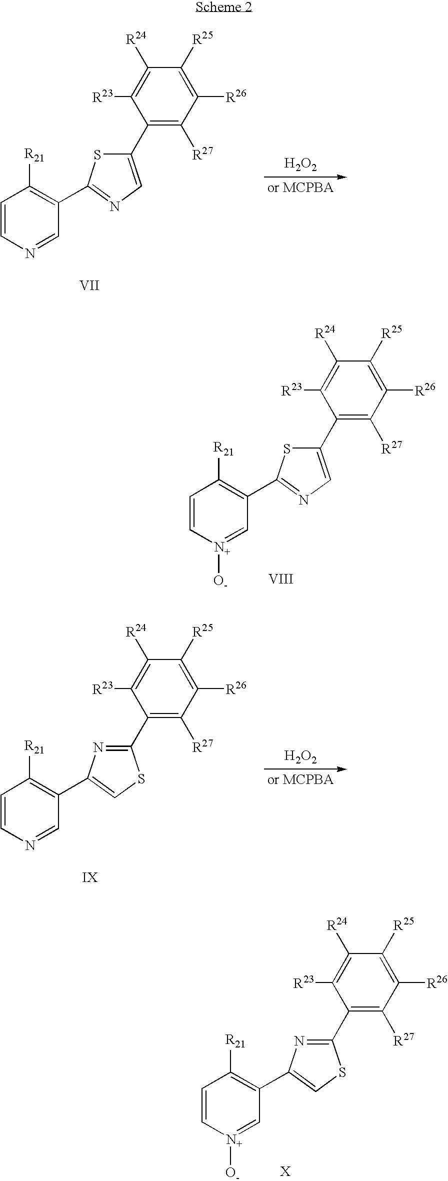

When A or G is 4-methyl pyridyl, such compounds can be treated with H₂O or MCPBA, as shown in Scheme 2, to yield 4-methyl pyridine N-oxides, which can be optionally converted to chloro derivatives XII and XVI as shown in Scheme 3. The N-oxide XI or XV is converted to chloride XII or XVI by treatment with tosyl chloride at elevated temperature. Treatment of chlorides XII or XVI with amines of the formula XIII results in the formation of 4-aminopyridines of the formulae XIV and XVII.

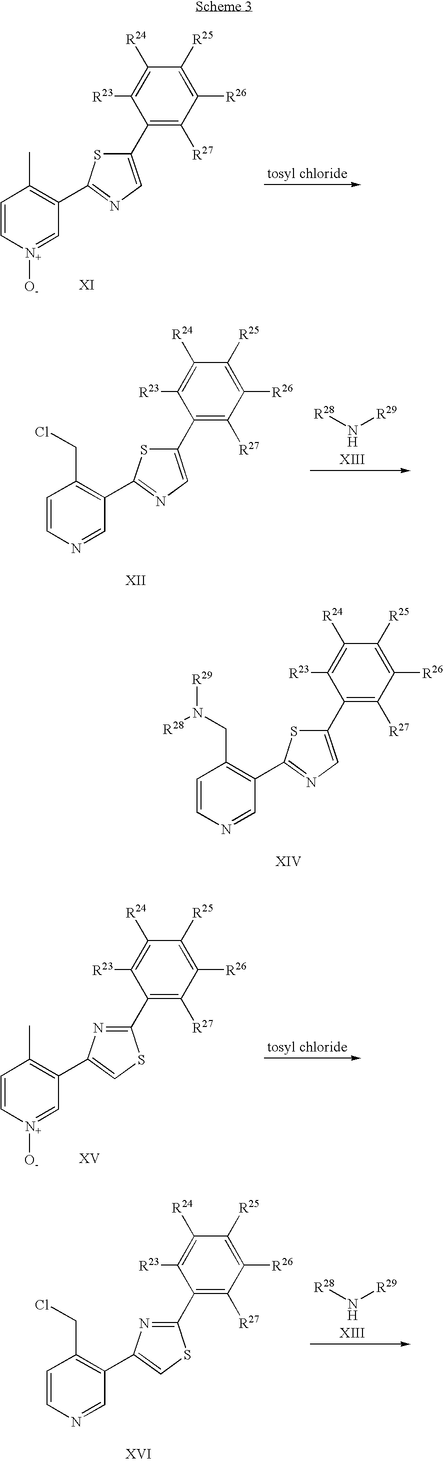

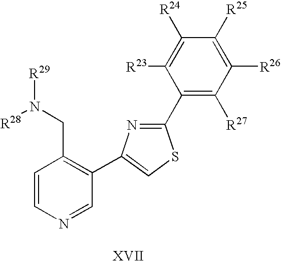

Compounds of Formula I, when A or G is a 4-methyl pyridyl, can be alkylated using a base, such as LDA, followed by treatment with an electrophilic reagent, such as an alkyl iodide, as shown in Scheme 4. Other bases commonly employed in the art, such as n-butyl lithium or tert-butyl lithium, and other electrophilic reagents commonly employed in the art, such as alkyl bromides, alkyl chlorides, akyl tosylates, or alkyl triflates, may also be utilized. Separation by chromatography (column chromatography, flash chromatography, preparative TLC, or HPLC) affords the alkylated thiazoles of Formulae XIX and XII.

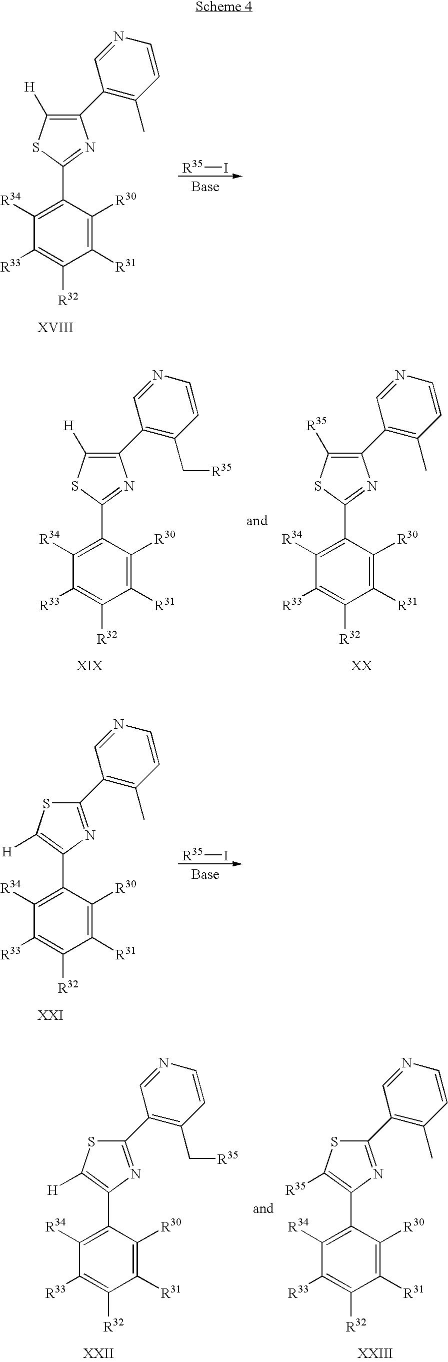

Compounds of Formula I, when A or G is a 4-chloropyridyl, can be treated with an amine, as shown in Scheme 5, to form 4-aminopyridines of formulae XXV and XXVII.

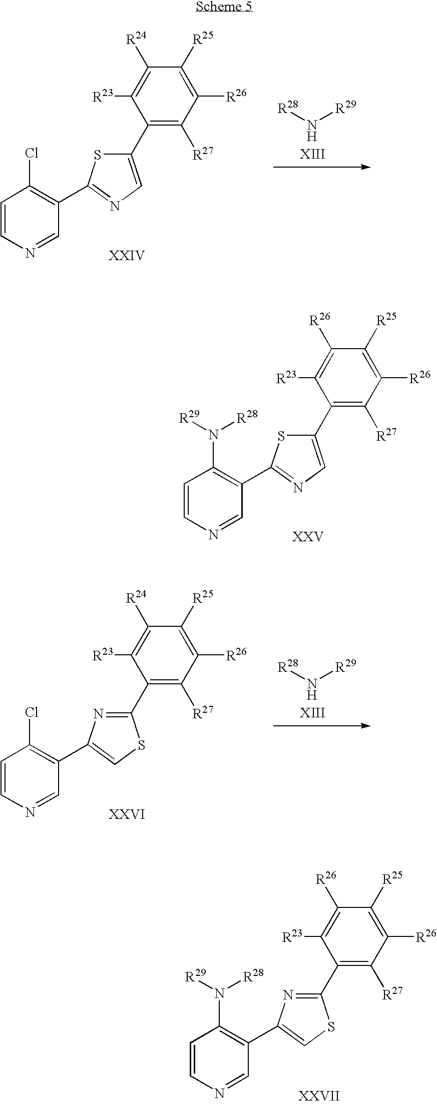

The present invention is further illustrated by the following examples which should not be construed as limiting in any way. The contents of all cited references (including literature references, issued patents, published patent applications as cited throughout this application) are hereby expressly incorporated by reference.

### EXAMPLES

### Preparation of the Compounds of the Invention

General. All reagents are commercially available unless otherwise specified. Reagents were used as received unless otherwise specified. Proton NMR data is reported downfield from TMS; coupling constants are in hertz. LCMS mass spectral data were obtained using a Hewlett-Packard 1100 HPLC equipped with a quaternary pump, a variable wavelength detector set at 254 nm, a YMC pro C-18 column (2×23 mm, 120 Å), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonitrile with 0.018% TFA. Gradient elution from 10% B to 95% B over 3.5 min at a flowrate of 1.0 muLmin was used with an initial hold of 0.5 min and a final hold at 95% B of 0.5 min. Total run time was 6.5 min. Purification by HPLC was performed using a Gilson HPLC system (TV/VIS-155 detector, 215 liquid handler, 306 pumps, 819 injection valve and an 811 C mixer, the column was a YMC Pro C 18 (75×30, 5 μm, 120A); the eluents were A: water with 0.1% TFA, and B: acetonitrile with 0.1% TFA; gradient elution from 10% B to 90% B over 12 min with a final hold at 90% B for 2 min; fiowrate was 25 mL per minute. NMR data are in agreement with the structure of all prepared compounds. Elemental analyses were obtained at Robertson Microlit Laboratories, Madison N.J. Melting points are uncorrected.

### Preparation of Intermediate A: 4-Methyl-3-cyanopyridine

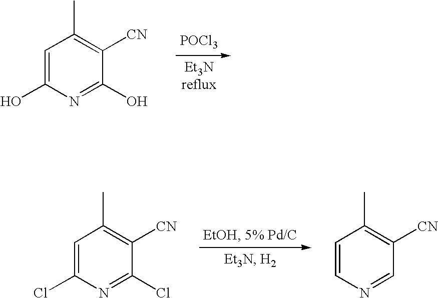

Step 1. 2,6-Dihydroxy-4-methyl-3-pyridinecarbonitrile (150 g, 1 mol) and phosphorus oxychloride (600 mL, 6.4 mol) were stirred under an Ar atmosphere and triethylamine (300 mL, 2.1 mol) was added. After refluxing for 16 h, the mixture was concentrated in vacuo, and the residue partitioned between ice water (6 L) and dichloromethane (2 L). The organic phase was washed with aqueous sodium bicarbonate solution, then brine, dried (Na₂SO₄), and then filtered through a pad of silica gel (465 g) on a sintered glass funnel. Elution with dichloromethane and concentration of the filtrate in vacuo afforded 109.6 g (58.6%) of 2,6-dichloro-4-methyl-3-cyanopyridine as a colorless crystalline solid, mp 108-110° C.: TLC Rf 0.23 (1:1 hexane-dichloromethane, Rf 0.31 (3:1 hexanes-EtOAc); ¹H NMR (CDCl₃) δ 7.3 (d, 1H), 2.3 (s, 3H); GCMS 187 (M+H⁺).

Step 2. 2,6-Dichloro-4-methyl-3-cyanopyridine (40.8 g, 0.22 mol) was dissolved in anhydrous ethanol (680 mL) and triethylamine (120 mL) by warming, and the solution hydrogenated over 5% palladium on carbon at 10 psi of hydrogen. Upon completion of the reaction, catalyst was removed by filtration. The filtrate was concentrated in vacuo. The resulting solid was triturated with ether, filtered, and then concentrated in vacuo to afford 16.3 g (63.1%) of 4-methyl-3-cyanopyridine as colorless needles: mp (40-45° C., slowly melts); ¹H NMR (CDCl₃) δ 8.8 (s, 1H), 8.5 (d, 1H, J=5 Hz), 7.3 (d, 1H, J=5 Hz), 2.6 (s, 3H); GCMS 118 (M⁺).

### General Method A: Preparation of 4-Substituted-3-cyanopyridines

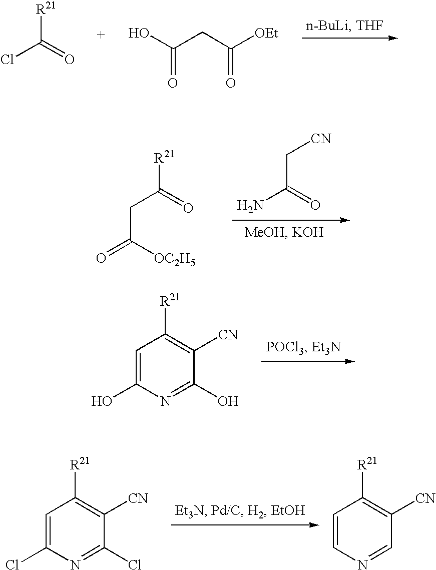

Step 1. mono-Ethyl malonate (35.0 g, 265 mmol) and THF (300 mL) were placed into a 500 mL round-bottomed flask and cooled to −70° C. under Ar. To this solution was added 330 mL of 1.6 M n-BuLi (2.0 equiv., 530 mmol) slowly and the solution allowed to stir for 10 min at −70° C. The acid chloride was added to the solution slowly, stirred for one more h at −70° C., and then the reaction temperature was allowed to go to rt overnight. The solution was concentrated in vacuo and the residue was partitioned between 1N HCl, (200 mL) and Et₂O (2×300 mL). The organic layer was washed sequentially with saturated NaHCO₃ solution (200 mL) and H₂O (200 mL), then dried over Na₂SO₄. The filtrate was concentrated and the crude product was purified by chromatography using hexanes-EtOAc (95:5). The average yields of the beta-ketoesters were 30-50%.

Step 2. The beta-ketoester (347 mmol) and 2-cyanoacetamide (347 mmol) were placed into a 500 mL round-bottomed flask and dissolved in 100 mL of THF under Ar. To this solution was slowly added a solution of KOH (1.1 equiv., 25.2 g, 382 mmol) in 150 mL MeOH. The solution allowed to stir at 70° C. for 8 h, during which time a solid slowly formed. The reaction mixture was cooled the solution to rt and the solid was filtered. The solid was dissolved in warm water (250 mL) and concentrated. HCl was added slowly until the pH was 1-2. The resulting solid was filtered and dried to afford the 4-substituted-2,6-dihydroxy-3-cyanopyridine. The average yields of the 4-substituted-2,6-dihydroxy-3-cyanopyridines were 30-90%.

Step 3. In a 500 mL round-bottomed flask were placed the 4-substituted-2,6-dihydroxy-3-cyanopyridine (314 mmol) and POCl₃ (3.3 equiv, 1035 mmol, 95.3 mL) under Ar. Triethylamine (471 mmol, 65.5 mL) was added very slowly using an ice bath for cooling. The reaction mixture was heated to 130° C. for 8 h under Ar after the addition was finished. After cooling to rt, the reaction mixture was concentrated in vacuo and poured into ice (150 g). The residue was partitioned between CH₂Cl₂ (3×200 mL) and ice water. The separated organic layer was washed sequentially with NaHCO₃ (saturated 200 mL) and H₂O (200 mL), then dried over Na₂SO₄. The filtrate was concentrated and purified by column chromatography using hexanes-EtOAc (80:20) as eluant. The average yields of the 4-substituted-2,6-dichloro-3-cyanopyridines were 35-50%.

Step 4. Into a 500 mL round-bottomed flask were placed the 4-substituted-2,6-dichloro-3-cyanopyridines (232 mmol), 10% Pd/C (2.0 g), Et₃N (927 mmol, 130 mL) and EtOH (300 mL). The mixture was hydrogenated at atmospheric pressure for 24 to 48 h at rt. The catalyst was removed by filtration and the filtrate was concentrated. The residue was partitioned between CH₂Cl₂ (3×200 mL) and H₂O (200 mL), and then the separated organic layer was dried over Na₂SO₄. Concentration and purification by column chromatography using hexanes-EtOAc (95:5) afforded the 4-substituted-3-cyanopyridines in average yields of 85-95%.

### Preparation of Intermediate B: 4-Ethyl-3-cyanopyridine

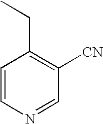

4-Ethyl-3-cyanopyridine was prepared according to General Method A: TLC Rf 0.50 (70:30 hexane-EtOAc); ¹H NMR (CDCl₃) δ 8.80 (d, 1H), 8.62 (d, 1H), 7.26 (dd, 1H), 2.84 (q, 2H), 1.30 (t, 3H); MS 133.1 (M+H⁺).

### Preparation of Intermediate C: 4-(2-Propyl)-3-cyanopyridine

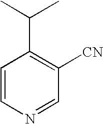

4-Propyl-3-cyanopyridine was prepared according to General Method A: TLC Rf 0.40 (70:30 hexane-EtOAc); ¹H NMR (CDCl₃) δ 8.80 (d, 1H), 8.75 (d, 1H), 7.26 (dd, 1H), 3.32 (q, 2H), 1.30 (t, 6H); MS 146 (M+H⁺).

### Preparation of Intermediate D: 4-2-Cyclopentyl)-3-cyanopyridine

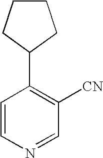

4-Cyclopentyl-3-cyanopyridine was prepared according to General Method A: TLC Rf 0.70 (70:30 hexane-EtOAc); ¹H NMR (CDCl₃) δ 8.72 (s, 1H), 8.60 (d, 1H), 7.24 (d, 1H), 3.36 (t, 1H), 2.18 (m, 2H), 1.80 (t, 6H); MS 173 (M+H⁺).

### Preparation of Intermediate E: 4-(1-Propyl)3-cyanopyridine

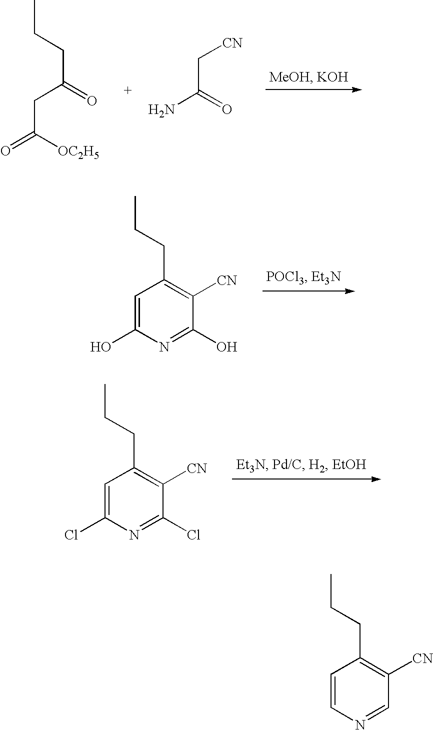

Step 1. Ethyl 3-oxohexanoate (50 g, 0.32 mol) and 2-cyanoacetamide (26.6 g, 0.32 mol) were dissolved in methanol (100 mL). A solution of KOH (20.7 g, 0.37 mol) in methanol (150 mL) was added slowly using an additional funnel. The resulting mixture was refluxed at 70° C. overnight. After the reaction, the white precipitate that formed was filtered and collected. The crude product was dissolved in warm water (250 mL, 50-60° C.). Concentrated HCl was added dropwise with stirring until the pH was 1-2. The white precipitate that formed upon addition of the HCl was filtered and collected, washed with ice water, and dried in a vacuum oven overnight. 2,6-Dihydroxy-4-propyl-3-cyanopyridine (33.1 g) was produced as a white solid.

Step 2. Under Ar, POCl₃ (56.5 mL, 0.614 mol) was added dropwise into an ice-bath cooled, three-neck round-bottomed flask containing 2,6-dihydroxy-4-propyl-3-pyridinecarbonitrile (33.1 g, 0.186 mol). Then Et₃N (38.86 mL, 0.279 mol) was added into the mixture very slowly with cooling. After the addition was complete, the mixture was warmed to rt, then heated under reflux at 140° C. overnight. After cooling to rt, the excess POCl₃ was evaporated. The brown residue that remained was added slowly into 500 g of crushed ice with stirring. Then concentrated NaOH solution was added dropwise with stirring until the pH reached 8. The aqueous solution was extracted with CH₂Cl₂ (3×500 mL). The organic extracts were combined and evaporated to give a brown solid. The crude product was purified by silica gel chromatography using 2% EtOActhexane as eluant to give 2,6-dichloro-4-propyl-3-cyanopyridine (24.1 g) as a light yellow solid.

Step 3. 2,6-Dichloro-4propyl-3-pyridinecarbonitrile (24.1 g, 0.112 mol) and 10% Pd on carbon (3.5 g) were mixed in a 500 mL round-bottomed flask. Denatured EtOH (300 mL) and Et₃N (62.4 mL, 0.448 mol) were then added. The reaction mixture was degassed, filled with Ar, and then degassed again. After this step was repeated 3 more times, H₂ was filled into the flask using a hydrogen balloon. Connected with the hydrogen balloon, the reaction mixture was stirred overnight. After the reaction, the mixture was degassed again. The Pd/C was filtered and the filtrate was evaporated until a light yellow precipitate formed inside. The turbid filtrate was cooled in the ice bath for about 10 min and then filtered. The filtrate was concentrated and the brownish oil that remained was purified by silica gel chromatography using 20% EtOAc/hexane as eluant. 4-(1-Propyl)-3-cyanopyridine (4.24 g) was produced as light yellow oil in an overall 9.1% yield (3 steps): LCMS t_{R}=2.11 min. 147.2 (M+H⁺); ¹H NMR δ 9.00 (s, 1H), 8.50 (d, 1H), 7.33 (d, 1H), 2.84 (t, 2H), 1.77 (m, 2H), 1.00 (t, 3H).

### Preparation of Intermediate F: 4-Phenyl-3-cyanopyridine

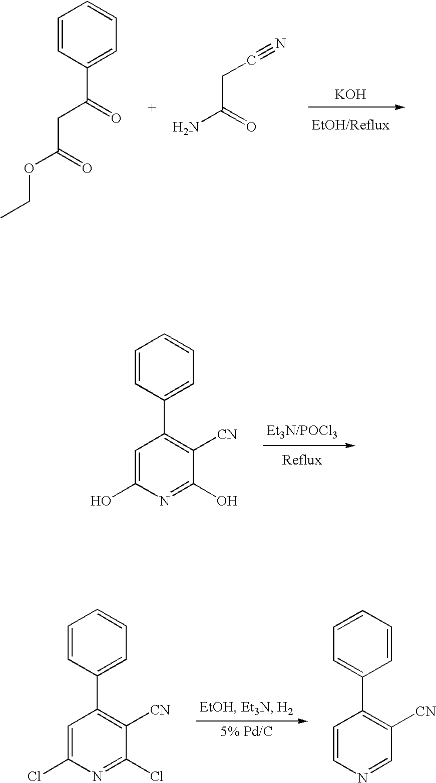

Step 1. Ethyl 3-oxo-3-phenylpropanoate (51.9 mL, 0.300 mol) and 2-cyanoacetamide (25.2 g, 0.300 mol) were dissolved in ethanol (100 mL). The mixture was heated to 50° C. under Ar. To this reaction mixture was added a solution of KOH (21.8 g, 0.330 mol) in ethanol (100 mL) via an additional funnel. The reaction was refluxed for approximately 17 h. After cooling to rt, the reaction mixture was filtered. The solid product was washed with ethanol and dried in vacuo overnight at 45° C., providing 12.5 g (19.6%) of 2,6-dihydroxy-4-phenyl-3-cyanopyridine as a white solid.

Step 2. 2,6-Dihydroxy-4-phenyl-3-cyanopyridine (6.0 g, 28.2 mmol) and triethylamine (4.2 mL, 30.6 mmol) were charged together into a round-bottomed flask. To this via syringe was added phosphorus oxychloride (8.2 mL, 90.4 mmol). The reaction mixture was refluxed for 17 h under Ar, then concentrated to an oil under reduced pressure to remove excess POCl₃. This oil was then poured slowly into a beaker with ice-water. The brown precipitate that formed was filtered, washed with copious amounts of water, then dried in vacuo overnight at 45° C. The solid was purified by silica gel chromatography (mobile phase dichloromethane), providing 3.83 g (54.5%) of 2,6-dichloro-phenyl-3-cyanopyridine as an off-white solid.

Step 3. Into a dry round-bottomed flask was charged 5% palladium on carbon (0.38 g) and anhydrous ethanol (5 mL). Into another flask was charged 2,6-dichloro-4:phenyl-3-cyanopyridine (3.83 g, 15.4 mmol), triethylamine (8.57 mL, 61.5 mmol) and anhydrous ethanol (80 mL). This solution was transferred to the reaction flask and this flask was then purged with Ar. The flask was evacuated and then purged with Ar; this process was repeated twice more. A balloon of H₂ was attached to the flask and the reaction was then purged with hydrogen, then evacuated. The H₂ was released into the reaction flask and the reaction mixture was hydrogenated for 48 h. The reaction mixture was filtered and washed with ethanol. The filtrate was concentrated and the resulting oil was purified by column chromatography (mobile phase 20% EtOAc/hexane), providing 2.0 g (72%) of 4phenyl-3-cyanopyndine as a white solid. TLC Rf=0.1618 (20% EtOAc/Hex); ¹H NMR (CD₂Cl₂) δ 7.50 (d, 1H, J=5.3 Hz) 7.58-7.55 (m, 3H), 7.63-7.62 (m, 3H), 8.80 (d, 1H, J=5.3 Hz), 8.94 (s, 1H); GCMS m/z 180 (M⁺), t_{R}=8.0 min.

### Preparation of Intermediate G: 4-Cyclopropyl-3-cyanopyridine

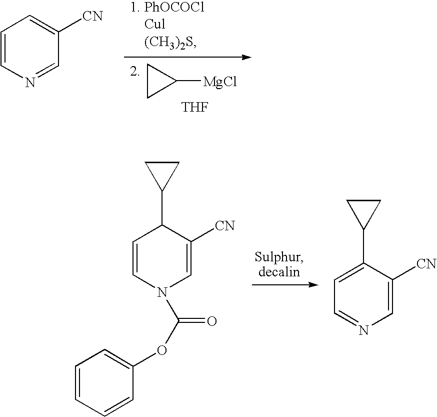

Step 1. To a mixture of CuI (1.37, 0.0072 mol), dimethyl sulphide (33.5 mL, 0.46 mol) and 3-cyanopyridine (15.0 g, 0.144 mol) in anhydrous THF (390 mL) at −25 to −20° C. was added phenyl chloroformate (23.9 mL, 0.19 mol) and the mixture was stirred at this temperature for 15-20 min. To this suspension at −25 to −20° C. was added cyclopropyl magnesium bromide (126 mL, 0.173 mol) over 20-30 min. The mixture was stirred at −25 to −20° C. for 15 min, then warmed slowly to rt over 45-50 min. The reaction mixture was quenched with 20% NH₄Cl (105 mL), followed by extraction of the aqueous layer with diethyl ether (300 mL). The organic layer was washed sequentially with an aqueous solution of 1:1 20% NH₄Cl/NH₄OH (2×45 mL), water (75 mL), 10% HCl (2×75 mL), water (75 mL) and brine (125 mL), then dried over anhydrous Na₂SO₄. The solution was concentrated to dryness to give the crude 3-cyano-4-cyclopropyl-1-phenoxycarbonyl-1,4-dihyropyridine.

Step 2. A mixture of the crude dihydropyridine and sulphur (3.9g, 0.144 mol) was heated in decalin (250 mL) for a period of 3 h. The reaction mixture was cooled to rt and vacuum distilled to give 1.73 g (8.5%) of 4-cyclopropyl-3-cyanopyridine: Rf 0.24 (25% EtOAc/hexane); LCMS t_{R}=1.50 min, 145.10 (M+H⁺); ¹H NMR (CDCl₃) δ 8.75 (1H, s), 8.60 (1H, d), 6.80 (1H, d), 2.30 (1H, m), 1.32 (2H, m), 0.97 (2H, m).

### Preparation of Intermediate H: 4-(tert-Butyl)-3-cyanopyridine

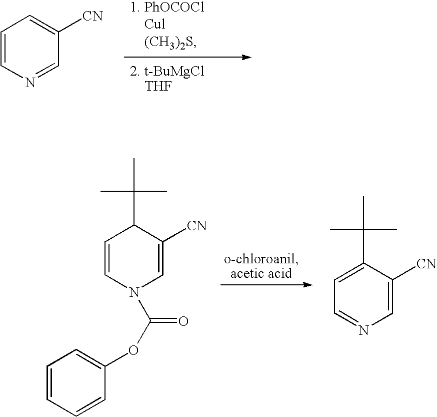

Step 1. In a 2000 mL, three-necked flask equipped with an overhead stirrer were placed 3-cyanopyridine (20.8 g, 0.2 mol), CuI (1.9 g, 0.01 mol), methyl sulfide (48 mL), and 600 mL of THF under Ar. The solution was cooled to −40° C. and phenylchloroformate (25.1 mL, 0.2 mol) was added via an additional funnel with stirring. After 25 min, 0.1 M solution of tert-butylmagnesium chloride in THF (200 mL, 0.2 mol) was added dropwise over 1 h. The mixture was stirred at −40° C. for 2 h, then at rt overnight. Aqueous 20% NH₄Cl (300 mL)and Ether (400 mL) was added into the mixture. After stirring for 5 min, the organic layer was collected and then washed sequentially with 200 mL of NH₄Cl/NH₄OH (50/50) twice, 200 mL of water once, 200 mL of 10% HCl twice, 200 mL of water once, and then 200 mL of brine once. After drying over MgSO₄, the solution was filtered and concentrated to yield a brown oil. The crude product was purified by silica gel chromatography (10% EtOAc/hexane) to give 10.0 g of the intermediate dihydropyridine as a brown oil.

Step 2. The intermediate dihydropyridine (10.0 g) was dissolved in dry toluene (100 mL). A solution of o-chloranil (12.3 g, 0.5 mol) in 70 mL of acetic acid was added dropwise. The mixture was stirred at rt for 8 h and then concentrated. Toluene (100 mL), ether (100 mL), celite (10 g), and 10% NaOH solution (200 mL) were then added. The mixture was stirred for 15 min and filtered through celite. The dark organic layer was washed with 100 mL portions of 10% NaOH and water, then extracted with 10% HCl (4×100 mL). The combined organic extracts were concentrated to approximately 100 mL, cooled, made basic with 20% NaOH, and then extracted with CH₂Cl₂ (3×100 mL). The combined organic layer was washed with brine, dried with K₂CO₃, and then concentrated to yield 3.8 g of 4-(tert-butyl)-3-cyanopyridine as a yellow oil (overall yield is 11.9%): LCMS t_{R}=2.23 min, 161.2 (M+H⁺); ¹H NMR 8.80 (s, 1H), 8.65 (d, 1H), 7.40 (d, 1H), 1.50 (s, 9H).

### Preparation of Intermediate I: 4-(4-Fluorophenyl)-3-cyanopyridine

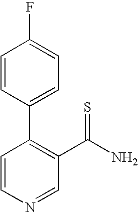

4-(4-Fluorophenyl)-3-cyanopyridine was prepared according to the method described for Intermediate H from 3-pyridinecarbonitrile (3.12 g, 0.03 mol), providing 1.08 g (overall yield 18.2%) of 4-(4-fluorophenyl)-3-cyanopyridine as a white solid: LCMS t_{R}=2.33 min, 199.3 (M+H⁺).

### Preparation of Intermediate J: 4-Methoxy-3-cyanopyridine

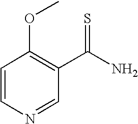

Step 1. A stirred mixture of (1-ethoxylidene)malononitrile (50 g, 0.36 mol), dimethylformamide dimethyl acetal (84.9 mL, 0.6 mol) and anhydrous methanol (110 mL) was refluxed under Ar for 1 h, then left to cool and stand at rt overnight. After concentration in vaccuo, the resulting solid was triturated with ice-cold methanol, filtered, and then dried to afford 41.78 g (65.5%) of 1,1-dicyano-2-methoxy-4-di&ethylamino-1,3-butadiene as reddish-pink crystals, mp 131-132° C.; TLC Rf 0.24 (dichloromethane), Rf 0.31 (2:1 hexane-acetone); ¹H NMR (CD₂Cl₂) δ 7.65 (d, 1H), 5.1 (d, 1H), 4.1 (s, 3 H), 3.2 (s, 3H), 2.9 (s, 3H); LCMS 178 (M+H⁺).

Step 2. Hydrogen chloride gas was vigorously bubbled into a stirred suspension of 1,1-dicyano-2-methoxy-4-dimethylamino-1,3-butadiene (8.29 g, 46.8 mmol) in anhydrous methanol (178 mL) for 5 min periods twice during the day, then left to stir at rt over the weekend. The yellow solution was concentrated in vacuo, and the resulting solid stirred in methanol while sodium bicarbonate was cautiously added until gas evolution ceased, and the pinkish-red liquid was basic to pH paper. The reaction mixture was concentrated to a solid, triturated with dichiloromethane, and then filtered. The filtrate was concentrated in vacuo to afford 2-chloro-3-cyano-4-methoxypyridine as a pink solid (7.0 g, 89%). The product could be recrystallized from methanol as fine, pastel yellow needles, mp 168.5-171° C.: ¹H NMR (CDCl₃) δ 8.4 (d, 1H), 6.9 (d, 1H), 4.0 (s, 3 H); LCMS 169 (M+H⁺). Anal. Calcd for C₇H₅ClN₂O: C, 49.87; H, 2.99; N, 16.62; Cl, 21.03. Found: C, 49.87; H, 2.97; N, 16.63; Cl, 20.95.

Step 3. A solution of 2-chloro-3-cyano-4-methoxypyridine (3.4 g, 20.0 mmol) in anhydrous ethanol (75 mL) was hydrogenated over 5% Pd/C (340 mg) at 10 psi. Upon completion of the reaction, catalyst was removed by filtration. The filtrate was in vacuo to afford 2.54 g (94.7%) of 4-methoxy-3-cyanopyridine as a colorless solid. A sample was recrystallized from dichloromethane/hexane, mp 124.5-126° C. (colorless needles): TLC Rf 0.2 (2% methanol/dichloromethane); TLC Rf 0.1 (1:1/hexane:EtOAc); ¹H NMR (CDCl₃) δ 8.7 (s,1H), 8.6 (d, 1H), 6.9 (d, 1H), 4.0 (s, 3H); GCMS 134 (M⁺). Anal. Calcd for C₇H₆N₂O: C, 62.68; H, 4.51; N, 20.88. Found: C, 62.43; H, 4.48; N, 20.75.

### Preparation of Intermediate K: 4-Methylpyridine-3-thiocarboxamide

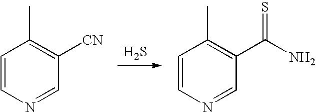

Hydrogen sulfide gas was bubbled into a solution of 4-methyl-3-cyanopyridine (40.8 g, 0.346 mol) in absolute-ethanol (680 mL) and triethylamine (0.33 mol) with ice cooling for 1 h. The reaction mixture was stirred overnight and then the solvent was removed in vacuo. The residue was dissolved in EtOAc (500 mL) and the solution was heated at 50-55° C. for 4.0-4.5 h, then allowed to cool to rt. The mixture was filtered, the solid was triturated and washed with more EtOAc, and then filtered. The filtrate was concentrated in vacuo to afford crude product. The crude was purified by taking it back up into dichloromethane (100 mL), heating the mixture to reflux, then allowing it to cool with stirring. The solid was filtered, washed with dichloromethane, and then dried to afford 24.1 g (72%) of 4-methylpyridine-3-thiocarboxamide as a sand-colored solid, mp 104.5-106° C.: TLC Rf 0.08 (5% methanol/dichloromethane); TLC R0.18 (EtOAc); ¹H NMR (DMSO-d₆) δ 10.1 (broads, 1H), 9.6 (broad s, 1H), 8.4 (d, 1H), 8.3 (s, 1H), 7.2 (d, 1H), 2.3 (s, 3H); LCMS 153 (M+H⁺).

### General Method B: Preparation of 4-Substituted Pyridine-3-thicarboxamides

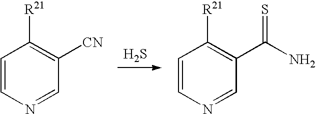

Hydrogen sulfide was bubbled for 30 min into a solution containing the 4-alkyl-3-cyanopyridines (178 mmol) in DMF (300 mL). Diethylamine (1.5 eq) was added and the mixture was heated at 60° C. for 1 h. The reaction mixture was concentrated and the residue was partitioned between CH₂Cl₂ (3×200 mL) and H₂0 (200 mL). The organic layer was dried (Na₂SO₄) and purified by column chromatography using 60:40 hexanes-EtOAc to afford the pyridine thiocarboxamides. The average yield was 80-95%.

### Preparation of Intermediate L: 4-Ethylpyridine-3-thiocarboxamide

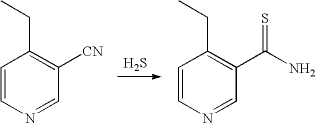

4-Ethylpyridine-3-thiocarboxamide was prepared according to General Method B: TLC Rf 0.55 (EtOAc); LCMS 167.1 (M+H⁺); ¹H NMR (CDCl₃) δ 8.50 (s, 1H), 8.46 (d, 2H), 7.96 (bs, 1H), 7.66 (bs, 1H), 2.86 (q, 2H), 1.30 (t, 3H).

### Preparation of Intermediate M: 4-(2-Propyl)pyridine-3-thiocarboxamide

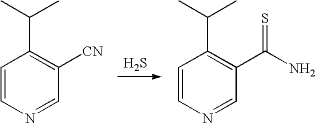

4-(2-Propyl)pyridine-3-thiocarboxamide was prepared according to General Method B: TLC Rf 0.10 (50% EtOAc/hexanes); LCMS 181 (M+H⁺); ¹H NMR (CDCl₃) δ 8.48 (d, 1H), 7.24 (s, 1H), 7.20 (d, 1H), 3.46 (m, 1H), 1.26 (d, 6H).

### Preparation of Intermediate N: 4-(2-Cyclopentyl)pyridine-3-thiocarboxamide

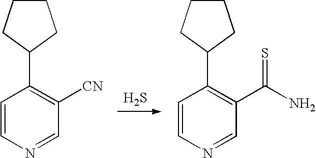

4-(Cyclopentyl)pyridine-3-thiocarboxamide was prepared according to General Method B: TLC Rf 0.30 (60/40 hexanes/EtOAc); LCMS 206.8 (M+H⁺); ¹H NMR (CDCl₃) δ 8.75 (s, 1H), 8.40 (d, 2H), 7.30 (d, 1H), 3.38 (t, 1H), 2.08 (m, 2H); 1.70 (m, 6H).

### Preparation of Intermediate O: 4-(1-Propyl)pyridine-3-thiocarboxamide

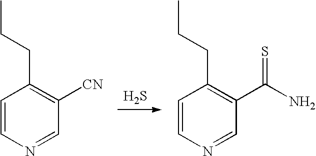

4-(1-Propyl)pyridine-3-thiocarboxamide was prepared according to General Method B: LCMS t_{R}=1.05 min, 181.1 (M+H⁺); ¹H NMR (CDCl₃) δ 8.05 (s, 1H), 8.00 (d, 1H), 7.15 (d, 1H), 2.80 (t, 2H), 1.66 (m, 2H); 0.98 (t, 3H).

### Preparation of Intermediate P: 4-Phenylpyridine-3-thiocarboxamide

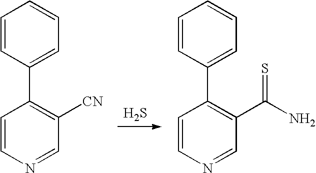

4-Phenyl-3-cyanopyridine (2.0 g, 11 mmol) was dissolved into DMF (40 mL). The reaction flask was attached to a scrubber (bleach). The reaction was cooled in an ice-water bath and hydrogen sulfide (excess) was bubbled in via needle for 40 min. To the mixture was added diethylamine (1.72 mL, 16.6 mmol). The mixture was heated to 60° C. for 45 min. The reaction was then concentrated under reduced pressure and purified by column chromatography (mobile phase 30% EtOAc/hexane to 60% EtOAc/hexane). This yielded 1.85 g (77.8%) of 4-phenylpyridine-3-thiocarboxarnide as a yellow solid: TLC Rf 0.05 (40% EtOAc/hexanes); t_{R}=1.37; ¹H NMR (CDCl₃) δ 6.57-6.50 (m, 2H), 7.45-7.44 (m, 4H), 7.52 (m, 2H), 8.63 (d, 1H), 9.01 (s, 1H); LCMS (ES) m/z,215.1 (M+H⁺).

### Preparation of Intermediate Q: 4-Cyclopropylpyridine-3-thiocarboxamide

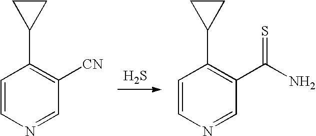

To a solution of 4-cyclopropyl-3-cyanopyridine (4.83 g, 34 mmol) in absolute ethanol (100 mL) upon cooling, was purged hydrogen sulphide gas for a period of 1 h. To this solution was added diethylamine (5.3 mL, 51 mmol) and the mixture was heated to 50-55° C. for a period of 4.0-4.5 h. The reaction mixture was then stirred for 16-18 h at rt in order to consume the remaining amount of starting material. The reaction mixture was Concentrated in vacuo and subjected to silica gel chromatography using 20-100% EtOAc-hexane to yield 5.01 g (82%) of 4-cyclopropylpyridine-3-thiocarboxamide: LCMS t_{R}=0.70 min, 179 (M+H⁺); ¹H NMR (DMSO-d₆) δ 10.21 (br s, 1H), 9.75 (br s, 1H), 8.32 (d, 1H), 8.26 (s, 1H), 6.83 (d, 1H), 2.12 (m, 1H), 1.04 (m, 2H), 0.80 (m, 2H).

### Preparation of Intermediate R: 4-(tert-Butyl)pyridine-3-thiocarboxamide

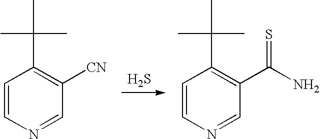

4-(tert-Butyl)pyridine-3-thiocarboxamide was prepared according to General Method B: LCMS t_{R}=0.91 min, 195.2 (M+H⁺); ¹H NMR (CDCl₃) δ 8.38 (d, 1H), 8.26 (s, 1H), 8.00 (br s, 1H), 7.60 (br s, 1H), 7.35 (d, 1H); 1.50 (s, 9H).

### Preparation of Intermediate S: 4-(4-Fluorophenyl)pyridine-3-thiocarboxamide

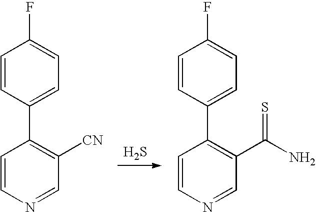

4(4-Fluorophenyl)pyridine-3-thiocarboxamide was prepared according to General Method B: LCMS t_{R}=1.52 min, 233.2 (M+H⁺).

### Preparation of Intermediate T: 4-Methoxypyridine-3-thiocarboxamide

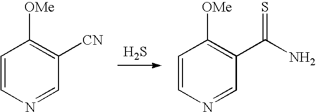

Hydrogen sulfide gas bubbled into a solution of 3-cyano-4-methoxypyridine (14.7 g, 87.1 mmol) in absolute ethanol (270 mL) and triethylamine (130 mmol) with ice cooling for 1 h. The reaction mixture was stirred overnight and then the solvent was removed in vacuo. The residue was dissolved in EtOAc (200 mL) and heated at 50-55° C. for 4.0-4.5 h, then allowed to cool to rt. The mixture was filtered, and the solid was triturated with more EtOAc and then filtered. The filtrate was concentrated in vacuo to afford the crude product. This was purified by taking it back up into dichloromethane (75 mL), heating the mixture to reflux, then allowing it to cool with stirring. The solid was filtered, washed with dichloromethane, and then dried to afford 9.6 g (65.3%) of 4-methoxypyridine-3-thiocarboxamide as a pale yellow solid: TLC Rf 0.21 (5% methanol/dichloromethane); TLC Rf 0.12 (EtOAc); ¹H NMR (DMSO-d₆) δ 10.1 (broad s, 1H), 9.4 (broad s, 1H), 8.6 (s, 1H), 8.4 (d, 1H), 7.1 (d, 1H), 3.9 (s, 3H); LCMS 153 (M+H⁺).

### Preparation of Intermediate U: 2-(3-Pyridyl)thioacetamide

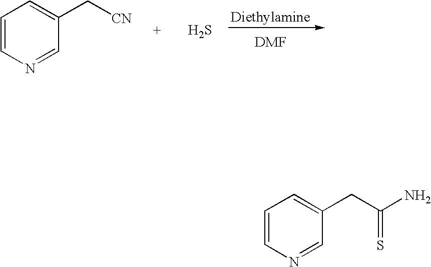

Hydrogen sulfide gas was bubbled into a solution of 6.0 g (51 mmol) of 3-pyridylacetonitrile in 100 mL anhydrous DMF under Ar at rt at a moderate rate for 20 min. The reaction was warmed to 60° C., then a solution of diethylamine (7.88 mL, 76.5 mmol) in 10 mL DMF was added in one portion. After 1.5 h, the reaction mixture was cooled and Ar was bubbled through the reaction for 1 h. The DMF was evaporated. The residue was dissolved in EtOAc and purified by flash chromatography using EtOAc as eluant. ¹H NMR and MS data were consistent with the product.

### Preparation of Intermediate V: 4-Cyanoisoquinoline

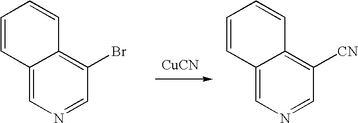

Into a 250 mL round-bottomed flask were placed 4-bromoisoquinoline (50.0 mmol, 10.4 g), CuCN (100.0 mmol, 9.0 g) and DMF (150 mL) under Ar. The solution was heated at 140° C. for 12 h. The reaction mixture was filtered over celite and the filtrate was concentrated. The residue was partitioned between CH₂Cl₂ (3×100 mL) and H₂O (100 mL), and then the organic layer was dried (Na₂SO₄). Concentration and purification of the crude product by column chromatography using 80:20 hexanes-EtOAc afforded 4-cyanoisoquinoline (45%).

### Preparation of Intermediate W: Isoquinoline-4-thiocarboxamide

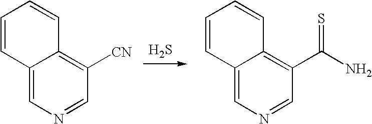

Isoquinoline-4-thiocarboxamide was prepared according to General Method B: TLC Rf 0.60 (EtOAc); ¹H NMR (DMSO-d₆) δ 10.4 (s, 1H), 9.9 (s, 1H), 8.6 (s, 1H), 9.3 (s, 1H), 8.4 (s, 1H), 8.2 (d, 1H), 7.8 (dd, 1H), 7.6 (dd, 1H); LCMS 189.1 (M+H⁺), t_{R} 1.08 min.

### Preparation of Intermediate X: 4-Methyl-3-acetylpyridine

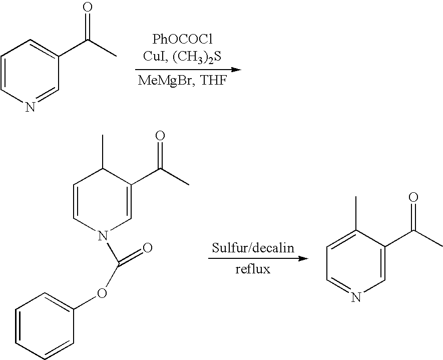

Step 1. A solution of 3-acetylpyridine (100 g, 0.82 mol), dimethyl sulfide (400 mL, 5.4 mol) and copper (I) iodide (7.94 g, 0.041 mol) in anhydrous THF (2 L) was stirred at rt under Ar. Phenyl chloroformate (0.4 mL, 0.82 mol) was then added, producing a dark brown precipitate. After 30 min, the mixture was cooled below −21° C. and methyl magnesium bromide (1.4 M in 3:1 toluene-THF, 586 mL, 0.82 mol) was added over 50 min, keeping the reaction temperature below −15° C. The color lightened as the mixture became a solution; a lime green precipitate formed near the end of the addition, but redissolved upon completion. The mixture was stirred and allowed to warm slowly, after 2 h it had warmed to 8.8° C. Saturated aqueous ammonium chloride solution (500 mL) was added. After stirring for 10 min, the mixture was poured into a separatory funnel containing water (500 mL). The organic phase was separated, washed with brine (500 mL), dried (Na₂SO₄), filtered and then concentrated in vacuo. The residue was purified by silica gel chromatography using a hexane-EtOAc gradient to afford 134.3 g (63.7) of the intermediate dihydropyridine.

Step 2. A solution of the intermediate dihydropyridine (134.3 g, 0.52 mol) in dichloromethane (100 mL) was added to a stirred suspension of sulfur (16.67 g, 0.52 mol) in decalin and slowly heated to reflux under an Ar sweep. After refluxing 1 h, the reaction mixture was allowed to cool to rt, then filtered through a pad of silica gel. After eluting the decalin with hexane, elution with a hexane-diethyl ether gradient afforded 49.4 g (70.3%) of 4-methyl-3-acetylpyridine as a reddish-brown oil: TLC Rf 0.19 (diethyl ether); TLC Rf 0.14 (1:1 hexane/EtOAc); ¹H NMR (CD₂Cl₂) δ 8.9 (s, 1H), 8.5 (d, 1H), 7.2 (dd, 1H), 2.6 (s, 3H); GCMS m/z 135 (M⁺).

### Preparation of Intermediate Y: 4-(2-Propyl)-3-acetylpyridine

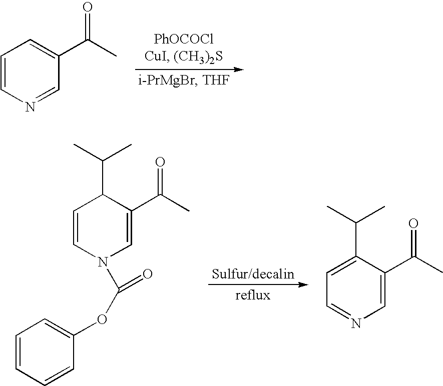

Step 1. To a mixture of CuI (78.5 g, 0.412 mol), dimethyl sulphide (203 mL, 2.76 mol) and 3-acetyl pyridine (50.0 g, 0.412 mol) in anhydrous THF (1100 mL) at rt was added phenyl chloroformate (55.2 mL, 0.44 mol) and the mixture was stirred for 40-50 min. To this suspension at −25 to −20° C. was added isopropyl magnesium chloride (220 mL, 0.44 mol, 2.0 M solution in THF) over 30-40 min. The mixture was stirred at this temperature for 30 min, then warmed slowly to rt over 1.0-1.5 h. The reaction mixture was quenched with 20% NH₄Cl (350 mL), followed by extraction of the aqueous layer with EtOAc (700 mL). The organic layer was washed with 20% NH₄Cl (350 mL), then brine (250 mL), and dried over anhydrous Na₂SO₄. Silica gel chromatography using a 3-10% EtOAc-hexane gradiant yielded 43.5 g of crude 3-acetyl-4-isopropyl-1-phenoxycarbonyl-1,4-dihydropyridine.

Step 2. A mixture of the crude dihydropyridine (43.5 g, 0.153 mol) and sulphur (4.9 g, 0.153 mol) were heated at reflux in decalin (175 mL) for a period of 3 h, then cooled to rt. Purification by silica gel chromatography, eluting first with hexanes, then with a 5-30% EtOAc-hexane gradiant, gave 19.3 g (78%) of the title compound: TLC Rf 0.19 (25% EtOAc/hexane); GCMS (EI) t_{R}=6.2 min; 163 (M⁺); ¹H NMR (CDCl₃) δ 8.76 (s, 1H), 8.57 (d, 1H), 7.30 (d, 1H), 3.55 (m, 1H), 2.60 (s, 3H), 1.22 (d, 6H).

### Preparation of Intermediate Z: 4-Ethyl-3-acetylpyridine

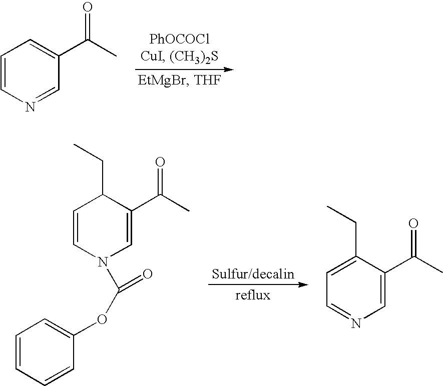

Step 1. 3-Acetylpyridine (5.0 g, 0.0413 mol), copper iodide (7.86 g, 0.0413 mol) and dimethyl sulfide (20.0 mL, 0.272 mol) were dissolved in THF (100 mL, anhydrous). This was stirred at rt for 15 min. To the reaction was added dropwise phenyl chloroformate (5.5 mL, 0.0441 mol) over 10 min. This reaction was then stirred under Ar for 1 h. The reaction was cooled to −25° C. and ethylmagnesium bromide (1M in THF, 44.1 mL, 0.0441 mol) was added dropwise over 40 min. The reaction was stirred at −25° C. for 30 min, then warmed to rt and quenched with 20% NH₄Cl (35 mL). The mixture was extracted with EtOAc, washed with 20% NH₄Cl, brine, and then dried over sodium sulfate. Regioisomers were produced in a 2:1 ratio (desired: undesired). The organic was concentrated to dryness and the crude oil was purified by column chromatography (mobile phase 5% EtOAc/hexane). Phenyl 3-acetyl-4-ethyl-1(4H) pyridine carboxylate was obtained as an orange oil in 40.6% yield, (4.55 g).

Step 2. Phenyl 3-acetyl-4-ethyl-1(4H)-pyridinecarboxylate (3.26 g, 0.0120 mol) and sulfur (0.385 g, 0.0120 mol) were dissolved into decalin (15 mL). The reaction mixture was heated to reflux for 17 h under Ar, then poured onto a silica gel column and washed with copious amounts of hexane. The product was then eluted with a gradient mobile phase (5% EtOAc/hexane to 30% EtOAc/hexane). The product containing fractions were concentrated to dryness to give an orange oil, 1.16 g (64.8%): Rf 0.12 (20% EtOAc/hexane).

### Preparation of Intermediate AA: 4-(1-Propyl)-3-acetylpyridine

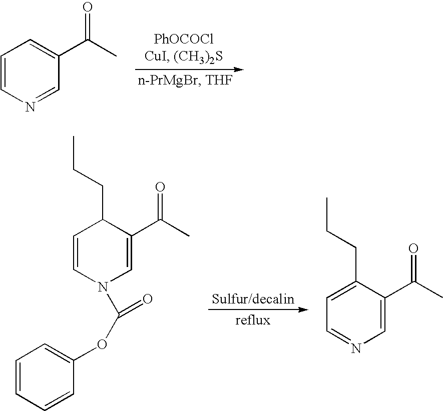

4-(1-Propyl)-3-acetylpyridine was prepared according to the method used to prepare 4-ethyl-3-acetylpyridine: LCMS t_{R}=0.82 min; 164 (M+H⁺); ¹H NMR (CDCl₃) δ 8.86 (s, 1H), 8.56 (d, J=5 Hz, 1H), 7.20 (d, J=5 Hz, 1H), 2.85 (t, J=8 Hz, 2H), 2.63 (s, 3H), 1.61 (m, 2H), 0.97 t, J=7 Hz, 3H).

### Preparation of Intermediate AB: 4-Cyclopropyl-3-acetylpyridine

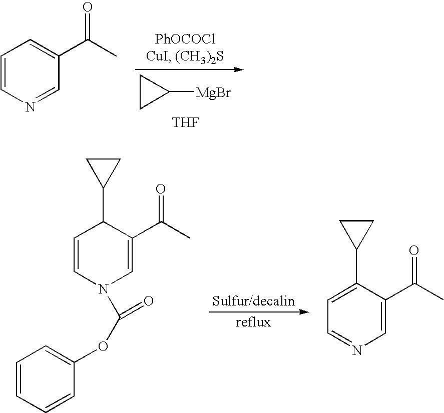

Step 1. Cyclopropyl bromide (50.0 g, 413 mmol) was dissolved in 500 mL of anhydrous THF. Dry magnesium (10.0 g, 411 mmol) was charged to a round-bottomed flask containing a catalytic amount of iodine. 20% of the solution of the cyclopropyl bromide solution was then charged into the flask. After observing bubble formation, the remaining cyclopropyl bromide solution was added over 15 min, thereby causing the reaction mixture to reflux. After 30 min, a 5.0 mL aliquot of the reaction mixture was taken to determine the concentration of the Grignard reagent. This analysis was performed according to the following procedure: 2 mg of 1,10 phenanthroline was added to a 50 mL flask with 10 mL of benzene; the 5.0 mL aliquot was then added; and the resulting mixture was titrated to the reddish-purple endpoint with 2.4 mL of 1.0 M butan-2-ol in p-xylene. Concentration was thus 0.48 M, which implied a 58% conversion to the desired Grignard reagent.

Step 2. 780 mg of CuI (4.10 mmol) was added to a round-bottomed flask under inert (Ar) conditions. A suspension was then formed by the addition of 100 mL of THF. 40 mL of dimethyl sulfide was added, yielding a clear yellow solution. 3-Acetylpyridine (10.0 g, 82.7 mmol) was then dissolved in 70 mL of THE and added to the yellow solution. Finally, 13.6 g (86.8 mmol) of phenyl chloroformate was dissolved in 50 mL of THF and the resulting solution was added slowly, resulting in the formation of a precipitate. The mixture was then cooled to −20° C. by packing the flask in dry ice. 172 mL (82.6 mmol) of the Grignard solution from above was then added dropwise over 20 min while maintaining the temperature below −5° C. The reaction mixture was allowed to warm to rt and then quenched with 400 mL of 20% aqueous ammonium chloride. Ethyl acetate (200 mL) was added. The organic layer was collected and the aqeuous layer was washed with 400 mL of ethyl acetate. The organic layers were combined, washed with brine, and then concentrated in vacuo. The residue was dissolved in dichloromethane and chromatographed on silica gel using a Biotage Flash 75L column, first eluting with 2 L of 10% EtOAc-hexane, and then with 4 L of 15% EtOAc-hexane. The fractions containing the desired compound were combined and concentrated in vacuo, providing 12.2 g of an oil: ¹H NMR (CDCl₃) δ 7.98 (s, 1H, broad) 7.44 (t, 2H), 7.31 (t, 1H), 7.21 (d, 2H), 6.99 (s, 1H, broad), 5.20 (s, 1H, broad), 3.23 (t, 1H, broad), 2.40 (s, 3H), 0.91 (m, 1H), 0.53-0.33 (m, 3H), 0.20 (m, 1H); LCMS (ES) m/z 284.0 (M+H⁺).

Step 3. 12.2 g (43.0 mmol) of the dihydropyridine was transferred into a round-bottomed flask containing 143 mL of decahydronaphthalene. Sulfur (1.38 g, 43.0 mmol) was added and the flask was heated in an oil bath at 180° C. Over 4 h, an additional 1.38 g of sulfur was added. The heat was then turned off and the reaction was diluted with 500 mL of MTBE. The organic layer was extracted twice with 250 mL portions of 1.0 N HCl. 500 mL of dichloromethane was added to the aqueous layer, which was then made basic with 1.0 N NaOH. The oragnic layer was then washed with 250 mL of brine, dried with sodium sulfate, filtered, and concentrated to obtain 2.13 g of an oil. The acidic aqueous layers were extracted again with 500 mL of dichloromethane. The organic layer was dried with sodium sulfate, filtered into the oil obtained from above, and concentrated in vacuo to obtain a total of 3.63 g, (27% from 3-acetylpyridine): ¹H NMR (CDCl₃) δ 8.83 (s, 1H), 8.54 (d, 1H), 6.93 (d, 1H), 2.71 (m, 1H), 2.71 (s, 3H), 1.28 (d, 2H), 0.92 (d, 2H); LCMS (ES) m/z 162.1 (M+H⁺); GCMS (CI) m/z 162 (M+H⁺).

### Preparation of Intermediate AC: 4-(tert-Butyl)-3-acetylpyridine

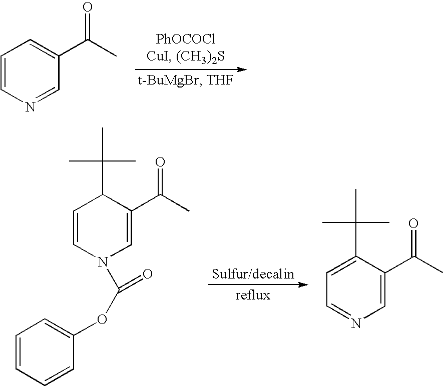

4-(tert-Butyl)-3-acetylpyridine was prepared according to the method used to prepare 4-ethyl-3-acetylpyridine to first give the intermediate phenyl 3-acetyl-4-tert-butyl-1(4H)-pyridinecarboxylate [HPLC t_{R}=3.32 min; TLC Rf=0.51 (5% EtOAc/hexane); ¹H NMR (CD₂Cl₂) δ 0.82 (s, 9H), 2.38 (s, 3H), 3.44 (d, 1H), 5.36-5.32 (m, 1H) 7.48-7.19 (m, 5H), 8.02 (s, 1H); LCMS (ES) m/z 300.3 (M+H⁺)], which was then aromatized with sulfur to give the desired product 4-(tert-butyl)-3-acetylpyridine: HPLC t_{R}=0.28; TLC Rf=0.31 (EtOAc); LCMS (ES) m/z 177.92 (M+H⁺).

### Preparation of Intermediate AD: 3-(2-Bromoacetyl)pyridine Hydrobromide

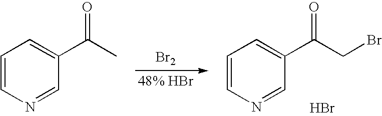

3-Acetylpyridine (4 g, 3.6 mL, 33 mmol) was added via syringe to a 3 necked round-bottomed flask that was equipped with a condenser, pressure equalizing dropping funnel and Ar inlet. 48% aqueous HBr (5.5 mL) was added and the solution was placed in a 70° C. oil bath. Bromine (5.3 g, 1.7 mL) was added to the dropping funnel. The bromine was then diluted with 48% aqueous HBr (1 mL) and then the bromine solution was added dropwise into the reaction over 30 min. TLC taken after 2 h revealed that the reaction was completed. The reaction mixture was cooled to rt, during which time crystals precipitated out of the reaction solution. The crystals were filtered and rinsed with 24% aqueous HBr. The crude yield was 7.19 g (77%). The material was recrystallized from 24% aqueous HBr, providing 5.18 g (56%) of the title compound.

### Preparation of Intermediate AE: 2-(2-Bromoacetyl)pyridine Hydrobromide

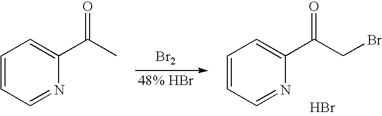

2-(2-Bromoacetyl)pyridine hydrobromide was prepared from 2-acetylpyridine according to the method used for 3-(2-bromoacetyl)pyridine hydrobromide, 23% yield.

### Preparation of Intermediate AF: 4-(2-Bromoacetyl)pyridine Hydrobromide

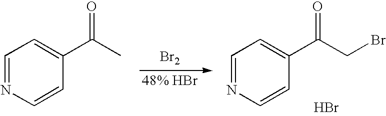

4-(2-Bromoacetyl)pyridine hydrobromide was prepared from 4-acetylpyridine according to the method used for 3-(2-bromoacetyl)pyridine hydrobromide, 44% yield.

### Preparation of Intermediate AG: 3-(2-Chloroacetyl)pyridine Hydrochloride

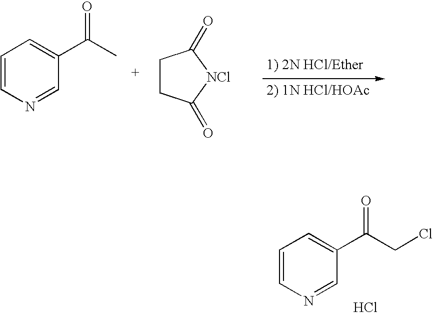

3-Acetylpyridine (5 g, 4.3 mL, 41.3 mmol) was dissolved in ether and the solution was cooled to 0° C. under Ar. A solution of 2N HCl/ether (1.2 eq, 25 mL) was added, and a white solid precipitated. The solid was rinsed with ether and dried, yielding 5.98 (92%) of the HCl salt. The 3-acetyl pyridinium hydrochloride was then dissolved in 1 eq of 1N HCl. An equivalent of N-chlorosuccinimide was added and the reaction was refluxed overnight. Ether was added to the reaction mixture; a solid precipitated. The solid was washed with ether and dried under vacuum, providing 6.52 g (83%) of the title compound. The product was used without further purification.

### Preparation of Intermediate AH: 4-Methyl-3-(2-chloroacetyl)pyridine

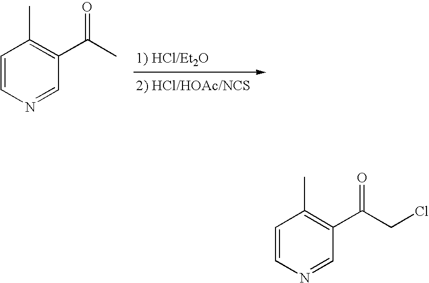

Into a 500 mL round-bottomed flask was placed 4-methyl-3-acetylpyridine (10.0 g, 74.1 mmol) in 90 mL of Et₂O. To this solution was added 88.9 mL of 1M HCl in Et₂O (1.2 eq, 88.9 mmol) and the solution allowed to stir for 1 h at rt, at which point, the precipitate was filtered and washed with Et₂O. The solid was then dried in vacuo at 60° C. The HCl salt of 4-methyl-3-acetylpyridine (12.0 g, 70.0 mmol) was then dissolved in 70.0 mL of 1M HCl in acetic acid. Then 9.34 g (1 eq, 70.0 mmol) of N-chlorosuccinimide (NCS) was added, and the reaction allowed to stir under Ar at rt overnight. At this point, 300 mL of Et₂O was added, resulting in an off-white precipitate. This was allowed to stir for 1 h, then filtered and rinsed with Et₂O to provide 11.9 g (83%) of 4-methyl-3-(2-chloroacetyl)pyridine: GCMS t_{R}=6.60 min, 169 (M⁺); ¹H NMR (DMSO-d₆) δ 2.51 (s, 3H), 5.15 (s, 2H), 7.68 (d, 1H), 8.68 (d, 1H), 9.06 (s, 1H).

### Preparation of Intermediate AI: 4-(2-Propyl)-3-(2-chloroacetyl)pyridine

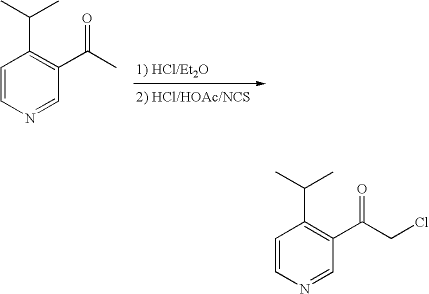

4-(2-Propyl)-3-(2-chloroacetyl)pyridine was prepared from 4-(2-propyl)-3-acetylpyridine according to the method used to prepare 4-methyl-3-(2-chloroacetyl)pyridine. MS and NMR data were consistent with the structure and the product was used without further purification.

### Preparation of Intermediate AJ: 4-Ethyl-3-2-chloroacetyl)pyridine

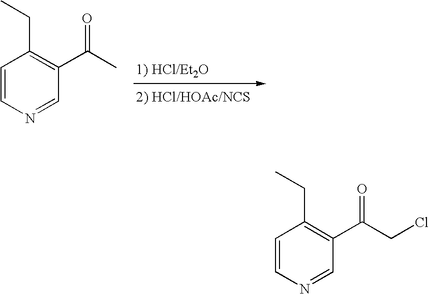

4-(2-Ethyl)-3-(2-chloroacetyl)pyridine was prepared from 4-(2-ethyl)-3-acetylpyridine according to the method used to prepare 4-methyl-3-(2-chloroacetyl)pyridine. MS and NMR data were consistent with the structure and the product was used without further purification.

### Preparation of Intermediate AK: 4-(1-Propyl)-3-(2-chloroacetyl)pyridine

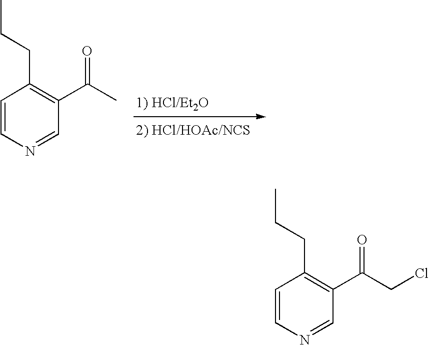

4-(1-Propyl)-3-(2-chloroacetyl)pyridine was prepared from 4-(1-propyl)-3-acetylpyridine according to the method used to prepare 4-methyl-3-(2-chloroacetyl)pyridine. MS and NMR data were consistent with the structure and the product was used without further purification.

### Preparation of Intermediate AL: 4-Cyclopropyl-3-(2-chloroacetyl)pyridine

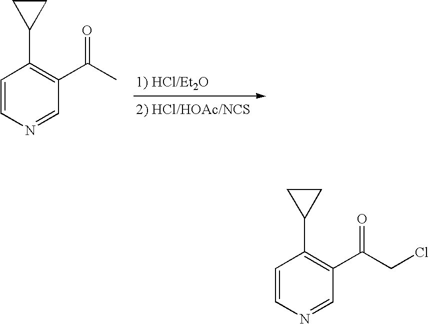

4-(Cyclopropyl)-3-(2-chloroacetyl)pyridine was prepared from 4-(cyclopropyl)-3-acetylpyridine according to the method used to prepare 4-methyl-3-(2-chloroacetyl)pyridine. MS and NMR data were consistent with the structure and the product was used without further purification.

### Preparation of Intermediate AM: 4-(tert-Butyl)-3-(2-chloroacetyl)pyridine

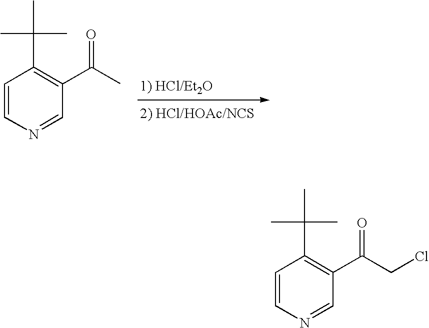

4-(tert-Butyl)-3-(2-chloroacetyl)pyridine was prepared from 4-(tert-butyl)-3-acetylpyridine according to the method used to prepare 4-methyl-3-(2-chloroacetyl)pyridine. MS and NMR data were consistent with the structure and the product was used without further purification.

### General Method C: Synthesis of Non-Commercially Available α-Bromo Aryl Ketones

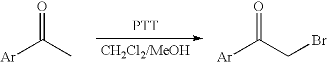

To a solution of aryl ketone (12 mmol) in dichloromethane (20 mL) and methanol (2 mL) was added a solution of phenyltrinethylammonium tribromide (PTT) (4.68 g, 12 mmol) in dichloromethane (20 mL) and methanol (2 mL) dropwise. The red-colored reaction was stirred 4 h at rt after which time the color had changed to light-yellow. The solvents were evaporated in vacuo and the residue was partitioned between EtOAc (75 mL) and H₂O (50 mL). The separated organic phase was washed with H₂O (50 mL), brine (50 mL), and then dried over Na₂SO₄. The solvent was evaporated in vacuo, giving the desired alpha bromo ketone intermediate, which was used in the next step without purification. NMR and MS spectral data were consistent with the structure.

### The Following Alpha Bromo Aryl Ketones were Prepared According to General Method C

Intermediate AN: 2-(Bromoacetyl)-5-chlorothiophene was synthesized from 2-acetyl-5-chlorothiopnene (87%).

Intermediate AO: 2-(Bromoacetyl)-5-methylfuran was synthesized from 2-acetyl-5-methylfuran (93%).

Intermediate AP: 2-Bromo-4′-chloropropiophenone was synthesized from 4′-chloropropiophenone (86%).

Intermediate AQ: 2-(Bromoacetyl)-4-phenoxybenzene was synthesiszed from 4-phenoxyacetophenone (62%).

Intermediate AR: 2-Bromo-4-(4-chlorophenyl)acetophenone was synthesized from 4-(4-chlorophenyl)acetophenone (69%).

Intermediate AS: 2-(2-Bromoacetyl)-5-methylfuran was synthesized from 2-acetyl-5-methylfuran (51%).

### Preparation of Intermediate AT: 2-Bromo-2′,4′-di(trifluoromethyl)acetophenone

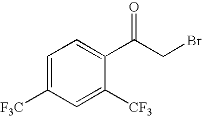

A solution of 2,4-di(trifluoromethyl)acetophenone (5.0 g, 19.52 mmol) in anhydrous tetrahydrofuran under Ar was treated with phenyltrimethylammonium tribromide (7.34 g, 19.52 mmol, 1.0 eq) at 0° C. The reaction mixture was stirred at ambient temperature for 17 h and then concentrated. The crude material was redissolved in EtOAc (250 mL). The organic layer was washed with water (2×250 mL) and brine (1×150 mL), dried (MgSO₄), filtered, and then evaporated in vacuo. Crystallization from hexane at 0° C. afforded a white crystalline solid. The product was filtered and rinsed well (3×) with hexane to give 3.78 g (57.8%) of a white solid: GCMS m/z 333 (M⁺), 335 (M+2⁺).

### General Method D: Synthesis of Non Commercially Available 3-Aryl-1-chloro-2-propanones

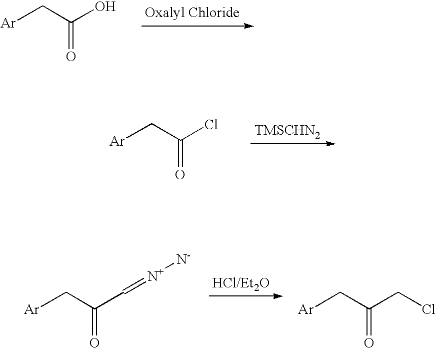

A solution of the arylacetic acid (13 mmol) in CH₂Cl₂ (30 mL) was treated with 2.0 M oxalyl chloride in CH₂Cl₂ (14 mmol) via syringe. This was treated with 2 drops of DMF, which caused a vigorous gas evolution. The reaction was stirred 3 h, then the CH₂Cl₂ was evaporated in vacuo. The residue was dissolved in TIE (15 mL) and acetonitrile (15 mL), cooled to 0° C., and then treated dropwise with 2.0 M (trimethylsilyl)diazomethane in hexanes (27 mmol). The mixture was stirred while warming to rt overnight. The solvents were removed in vacuo. The residue was dissolved in diethyl ether (30 mL), cooled to 0° C., and then treated dropwise with 2.0 M HCl in ether (27 mmol), which caused a vigorous gas evolution. The reaction was stirred 30 min, the solvent removed in vacuo, and the residue purified via flash chromatography (0-1% EtOAc/hexane), providing the desired 3-aryl-1-chloropropanone intermediate. The NMR and MS spectral data were consistent with the structure. In some cases, the necessary intermediate acid chloride was commerically available, which made its preparation from the arylacetic acid unneccessary.

### The Following Intermediates were Prepared Using Method D

Intermediate AU: 1-(4-Methylphenyl)-3-chloro-2-propanone was synthesized from 1-methylphenyl acetic acid (90%).

Intermediate AV: 1-(4-Chlorophenyl)-3-chloro-2-propanone was synthesized synthesized from 4-chlorophenylacetyl chloride (82%).

Intermediate AW: 1-(3-Chlorophenyl)-3-chloro-2-propanone was synthesized synthesized from 3-chlorophenylacetic acid (70%).

Intermediate AX: 1-(3-Methylphenyl)-3-chloro-2-propanone was synthesized synthesized from 3-methylphenylacetic acid (58%).

Intermediate AY: 1-(4-Fluorophenyl)-3-chloro-2-propanone was synthesized synthesized from 4-fluorophenylacetic acid (79%).

Intermediate AZ: 1-(3,4-Dichlorophenyl)-3-chloro-2-propanone was synthesized synthesized from 3,4-dichlorophenylacetic acid (45%).

Intermediate BA: 1-(3-Nitrophenyl)-3-chloro-2-propanone was synthesized from 3-nitrophenylacetic acid (62%).

Intermediate BB: 1-(4-Bromophenyl)-3-chloro-2-propanone was synthesized synthesized from 4-bromophenylacetic acid.

### Preparation of Intermediate BC: 1-(4-Chlorophenyl)-3-chloro-2-propanone

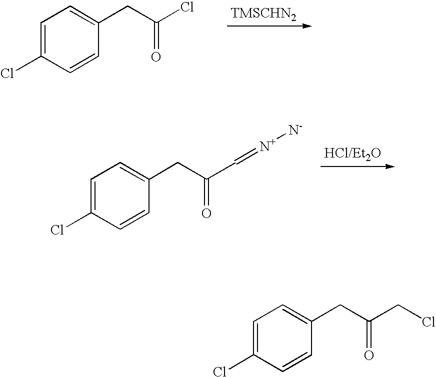

A solution of trimethylsilyldiazomethane in hexane (2.0 M, 23 mL, 46.1 mmol) was added dropwise, over a 9 min period, to a solution of 4-chlorophenylacetyl chloride (8.89 g, 46.1 mmol) in a mixture of anhydrous acetonitrile (135 mL) and anhydrous THF (135 mL) that was held at 0° C. under Ar. After stirring overnight at rt, concentration in vacuo gave a pale yellow oil, which was purified by silica gel chromatography (hexane-dichloromethane solvent gradient) to afford 8.44 g (94.2%) of pale yellow solid intermediate. A stirred solution of the diazo intermediate (8.4 g, 43.4 mmol) in diethyl ether (240 mL) was treated dropwise with hydrogen chloride (2M) in ether over a 10 min period. Gentle bubbling was observed, as well as a mild rise in the reaction temperature. After stirring overnight at rt, TLC showed no remaining intermediate. The mixture was concentrated in vacuo to afford 4.73 g (53.7%) of the title compound as tan, opaque crystals, mp 40.5-45.5° C.: ¹H NMR (CDCl₃) δ 7.3 (d, 2H), 7.2 (d, 2H), 4.1, (s, 2H), 3.9 (s, 2H); GCMS m/z 202 (M⁺).

### General Method E: Synthesis of Cycloalkyl and Bicycloalkyl Methyl Ketones as Exemplified by the Preparation of Acetylcycloheptane (Intermediate BD)

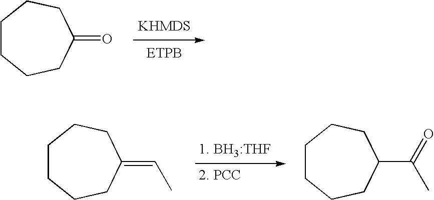

Step 1. To a stirring suspension of ethyltriphenylphosphonium bromide (ETPB) (25.0 g, 67.34 mmol) in anhydrous THF (80 mL) at 0° C. was added KHMDS (135 mL of a 0.5 M/toluene solution, 67.34 mmol) dropwide over 30 min. The red suspension was stirred 15 min at 0°, then a solution of cycloheptanone (6.87 g, 61.22 mmol) in THF (10 mL) was added over 30 min. The orange suspension was stirred to rt over 3 h with the ice bath removed then at rt for 16 h. The reaction was quenched with water (200 mL) and extracted with hexane (2×400 mL). The organic was dried (Na₂SO₄) and concentrated in vacuo to give an oil with solids (triphenylphosphonium oxide). The oil was triturated in hexane and filtered to remove the solid repeatedly until a yellow oil remains. This was purified by a silica gel plug (hexane) to give the product as a clear oil in 22% yield (1.68 g, 13.55 mmol): ¹H NMR (CDCl₃) δ 4.96 (1H, m), 1.95 (4H, m), 1.18-1.37 (11H, m).

Step 2. To a solution of cyclohexylethylidene (1.60 g, 12.88 mmol) in dry THF (75 mL) at 0° C. was added BH₃:THF complex (9.02 mL of a 1.5 M THF/ether solution, 13.52 mmol) over 5 min. The solution was stirred at 0° C. for 1 h then quenched by slow dropwise addition of water (H₂ evolution). The quenched reaction was further diluted with water (100 mL) and extracted with Et₂O (2×250 mL). The organic was dried (MgSO₄) and concentrated and the residue dried under P₂O₅ in vacuo. The crude intermediate was dissolved in CH₂Cl₂ (100 mL) and PCC added (5.55 g, 25.76 mmol) followed by 4 Å molecular sieves activated powder (5.55 g). This was refluxed vigorously for 3 h. More CH₂Cl₂ (50 mL), PCC (14.0 g, 64.95 mmol), and 4 Å molecular sieves powder (11 g) were added and the reaction refluxed for 16 h. The reaction was diluted with more water (200 mL) and extracted with CH₂Cl₂ (3×300 mL). The organic layer was dried (Na₂SO₄) and filtered directly through a plug of silica gel to give the product as a clear oil in 83% yield (1.66 g, 10.70 mmol): TLC Rf 0.18 (5% EtOAc/hexane); GCMS (EI) m/z 140 (M)⁺, t_{R}=5.30 min.

### General Method F: Synthesis of 2-Bromomethyl Cycloalkyl Ketones and 2-Bromomethyl Bicycloakyl Ketones as Exemplified by the Preparation of 2-Bromoacetylcyclohexane (Intermediate BE)

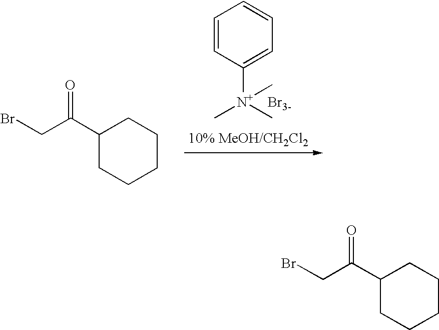

A solution of cyclohexylmethyl ketone (2.50 g, 19.8 mmol) in dry CH₂Cl₂ (20 mL) and MeOH (2 mL) was treated with a solution of phenyltrimethylammonium tribromide (7.45 g, 19.8 mmol) in dry CH₂Cl₂ (20 mL) and MeOH (2 mL) dropwise over 2 h at rt. The reaction was stirred an additional 2 h at rt, then the reaction was concentrated and redissolved in Et₂O (200 mL). This was washed with water (2×100 mL) and dried (Na₂SO₄). The crude product was purified by silica gel chromatography to give the product as a clear oil in 32% yield (1.29 g, 6.31 mmol): TLC Rf 0.35 (5% EtOAc/hexane); GCMS (CI) 205 m/z (M+H)⁺, t_{R}=6.08 min.

### Preparation of Intermediate BF: 3-Bromobicyclo[3.2.1]octan-2-one

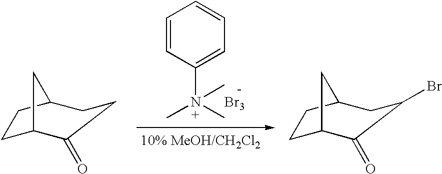

3-Bromobicyclo[3.2.1]octan-2-one was prepared according to General Method F from bicyclo[3.2.1]octanone: TLC Rf 0.30 (10% EtOAc/hexane); GCMS (EI) m/z 202 (M+)⁺, t_{R}=7.00 min.

### Preparation of Intermediate BG: 2-Bromocycloheptanone

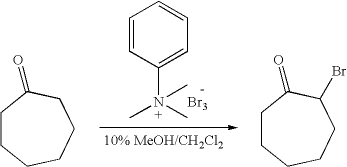

2-Bromocycloheptanone was prepared according to General Method F from cycloheptanone: TLC Rf 0.25 (100% hexane); GCMS (EI) m/z 190 (M)⁺, t_{R}=5.91 min.

### Preparation of Intermediate BH: 2-Bromo-7-phenylcycloheptanone

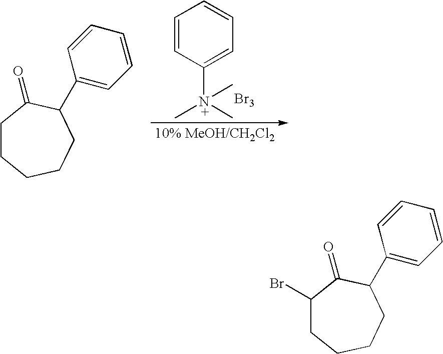

2-Bromo-7-phenylcycloheptanone was prepared according to General Method F from 2-phenylcycloheptanone: TLC Rf 0.33 (5% EtOAc hexane); GCMS (EI) 266 (M)⁺, t_{R}=8.82 min.

### Preparation of Intermediate BI: 2-Bromo-7-methyoxy-1-tetralone

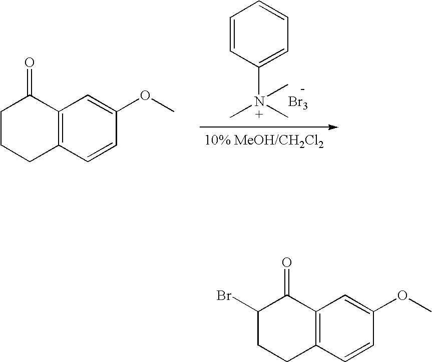

2-Bromo-7-methoxy-1-tetralone was prepared according to General Method F from 7-methoxy-1-tetralone: TLC Rf 0.55 (15% EtOAc/hexane); GCMS (EI) m/z 254/255 (M)⁺, t_{R}=8.50 min.

### Preparation of Intermediate BJ: 2-Bromo-6-methoxy-1-tetralone

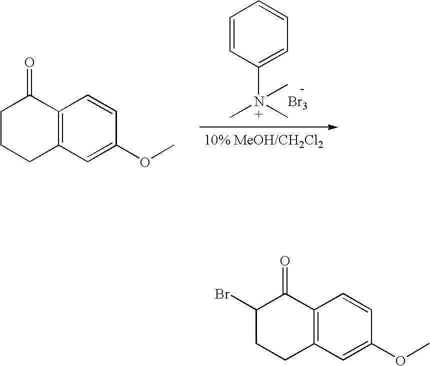

2-Bromo-6-methoxy-1-tetralone was prepared according to General Method F from 6-methoxy-1-tetralone: TLC Rf 0.20 (40% CH₂Cl₂/hexane); GCMS (EI) m/z 254/255 (M)⁺, t_{R}=9.05 min.

### Preparation of Intermediate BK: 2-Bromo-1-tetralone

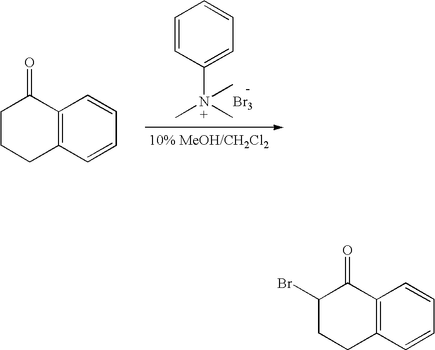

2-Bromo-1-tetralone was prepared according to General Method F from α-tetralone: TLC Rf 0.50 (5% EtOAc/hexane); GCMS (EI) m/z 224/225 (M)⁺, t_{R}=8.00 min.

### Example 1

### Preparation of 2-(4-Methyl-3-pyridyl)-4-(4-chlorophenyl)thiazole Hydrobromide

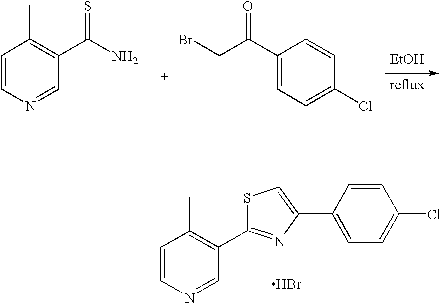

A mixture of 4-methylpyridine-3-thiocarboxamide (2.0 g, 13.1 mmol), 4-chlorophenacyl bromide (3.12 g, 13.1 mmol) and absolute ethanol (100 mL) was refluxed overnight under an Ar atmosphere. After cooling in ice water, the solid was filtered, sequentially washed with ethanol and hexane, and then dried to afford 4.26 g (88.4%) of a pale yellow solid. A 1.0 g portion was recrystallized from distilled water to afford 0.37 g as pale yellow crystals, mp 279.5-286° C.: TLC Rf 0.45 (5% methanol/dichloromethane); TLC Rf 0.41 (EtOAc); ¹H NMR (DMSO-d₆) δ 9.2 (s, 1H), 8.7 (d, 1H), 8.5 (s, 1H), 8.1 (dd, 2H), 7.9 (d, 1H), 75. (dd 2H); 5.2 (broad exchangeable, 1H); 2.8 (s, 3H); LCMS 287 (M+H⁺), 289 (M+H+2⁺). Anal. Calcd for C₁₅H₁₁ClN₂.HBr: C, 49.00; H, 3.29; N, 7.62; Br, 21.73; Cl, 9.64; S, 8.72. Found: C, 49.73; H, 3.24; N, 7.6; Br, 20.38; Cl, 9.84; S, 8.8.

### Example 2

### Preparation of 2-(4-Methyl-3-pyridyl)-4-(2,4-dichlorophenyl)thiazole Hydrochloride

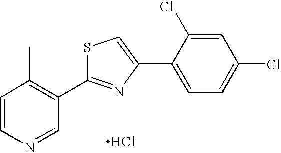

2-(4-Methyl-3-pyridyl)-4-(2,4-dichlorophenyl)thiazole hydrochloride was prepared from 4-methylpyridine-3-thiocarboxamide and 2,2′,4′-trichloroacetophenone according to the procedure used in Example 1 to afford 1.78 g (61.1%) ofthetitle compound: TLC Rf 0.42 (5% methanol/dichloromethane); TLC Rf 0.43 (EtOAc); ¹H NMR (DMSO-d₆) δ 9.2 (s, 1H), 8.7 (d, 1H), 8.4 (s, 1H), 8.0 (d, 1H), 7.9 (d, 1H), 7.8 (d, 1H), 7.58 (d, 1H), 7.55 (d, 1H), 7.0 (broad exchangeable, 1H), 2.8 (s, 3H); LCMS 321 (M+H⁺); 323 (M+2+H⁺). Anal. Calcd for C₁₅H₁₀Cl₂N₂S.HCl: C, 50.37; H, 3.1%; N, 7.83; Cl, 29.74; S, 8.96. Found: C, 50.43; H, 3.1; N, 7.85; Cl, 29.5; S, 8.99.

### Example 3

### Preparation of 2-(4-Methyl-3-pyridyl)-4-(4-chlorophenylmethyl)thiazole Hydrochloride

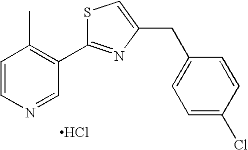

2-(4-Methyl-3-pyridyl)-4-(4-chlorophenylmethyl)thiazole was prepared according from 4-methylpyridine-3-thiocarboxamide and 1-chloro-3-(4-chlorophenyl)-2-propanone according to the procedure used in Example 1 to yield, after chromatography, 1.88 g (47.7%). This material was dissolved in dichloromethane, filtered, and then the filtrate was stirred while hydrogen chloride (2M in diethyl ether) was added. After removal of solvent in vacuo, the solid was triturated with ether, filtered and washed to afford 1.60 g (36.9%) of 2-(4methyl-3-pyridyl)-4-(4-chlorophenylmethyl)thiazole hydrochloride as a tan-brown solid, mp 165.5-170° C.: TLC Rf 0.12 (2% methanol in dichloromethane); TLC Rf 0.39 (EtOAc); ¹H NMR (DMSO-d₆) δ 9.1 (s, 1H), 8.7 (d, 1H), 7.9 (d, 1H), 7.7 (s, 1H), 7.3 (s, 4H), 4.2 (s, 2H), 2.7 (s, 3H); LCMS 301 (M+H⁺); 303 (M+H+2⁺). Anal. Calcd for C₁₆H₁₃ClN₂S.HCl: C, 56.98; H, 4.18; N, 8.31; Cl, 21.02; S. 9.51. Found: C, 56.86; H, 4.18; N, 8.02; Cl, 21.28; S, 9.11.

### Example 4

### Preparation of 2-(4-Cyclopropyl-3-pyridyl)-4-(4-chlorophenyl)thiazole

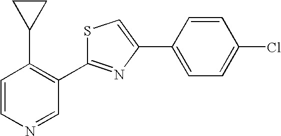

A solution of 4-cyclopropyl-3-pyridinecarbothioamide (1.53 g, 8.6 mmol), 4-chlorophenacyl bromide (2.25 g, 9.5 mmol) in absolute ethanol (30 mL) was heated to reflux for 16-18 h. The resulting precipitate was cooled in an ice bath for 2-2.5 h, filtered, and then washed with cold absolute ethanol (5 mL). The hydrochloride salt so obtained was converted to the free base with sodium bicarbonate, then extracted with dichloromethane and concentrated. Silica gel chromatography, using 5-20% EtOAc-hexane, yielded 1.5 g (56%) of the pure product: LCMS t_{R} 2.65 min, 313 (M+H⁺); ¹H NMR (CDCl₃) δ 8.95 (1H, s), 8.52 (1H, d), 7.92 (2H, d), 7.64 (1H, s), 7.43 (2H, d), 6.94 (1H, d), 2.68 (1H, m), 1.23 (2H, m), 0.94 (2H, m). Anal. Calcd for C₁₇H₁₃N₂ClS: C, 65.27; H, 4.19; N, 8.96. Found: C, 65.02; H, 4.35; N, 8.85.

### Example 5

### General Method G, as Exemplified by the Preparation of 2-(3-Pyridyl)-4-(cyclohexyl)thiazole

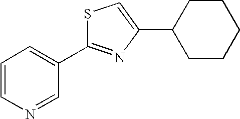

To a solution of thionicotinamide (202 mg, 1.46 mmol) in abs. ethanol (10 mL) was added 2-bromoacetyl cyclohexane (300 mg, 1.46 mmol) and the solution refluxed for 2.5 h. The reaction was concentrated in vacuo, and the residue suspended in CH₂Cl₂. The crude product was free-based with triethylamine (0.24 mL), and purified by silica gel chromatography to give 256 mg (72%) of the title product in 72% yield as a clear oil: TLC Rf 0.24 (25% EtOAc/hexane); LCMS (ES) 245 (M+H)⁺, t_{R}=2.46 min.

### Example 6

### General Procedure H, as Exemplified by the Preparation of 2-(3-pyridyl)-4-(phenylamino)-5-methylthiazole

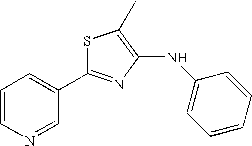

A homogenous mixture of thionicotinamide (1.00 g, 7.236 mmol) and 2-bromo-N-phenylpropionamide (1.65 g, 7.24 mmol) was melted at 110° C. for 20 h. The melt was suspended in CH₂Cl₂ (50 mL) and free-based with triethylamine (1.01 mL). The suspension was filtered to remove starting material and the filtrate purified by silica gel chromatography to give the product as light yellow crystals in 3% yield (53 mg, 0.20 mmol): TLC Rf 0.33 (50% EtOAc/hexane); LCMS (ES) 268 (M+H)⁺, t_{R}=2.29 min.

### Example 7

### Preparation of 2-(4-Methyl-3-pyridyl)-4-(N-methylcyclohexylamino)thiazole

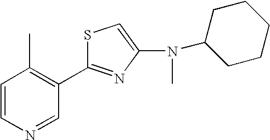

To a solution of 4-methyl thionicotinamide (602 mg, 3.95 mmol) in dry DMF (15 mL) at 100° C. was added 2-chloro-N-cyclohexyl-N-methylacetamide (600 mg, 3.16 mmol) dropwise as a solution in dry DMF (5 mL) over 10 min. The reaction was stirred for 1.5 h at 100° C., then diluted with water (100 mL) and extracted with Et₂O (2×200 mL). The organic layer was washed with water (50 mL), dried (Na₂SO₄) and concentrated in vacuo. The residue was purified by silica gel chromatography to give the product as an oil in 0.5% yield (4 mg, 0.014 mmol): TLC Rf 0.52 (50% EtOAc/hexane); LCMS (ES) 288 (M+H)⁺, t_{R}=2.59 min.

### Example 8

### General Method I, as Exemplified by the Preparation of 2-(3-Pyridyl)-4-(isopropoxy)thiazole

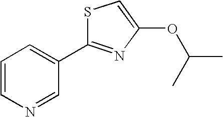

A suspension of thionicotinamide (977 mg, 7.07 mmol) and N-(bromoacetyl)-3,5-dichloroaniline (1.00 g, 3.53 mmol) in isopropanol (30 mL) was refluxed for 16 h. The solvent was then boiled off and the solid residue suspended in CH₂Cl₂ (20 mL). The crude suspension was free-based with triethylamine (0.985 mL), and filtered to remove the pure 4-(3,5-dichlorophenyl)aminothiazole side-product. Purification by silica gel chromatography gave the product as a clear oil in 19% yield (146 mg, 0.663 mmol): TLC Rf 0.28 (25% EtOAc/hexane); LCMS (ES) 221 (M+H)⁺, t_{R}=1.96 min.

### Example 9

### General Method J, as Exemplified by the Preparation of 2-(4-Methyl-3-pyridyl)-4-(cyclohexyl)-5-methylthiazole

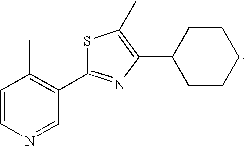

To an LDA solution (0.581 mmol) in dry THF (5 mL) at −78 C. was added 2-(4-methylpyridyl)-4-cyclohexylthiazole (100 mg, 0.387 mmol) as a solution in dry THF (5 mL) dropwise over 10 min. The red suspension was stirred for 30 min at −78 C, then iodomethane (549 mg, 3.87 mmol) was added. The reaction was warmed to rt over 1 h with the ice bath removed. The clear reaction was then concentrated in vacuo and the residue purified by silica gel chromatography to give the product as an amber oil in 91% yield (96 mg, 0.35 mmol): TLC Rf 0.63 (50% EtOAc/hexane); LCMS (ES) 273 (M+H)⁺, t_{R}=2.65 min.

### Example 10

### General Method K, as Exemplified by the Preparation of 2-(3-Pyridyl)-4-(benzyloxy)thiazole

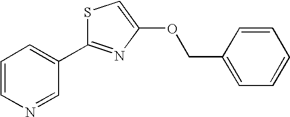

Thionicotinamide (1.00 g, 7.236 mmol) was heated in neat benzyl bromoacetate (8.29 g, 36.2 mmol) at 90° C. for 1 h. The reaction was diluted with CH₂Cl₂ (30 mL) and quenched with triethylamine (2.02 mL). This was purified by silica gel chromatography to give the product as an orange solid in 3% yield (51 mg): Rf 0.40 (50% EtOAc/hexane); LCMS (ES) 269 (M+H)⁺, t_{R}=3.10 min.

### Example 11

### Preparation of 2-(4-Chloro-3-pyridyl)-4-(4-chlorophenyl)thiazole (Intermediate BF)

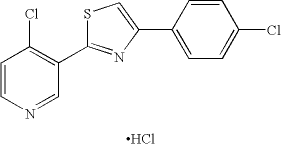

Step 1. A mixture of 4-methoxypyridine-5-thiocarboxamide (0.50 g, 3 mmol) and 2-bromo-4′-chloroacetophenone (0.69 g, 3 mmol) in ethanol (40 mL) was refluxed overnight, during which time a yellow precipitate formed. The reaction mixture was cooled and the solvent evaporated in vacuo. The residue was triturated in CH₂Cl₂, filtered, and then washed with CH₂Cl₂ (2×50 mL). The material was triturated a second time with 20% MeOH in CH₂Cl₂, filtered, and washed with CH₂Cl₂. Drying under vacuum gave 0.47 g (54%) of 2-(3-pyridin-4-one)-4-(4-chlorophenyl)thiazole as a tan solid.

Step 2. A stirred mixture of 2-(3-pyridin-4-one)4-(4-chlorophenyl)thiazole (4.06 g, 14.1 mmol) and phosphorus oxychloride (66 mL, 703 mmol) was heated under an Ar atmosphere and allowed to reflux for 16.5 h. After allowing the mixture to cool to rt, the solid was filtered and triturated on the funnel twice with dichlioromethane. After drying, 4.6 g of the title compound was obtained as a pale yellow solid, mp 176.5-183.5 ° C.: TLC Rf 0.33 (2% methanol in dichloromethane); TLC Rf 0.45 (1:1 hexane-EtOAc); ¹H NMR (DMSO-d₆) δ 9.4 (s, 1H), 8.6 (d, 1H), 8.5 (s, 1H), 8.1 (d, 2H), 7.8 (d, 1H), 7.5 (d, 2H), 7.2 (broad exchangeable, 1H); LC MS 307 (M+H⁺), 309 (M+2+H⁺). Anal. Caled for C₁₄H₈Cl₂N₂S: C, 48.93; H, 2.64; N, 8.15; Cl, 30.95; S, 9.33. Found: C, 48.75; H, 2.43; N, 7.73; Cl 31.44; S, 8.98.

### Example 12

### General Method L, as Exemplified by the Preparation of 3-[4-(4Chlorophenyl)-1,3-thiazol-2-yl]-4-(1-piperidinyl)pyridine

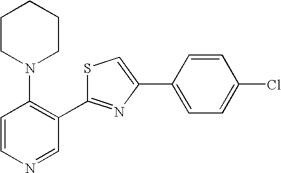

4-Chloro-3-[4-(4-chlorophenyl)-1,3-thiazol-2-yl]pyridine (70.0 mg, 0.2 mmol) and piperidine (80.6 μL, 0.8 mmol) were dissolved in THF (4 mL). To this solution was added 1% v/v HCl(0.1 mL). The reaction was refluxed overnight. The mixture was concentrated under reduced pressure. The compound was purified by Gilson HPLC to yield 58.0 mg (81.5%) of a pale yellow oil.

### General Method M: Synthesis of 2-(3-Pyridyl)Thiazoles

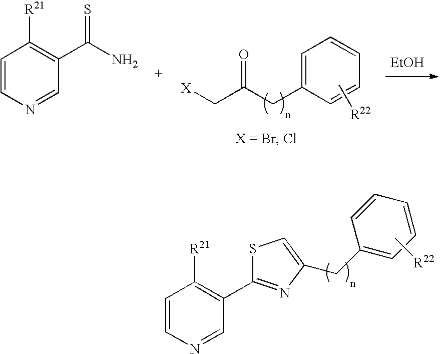

A mixture of the pyridine thiocarboxamide (1 mmol) and the alpha-bromo or alpha-chloro ketone (1 mmol) in ethanol (15 mL) was refluxed together overnight. The reaction was cooled and the solvent evaporated in vacuo. The residue was treated with triethylamine to liberate the free base of the product, and the residue was purified by flash chromatography (10-20% EtOAc/hexane) to provide the desired 2-(3-pyridyl)thiazole derivative. The yields ranged from 45-90%.

### General Method N: Synthesis of 2-(3-Pyridyl)-Thiazoles and 2-(4-Isoquinolinyl)-Thiazoles

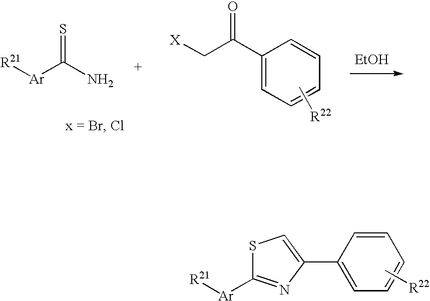

In a 250 mL round-bottomed flask were placed the pyridine thiocarboxamides (18.0 mmol) and the requisite bromoketone (1.1 eq, 19.9 mmol) in 100 mL EtOH. The reaction mixture was heated at 70° C. for 8 h under Ar and then concentrated. The residue was partitioned between CH₂Cl₂ (3×100 mL), H₂O (100 mL), and Et₃N (5 mL). The organic layer was dried over Na₂SO₄ and concentrated. Purification by chromatography using 80/20 hexanes-EtOAc afforded the target thiazole derivatives. The yield ranged from 50-85%.

### General Method O: Synthesis of 2-(3-Pyridyl)-Thiazoles by Parallel Methods

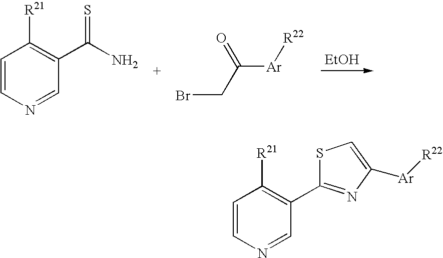

An EPA vial was charged with 4-cyclopropyl-3-pyridinecarbothioamide (11.2 mmol) and the α-halo ketone (13.5 mmol, 1.20 eq). To this was added 15 mL of anhydrous ethanol. In the event that the α-halo ketone was a salt, then pyridine (1.2 eq) was also added to the vial. The vial was capped tightly and shaken in a heating block overnight at 82° C. The reaction mixture was concentrated down and taken up in 2 mL of dichloromethane and 2 mL of water. It was basified with triethylarnine (˜10 drops) and extracted twice with dichloromethane. The organic layers were combined and concentrated to dryness, and the crude residue was dissolved in hot DMSO. The compound was purified optionally by chromatography, recrystallization, or by Gilson HPLC to yield the desired thiazole derivative.

### General Method P: Salt Formation

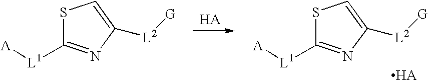

A solution of the pyridyl thiazole derivative (3.5 mmol) in Et₂O (50 mL) was treated dropwise at rt with an ethereal solution of a protic acid (4.4 mmol). A solid formed immediately and the reaction was stirred 1.5 h. The solid was collected by filtration and washed with Et₂O (2×50 mL). Drying under vacuum gave the desired salt.

### Example 13

### Preparation of 4-Methyl-3-[4-(1-piperidinylcarbonyl)-1,3-thiazol-2-yl]pyridine

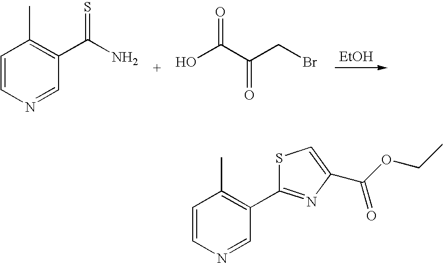

Step 1. Preparation of Ethyl 2-(4-methyl-3-pyridinyl)-1,3-thiazole-4-carboxylate: Bromopyruvic acid 4.09 g (0.0245 mmol) was diluted with ethanol (100 mL). Solid 4-methy-3-pyridinecarbothioamide (2.86 g, 18.8 mmol) was added and the reaction mixture was heated at 82° C. overnight. After cooling to rt, triethylamine (2.47 g, 2.45 mmol) was added. The reaction mixture was adsorbed onto silica gel and purified by chromatography using 2% methanol in dichloromethane, yielding 3.33 g (55%) of the title compound as an off white solid: LCMS 249 (M+H⁺), t_{R}=0.75 min.

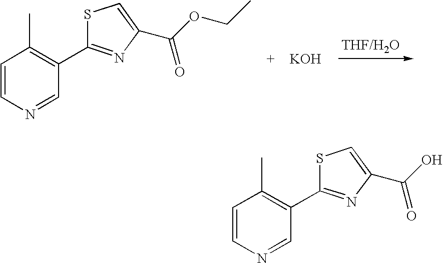

Step 2. Preparation of 2-(4-Methyl-3-pyridinyl)-1,3-thiazole-4-carboxylic acid: Ethyl 2-(4-methyl-3-pyridinyl)-1,3-thiazole-4-carboxylate 1.44 g (5.5 mmol) was dissolved in 40 mL of tetrahydrofuran. A solution of potassium hydroxide (0.962 g, 16.5 mmol) in water (10 mL) was added and the reaction mixture was heated at 70° C. under Ar for 1.5 h. The reaction mixture was cooled, water was added, then the THF was removed under vacuum. The residue was then partitioned between dichloromethane and water. The organic layer, presumed to contain traces of unreacted starting material, was discarded. The aqueous layer was brought to pH 2 using 5% aqueous HCl. The material did not extract into ethyl acetate or dichloromethane. The product was precipitated from the aqueous layer using ether, then collected by filtration. The material obtained contained about three equivalents of KCl: yield 1.49 g (63%); white solid; LCMS (M+H⁺) 221, t_{R}=0.69 min.

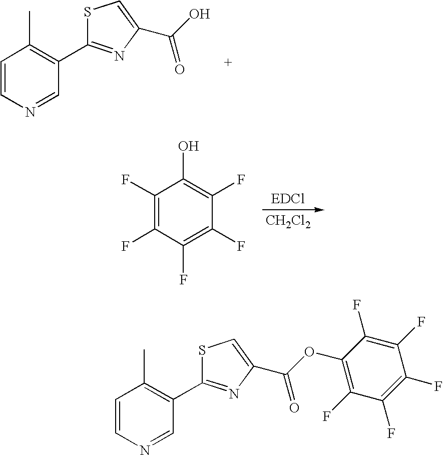

Step 3: Preparation of 2,3,4,5,6-Pentafluorophenyl 2-(4-methyl-3-pyridinyl)-1,3-thiazole-4-carboxylate:

2-(4-Methyl-3-pyridinyl)-1,3-thiazole-4-carboxylic acid*3KCl 1.92 g (4.3 mmol) was suspended in dichloromethane (20 mL). Pentafluorophenol (1.40 g, 7.5 mmol) and EDCI (1.44 g, 8.25 mmol) were then added. The reaction mixture became homogenous upon the addition of triethylamine (2.3 g, 2.25 mmol). After stirring at rt overnight under Ar, dichloromethane and water were added. The material was partitioned between the two layers. The separated organic layer was washed three times with aqueous sodium carbonate solution followed by brine, then dried over sodium sulfate. Filtration and concentration afforded 100 mg of white solid (3.4%): LCMS (M+H⁺) 387, t_{R}=2.60 min.

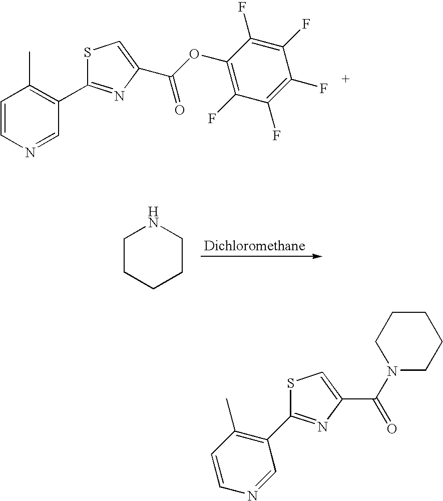

Step 4. Preparation of 4-Methyl-3-[4-(1-piperidinylcarbonyl)-1,3-thiazol-2-yl]pyridine: 2,3,4,5,6-Pentafluorophenyl 2-(4methyl-3-pyridinyl)-1,3-thiazole-4-carboxylate (97 mg, 0.25 mmol) was dissolved in dichloromethane. Piperidine (64 mg, 0.5 mmol) was added and the reaction mixture was stirred under Ar at rt for 2 h. The reaction mixture was then concentrated and purified by preparative TLC, using 5% (2N ammonia in methanol)/dichloromethane as eluent: yield 7%; brown oil; R_{f} 0.16 (70% EtOAc/hexanes) LCMS (M+H⁺) 288, t_{R}=1.10 min.

### General Method Q: Preparation of 2-(4-methyl-3-pyridinyl)-1,3-thiazole4-carboxamides

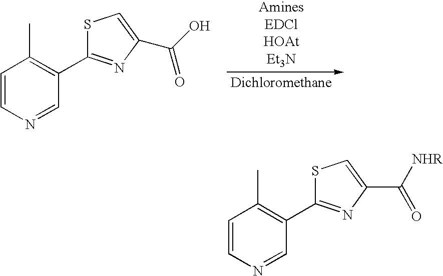

Amines (1.5 mmol) were weighed into EPA vials. A stock suspension of 4-methyl-3-pyridinyl)-1,3-thiazole-4-carboxylic acid*3KCl (801 mg, ˜1.8 mmol) was prepared by suspending it in dichloromethane (60 mL). N-Hydroxyazatriazole (0.280 g, 21.6 mmol) was added to each vial, followed by EDCI (0.438 g, 21.6 mmol) and triethylamine (0.55 g, 54 mmol). After stirring at rt for 30 min, 5 mL of stock solution was added to each vial. The reaction mixture was stirred at rt overnight. The products were purified by a variety of methods including, preparatory TLC, flash chromatography using the Biotage, or Gilson HPLC.

### General Method R: Synthesis of Pyridine n-Oxides

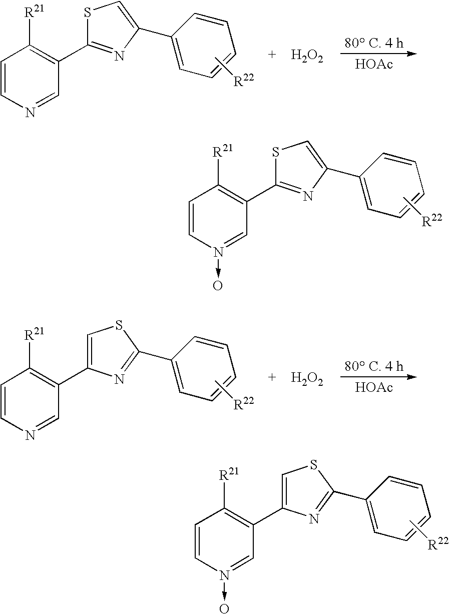

In a 25 mL flask, 1.39 g thiazole (0.005 mol) was mixed with 10 mL HOAc. After the mixture was cooled in ice bath, 1 mL H₂O₂ (about 0.017 mol) was added slowly with syringe. After the addition, the mixture was heated at 80° C. for 4 h then cooled to rt. Distilled water was added into the reaction mixture gradually till a lot of gray precipitate formed inside the solution. The precipitate was collected by filtration and washed with small amount of cold water. The product was dried in a vacuum oven, providing the target pyridine n-oxides. Yields averaged about 80%.

### Example 14

### Preparation of 2-(4-Chloromethyl-3-pyridyl)-4-(4-cyanophenyl)thiazole

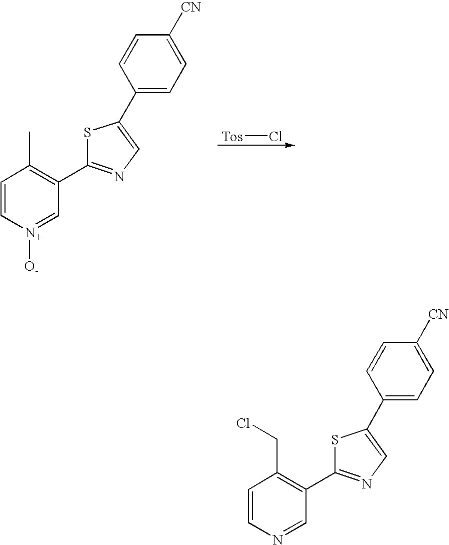

A mixture of the n-oxide (0.24 mmol) and tosyl chloride (0.26 mmol) in dioxane (5 mL) was heated to 80° C. and stirred for 3 h. The reaction mixture was evaporated to dryness and purified by silica gel chromatography. LCMS and ¹H NMR were consistent with the formation of the title compound (0.14 mmol, 58%).

### Example 15

### General Method S, as Exemplified by the Preparation of 2-(4-((dimethylamino)-3-pyridyl)-4-(4-cyanophenyl)thiazole

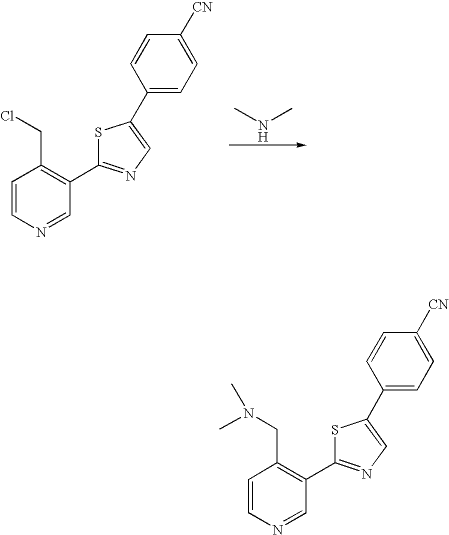

A mixture of 2-(4-chloromethyl-3-pyridyl)4(4-cyanophenyl) thiazole (0.052 mmol) and dimethyl amine (0.70 mmol) in THF was heated to 50° C. for 8 h. The mixture was evaporated to dryness and purified by silica gel column chromatography, providing the 0.021 mmol (40%) of the title compound: R_{f} 0.15 (60% EtOAc/hexane); LCMS (M+H)⁺ 321.4. ¹H NMR was consistent with the assigned structure.

TABLE II
2-(3-Pyridyl) thiazoles

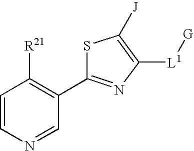

| Ex. | | | | | | t_{R}^{a,b} | MS^{a,b} | TLC R_{f} | General |
|---|---|---|---|---|---|---|---|---|---|
| No. | R^{21c} | J | L¹ | G^{c} | Salt | min | (M + H⁺) | d(Solvent) | Method |
| 16 | 4-t-Bu | H | Bond | 4-NO₂Ph | | | | 0.80 | O |
| | | | | | | | | (EtOAc) | |
| 17 | 4-iPr | H | Bond | 3-NO₂Ph | | | 326.2 | 0.60(50% | N |
| | | | | | | | | EtOAc/Hex) | |
| 18 | 4-Et | H | Bond | 3-CNPh | | | 292.2 | 0.50(50% | N |
| | | | | | | | | EtOAc/Hex) | |
| 19 | 4-Et | H | Bond | 2-NO₂Ph | | | 312.2 | 0.50(50% | N |
| | | | | | | | | EtOAc/Hex) | |
| 20 | 4-Et | H | Bond | 3-NO₂Ph | | | 312.1 | 0.50(50% | N |
| | | | | | | | | EtOAc/Hex) | |
| 21 | 4-Me | H | Bond | 3-NO₂Ph | | 2.44 | 298.1 | 0.18(50% | O |
| | | | | | | | | EtOAc/Hex) | |
| 22 | 4-Et | H | Bond | 3-thienyl | HBr | 2.45 | | | O |
| 23 | 4-Me | H | Bond | 2-thiophenecarbonitrile | TFA | 2.22 | 284 | 0.13(40% | O |
| | | | | | | | | EtOAc/Hex) | |
| 24 | 4-CyPr | H | Bond | 3-NO₂Ph | | 2.48 | 324.2 | 0.06 (20% | O |
| | | | | | | | | EtOAc/Hex) | |
| 25 | 4-Me | H | Bond | 2,4-diMeO | TFA | 3.05 | 313.2 | 0.34 (40% | O |
| | | | | | | | | EtOAc/Hex) | |
| 26 | 4-Me | H | Bond | 2-NO₂Ph | | 2.12 | 298.1 | 0.15 (40% | O |
| | | | | | | | | EtOAc/Hex) | |
| 27 | 4-CyPr | H | Bond | 4-Cl-3-NO₂ | | | 358.2 | 0.50 (50% | N |
| | | | | Ph | | | | EtOAc/Hex) | |
| 28 | 4-Me | H | Bond | 3,4-diFPh | | 2.57 | 289.2 | 0.24 (40% | O |
| | | | | | | | | EtOAc/Hex) | |
| 29 | 4-Me | H | Bond | 3-MeOPh | | 2.38 | 283.2 | 0.26 (30% | O |
| | | | | | | | | EtOAc/Hex) | |
| 30 | 4-iPr | H | Bond | 3-pyridinyl | | 1.32 | 282.3 | 0.25 3% | O |
| | | | | | | | | (2M NH₃ in | |
| | | | | | | | | MeOH)/CH₂Cl₂ | |
| 31 | 4-Me | H | Bond | 5-Cl thien-2-yl | HCl | 2.70 | 293.3 | 0.40 (40% | O |
| | | | | | | | | EtOAc/Hex) | |
| 32 | 4-Me | H | CH₂ | 4-MePh | | | | 0.32 (40% | M |
| | | | | | | | | EtOAc/Hex) | |
| 33 | 4-Me | H | Bond | 3-CNPh | | 2.25 | 278.29 | 0.24 (50% | O |
| | | | | | | | | EtOAc/Hex) | |
| 34 | 4-Pr | H | Bond | 2-FPh | | | | 0.79 (50% | O |
| | | | | | | | | EtOAc/Hex) | |
| 35 | 4-Pr | H | Bond | Ph | | | | 0.77 (50% | O |
| | | | | | | | | EtOAc/Hex) | |
| 36 | 4-Me | H | Bond | 4-ClPh | | | 287 | 0.33 (25% | O |
| | | | | | | | | EtOAc/Hex) | |
| 37 | 4-CyPr | H | Bond | Ph | | 2.35 | 279.2 | 0.14 (20% | O |
| | | | | | | | | EtOAc/Hex) | |
| 38 | 4-CyPr | H | Bond | 2-NO₂Ph | | | 324.2 | 0.55 (50% | N |
| | | | | | | | | EtOAc/Hex) | |
| 39 | 4-Me | H | Bond | 4-BrPh | | | | 0.30 (40% | O |
| | | | | | | | | EtOAc/Hex) | |
| 40 | 4-Et | H | Bond | 4-MePh | | | 281.3 | 0.55 (50% | N |
| | | | | | | | | EtOAc/Hex) | |
| 41 | 4-Pr | H | Bond | 2-NO₂Ph | | | | 0.70 (50% | |
| | | | | | | | | EtOAc/Hex) | |
| 42 | 4-CyPr | H | Bond | 4-FPh | | | 297.3 | 0.55 (50% | N |
| | | | | | | | | EtOAc/Hex) | |
| 43 | 4-Pr | H | Bond | 4-FPh | | | | 0.77 (50% | O |
| | | | | | | | | EtOAc/Hex) | |
| 44 | 4-Et | H | Bond | 3-FPh | | | 285.2 | 0.55 (50% | N |
| | | | | | | | | EtOAc/Hex) | |
| 45 | 4-Et | H | Bond | 2,3-dihydro-1,4- | | | 339.3 | 0.55 (50% | N |
| | | | | benzodioxin-6-yl) | | | | EtOAc/Hex) | |
| 46 | 4-Me | H | Bond | Ph | | 2.36 | 253.3 | 0.36 (25% | O |
| | | | | | | | | EtOAc/Hex) | |
| 47 | 4-Me | H | Bond | 2,4-diMePh | HBr | 2.62 | 281.3 | 0.39 (40% | O |
| | | | | | | | | EtOAc/Hex) | |
| 48 | 4-Me | H | Bond | 4-FPh | | 2.43 | 271.2 | 0.18 (40% | O |
| | | | | | | | | EtOAc/Hex) | |
| 49 | 4-Me | H | Bond | 5-Cl-thien-2-yl | | 2.73 | 293.1 | 0.29 (40% | O |
| | | | | | | | | EtOAc/Hex) | |
| 50 | 4-Me | H | O | cyclopentyl | | 2.21 | 261 | 0.50 (50% | K |
| | | | | | | | | EtOAc/Hex) | |
| 51 | 4-Pr | H | Bond | 3-CNPh | | | | 0.53 (100% | O |
| | | | | | | | | EtOAc) | |
| 52 | 4-Et | H | Bond | 4-Cl-3-NO₂ | | | 346.1 | 0.55 (50% | N |
| | | | | Ph | | | | EtOAc/Hex) | |
| 53 | 4-Me | H | Bond | 4-MeOPh | | | 283 | 0.30 (25% | O |
| | | | | | | | | EtOAc/Hex) | |
| 54 | 4-iPr | H | Bond | 2-NO₂Ph | | | 326.3 | 0.60 (50% | N |
| | | | | | | | | EtOAc/Hex) | |
| 55 | 4-Et | H | Bond | 4-ClPh | MSA | 2.84 | 301.2 | — | O/X |
| 56 | 4-Et | Me | Bond | Ph | | | 281.2 | 0.55 (50% | N |
| | | | | | | | | EtOAc/Hex) | |
| 57 | 4-Et | H | Bond | Ph | | | 267.2 | 0.65 (50% | N |
| | | | | | | | | EtOAc/Hex) | |
| 58 | 4-Me | H | Bond | 4-ClPh | | | 287 | 0.33 (25% | O |
| | | | | | | | | EtOAc/Hex) | |
| 59 | 4-Me | H | Bond | t-Bu | | 2.31 | 233 | 0.55 (50% | G |
| | | | | | | | | EtOAc/Hex) | |
| 60 | 4-CyPr | H | Bond | 3-pyridinyl | | 0.81 | 280.2 | 0.36 (100% | O |
| | | | | | | | | EtOAc/Hex) | |
| 61 | 4-Me | H | O | cyclohexyl | | 2.47 | 275 | 0.6 (50% | K |
| | | | | | | | | EtOAc/Hex) | |
| 62 | 4-CyPr | H | Bond | 4-NO₂Ph | | 2.51 | 324.2 | 0.06 (20% | O |
| | | | | | | | | EtOAc/Hex) | |
| 63 | 4-CyPr | H | Bond | 2,4-diMePh | | | 307.2 | 0.50 50% | N |
| | | | | | | | | EtOAc/Hex) | |
| 64 | 4-Et | H | Bond | 2-MeOPh | | | 297.2 | 0.55 (50% | N |
| | | | | | | | | EtOAc/Hex) | |
| 65 | 4-Pr | H | Bond | 4-MePh | | | | 0.78 (100% | O |
| | | | | | | | | EtOAc/Hex) | |
| 66 | 4-Et | H | Bond | cyclohexyl | | | 273.3 | 0.65 (50% | N |
| 67 | 4-iPr | H | Bond | Ph | | | 281.2 | 0.40 (30% | |
| 68 | 4-Me | H | OCH₂ | Ph | | 2.29 | 283 | 0.35 (50% | K |
| | | | | | | | | EtOAc/Hex) | |
| 69 | 4-Me | H | Bond | 1-cyclopenten-1-yl | | 2.39 | 243 | 0.5 (50% | G |
| | | | | | | | | EtOAc/Hex) | |
| 70 | 4-Me | H | Bond | cycloheptyl | | 2.72 | 273 | 0.29 (25% | G |
| | | | | | | | | EtOAc/Hex) | |
| 71 | 4-Me | H | O | (2R)- | | 2.53 | 287 | 0.5 (50% | K |
| | | | | bicyclo[2.2.1]hept-2-yl | | | | EtOAc/Hex) | |
| 72 | 4-CyPr | H | Bond | 2-MeOPh | | | 309.2 | 0.50 (50% | N |
| | | | | | | | | EtOAc/Hex) | |
| 73 | 4-Pr | H | Bond | 4-CF₃Ph | | | | 0.75 (100% | O |
| wlf | | | | | | | | EtOAc/Hex) | |
| 74 | 4-CyPr | H | Bond | 3-FPh | | 2.48 | 297.2 | 0.11 (20% | O |
| | | | | | | | | EtOAc/Hex) | |
| 75 | 4-Et | H | Bond | 2-ClPh | | | 301.2 | 0.60 (50% | N |
| | | | | | | | | EtOAc/Hex) | |
| 76 | 4-Me | H | O | iPr | | 1.99 | 235 | 0.28 (25% | I |
| | | | | | | | | EtOAc/Hex) | |
| 77 | 4-Me | H | Bond | 3-Br thien-2-yl | TFA | 2.62 | 339.6 | 0.34 (40% | O |
| | | | | | | | | EtOAc/Hex) | |
| 78 | 4-Me | H | Bond | cyclopentyl | | 2.29 | 245 | 0.11 (20% | G |
| | | | | | | | | EtOAc/Hex) | |
| 79 | 4-Me | H | CH₂ | 4-ClPh | | 2.56 | 301.3 | 0.24 (40% | M |
| 80 | 4-iPr | H | Bond | 3-FPh | | | 299.5 | 0.55 (50% | N |
| | | | | | | | | EtOAc/Hex) | |
| 81 | 4- iPr | H | Bond | 4-Me | 2TFA | 2.70 | | 0.55 (50% | O |
| | | | | pyridin-3-yl | | | | EtOAc/Hex) | |
| 82 | 4-Me | H | O | cycloheptyl | | 2.57 | 289 | 0.66 (50% | K |
| | | | | | | | | EtOAc/Hex) | |
| 83 | 4-iPr | H | Bond | 4-NO₂Ph | | | 326.3 | 0.60 (50% | N |
| | | | | | | | | EtOAc/Hex) | |
| 84 | 4-Pr | H | Bond | 3-NO₂Ph | | | | 0.69 (100% | O |
| | | | | | | | | EtOAc/Hex) | |
| 85 | 4-Me | H | Bond | 4-NO₂Ph | TFA | 2.38 | 298.3 | 0.17 (40% | O |
| | | | | | | | | EtOAc/Hex) | |
| 86 | 4-Et | H | Bond | 2,4-diMePh | | | 294.2 | 0.55 (50% | N |
| | | | | | | | | EtOAc/Hex) | |
| 87 | 4-Me | H | Bond | cyclohexyl | | 2.63 | 259 | 0.33 (33% | G |
| | | | | | | | | EtOAc/Hex) | |
| 88 | 4-Et | H | Bond | 4-FPh | | | 285.2 | 0.60 (50% | N |
| | | | | | | | | EtOAc/Hex) | |
| 89 | 4-Me | H | Bond | 4-NO₂Ph | | 2.34 | 298.2 | 0.20 (40% | O |
| | | | | | | | | EtOAc/Hex) | |
| 90 | 4-CyPr | H | Bond | 3-thienyl | HBr | 2.29 | | 0.55 (50% | O |
| | | | | | | | | EtOAc/Hex) | |
| 91 | 4-Et | H | Bond | 4-NO₂Ph | | | 312.2 | 0.45 (50% | N |
| | | | | | | | | EtOAc/Hex) | |
| 92 | 4-Me | H | Bond | 3-ClPh | | 2.66 | 287.29 | 0.32 (50% | O |
| 93 | 4-t-Bu | H | Bond | 4-FPh | | | | 0.80 (50% | O |
| | | | | | | | | EtOAc/Hex) | |
| 94 | 4-iPr | H | Bond | 3-ClPh | | | 315.6 | 0.50 (50% | N |
| | | | | | | | | EtOAc/Hex) | |
| 95 | 4-Me | H | Bond | 3-Cl thien-2-yl | TFA | | 256 | 0.34 (40% | O |
| | | | | | | | | EtOAc/Hex) | |
| 96 | 4-Me | H | Bond | 3-FPh | | 2.44 | 271.27 | 0.23 (50% | O |
| | | | | | | | | EtOAc/Hex) | |
| 97 | 4-Pr | H | Bond | 2-naphthyl | | | | 0.75 (100% | O |
| | | | | | | | | EtOAc/Hex) | |
| 98 | 4-Et | H | Bond | 4-MeOPh | | | 297.2 | 0.60 (50% | N |
| | | | | | | | | EtOAc/Hex) | |
| 99 | 4-t-Bu | H | Bond | 4-MePh | | | | 0.83 (100% | O |
| | | | | | | | | EtOAc/Hex) | |
| 100 | 4-Me | H | Bond | 3-pyridinyl | | 0.64 | 254.4 | 0.13 (3% | O |
| | | | | | | | | MeOH/ | |
| | | | | | | | | CH₂CL₂) | |
| 101 | 4-t-Bu | H | Bond | 4-MeOPh | | | | 0.83 (100% | O |
| | | | | | | | | EtOAc/Hex) | |
| 102 | 4-Et | H | Bond | 3-ClPh | | | 301.2 | 0.50 (50% | N |
| | | | | | | | | EtOAc/Hex) | |
| 103 | 4-Pr | H | Bond | 3-ClPh | | | | 0.74 (100% | O |
| | | | | | | | | EtOAc) | |
| 104 | 4-Et | H | Bond | 3-BrPh | | | 345.2 | 0.50( 50% | N |
| | | | | | | | | EtOAc/Hex) | |
| 105 | 4-t-Bu | H | Bond | 3-NO₂Ph | | | | 0.74 (100% | O |
| | | | | | | | | EtOAc/Hex) | |
| 106 | 4-iPr | H | Bond | 2-MeOPh | | | 311.3 | 0.55 (50% | N |
| | | | | | | | | EtOAc/Hex) | |
| 107 | 4-iPr | H | Bond | 3,4-diFPh | | | 317.5 | 0.50 (50% | N |
| | | | | | | | | EtOAc/Hex) | |
| 108 | 4-t-Bu | H | Bond | Ph | | 2.96 | 295.1 | 0.82 (100% | O |
| | | | | | | | | EtOAc) | |
| 109 | 4-Pr | H | Bond | 4-ClPh | | 3.15 | 315 | 0.74 (100% | N |
| | | | | | | | | EtOAc/Hex) | |
| 110 | 4-Et | H | Bond | 2,5-diMeO | | | 327.2 | 0.50 (50% | N |
| | | | | Ph | | | | EtOAc/Hex) | |
| 111 | 4-Me | H | Bond | 4-pyridinyl | 2TFA | 0.77 | 254 | 0.19 (3% MeOH/ | O |
| | | | | | | | | CH₂Cl₂) | |
| 112 | 4-Pr | H | Bond | 3-BrPh | | 2.05 | 361.2 | 0.77 (100% | O |
| | | | | | | | | EtOAc) | |
| 113 | 4-t-Bu | H | Bond | 4-Cl-3-NO₂ | | 3.22 | 374.2 | 0.78 (100% | O |
| | | | | Ph | | | | EtOAc) | |
| 114 | 4-iPr | H | Bond | 2,4-diMePh | | | 309.7 | 0.50 (50% | N |
| | | | | | | | | EtOAc/Hex) | |
| 115 | 4-Me | H | Bond | 4-COOHPh | NH₄Cl | 2.30 | 295.97 | 0.48 (10% MeOH/ | O |
| | | | | | | | | CH₂Cl₂) | |
| 116 | 4-Me | H | Bond | 3-BrPh | | 2.73 | 331.28 | 0.25 (50% | O |
| | | | | | | | | EtOAc/Hex) | |
| 117 | 4-Me | H | OCH₂ | exo/endonorbornyl | | 2.74 | 301 | 0.54 (50% | K |
| | | | | | | | | EtOAc/Hex) | |
| 118 | 4-t-Bu | H | Bond | 2-BrPh | | 3.07 | 312.9 | 0.78 (100% | O |
| | | | | | | | | EtOAc) | |
| 119 | 4-iPr | H | Bond | 4-pyridinyl | 2TFA | 0.99 | 282 | 0.27 (100% | O |
| | | | | | | | | EtOAc) | |
| 120 | 4-iPr | H | Bond | 3-CNPh | | | 306.6 | 0.50 (50% | N |
| | | | | | | | | EtOAc/Hex) | |
| 121 | 4-iPr | H | Bond | 7-heptyl | | 3.03 | 301 | 0.65 (50% | N |
| | | | | | | | | EtOAc/Hex) | |
| 122 | 4-CyPr | H | Bond | 4-ClPh | | | 313.6 | 0.40 (30% | N |
| | | | | | | | | EtOAc/Hex) | |
| 123 | 4-Pr | H | Bond | 4-MeOPh | | 1.29 | 311.3 | 0.76 (100% | O |
| | | | | | | | | EtOAc/Hex) | |
| 124 | 4-iPr | H | Bond | 4-Cl-3-NO₂ | | | 360.8 | 0.55 (50% | N |
| | | | | Ph | | | | EtOAc/Hex) | |
| 125 | 4-CyPr | H | Bond | 4-cyclohexyl | | | 285.2 | 0.65 (50% | |
| | | | | | | | | EtOAc/Hex) | |
| 126 | 4-t-Bu | H | Bond | 2-ClPh | | 3.14 | 328.9 | 0.84 (100% | O |
| | | | | | | | | EtOAc/Hex) | |
| 127 | 4-iPr | H | Bond | 3-thienyl | HBr | 2.61 | | | O |
| 128 | 4-Pr | H | Bond | 5-Me-3-Ph- | | 1.33 | 362.3 | 0.72 (100% | O |
| | | | | 4-isoxazolyl | | | | EtOAc) | |
| 129 | 4-iPr | H | Bond | 4-MeOPh | | | 311.2 | 0.40 (30% | N |
| | | | | | | | | EtOAc/Hex) | |
| 130 | 4-t-Bu | H | Bond | 3-CNPh | | 3.00 | 319.9 | 0.77 (100% | O |
| | | | | | | | | EtOAc) | |
| 131 | 4-Pr | H | Bond | 2-MeOPh | | 1.33 | 311.3 | 0.78 (100% | O |
| | | | | | | | | EtOAc) | |
| 132 | 4-CyPr | H | Bond | 4-MeOPh | | | 309.2 | 0.40 (30% | N |
| | | | | | | | | EtOAc/Hex) | |
| 133 | 4-Me | H | Bond | 2-MeOPh | TFA | 3.02 | 283.3 | 0.29 (40% | O |
| | | | | | | | | EtOAc/Hex) | |
| 134 | 4-iPr | H | Bond | 4-FPh | | | 299.3 | 0.60 (30% | N |
| | | | | | | | | EtOAc/Hex) | |
| 135 | 4-Me | H | O | cyclobutyl | | 1.94 | 247 | 0.55 (50% | I |
| | | | | | | | | EtOAc/Hex) | |
| 136 | 4-Pr | H | Bond | 4-(diFMeO) | | 1.28 | 347.3 | 0.78 (100% | O |
| | | | | Ph | | | | EtOAc) | |
| 137 | 4-t-Bu | H | Bond | 4-CNPh | | 2.96 | 319.9 | 0.77 (100% | O |
| | | | | | | | | EtOAc) | |
| 138 | 4-Et | H | Bond | 4-CNPh | | | 292.2 | 0.50 (50% | N |
| | | | | | | | | EtOAc/Hex) | |
| 139 | 4-Et | H | Bond | 4-(diFMeO) | | | 333.2 | 0.55 (50% | N |
| | | | | Ph | | | | EtOAc/Hex) | |
| 140 | 4-CyPr | H | Bond | 2-FPh | | | 297.2 | 0.60 (50% | N |
| | | | | | | | | EtOAc/Hex) | |
| 141 | 4-t-Bu | H | Bond | 3-FPh | | 3.11 | 312.9 | 0.55 (50% | O |
| | | | | | | | | EtOAc/Hex) | |
| 142 | 4-t-Bu | H | Bond | 2,4-diMePh | | 3.55 | 323 | 0.83 (100% | O |
| | | | | | | | | EtOAc) | |
| 143 | 4-t-Bu | Me | Bond | Ph | | 3.00 | 308.5 | 0.79 (100% | J |
| | | | | | | | | EtOAc) | |
| 144 | 4-Me | Me | Bond | 4-ClPh | | | 301 | 0.33 (25% | J |
| | | | | | | | | EtOAc/Hex) | |
| 145 | 4-iPr | H | Bond | 2,5-diMeO | | | 341.6 | 0.50 (50% | N |
| | | | | | | | | EtOAc/Hex) | |
| 146 | 4-iPr | H | Bond | 4-MePh | | | 295.3 | 0.60 (50% | N |
| | | | | | | | | EtOAc/Hex) | |
| 147 | 4-Pr | H | Bond | 4-BrPh | | 3.18 | 361.2 | 0.76 (100% | O |
| | | | | | | | | EtOAc/Hex) | |
| 148 | 4-Pr | H | Bond | 2-ClPh | | 3.00 | 315.3 | 0.55 (50% | O |
| | | | | | | | | EtOAc/Hex) | |
| 149 | 4-t-Bu | H | Bond | 4-(diFMeO) | | 3.11 | 361 3 | 0.83 (100% | O |
| | | | | Ph | | | | EtOAc) | |
| 150 | 4-t-Bu | H | Bond | 2-MeOPh | | 3.00 | 325 | 0.83 (100% | O |
| | | | | | | | | EtOAc) | |
| 151 | 4-t-Bu | H | Bond | 3,4-diClPh | | 3.51 | 363.2 | 0.77 (100% | O |
| | | | | | | | | EtOAc) | |
| 152 | 4-Et | H | Bond | 4-CF₃Ph | | | 335.2 | 0.50 (50% | N |
| | | | | | | | | EtOAc/Hex) | |
| 153 | 4-iPr | H | Bond | cyclohexyl | | 2.88 | 287 | 0.48 (25% | G |
| | | | | | | | | EtOAc/Hex) | |
| 154 | 4-Et | H | Bond | 4-ClPh | | | 301.2 | 0.60 (50% | N |
| | | | | | | | | EtOAc/Hex) | |
| 155 | 4-Et | H | Bond | 2-FPh | | | 285.2 | 0.60 (50% | N |
| | | | | | | | | EtOAc/Hex) | |
| 156 | 4-iBu | H | Bond | 4-ClPh | | 3.37 | 329 | 0.45 (50% | N |
| | | | | | | | | EtOAc/Hex) | |
| 157 | 4-Pr | H | Bond | 2,4-diMePh | | 1.36 | 309.3 | 0.77 (100% | O |
| | | | | | | | | EtOAc) | |
| 158 | 4-iPr | H | Bond | cyclopentyl | | 2.66 | 273 | 0.63 (50% | G |
| | | | | | | | | EtOAc/Hex) | |
| 159 | 4-t-Bu | H | Bond | 3-ClPh | | 3.55 | 329 | 0.81 (100% | O |
| | | | | | | | | EtOAc) | |
| 160 | 4-Me | H | Bond | 4-Cl-3-NO₂ | | 2.62 | 332.2 | 0.35 (50% | O |
| | | | | Ph | | | | EtOAc/Hex) | |
| 161 | 4-Et | Me | Bond | 4-Cl-Ph | | | 315.2 | 0.55 (50% | N |
| | | | | | | | | EtOAc/Hex) | |
| 162 | 4-CyPr | H | Bond | 3-BrPh | | | | 0.55 (50% | N |
| | | | | | | | | EtOAc/Hex) | |
| 163 | 4-iPr | H | O | iPr | | 2.29 | 263 | 0.50 (50% | I |
| | | | | | | | | EtOAc/Hex) | |
| 164 | 4-Me | H | Bond | 2-naphthyl | | 2.81 | 303.2 | 0.32 (40% | O |
| | | | | | | | | EtOAc/Hex) | |
| 165 | 4-Me | H | Bond | 2-ClPh | | 2.43 | 287.3 | 0.48 (50% | O |
| | | | | | | | | EtOAc/Hex) | |
| 166 | 4-Et | H | Bond | 4-BrPh | | | 245.2 | 0.55 (50% | N |
| | | | | | | | | EtOAc/Hex) | |
| 167 | 4-Me | H | Bond | 4-(diFMeO) | | 2.49 | 319.3 | 0.20 (50% | O |
| | | | | Ph | | | | EtOAc/Hex) | |
| 168 | 4-CyPr | Me | Bond | Ph | | | 293.2 | 0.60 (50% | N |
| | | | | | | | | EtOAc/Hex) | |
| 169 | 4-Me | H | Bond | 3,5-diCF₃ | | | | 0.30 (40% | O |
| | | | | | | | | EtOAc/Hex) | |
| 170 | 4-CyPr | H | Bond | 4-MePh | | 2.56 | 293.2 | 0.15 (20% | O |
| | | | | | | | | EtOAc/Hex) | |
| 171 | 4-iPr | H | Bond | 4-ClPh | | | 315.6 | 0.50 (30% | N |
| | | | | | | | | EtOAc/Hex) | |
| 172 | 4-CyPr | H | Bond | 2,5-diMeO | | | 339.2 | 0.50 (30% | N |
| | | | | Ph | | | | EtOAc/Hex) | |
| 173 | 4-CyPr | H | Bond | 3-ClPh | | 2.69 | 313.1 | 0.12 (20% | O |
| | | | | | | | | EtOAc/Hex) | |
| 174 | 4-CyPen | H | Bond | 3-NO₂Ph | | | 352.3 | 0.50 (50% | N |
| | | | | | | | | EtOAc/Hex) | |
| 175 | 4-Me | H | Bond | 2,4-diClPh | | 2.73 | 321 | 0.35 (50% | O |
| | | | | | | | | EtOAc/Hex) | |
| 176 | 4-iPr | H | Bond | 2-ClPh | | | 315.6 | 0.50 (50% | N |
| | | | | | | | | EtOAc/Hex) | |
| 177 | 4-CyPr | H | Bond | 4-pyridinyl | | 0.75 | 280.2 | 0.34 (50% | O |
| | | | | | | | | EtOAc/Hex) | |
| 178 | H | H | CH₂ | 4-ClPh | | | 287 | 0.27 (25% | M |
| | | | | | | | | EtOAc/Hex) | |
| 179 | 4-Pr | H | Bond | 4-NO₂Ph | | 1.31 | 326.2 | 0.70 (100% | O |
| | | | | | | | | EtOAc/Hex) | |
| 180 | 4-Et | H | Bond | 1-adamantyl | | | 325.4 | 0.50 (50% | N |
| | | | | | | | | EtOAc/Hex) | |
| 181 | 4-iPr | H | Bond | 3,4-diClPh | | | 349.2 | 0.50 (50% | N |
| | | | | | | | | EtOAc/Hex) | |
| 182 | 4-Me | H | Bond | 2,4-diClPh | HCl | | 321 | 0.42 (5% | O |
| | | | | | | | | MeOH/CH₂Cl₂) | |
| 183 | 4-Me | H | Bond | N,N-diethyl- | | | | 0.65 (100% | Y |
| | | | | 3-aniline | | | | EtOAc) | |
| 184 | 4-Bu | H | Bond | 4-ClPh | | 3.24 | 329 | 0.38 (10% | N |
| | | | | | | | | EtOAc/CH₂Cl₂) | |
| 185 | H | H | CH₂ | 4-MePh | | | 267 | 0.41 (25% | M |
| | | | | | | | | EtOAc/Hex) | |
| 186 | 4-Me | H | Bond | 2-FPh | | 2.48 | 271.27 | 0.45 (50% | O |
| | | | | | | | | EtOAc/Hex) | |
| 187 | 4-t-Bu | H | Bond | 4-ClPh | | 3.25 | 328.9 | 0.82 (100% | O |
| | | | | | | | | EtOAc) | |
| 188 | 4-t-Bu | H | Bond | 4-CF₃Ph | | 3.36 | 363.2 | 0.83 (100% | O |
| | | | | | | | | EtOAc) | |
| 189 | 4-CyPen | H | Bond | 3-FPh | | | 325.3 | 0.60 (50% | N |
| | | | | | | | | EtOAc/Hex) | |
| 190 | 4-Ph | H | Bond | 4-FPh | | | 0.55 (50% | N | |
| | | | | | | | | EtOAc/Hex) | |
| 191 | H | H | CH₂ | 4-BrPh | | | 330 | 0.36 (25% | M |
| | | | | | | | | EtOAc/Hex) | |
| 192 | 4-t-Bu | H | Bond | 4-BrPh | | 3.29 | 373.2 | 0.83 (100% | O |
| | | | | | | | | EtOAc/Hex) | |
| 193 | 4-CyPr | H | Bond | 4-Me-3- | | 0.79 | 294.2 | 0.30 (100% | O |
| | | | | pyridinyl | | | | EtOAc) | |
| 194 | 4-Pr | H | Bond | t-Bu | | 1.38 | 261.3 | 0.90 (100% | G |
| | | | | | | | | EtOAc) | |
| 195 | 4-t-Bu | H | Bond | 3-BrPh | | 3.29 | 373.2 | 0.83 (100% | O |
| | | | | | | | | EtOAc) | |
| 196 | 4-t-Bu | Me | Bond | 4-BrPh | | 3.25 | 387.2 | 0.82 (100% | W |
| | | | | | | | | EtOAc) | |
| 197 | 4-iPr | H | Bond | 4-BrPh | | | 359.2 | 0.60 (50% | N |
| | | | | | | | | EtOAc/Hex) | |
| 198 | H | H | Bond | CyBu | | 2.75 | 231 | 0.24 (25% | G |
| | | | | | | | | EtOAc/Hex) | |
| 199 | 4-Et | H | Bond | 2,4-diClPh | | | 335.2 | 0.50 (50% | N |
| | | | | | | | | EtOAc/Hex) | |
| 200 | 4-Pr | H | Bond | 4-CNPh | | 1.37 | 357.3 | 0.71 (100% | O |
| | | | | | | | | EtOAc) | |
| 201 | 4-CyPen | H | Bond | 2-NO₂Ph | | | | 0.55 (50% | N |
| | | | | | | | | EtOAc/Hex) | |
| 202 | 4-Me | H | Bond | 4-CNPh | | 2.89 | 278.3 | 0.17(40% | O |
| | | | | | | | | EtOAc/Hex) | |
| 203 | 4-Me | H | Bond | 1-adamantyl | | 3.16 | 311 | 0.47 (50% | G |
| | | | | | | | | EtOAc/Hex) | |
| 204 | 4-Et | Me | Bond | 4-MeOPh | | | 311.2 | 0.50 (50% | N |
| | | | | | | | | EtOAc/Hex) | |
| 205 | 4-t-Bu | H | Bond | 2-NO₂Ph | | 2.81 | 339.9 | 0.71 (100% | O |
| | | | | | | | | EtOAc) | |
| 206 | 4-iPr | H | Bond | t-Bu | | 2.76 | 261 | 0.50 (25% | G |
| | | | | | | | | EtOAc/Hex) | |
| 207 | H | H | CH₂ | 3-ClPh | | | 287 | 0.32 (25% | M |
| | | | | | | | | EtOAc/Hex) | |
| 208 | piperidinyl | H | Bond | 3-ClPh | | 2.31 | 356.3 | 0.06 (20% | L |
| | | | | | | | | EtOAc/Hex) | |
| 209 | 4-Me | H | Bond | 4(trifluoromethoxy)Ph | | 2.8 | 337.3 | 0.50 (50% | O |
| | | | | | | | | EtOAc/Hex) | |
| 210 | 4-Et | H | Bond | 3,4-diClPh | | | 335.2 | 0.55 (50% | N |
| | | | | | | | | EtOAc/Hex) | |
| 211 | 4-CyPen | H | Bond | 3-CNPh | | | 332.2 | 0.50 (50% | N |
| | | | | | | | | EtOAc/Hex) | |
| 212 | 4-iBu | H | Bond | CyHex | | 3.75 | 301 | 0.70 (50% | W |
| | | | | | | | | EtOAc/Hex) | |
| 213 | 4-iPr | H | Bond | 4-CNPh | | | 306.6 | 0.55 (50% | N |
| | | | | | | | | EtOAc/Hex) | |
| 214 | 4-iPr | H | Bond | 4-(diFMeO) | | | 347.7 | 0.50 (50% | N |
| | | | | | | | | EtOAc/Hex) | |
| 215 | 4-t-Bu | Me | Bond | 4-MeOPh | | 2.96 | 339 | 0.75 (100% | W |
| | | | | | | | | EtOAc) | |
| 216 | 4-FPh | H | Bond | 3-NO₂Ph | | 3.26 | 378.2 | 0.81(100% | O |
| | | | | | | | | EtOAc) | |
| 217 | H | H | CH₂ | 3-MePh | | | 267 | 0.39 (25% | M |
| | | | | | | | | EtOAc/Hex) | |
| 218 | 4-iPr | H | Bond | 2-naphthyl | | | 331.6 | 0.50 (50% | N |
| | | | | | | | | EtOAc/Hex) | |
| 219 | 4-FPh | H | Bond | 3-ClPh | | 3.49 | 367.3 | 0.50 (50% | O |
| | | | | | | | | EtOAc/Hex) | |
| 220 | 4-CyPr | H | Bond | 2-naphthyl | | | 329.2 | 0.55 (50% | N |
| | | | | | | | | EtOAc/Hex) | |
| 221 | 4-Et | H | Bond | 4-(trifluoromethoxy)Ph | | | 351.2 | 0.55 (50% | N |
| | | | | | | | | EtOAc/Hex) | |
| 222 | 4-iPr | H | Bond | 2-pyridinyl | 2TFA | 1.8 | 282.2 | 0.27 (3% | O |
| | | | | | | | | MeOH/CH₂Cl₂) | |
| 223 | 4-t-Bu | H | Bond | 2,4-diClPh | | 3.47 | 363.1 | 0.83 (100% | O |
| | | | | | | | | EtOAc) | |
| 224 | 4-iPr | H | Bond | 1-cyclopentene | | 2.76 | 270 | 0.60 (50% | G |
| | | | | | | | | EtOAc/Hex) | |
| 225 | 4-Ph | H | Bond | 3-NO₂Ph | | 3.16 | 360.2 | 0.68 (50% | O |
| | | | | | | | | EtOAc/Hex) | |
| 226 | 4-CyPr | H | Bond | 4-Br-Ph | | 2.82 | 357.1 | 0.15 (20% | O |
| | | | | | | | | EtOAc/Hex) | |
| 227 | 4-FPh | H | Bond | 3-CNPh | | 3.13 | 358.3 | 0.60 (50% | O |
| | | | | | | | | EtOAc/Hex) | |
| 228 | H | H | O | cycloheptyl | | 2.89 | 275 | 0.66 (50% | K |
| | | | | | | | | EtOAc/Hex) | |
| 229 | 4-iPr | H | Bond | 2-FPh | | | 299.7 | 0.50 (50% | N |
| | | | | | | | | EtOAc/Hex) | |
| 230 | 4-Me | H | Bond | 4-CNPh | MSA | | | 0.50 (50% | O/P |
| | | | | | | | | EtOAc/Hex) | |
| 231 | 4-Ph | H | Bond | 4-MePh | | 3.24 | 329.3 | 0.74 (100% | O |
| | | | | | | | | EtOAc | |
| 232 | 4-Me | I | Bond | 4-ClPh | | 3.23 | 413 | 0.50 (50% | X |
| | | | | | | | | EtOAc/Hex) | |
| 233 | 4-FPh | H | Bond | 3-FPh | | 3.3 | 351.3 | 0.73 (50% | O |
| | | | | | | | | EtOAc/Hex) | |
| 234 | 4-CyPen | H | Bond | 4-NO₂Ph | | 3.11 | 352.3 | 0.53 (50% | N |
| | | | | | | | | EtOAc/Hex) | |
| 235 | 4-morpholinyl | H | Bond | 4-CNPh | | | | 0.76 (10% | S |
| | Me | | | | | | | EtOAc) | |
| 236 | 4-Ph | H | Bond | Ph | | | | 0.50 (50% | N |
| | | | | | | | | EtOAc/Hex) | |
| 237 | H | H | O | Exo-norborn-2-yl | | | | 0.50 (50% | K |
| | | | | | | | | EtOAc/Hex) | |
| 238 | 4-CyPr | H | Bond | 4-(diFMeO) | | | | 0.60 (50% | N |
| | | | | Ph | | | | EtOAc) | |
| 239 | H | H | Bond | t-Bu | | | | 0.50 (50% | G |
| | | | | | | | | EtOAc/Hex) | |
| 240 | 4-t-Bu | H | Bond | 2-Me-3- | | | | 0.15 (100% | O |
| | | | | pyridinyl | | | | EtOAc) | |
| 241 | 4-Me | H | Bond | 2-pyridinyl | | | | 0.50 (5% | O |
| | | | | | | | | MeOH/CH₂Cl₂) | |
| 242 | 4-CyPr | H | Bond | 4-CNPh | | | | 0.55 (50% | N |
| | | | | | | | | EtOAc/Hex) | |
| 243 | 4-Et | Me | Bond | 4-BrPh | | | | 0.55 (50% | N |
| | | | | | | | | EtOAc/Hex) | |
| 244 | 4-Me | H | carboxamide | N-piperidin-1-yl | | | | 0.17 (3% MeOH/ | Q |
| | | | | | | | | CH₂Cl₂) | |
| 245 | 4-Pr | Me | Bond | 4-BrPh | | | | 0.77 (100% | O |
| | | | | | | | | EtOAc) | |
| 246 | 4-CyPr | H | Bond | 4-CF₃Ph | | | | 0.60 (50% | N |
| | | | | | | | | EtOAc/Hex) | |
| 247 | H | H | O | cyclohexyl | | | | 0.50 (50% | K |
| | | | | | | | | EtOAc/Hex) | |
| 248 | 4-CyPr | Me | Bond | 4-ClPh | | | | 0.60 (50% | N |
| | | | | | | | | EtOAc/Hex) | |
| 249 | H | H | Bond | ethyl 3- | | | | 0.50 (50% | G |
| | | | | methylbutaneoate | | | | EtOAc/Hex) | |
| 250 | 4-Me | H | Bond | 4-CNPh | MSA | | | 0.50 (50% | O/X |
| | | | | | | | | EtOAc/Hex) | |
| 251 | 4-Ph | H | Bond | 3-CNPh | | | | 0.50 (50% | O |
| | | | | | | | | EtOAc/Hex) | |
| 252 | 4-Me | H | Bond | 4-CNPh | HCl | | | 0.50 (50% | O/X |
| | | | | | | | | EtOAc/Hex) | |
| 253 | 4-Et | H | Bond | 3-pyridinyl | | | | 0.50 (50% | N |
| | | | | | | | | EtOAc/Hex) | |
| 254 | 4-Pr | H | Bond | 3-phenyl | | 1.34 | 401.3 | 0.80 (100% | O |
| | | | | benzoate | | | | EtOAc) | |
| 255 | 4-Me | I | Bond | cyclohexyl | | | | 0.50 (50% | X |
| | | | | | | | | EtOAc/Hex) | |
| 256 | 4-CyPen | H | Bond | 3-ClPh | | | | 0.55 (50% | N |
| | | | | | | | | EtOAc/Hex) | |
| 257 | 4-Et | H | Bond | 2-naphthyl | | | | 0.50 (50% | N |
| | | | | | | | | EtOAc/Hex) | |
| 258 | 4-t-Bu | H | Bond | 4-(trifluoromethoxy)Ph | | | | 0.83 (100% | O |
| | | | | | | | | EtOAc) | |
| 259 | H | H | O | CyPen | | | | 0.50 (50% | K |
| | | | | | | | | EtOAc/Hex) | |
| 260 | 4-CyPr | H | Bond | 3,4-diClPh | | | | 0.55 (50% | K |
| | | | | | | | | EtOAc/Hex) | |
| 261 | H | H | Bond | 1-adamantyl | | | | 0.50 (50% G | |
| | | | | | | | | EtOAc/Hex) | |
| 262 | H | Me | Bond | Ph | | | | 0.44 (25% | O |
| | | | | | | | | EtOAc/Hex) | |
| 263 | 4-iPr | H | Bond | 3-furanl | TFA | | | 0.50 (50% | O |
| | | | | | | | | EtOAc/Hex) | |
| 264 | 4-Me | H | Bond | 4-CNPh | maleate | | | 0.50 (50% | O/P |
| | | | | | | | | EtOAc/Hex) | |
| 265 | 4-Me | H | Bond | 3,4-diClPh | | | | 0.50 (50% | O |
| | | | | | | | | EtOAc/Hex) | |
| 266 | 4-CyPr | H | Bond | 4-CNPh | MSA | | | 0.50 (50% | O/X |
| | | | | | | | | EtOAc/Hex) | |
| 267 | H | H | OCH₂ | Endo/Exo | | | | 0.50 (50% | K |
| | | | | norbornyl | | | | EtOAc/Hex) | |
| 268 | 4-CyPr | H | Bond | 2-ClPh | | | | 0.13 (20% | O |
| | | | | | | | | EtOAc/Hex) | |
| 269 | 4-Ph | H | Bond | 3-FPh | | | | 0.50 (50% | O |
| | | | | | | | | EtOAc/Hex) | |
| 270 | H | Me | Bond | 4-ClPh | | | | 0.34 (25% | J |
| | | | | | | | | EtOAc/Hex) | |
| 271 | 4-CyPr | Me | Bond | 4-MeOPh | | | | 0.55 (50% | N |
| | | | | | | | | EtOAc/Hex) | |
| 272 | 4-t-Bu | Me | Bond | 3-Cl-4-Me | | | | 0.75 (100% | O |
| | | | | Ph | | | | EtOAc) | |
| 273 | 4-Me | H | Carbonyl | Endo/exo N- | | 2.25 | 314 | 0.25 (3% | Q |
| | | | | norbornylamine | | | | MeOH/CH₂Cl₂) | |
| 274 | 4-FPh | H | Bond | 4-FPh | | | | 0.66 (50% | O |
| | | | | | | | | EtOAc/Hex) | |
| 275 | 4-FPh | H | Bond | 3,4-diClPh | | | | 0.50 (50% | O |
| | | | | | | | | EtOAc/Hex) | |
| 276 | 4-CyPen | H | Bond | 4-FPh | | | | 0.60 (50% | N |
| | | | | | | | | EtOAc/Hex) | |
| 277 | 4-CyPen | H | Bond | 3-BrPh | | | | 0.55 (50% | N |
| | | | | | | | | EtOAc/Hex) | |
| 278 | 4-thiomorpholine | H | Bond | 4-ClPh | | 3.21 | 333.3 | 0.59 (50% | L |
| | | | | | | | | EtOAc/Hex) | |
| 279 | H | H | CH₂ | 3,4-diClPh | | | | 0.27 (25% M | |
| | | | | | | | | EtOAc/Hex) | |
| 280 | 4-Me | H | Bond | 2-pyridinlyl | TFA | 0.94 | 254.2 | 0.49 (5% | O |
| | | | | | | | | 2M NH₃ in | |
| | | | | | | | | MeOH/CH₂Cl₂₎ | |
| 281 | 4-Me | I | Bond | 3,4-diFPh | | 3.1 | 415 | 0.54 (50% | |
| | | | | | | | | EtOAc/Hex) | |
| 282 | 4-Me | H | NH | 3,5-diClPh | | 2.9 | 336 | 0.63 (100% | H |
| | | | | | | | | EtOAc/Hex) | |
| 283 | 4-N-morpholino | H | Bond | 4-ClPh | | 2.07 | 358.3 | 0.36 (50% | L |
| | | | | | | | | EtOAc/Hex) | |
| 284 | 4-CyPen | H | Bond | 4-MeOPh | | | | 0.55 (50% | N |
| | | | | | | | | EtOAc/Hex) | |
| 285 | 4-iPr | Me | Bond | 4-BrPh | | | | | N |
| 286 | 4-iPr | H | Bond | 2,4-diClPh | | | | 0.40 (30% | N |
| | | | | | | | | EtOAc/Hex) | |
| 287 | 4-iPr | H | Bond | 3-Ph | | | | | N |
| | | | | benzoate | | | | | |
| 288 | 4-iPr | H | Bond | 5-Me-3-Ph- | | | | 0.53 (50% | N |
| | | | | | | | | EtOAc/Hex) | |
| 289 | 4-Ph | H | Bond | 3-pyridinyl | | 1.91 | 316.09 | 0.22(50% | O |
| | | | | | | | | EtOAc/Hex) | |
| 290 | 4-iPr | Me | Bond | 4-ClPh | | | | 0.60 (50% | N |
| | | | | | | | | EtOAc/Hex) | |
| 291 | 4-Pr | Et | Bond | 4-ClPh | | | 344 | | W |
| 292 | 4-(4-FPh) | H | Bond | 4-NO₂Ph | | 3.29 | 378.2 | 0.42 (50% | O |
| | | | | | | | | EtOAc/Hex) | |
| 293 | H | H | CH₂ | 4-FPh | | | | 0.32 (25% | M |
| | | | | | | | | EtOAc/Hex) | |
| 294 | 4-CyPen | H | Bond | 4-MePh | | | | 0.55 (50% | N |
| | | | | | | | | EtOAc/Hex) | |
| 295 | 4-CyPen | H | Bond | 4-CF₃Ph | | | | 0.55 (50% | N |
| | | | | | | | | EtOAc/Hex) | |
| 296 | H | H | Bond | 4-pyridinyl | 2TFA | | | 0.45 (3% | O |
| | | | | | | | | MeOH/CH₂Cl₂) | |
| 297 | 4-FPh | H | Bond | 4-diFMeOPh | | | | 0.62 (50% | O |
| | | | | | | | | EtOAc/Hex) | |
| 298 | 4-CyPr | H | Bond | 2-pyridinyl | | 1.04 | 280.2 | 0.31 (EtOAc) | O |
| 299 | 4-Pr | H | Bond | 4-CF₃OPh | | 1.32 | 365.3 | 0.77 (EtOAc) | O |
| 300 | H | H | CH₂ | 3-NO₂Ph | | | | 0.16 (25% | M |
| | | | | | | | | EtOAc/Hex) | |
| 301 | 4-Me | H | O | Et | | | 221 | | I |
| 302 | 4-(4-FPh) | H | Bond | 4-ClPh | | 3.48 | 367.3 | 0.69 (50% | O |
| | | | | | | | | EtOAc/Hex) | |
| 303 | H | H | Bond | N,N-diEtN | | | 234 | | H |
| 304 | 4-Ph | H | Bond | 2-NO₂Ph | | 2.92 | 360.08 | 0.65 (50% | O |
| | | | | | | | | EtOAc/Hex) | |
| 305 | 4-iPr | H | CH₂ | 4-ClPh | | | | | N |
| 306 | H | H | Bond | 2H-1,4-benzoxazin- | | | 310 | 0.11 (50% | M |
| | | | | 3(4H)-one | | | | EtOAc/Hex) | |
| 307 | H | H | Bond | 2-Ph | | | 359 | 0.26 (25% | O |
| | | | | benzoate | | | | EtOAc/Hex) | |
| 308 | 4-CyPr | H | Bond | 4-CNPh | HCl | | | 0.08 (20% | O |
| | | | | | | | | EtOAc/Hex) | |
| 309 | 4-CyPr | Me | Bond | 4-BrPh | | | | 0.60 (50% | N |
| | | | | | | | | EtOAc/Hex) | |
| 310 | 4-Ph | H | Bond | 4-ClPh | | | | | N |
| 311 | 4-Bu | Pr | Bond | 4-ClPh | | | | | N |
| 312 | 4-CyPr | H | Bond | 1-adamantyl | | | | 0.55 (50% | N |
| | | | | | | | | EtOAc/Hex) | |
| 313 | H | H | Bond | 3-pyridinyl | | | | 0.50 (5% | O |
| | | | | | | | | MeOH/CH₂Cl₂) | |
| 314 | 4-(4-FPh) | H | Bond | 4-CF₃Ph | | | | 0.69 (50% | O |
| | | | | | | | | EtOAc/Hex) | |
| 315 | H | H | Bond | 3-pyridinyl | | | | 0.50 (5% | O |
| | | | | | | | | MeOH/CH₂Cl₂) | |
| 316 | 4-(4-FPh) | Bond | 4-CF₃Ph | | | | | 0.69 (50% | O |
| | | | | | | | | EtOAc/Hex) | |
| 317 | 4-Ph | H | Bond | 4-iPr | | | | 0.21 (50% | O |
| | | | | pyridin-3-yl | | | | EtOAc/Hex) | |
| 318 | 3-Me | H | Bond | 1-adamantyl | | | 311 | | G |
| 319 | 4-(4-FPh) | H | Bond | 4-CNPh | | | | 0.49 (50% | O |
| | | | | | | | | EtOAc/Hex) | |
| 320 | 4-iPr | H | Bond | 1-adamantyl | | | 339 | | G |
| 321 | H | H | Bond | 1-CyPen-1-yl | | | | | G |
| 322 | 4-CyPen | H | Bond | 4-BrPh | | | | 0.55 (50% | N |
| | | | | | | | | EtOAc/Hex) | |
| 323 | 4-CyPen | H | Bond | 4-ClPh | | | | 0.55 (50% | N |
| | | | | | | | | EtOAc/Hex) | |
| 324 | 4-Ph | H | Bond | 4-pyridinyl | | | | | O |
| 325 | 4-(4-pyridinyl)-1- | H | Bond | 4-CNPh | 4 TFA | | | 0.53 (10% | S |
| | piperazinyl | | | | | | | 2M NH₃ in | |
| | | | | | | | | MeOH/EtOAc) | |
| 326 | 4-iPr | H | 1Bond | 2,6-diMeOPh | | | | | N |
| 327 | 4-Ph | H | Bond | 4-NO₂Ph | | | | 0.67 (50% | O |
| | | | | | | | | EtOAc/Hex) | |
| 328 | H | H | Bond | CyBu | — | 2.14 | 217 | 0.22 (25% | G |
| | | | | | | | | EtOAc/Hex) | |
| 329 | 4-CyPen | H | Bond | Ph | | 3.01 | 307.3 | 0.65 (50% | N |
| | | | | | | | | EtOAc/Hex) | |
| 330 | 4-Me | Et | Bond | 4-ClPh | | 2.94 | 315 | 0.65 (50% | W |
| | | | | | | | | EtOAc/Hex) | |
| 331 | 4-iPr | H | Bond | 4-(trifluoromethoxy)Ph | | | 365.7 | 0.50 (50% | N |
| | | | | | | | | EtOAc/Hex) | |
| 332 | H | H | O | CyBu | | 2.17 | 233 | 0.56 (50% | I |
| | | | | | | | | EtOAc/Hex) | |
| 333 | 4-CyPen | H | Bond | 2-ClPh | | 3.16 | 341.3 | 0.63 (50% | N |
| | | | | | | | | EtOAc/Hex) | |
| 334 | 4-iPr | H | Bond | 2H, 3H, 4H- | | | | 0.55 (50% | N |
| | | | | benzo-[b]1, | | | | EtOAc/Hex) | |
| | | | | 4 dioxepin-7-yl | | | | | |
| 335 | 4-Ph | H | Bond | 3-BrPh | | 3.46 | 393 | 0.79 (50% | O |
| | | | | | | | | EtOAc/Hex) | |
| 336 | 4-CyPen | H | Bond | 2,4-(dimethyl)Ph | | 3.27 | 335.3 | 0.55 (50% | N |
| | | | | | | | | EtOAc/Hex) | |
| 337 | 4-Ph | H | Bond | 4-Me-3-pyridyl | | 1.81 | 330.1 | 0.14 (50% | O |
| | | | | | | | | EtOAc/Hex) | |
| 338 | 4-CyPr | H | Bond | 4-(trifluoromethoxy)Ph | | | 363.2 | 0.60 (50% | N |
| | | | | | | | | EtOAc/Hex) | |
| 339 | 4-CyPen | H | Bond | 2,5-(dimethoxy)Ph | | 3.06 | 367.3 | 0.55 (50% | N |
| | | | | | | | | EtOAc/Hex) | |
| 340 | 4-iPr | H | NH | 3,5-diClPh | | | | | H |
| 341 | 4-n-Pr-Amino | H | Bond | 4-ClPh | | 2.52 | 330.3 | 0.57 (5% MeOH/ | L |
| | | | | | | | | EtOAc) | |
| 342 | 4-n-Pr | H | Bond | 4-PhPh | | 1.37 | 357.3 | 0.58 (100% | O |
| | | | | | | | | EtOAc) | |
| 343 | 4-(2-Methyl) | H | Bond | 4-ClPh | | 2.62 | 344.2 | 0.60 (5% | L |
| | Pr | | | | | | | MeOH/EtOAc) | |
| | Amino | | | | | | | | |
| 344 | 4-CyPen | H | Bond | 2-Methoxy | | 3.05 | 337.3 | 0.60 (50% | N |
| | | | | Ph | | | | EtOAc/Hex) | |
| 345 | 4-Ph | H | Bond | 3-ClPh | | 3.4 | 349 | 0.72 (50% | O |
| | | | | | | | | EtOAc/Hex) | |
| 346 | 4-CyPen | H | Bond | 2-naphthyl | | 3.41 | 357.3 | 0.50 (50% | N |
| | | | | | | | | EtOAc/Hex) | |
| 347 | 4-Ph | H | Bond | 2-FPh | | 3.24 | 333.1 | 0.77 (50% | O |
| | | | | | | | | EtOAc/Hex) | |
| 348 | 4-Me | H | Bond | 1,3,4-trihydro- | | | | 0.14 (40% | N |
| | | | | quinolin-2-one-6-yl | | | | EtOAc/Hex) | |
| 349 | 4-Ph | H | Bond | 2,4-diClPh | | | | 0.50 (40% | N |
| | | | | | | | | EtOAc/Hex) | |
| 350 | 4-iPr | Me | Bond | 4-Methoxy | | | | 0.58 (50% | N |
| | | | | Ph | | | | EtOAc/Hex) | |
| 351 | 4-Me | H | Carbonyl | 2-furanyl | | | | 0.25 | Q |
| | | | | methyl | | | | (3% MeOH/ | |
| | | | | amino | | | | CH₂Cl₂) | |
| 352 | 4-(4-methyl | H | Bond | 4-CNPh | 3TFA | | | 0.45 (10% 2M | S |
| | piperazin-lyl | | | | | | | NH₃ in | |
| | Me) | | | | | | | MeOH/CH₂Cl₂) | |
| 353 | 4-CyPen | H | Bond | 3-NO₂-4-Cl | | 3.30 | 386.2 | 0.53 (50% | N |
| | | | | Ph | | | | EtOAc/Hex) | |
| 354 | 4-t-Bu | H | Bond | 1-pyrrolidinyl | | 3.29 | 364.3 | 0.85 (100% | O |
| | | | | | | | | EtOAc) | |
| 355 | 4-CyPen | H | Bond | 2H, 3H, 4H- | | 2.99 | 379.3 | 0.55 (50% | N |
| | | | | benzo-[b]1, | | | | EtOAc/Hex) | |
| | | | | 4 dioxepin-7-yl | | | | | |
| 356 | 4-(2-Methoxyethyl | H | Bond | 4-CNPh | | | | 0.375 (10% 2M | S |
| | amino) | | | | | | | NH₃ in | |
| | | | | | | | | MeOH/CH₂Cl₂) | |
| 357 | 4-Ph | H | Bond | 2-Pyridyl | | 2.17 | 316.1 | 0.48 (50% | O |
| | | | | | | | | EtOAc/Hex) | |
| 358 | 4-Me | H | Carbonyl | 4-F-Ph | | | | 0.29 (3% MeOH/ | Q |
| | | | | | | | | CH₂Cl₂) | |
| 359 | 4-Ph | H | Bond | 4-(difluoromethoxy)Ph | | 3.24 | 381.1 | 0.72 (50% | O |
| | | | | | | | | EtOAc/Hex) | |
| 360 | 4-Me | n-Pr | Bond | 4-ClPh | | 3.12 | 329 | 0.25 (10% EtOAc/ | W |
| | | | | | | | | CH₂Cl₂) | |
| 361 | 4-(4-F) | H | Bond | 2,4-diClPh | | 3.73 | 401.3 | 0.84 (100% | O |
| | | | | | | | | EtOAc) | |
| 362 | 4-CyPen | H | Bond | 3,4-diClPh | | 3.60 | 375.2 | 0.58 (50% | N |
| | | | | | | | | EtOAc/Hex) | |
| 363 | 4-CyPen | H | Bond | 4-(difluoromethoxy)Ph | | 3.15 | 373.3 | 0.60 (50% | N |
| | | | | | | | | EtOAc/Hex) | |
| 364 | 4-CyPen | H | Bond | 3,5-(difluoromethyl)Ph | | 3.79 | 443.2 | 0.55 (50% | N |
| | | | | | | | | EtOAc/Hex) | |
| 365 | 4-iPr | H | Bond | 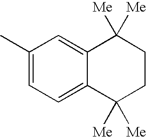 | TFA | | | 0.45 (40%) EtOAc/Hex) | N |
| 366 | 4-(dimethyl | H | Bond | 4-CNPh | | | | 0.15 (60 | S |
| | amino) | | | | | | | EtOAc/Hex) | |
| | methyl | | | | | | | | |
| 367 | 4-iPr | H | Bond | 4-(pyrrolidine)Ph | | | 350.7 | 0.5 (50% | N |
| | | | | | | | | EtOAc/Hex) | |
| 368 | 4-CyPen | Me | Bond | 4-BrPh | | 3.38 | 401.2 | 0.61 (50% | N |
| | | | | | | | | EtOAc/Hex) | |
| 369 | 5-Br | H | Bond | 3-Pyridyl | | 2.23 | 318.2 | (100% | O |
| | | | | | | | | EtOAc) | |
| 370 | 5-Br | H | Bond | 3-FPh | | 3.58 | 366.7 | 0.92 (100% | O |
| | | | | | | | | EtOAc) | |
| 371 | 4-(N-pyrolinomethyl) | H | Bond | 4-CNPh | | | | 0.72 (10% 2M | S |
| | | | | | | | | NH₃ in | |
| | | | | | | | | MeOH/EtOAc | |
| 372 | 4-Ph | H | Bond | 4-(trifluoromethyl)Ph | | 3.51 | 383.1 | 0.78 (50% | O |
| | | | | | | | | EtOAc/Hex) | |
| 373 | 5-Br | H | Bond | t-Bu | | 4.06 | 298.9 | 0.92 (100% | O |
| | | | | | | | | EtOAc) | |
| 374 | 4-Et | H | Bond | 4-phenylPh | | | 343.3 | 0.50 (50% | N |
| | | | | | | | | EtOAc/Hex) | |
| 375 | 4-CyPen | H | Bond | 4-(trifluoromethoxy)Ph | | 3.46 | 391.3 | 0.60 (50% | N |
| | | | | | | | | EtOAc/Hex) | |
| 376 | 4-Ph | H | Bond | 2-ClPh | | 3.27 | 349 | 0.69 (50% | O |
| | | | | | | | | EtOAc/Hex) | |
| 377 | 4-Ph | H | Bond | 4-methoxy | | | | 0.50 (40% | O |
| | | | | Ph | | | | EtOAc/Hex) | |
| 378 | 5-Br | H | Bond | 2,4-dimethyl | | 3.77 | 345.1 | 0.91 (100% | O |
| | | | | Ph | | | | EtOAc) | |
| 379 | 4-Et | Me | Bond | 4-iBuPh | | | 337.3 | 0.50 (50% | N |
| | | | | | | | | EtOAc/Hex) | |
| 380 | 5-Br | H | Bond | 3-ClPh | | 3.77 | 353 | 0.91 (100% | O |
| | | | | | | | | EtOAc) | |
| 381 | 4-Cy | H | Bond | 4-ClPh | | 2.59 | 342.2 | 0.56 (5% MeOH/ | L |
| | | | | | | | | EtOAc) | |
| 382 | 4-CyPen | H | Bond | 4-Phenyl Ph | | | 383.4 | 0.50 (50% | N |
| | | | | | | | | EtOAc/Hex) | |
| 383 | 4-iPr | H | Bond | 3,5-(ditrimethyl) | | 3.61 | 417.2 | 0.55 (50% | N |
| | | | | Ph | | | | EtOAc/Hex) | |
| 384 | 4-CyPen | Me | Bond | 4-methoxy | | 2.98 | 351.3 | 0.58 (50% | N |
| | | | | Ph | | | | EtOAc/Hex) | |
| 385 | 4-CyPen | Me | Bond | 4-ClPh | | 3.34 | 355.2 | 0.63 (50% | N |
| | | | | | | | | EtOAc/Hex) | |
| 386 | 4-Me | H | Carbonyl | 4-(4-ClPh) | | 2.51 | 399 | 0.18 (3% | Q |
| | | | | Piperazin-1-yl | | | | MeOH/CH₂Cl₂) | |
| 387 | 4-CyPen | H | Bond | 2,4-dichloroPh | | 3.56 | 375.2 | 0.58 (3% | |
| | | | | | | | | MeOH/CH₂Cl₂) | |
| 388 | 4-iPr | H | Bond | 3-BrPh | | | 359.7 | 0.55 (50% | N |
| | | | | | | | | EtOAc/Hex) | |
| 389 | 4-Me | H | Bond | Et | | | | | G |
| 390 | 4-Me | H | Bond | 4-phenylPh | | | | 0.33 (40% | N |
| | | | | | | | | EtOAc/Hex) | |
| 391 | 4-iPr | H | Bond | 4-trifluoromethylPh | | 3.24 | 349.2 | 0.60 (50% | N |
| | | | | | | | | EtOAc/Hex) | |
| 392 | 4-Ph | H | Bond | 4-CNPh | | 3.03 | 340.1 | 0.68 (50% | O |
| | | | | | | | | EtOAc/Hex) | |
| 393 | 4-Ph | H | Bond | 4-(N-PyrrolidinoPh) | | 3.36 | 384.1 | 0.43 (50% | O |
| | | | | | | | | EtOAc/Hex) | |
| 394 | 4-Me | H | Carbonyl | (CyHex) | | 2.15 | 302.2 | 0.58 (3% MeOH/ | Q |
| | | | | amino | | | | CH₂Cl₂) | |
| 395 | 4-Me | H | Carbonyl | 4-amino | | 0.81 | 297.1 | 0.58 (3% MeOH/ | Q |
| | | | | | | | | CH₂Cl₂) | |
| 396 | 4-Me | H | Carbonyl | Ph amino | | 2.13 | 296.2 | 0.34 (3% MeOH/ | Q |
| | | | | | | | | CH₂Cl₂) | |
| 397 | 4-Me | H | Carbonyl | 3-amino | | | | 0.34 (3% MeOH/ | Q |
| | | | | | | | | CH₂Cl₂) | |
| 398 | 4-CyPr | H | Bond | 4-phenyl Ph | | | 355.2 | 0.50 (50% | N |
| | | | | | | | | EtOAc/Hex) | |
| 399 | 4-Me | H | Carbonyl | 3,4-diFPh | | 2.45 | 332 | 0.32 (3% MeOH/ | Q |
| | | | | amino | | | | CH₂Cl₂) | |
| 400 | 4-Me | H | Carbonyl | 1-morpholino | | 0.83 | 290.2 | 0.25 (3% MeOH/ | Q |
| | | | | | | | | CH₂Cl₂) | |
| 401 | 4-Me | Me | Bond | Me | | | | | G |
| 402 | 4-iPr | H | Bond | 4-phenyl Ph | | | 357.6 | 0.55 (50% | N |
| | | | | | | | | EtOAc/Hex) | |
| 403 | 4-(piperazin-1-yl) | H | Bond | 4-ClPh | | 1.11 | 357.3 | 0.10 (5% MeOH/ | L |
| | | | | | | | | CH₂Cl₂) | |
| 404 | 4-(4-fluorophenyl | H | Bond | 4-ClPh | | 2.63 | 382.4 | 0.49 (50% | L |
| | amino) | | | | | | | AtOAc/Hex | |
| 405 | 4-(phenyl | H | Bond | 4-ClPh | | 2.62 | 364.4 | 0.55 (50% | L |
| | amino | | | | | | | EtOAc/Hex) | |
| 406 | 4-Me | H | Carbonyl | 4-(MePh | | 2.37 | 310.2 | 0.25 (3% MeOH/ | Q |
| | | | | | | | | CH₂Cl₂) | |
| 407 | 4-Me | H | Carbonyl | 4-(MeOPh | | 2.14 | 326.2 | 0.25 (3% MeOH/ | Q |
| | | | | | | | | CH₂Cl₂) | |
| 408 | 4-Me | H | Carbonyl | 3-Cl-4-FPh | TFA | 2.6 | 348 | 0.29 (3% MeOH/ | Q |
| | | | | | | | | CH₂Cl₂) | |
| 409 | 4-Me | H | Carbonyl | 4-(N-(4-CN | | 2.19 | 390 | 0.25 (3% MeOH/ | Q |
| | | | | piperazin-1-yl | | | | CH₂Cl₂) | |
| 410 | 4-Me | H | Carbonyl | 4-ClPh | | 2.52 | 330 | 0.38 (3% MeOH/ | Q |
| | | | | | | | | CH₂Cl₂) | |
| 411 | 4-(1-Imidazolylmethyl) | H | Bond | 4-CNPh | | | | 0.60 (10% 2M | S |
| | | | | | | | | NH₃ in | |
| | | | | | | | | MeOH/EtOAc) | |
| 412 | 4-Me | H | Bond | 4-amidine) | | 1.82 | 295 | 0.10 (20% NH₃ in | O |
| | | | | Ph | | | | EtOH/ | |
| | | | | | | | | 80% CH₂Cl₂) | |
| 413 | 4-nPr | 4-Me | Bond | 4-ClPh | | 1.45 | 405.3 | 0.83 (100% | O |
| | | Ph | | | | | | CH₂Cl₂) | |
| 414 | 4-CyPr | H | Bond | 3-CNPh | | | 304.2 | 0.55 (50% | N |
| | | | | | | | | EtOAc/Hex) | |
| 415 | 4-Me | H | Bond | 4-Pyridyl | | | | 0.31 (3% MeOH/ | O |
| | | | | | | | | CH₂Cl₂) | |
| 416 | 4-Me | H | Carbonyl | 3-CNPh | TFA | 2.22 | 321 | 0.25 (3% MeOH/ | Q |
| | | | | amino | | | | CH₂Cl₂) | |
| 417 | 4-CyPr | H | Bond | 3-(3,4-diClPh) | | | | 0.20 (40% | O |
| | | | | Isoxazol-5-yl | | | | EtOAc/Hex) | |
| 418 | 4-t-Bu | Me | Bond | 4-i-Bu | | 3.69 | 365.3 | 0.79 (100% | O |
| | | | | | | | | EtOAc) | |
| 419 | 4-(4-iPr | H | Bond | 4-CNPh | 3TFA | | | 0.32 (10% 2M | S |
| | piperazin-1- | | | | | | | NH₃ in | |
| | ylmethyl) | | | | | | | MeOH/CH₂Cl₂) | |
| 420 | 4-(di | H | Bond | 4-CNPh | | | | 0.13 (10% 2M | S |
| | Me | | | | | | | NH₃ in | |
| | Amino | | | | | | | MeOH/CH₂Cl₂ | |
| | Ethyl | | | | | | | | |
| | Amino | | | | | | | | |
| | methyl) | | | | | | | | |
| 421 | 4-(Et | H | Bond | 4-CNPh | | | | 0.30 (10% 2M | S |
| | amino | | | | | | | NH₃ in | |
| | methyl) | | | | | | | MeOH/CH₂Cl₂) | |
| 422 | 4-(Et- | H | Bond | 4-CNPh | | | | 0.30 (10% 2M | S |
| | amino | | | | | | | NH₃ in | |
| | methyl | | | | | | | MeOH/CH₂Cl₂) | |
| 423 | 4-(iPr | H | Bond | 4-CNPh | | | | 0.35 (10% 2M | S |
| | amino) | | | | | | | NH₃ in | |
| | | | | | | | | MeOH/CH₂Cl₂) | |
| Note^{a}-HPLC-electrospray mass spectra (HPLC ES-MS) were obtained using a Hewlett-Packard 1100 HPLC equipped with a quatemaxy pump, a variable wavelength detector set at 254 nm, a YMC pro C-18 column (2 × 23 mm, 120A), and a Finmgan LCQ ion trap mass spectrometer with | | | | | | | | | |
| #electrospray ionization. Spectra were scanned from 120-1200 amu using a variable ion time according to the number of ions in the source. The eluents were A: 2% acetonitrile in water with 0.02% TFA and B: 2% water in acetonitrile with 0.018% TFA. Gradient elution from 10% B to 95% over 3.5 minutes at a flowrate of 1.0 mL/min was used with an initial hold of 0.5 minutes and a final hold at 95% B of 0.5 minutes. Total run time was 6.5 minutes. | | | | | | | | | |

Note^{b}-Molecular ion data obtained via electrospray ionization.
Note^{c}-The following abbreviations were used; Ac-acetyl, Cl-chloro, CF₃-trifluoromethyl, CN-cyano, COOH-carboxylic acid, COOEt-ethyl ester, CyPen-cyclopentyl, CyPr-cyclopropyl, diCl-dichloro, diCF₃ -ditrifluoromethyl, diF-difluoro, Et-ethyl, F-fluoro, iBu-isobutyl, iPr-isopropyl, Me-methyl, MeO-methoxy, n-Bu-n-butyl NMe₂-dimethylamine, NO₂-nitro, Ph-phenyl, Pr-propyl, t-Bu-t-butyl
Note^{d}The following abbreviation was used: Hex - hexanes
Note^{e}NMR spectra data was in agreement with the assigned structure

### General Method T: Synthesis of 4-(3-Pyridyl)-Thiazoles by Parallel Methods

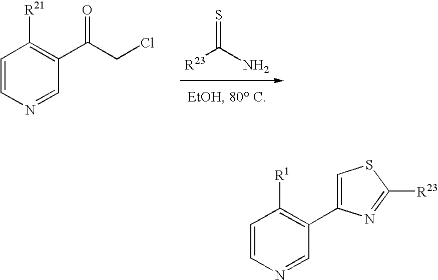

In a 7 mL brown vial were placed the chloroketone (1.0 mmol) and the thioamide (1 eq, 1 mmol) in 3 mL of absolute EtOH. The vial was capped under Ar and shaken at 80° C. overnight. Upon cooling, in several examples the desired product crystallized out of solution and was simply removed by filtration. In other cases, where no crystalization occurred, or where impurities remained, the EtOH was removed and the desired solid heated in a minimal amount of CH₃CN and filtered to yield the pure product. Yields for this reaction were typically 60-90% of the HCl salt. Several examples were then suspended in saturated NaHCO₃ and extracted with CH₂Cl₂ to provide the free base.

### General Method U: Synthesis of 4-(3-Pyridyl)-2-aminothiazoles by Parallel Methods

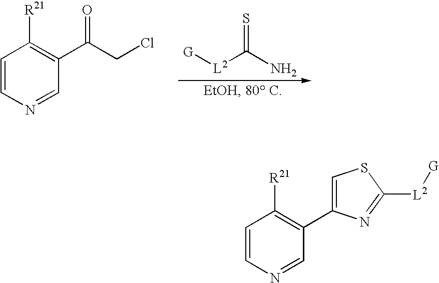

In a 7 mL brown vial were placed the chloroketone (1.0 mmol) and the thiourea (1 equiv., 1 mmol) in 3 mL of absolute EtOH. The vial was capped under Ar and shaken at 80° C. overnight. Upon cooling, in several examples the desired product crystallized out of solution and was simply removed by filtration. In other cases, where no crystalization occurred, or where impurities remained, the EtOH was removed and the desired solid heated in a minimal amount of CH₃CN and filtered to yield the pure product. Yields for this reaction were typically 60-90% of the HCl salt. Several examples were then suspended in saturated NaHCO₃ and extracted with CH₂Cl₂ to provide the free base.

### General Method V: Synthesis of 4-(3-Pyridyl)-Thiazoles by Parallel Methods

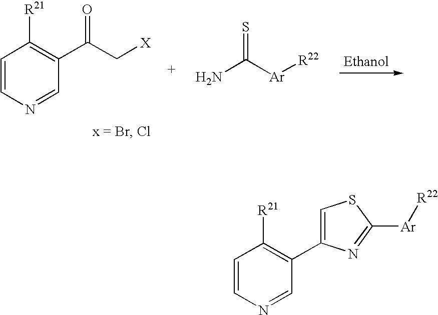

An EPA vial was charged the thioamide (11.2 mmol) and the α-halo ketone (13.5 mmol, 1.20 eq.). To this was added 15 mL of anhydrous ethanol. In the event that the α-halo ketone was a salt, then pyridine (1.2 eq.) was also added to the vial. The vial was capped tightly and shaken in a heating block overnight at 82° C. The reaction mixture was concentrated down and taken up in 2 mL of dichloromethane and 2 mL of water. It was basified with triethylamine (˜10 drops) and extracted twice with dichloromethane. The organic layers were combined and concentrated to dryness, and the crude residue was dissolved in hot DMSO. The compound was purified by Gilson HPLC to yield the desired thiazole derivative.

### Example 424

### General Method W: Alkylation of 4-Methyl Pyridine Containing Thiazoles, as Exemplified by the Preparation of 4-(4-(2-methyl-1-propyl)-3-pyridyl)-2-(4-chlorophenyl)thiazole and 4-(4methyl-3-pyridyl)-5-(2-methyl-1-propyl)-2-(4-chlorophenyl)thiazole

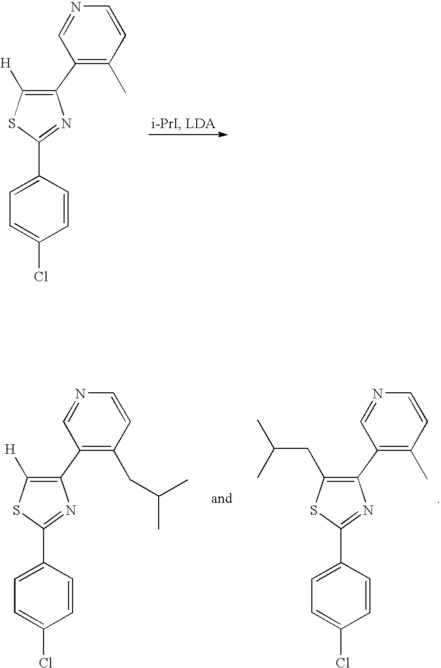

To a solution of LDA 1.05 mmol) in THF, at −78° C., was added the 4-methyl pyridine-containing thiazole (0.70 mmol) followed by the isopropyl iodide. The mixture stirred at −78° C. for 1 h, at 0° C. for 1 h, then at rt for 2 h. MeOH was then added to the reaction and the mixture was evaporated to dryness. The residue was purified by silica gel chromatography to afford the alkylated pyridine-containing thiazoles (0.062 mmol). NMR and MS are consistant with the assigned structures.

TABLE III
4-(3-Pyridyl)thiazoles

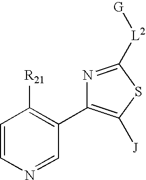

| Ex. | | | | | | t_{R}^{a,b} | MS^{a,b} | TLC R_{f} | General |
|---|---|---|---|---|---|---|---|---|---|
| No. | R^{21c} | J | L² | G^{c} | Salt | min | (M + H⁺) | (Solvent)^{d} | Method^{e} |
| 587 | 4-Me | H | NH | 2-Me-5-F | HCl | 2.08 | 300.3 | | U |
| | | | | Ph | | | | | |
| 587 | 4-Me | H | NH | Ph | | 1.86 | 268.2 | | U |
| 587 | 4-Me | H | Bond | 2-FPh | | 1.91 | 271.2 | | T |
| 587 | 4-Me | H | NH | 2-CF₃Ph | HCl | 2.06 | 336.2 | | U |
| 587 | 4-Me | H | NH | 4-FPh | HCl | 1.91 | 286.3 | | U |
| | | | | | | | [M + 2]⁺ | | |
| 587 | 4-Me | H | Bond | 4-MePh | HCl | 2.16 | 267.3 | | T |
| 587 | 4-Me | H | Bond | 2-MeOPh | HCl | 2.01 | 282.9 | | T |
| 587 | 4-Me | H | NH | 3-FPh | HCl | 1.96 | 286.2 | | U |
| 587 | 4-Me | H | Bond | 3-FPh | HCl | 2.00 | 271.3 | | T |
| 587 | 4-Me | H | NH | 2-FPh | | 1.90 | 286.2 | | U |
| 587 | 4-Me | H | Bond | 3-NO₂Ph | HCl | 2.00 | 298.2 | | T |
| 587 | 4-Me | H | Bond | 3-CF₃Ph | HCl | 2.36 | 321.2 | | T |
| 587 | 4-Me | H | Bond | 5-NO₂ | HCl | 2.48 | 324.2 | 0.70 (4% | V |
| | | | | | | | | 2M NH₃— | |
| | | | | | | | | MeOH/CH₂CL₂) | |
| 587 | 4-Me | H | NH | 4-MePh | HCl | 2.70 | 282.3 | | U |
| 587 | 4-Me | H | NH | 2,4-diFPh | HCl | 1.96 | 304.3 | | U |
| 587 | 4-Me | H | NH | 4-FPh | | 1.94 | 286.2 | | U |
| 587 | 4-Me | H | Bond | 3-MeOPh | HCl | 2.00 | 283.3 | | T |
| 587 | 4-Me | H | Bond | 4-[(4,5-diCl-1H- | HCl | 2.18 | 401.1 | | T |
| | | | | imidazol-1- | | | [M]⁺ | | |
| | | | | yl)Me]Ph | | | | | |
| 587 | 4-Me | H | NH | 2-ClPh | HCl | 2.02 | 302.2 | | U |
| 587 | 4-Me | H | NH | Ph | HCl | 2.38 | 268.3 | | U |
| 587 | 4-Me | H | NH | 2-MeO-4- | HCl | 2.14 | 332.2 | | U |
| | | | | ClPh | | | | | |
| 587 | 4-Me | H | NHCH₂CH₂ | Ph | | 1.99 | 296.3 | | U |
| 587 | 4-Me | H | NH | N-n-Bu | TFA | 1.54 | 248.2 | | U |
| 587 | 4-Me | H | Bond | 4-Me | 2 HCl | 0.77 | 268.2 | 0.40 (% | V |
| | | | | Pyrid-3-yl | | | | 2M-NH₃— | |
| | | | | | | | | MeOH/CH₂CL₂) | |
| 587 | 4-Me | H | NH | 3-MeOPh | HCl | 1.90 | 298.2 | | U |
| 587 | 4-Me | H | NH | 2-FPh | HCl | 1.83 | 286.2 | | U |
| 587 | 4-Me | H | Bond | 2,3-dihydro-1- | HCl | 2.01 | 295.3 | | T |
| | | | | benzofuran-5-yl | | | | | |
| 587 | 4-Me | H | Bond | 2-ClPh | HCl | 2.15 | 287.1 | | T |
| 587 | 4-Me | H | Bond | 2-MeOPh | | 2.06. | 283.2 | | T |
| 587 | 4-Me | H | Bond | 4-MeOPh | HCl | 1.92 | 283.3 | | T |
| 587 | 4-Me | H | NH | 3-ClPh | HCl | 2.16 | 302.2 | | U |
| 587 | 4-Me | H | Bond | 3-ClPh | HCl | 2.22 | 287.1 | | T |
| 587 | 4-Me | H | Bond | 2-NO₂Ph | HCl | 1.80 | 298.2 | | T |
| 587 | 4-Me | H | NH | 2-MeOPh | HCl | 1.96 | 286.2 | | U |
| 587 | 4-Me | H | NH | 4-ClPh | HCl | 2.14 | 302.2 | | U |
| 587 | 4-Me | H | NH | pyridin-3-yl | HCl | 0.72 | 269.3 | | U |
| 587 | 4-Pr | H | Bond | thien-2-yl | | 2.2 | 287.3 | 0.68 (EtOAc) | |
| 587 | 4-Me | H | NH | N-benzyl | | 1.85 | 282.2 | | U |
| 587 | 4-Me | H | NH | 3-CNPh | HCl | 1.86 | 293.3 | | U |
| 587 | 4-Me | H | Bond | 2,3-diClPh | HCl | 2.40 | 321.3 | | T |
| 587 | 4-Me | H | Bond | naphth-2-yl | HCl | | | | T |
| 587 | 4-Pr | H | Bond | 5-NO₂ | | 2.31 | 332.3 | 0.61 | V |
| 587 | 4-Me | H | Bond | 3-F-4-Me | HCl | 2.25 | 285.3 | | T |
| 587 | 4-Me | H | Bond | 4-ClPh | HCl | 2.22 | 287.2 | 0.50 (4% | V |
| | | | | | | | | 2M NH₃— | |
| | | | | | | | | MeOH/CH₂Cl₂) | |
| 587 | 4-Me | H | Bond | 4-NO₂ | HCl | 2.04 | 298.2 | | T |
| 587 | 4-Pr | H | Bond | pyridin-3-yl | | 1.75 | 283.3 | 0.22 (EtOAc) | V |
| 587 | 4-Me | H | Bond | naphth-2-yl | | 2.34 | 303.2 | | T |
| 587 | 4-Me | H | NH | cyclohexyl | | 1.86 | 274.2 | | U |
| 587 | 4-Me | H | NHCH₂ | 2-furyl | TFA | 0.66 | 272.2 | | U |
| 587 | 4-Me | H | Bond | 3-CNPh | TFA | | | .34 (50% | V |
| | | | | | | | | EtOAc/Hex) | |
| 587 | 4-Pr | H | Bond | 3-NO₂Ph | | 2.35 | 326.3 | 0.6 (EtOAc) | V |
| 587 | 4-Me | H | NH | 2,4-diClPh | HCl | 2.30 | 336.2 | | U |
| | | | | | | | [M]⁺ | | |
| 587 | 4-Me | H | Bond | 3-Cl-4-F | HCl | 2.30 | 305.3 | | T |
| | | | | Ph | | | | | |
| 587 | 4-Me | H | Nme | Ph | HCl | 1.99 | 282.3 | | U |
| 587 | 4-Me | H | Bond | 6-Me | 2HCl | | | 0.50 (4% | V |
| | | | | pyridin-3-yl | | | | 2MNH₃— | |
| | | | | | | | | MeOH/CH₂Cl₂) | |
| 587 | 4-Me | H | NH | 2,5-diMeO | HCl | 1.97 | 328.2 | | U |
| 587 | 4-Me | H | Bond | 2-FPh | HCl | 2.00 | 271.3 | | T |
| 587 | 4-Me | H | Nme | Ph | | 2.02 | 282.2 | | U |
| 587 | 4-Pr | H | Bond | Ph | | 2.28 | 281.3 | 0.68 (EtOAc) | V |
| 587 | 4-Pr | H | Bond | pyrid-3-yl | | | | 0.45 (EtOAc) | V |
| 587 | 4-Pr | H | Bond | thien-3-yl | | 2.16 | 287.3 | 0.66 (EtOAc) | V |
| 587 | 4-Me | H | NH | N-[3-(Me | HCl | 2.16 | 314.2 | | U |
| | | | | sulfanyl)Ph] | | | | | |
| 587 | 4-Me | H | Bond | 4-CF₃Ph | HCl | 2.40 | 321.7 | | T |
| 587 | 4-Me | H | Bond | 2,4-diClPh | HCl | 2.40 | 321.7 | | T |
| 587 | 4-Me | H | Bond | thien-2-yl | | | | 0.49 (5% | V |
| | | | | | | | | MeOH/CH₂Cl₂) | |
| 587 | 4-Me | H | NH | N-4-Me | | 2.06 | 296.2 | | U |
| | | | | benzyl | | | | | |
| 587 | 4-Pr | H | Bond | Pyrid-4-yl | | 0.91 | 282.3 | 0.21 (EtOAc) | V |
| 587 | 4-Me | H | NH | 3,5-diClPh | HCl | 3.06 | 336.3 | | U |
| 587 | 4-Pr | H | Bond | 4-CNPh | | 2.23 | 306.4 | 0.64 (EtOAc) | V |
| 587 | 4-Pr | H | Bond | 4-NO₂Ph | | 2.36 | 326.3 | 0.62 (EtOAc) | V |
| 587 | 4-Pr | H | Bond | 4-ClPh | | 2.55 | 315.5 | 0.65(EtOAc) | V |
| 587 | 4-Me | H | Bond | 4-CF₃Ph | | 2.33 | 321.2 | | T |
| 587 | 4-Me | H | NH | 3-CF₃Ph | HCl | 2.26 | 336.3 | | U |
| 587 | 4-Me | H | NH | pyrid-3-yl | | 0.68 | 269.2 | | U |
| 587 | 4-Me | H | Bond | 3-NO₂-4 | HCl | 2.06 | 328.1 | | T |
| | | | | MeOPh | | | | | |
| 587 | 4-Me | H | NH | 4-CF₃Ph | HCl | 2.28 | 336.3 | | U |
| 587 | 4-Me | H | Bond | 4-CNPh | HCl | 1.85 | 278.2 | | T |
| 587 | 4-Me | H | NH | 4-ClPh | | 1.91 | 302.2 | | U |
| 587 | 4-Me | H | CH₂O | 2-ClPh | TFA | 2.19 | 317.2 | | T |
| 587 | 4-Me | H | NH | N-benzhydryl | | 2.36 | 358.2 | | U |
| 587 | 4-Me | H | Bond | 3,5-diCF₃ | HCl | 2.65 | 389.2 | | T |
| | | | | Ph | | | | | |
| 587 | 4-Me | H | Bond | 4-CNPh | | 1.85 | 278.2 | | T |
| 587 | 4-Me | H | NH | 4-MeOPh | HCl | 1.83 | 298.2 | | U |
| | | | | | | | [M + 2]⁺ | | |
| 587 | 4-Me | H | Bond | 4-CNPh | MSA | | | | V |
| 587 | 4-Pr | H | Bond | 2-pyrazine | | 1.75 | 283.3 | 0.55 (EtOAc) | V |
| 587 | 4-Me | H | NH | N-4-Cl | | 2.12 | 316.2 | | U |
| | | | | benzyl | | | [M]⁺ | | |
| 587 | 4-Me | Cl | Bond | 4-ClPh | | | | 0.20 (33% | V |
| | | | | | | | | EtOAc/Hex) | |
| 587 | 4-Me | H | NHCH₂ | 2-tetrahydrofuranyl | TFA | 0.64 | 276.2 | | U |
| 587 | 4-IsoBu | H | Bond | 4-ClPh | | | | 0.21 (33% | W |
| | | | | | | | | EtOAc/Hex) | |
| 587 | 4-Me | H | NH | 2,4-diMeO | HCl | 1.95 | 328.2 | | U |
| | | | | Ph | | | | | |
| 587 | 4-Me | H | 4-diMeN | HCl | 0.18 | 311.3 | | | U |
| | | | | Ph | | | | | |
| 587 | 4-Me | H | NH | 4-CNPh | HCl | 2.35 | 293.3 | | U |
| 587 | 4-Me | H | Bond | 2-Pyrazine | | | | 0.38 (5% | V |
| | | | | | | | | 2N NH₃ | |
| | | | | | | | | MeOH/CH₂Cl₂) | |
| 587 | 4-Me | H | NH | 4-MeOPh | | 1.85 | 298.2 | | U |
| 587 | 4-Me | H | Bond | thiadiazol- | HCl | 2.03 | 337.1 | | T |
| | | | | 4-yl)Ph | | | | | |
| 587 | 4-Me | H | Bond | isoxazol-5-yl | HCl | 0.96 | 244.1 | | T |
| 587 | 4-Me | H | NHCH₂CH₂ | N-piperdinyl | | 0.64 | 303.2 | | U |
| 587 | 4-Me | H | CH₂O | 4-ClPh | HCl | 2.26 | 317.1 | | T |
| 587 | 4-Me | H | NHCH₂CH₂ | N-morpholino | 2 TFA | 0.65 | 305.1 | | U |
| 587 | 4-Me | H | NHCH₂ | 4-MeOPh | | 1.92 | 312.2 | | U |
| 587 | 4-Me | H | Bond | 4-t-BuPh | | 2.59 | 309.4 | | T |
| 587 | 4-Me | H | NHCH₂CH₂CH₂ | NMe₂ | 2 TFA | 0.65 | 277.2 | | U |
| 587 | 4-Me | H | NH | 4-AcPh | HCl | 1.79 | 310.3 | | U |
| 587 | 4-Me | H | NH | 3-CO₂EtPh | HCl | 2.15 | 340.2 | | U |
| 587 | 4-Me | H | NH | 4-NO₂Ph | HCl | 1.96 | 313.2 | | U |
| 587 | 4-Me | H | NH | 4-CO₂EtPh | HCl | 2.14 | 340.2 | | U |
| 587 | 4-Me | H | NHCH₂CH₂CH₂ | N-morpholine | | 0.66 | 319.2 | | U |
| 587 | 4-Me | H | Bond | 2,6-diCl-4- | HCl | 2.52 | 389.2 | | T |
| | | | | CF₃Ph | | | | | |
| 587 | 4-Me | H | Bond | Benzene-4- | | 0.85 | 295 | 0.01 (15% 2M | V |
| | | | | carboximid | | | | NH₃/EtOAc) | |
| | | | | amide | | | | | |
| 587 | 4-Me | H | Bond | 1,1′-biphenyl | HCl | 2.54 | 329.3 | | T |
| 587 | 4-Pr | Et | Bond | 4-ClPh | | | | 0.36 (20% | W |
| | | | | | | | | EtOAc/Hex) | |
| 587 | 4-Me | H | NH | 4-(Benzyloxy)Ph | HCl | 2.43 | 374.2 | | U |
| 587 | 4-Me | H | NH | 4-COOH | TFA | 1.51 | 312.3 | | U |
| | | | | Ph | | | | | |
| 587 | 4-Me | H | Bond | 4-(5-CF₃- | 2 TFA | 2.53 | 414.3 | | T |
| | | | | 2-pyridinyl-2-oxy) | | | | | |
| | | | | Ph | | | | | |
| 587 | 4-Me | H | NH₂ | — | HCl | | | | U |
| 587 | 4-Me | H | Bond | 2,4-diClPh | | 2.41 | 321.2 | | T |
| | | | | | | | [M]⁺ | | |
| 587 | 4-Me | H | Bond | 4-COOH | HCl | 2.59 | | | V |
| | | | | Ph | | | | | |
| 587 | 4-CF₃ | H | Bond | 4-CNPh | | 3.08 | | | V |
| 587 | 4-iPr | H | Bond | 1-isoquinoline | TFA | | | 0.1571 (20% | V |
| | | | | | | | | EtOAc/Hex) | |
| 587 | 4-iPr | H | Bond | 2,6-diCl-4- | TFA | | | 0.1440 (20% | V |
| | | | | pyridine | | | | EtOAc/Hex) | |
| 587 | 4-Et | H | Bond | 3-ClPh | TFA | | | 0.18 (20% | V |
| | | | | | | | | EtOAc/Hex) | |
| 587 | 4-CyPr | H | Bond | 3-ClPh | TFA | | | 0.17 (20% | V |
| | | | | | | | | EtOAc/Hex) | |
| 587 | 4-iPr | H | Bond | 3-Cl-Ph | TFA | | | 0.17 (20% | V |
| | | | | | | | | EtOAc/Hex) | |
| 587 | 4-CyPr | H | Bond | Ph | TFA | | | 0.16 (20% | R |
| | | | | | | | | EtOAc/Hex) | |
| 587 | 4-iPr | H | Bond | Ph | TFA | | | 0.20 (20% | R |
| | | | | | | | | EtOAc/Hex) | |
| 587 | 4-iPr | H | Bond | 4-ClPh | TFA | | | 0.16 (20% | V |
| | | | | | | | | EtOAc/Hex) | |
| 587 | 4-Et | H | Bond | 3-NO₂Ph | TFA | | | 0.10 (20% | V |
| | | | | | | | | EtOAc/Hex) | |
| 587 | 4-CyPr | H | Bond | 4-ClPh | TFA | | | 0.14 (20% | V |
| | | | | | | | | EtOAc/Hex) | |
| 587 | 4-CyPr | H | Bond | 3-NO₂Ph | TFA | | | 0.08 (20% | V |
| | | | | | | | | EtOAc/Hex) | |
| 587 | 4-iPr | H | Bond | 3-NO₂Ph | TFA | | | 0.10 (20% | V |
| 587 | 4-Et | H | Bond | 3-CNPh | TFA | | | 0.07 (20% | V |
| | | | | | | | | EtOAc/Hex) | |
| 587 | 4-iPr | H | Bond | 3-CNPh | TFA | | | 0.03 (20% | V |
| | | | | | | | | EtOAc/Hex) | |
| 587 | 4-CyPr | H | Bond | 3CNPh | TFA | | | 0.03 (20% | V |
| | | | | | | | | EtOAc/Hex) | |
| 587 | 4-Et | H | Bond | 4-NO₂Ph | TFA | | | 0.05 (20% | V |
| | | | | | | | | EtOAc/Hex) | |
| 587 | 4-CyPr | H | Bond | 4-NO₂Ph | TFA | | | 0.04 (20% | V |
| | | | | | | | | EtOAc/Hex) | |
| 587 | 4-iPr | H | Bond | 4-NO₂Ph | TFA | | | 0.06 (20% | V |
| | | | | | | | | EtOAc/Hex) | |
| 587 | 4-CyPr | H | Bond | 3-(6-Me | TFA | | | 0.0 (20% | V |
| | | | | pyridin-2-ol) | | | | EtOAc/Hex) | |
| 587 | 4-iPr | H | Bond | 3-(6-Me | TFA | | | 0.0 (20% | V |
| | | | | pyridin-2-ol | | | | EtOAc/Hex) | |
| 587 | 4-Et | H | Bond | 2-Me | TFA | | | 0.05 (20% | V |
| | | | | pyridin-5-yl | | | | EtOAc/Hex) | |
| 587 | 4-CyPr | H | Bond | 2-Me | TFA | | | 0.038 (20% | V |
| | | | | pyridin-5-yl | | | | EtOAc/Hex) | |
| 587 | 4-iPr | H | Bond | 2-Me | TFA | | | 0.04 (20% | V |
| | | | | pyridin-5-yl | | | | EtOAc/Hex) | |
| 587 | 4-CyPr | H | Bond | 4-MePh | TFA | | | 0.09 (20% | V |
| | | | | | | | | EtOAc/Hex) | |
| 587 | 4-iPr | H | Bond | 4-MePh | TFA | | | 0.11 (20% | V |
| | | | | | | | | EtOAc/Hex) | |
| 587 | 4-CyPr | H | Bond | 4-MeOPh | TFA | | | 0.06 (20% | V |
| | | | | | | | | EtOAc/Hex) | |
| 587 | 4-iPr | H | Bond | 4-MeOPh | TFA | | | 0.08 (20% | V |
| | | | | | | | | EtOAc/Hex) | |
| 587 | 4-Et | H | Bond | 4-CF₃Ph | TFA | | | 0.09 (20% | V |
| | | | | | | | | EtOAc/Hex) | |
| 587 | 4-CyPr | H | Bond | 4-CF₃Ph | TFA | | | 0.08 (20% | V |
| | | | | | | | | EtOAc/Hex) | |
| 587 | 4-iPr | H | Bond | 4-CF₃Ph | TFA | | | 0.09 (20% | V |
| | | | | | | | | EtOAc/Hex) | |
| 587 | 4-CyPr | H | Bond | 2-MeO | TFA | | | 0.07 (20% | V |
| | | | | pyridin-5-yl | | | | EtOAc/Hex) | |
| 587 | 4-iPr | H | Bond | 2-MeO | TFA | | | 0.09 (20% | V |
| | | | | | | | | EtOAc/Hex) | |
| 587 | 4-Et | H | Bond | 3,4-diClPh | TFA | | | 0.09 (20% | V |
| | | | | | | | | EtOAc/Hex) | |
| 587 | 4-CyPr | H | Bond | 3,4-diClPh | TFA | | | 0.07 (20% | V |
| | | | | | | | | EtOAc/Hex) | |
| 587 | 4-iPr | H | Bond | 3,4-diClPh | TFA | | | 0.07 (20% | V |
| | | | | | | | | EtOAc/Hex) | |
| 587 | 4-Et | H | Bond | 4-CNPh | TFA | | | 0.04 (20% | V |
| | | | | | | | | EtOAc/Hex) | |
| 587 | 4-CyPr | H | Bond | 4-CNPh | TFA | | | 0.034 (20% | V |
| 587 | 4-iPr | H | Bond | 4-CNPh | TFA | | | 0.05 (20% | V |
| 587 | 4-Me | H | Bond | 5-Br | | | | 0.31 (100% | V |
| | | | | pyridin-3-yl | | | | EtOAc/Hex) | |
| 587 | 4-Me | H | Bond | 4-pyridinyl | | | | 0.35 (5% | V |
| | | | | | | | | 2N NH₃ | |
| | | | | | | | | MeOH/CH₂Cl₂) | |
| Note^{a} HPLC-electrospray mass spectra (HPLC ES-MS) were obtained using a Hewlett-Packard 1100 HPLC equipped with a quaternary pump, a variable wavelength detector set at 254 nm, a YMC pro C-18 column (2 × 23 mm, 120A), and a Finnigan LCQ ion trap mass | | | | | | | | | |
| # spectrometer with electrospray ionization. Spectra were scanned from 120-1200 amu using a variable ion time according to the number of ions in the source. The eluents were A: 2% acetonitrile in water with 0.02% TFA and B: 2% water in acetonitrile with 0.018% TFA. Gradient elution from 10% B to 95% over 3.5 minutes at a flowrate of 1.0 mL/min was used with an initial hold of 0.5 minutes and a final hold at 95% B of 0.5 minutes. Total run time was 6.5 minutes. | | | | | | | | | |

Note^{b}-Molecular ion data obtained via electrospray ionization.
Note^{c}-The following abbreviations were used; Ac-acetyl, Cl-chloro, CF₃-trifluoromethyl, CN-cyano, COOH-carboxylic acid, COOEt-ethyl ester, CyPr-cyclopropyl, DiCl-dichioro, diCF₃-ditrifluoromethyl, diF-difluoro, Et-ethyl, F-fluoro, iPr-isopropyl, Me-methyl, MeO-methoxy, n-Bu-n-butyl NMe₂-dimethylamine, NO₂-nitro, Ph-phenyl, Pr-propyl, t-Bu-t-butyl
Note^{d}-The following abbreviation was used: Hex-hexanes
Note^{e}-NMR spectra data was in agreement with the assigned structure

### Example 584

### General Method X, as Exemplified by the Preparation of 3-(4-cyclohexyl-5-iodo-1,3-thiazol-2-yl)4-methylpyridine

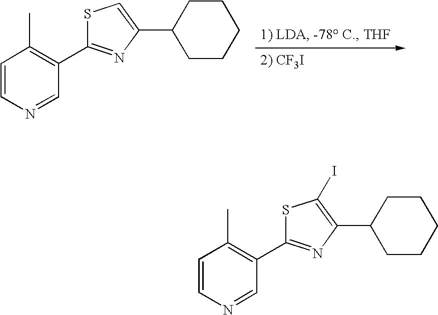

To a solution of LDA (0.581 mmol) in dry THF (5 mL) at −78° C. was added 3-(4-cyclohexyl-1,3-thiazol-2-yl)-4-methylpyridine (100 mg, 0.387 mmol) as a solution in dry THF (2 mL) over 10 min. The red solution was stirred at −78° C. for 30 min. Excess CF₃I gas was condensed in the reaction with a fritted bubbler slowly until the reaction turns clear. The reaction was stirred at −78° C. for 5 min, then warmed up to −25° C. and stirred for 25 min. The reaction was then allowed to warm to rt over 1 h with the ice bath removed. The reaction mixture was concentrated in vacuo and the crude orange oil purified directly by silica gel chromatography (EtOAc/hexanes). The product was isolated as a yellow solid in 68% yield (86 mg, 0.263 mmol): TLC R_{f} 0.54 (50% EtOAc/hexanes); LCMS (M+H)⁺ 385 t_{R}=3.26 min. ¹H NMR (DMSO-d₆) 8.81 (s, 1H), 8.48 (d, 1H, J=5 Hz), 7.39 (d, 1H, J=5 Hz), 2.80 (m, 1H), 2.52 (s, 3H), 1.74 (m, 5H), 1.58 (m, 2H), 1.2-1.42 (m, 3H).

### Example 585

### Preparation of 3-[4-(4Chlorophenyl)-5-iodo-1,3-thiazol-2-yl]4-methylpyridine

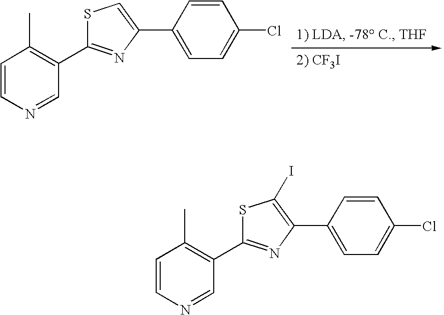

3-[4-(4-chlorophenyl)-5-iodo-1,3-thiazol-2-yl]-4-methylpyridine was prepared from 3-[4-(4-chlorophenyl)-1,3-thiazol-2-yl]-4-methylpyridine according to General Method X: TLC R_{f} 0.50 (50% EtOAc/hexanes); LCMS (M+H)⁺=413, t_{R}=3.23 min.

### Example 586

### Preparation of 3-[4-(3,4-Difluorophenyl-5-iodo-1,3-thiazol-2-yl)-4-methylpyridine

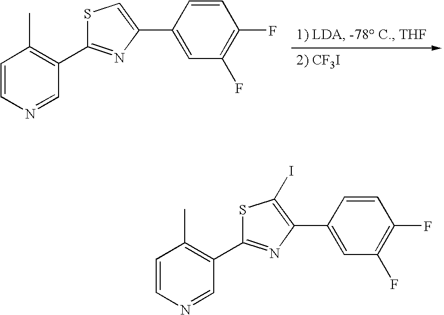

3-[4-(3,4difuorophenyl)-5-iodo-1,3-thiazol-2-yl]-4-methylpyridine (BAY 65-6863) was prepared from 3-[4-(3,4-difluorophenyl)-1,3-thiazol-2-yl]-4-methylpyridine according to General Method X: TLC R_{f} 0.54 (50% EtOAc/hexanes); LCMS 415 (M+H⁺), t_{R}=3.10 min.

TABLE IV
Other Thiazoles

| Ex. | | | t_{R}^{a,b} | MS^{a,b} | TLC R_{f} | General |
|---|---|---|---|---|---|---|
| No. | Structure^{d} | Salt | min | (M + H⁺) | (Solvent)^{c} | Method |
| 587 | 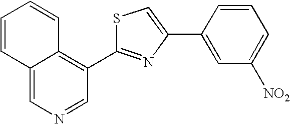 | | | 334.2 | 0.55 (50% EtOAc/hex) | N |
| 588 | 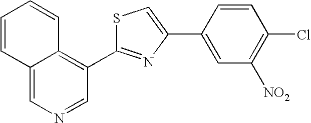 | | | 368.2 | 0.50 (50% EtOAc/hex) | N |
| 589 | 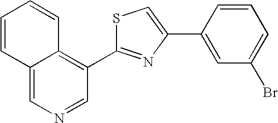 | | | 367.3 | 0.55 (50% EtOAc/hex) | N |
| 590 | 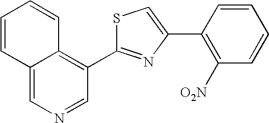 | | | 334.2 | 0.50 (50% EtOAc/hex) | N |
| 591 | 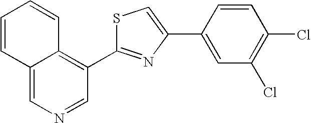 | | | 357.3 | 0.55 (50% EtOAc/hex) | N |
| 592 | 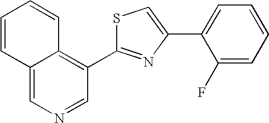 | | | 307.3 | 0.60 (50% EtOAc/hex) | N |
| 593 | 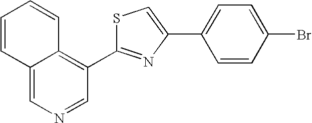 | | | 367.2 | 0.55 (50% EtOAc/hex) | N |
| 594 | 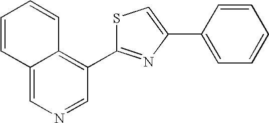 | | | 289.3 | 0.55 (50% EtOAc/hex) | N |
| 595 | 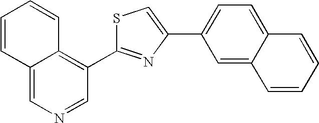 | | | 337.3 | 0.50 (50% EtOAc/hex) | N |
| 596 | 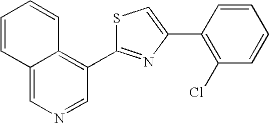 | | | 323.3 | 0.55 (50% EtOAc/hex) | N |
| 597 | 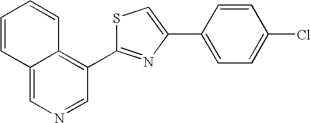 | | | 323.3 | 0.60 (40% EtOAc/hex) | N |
| 598 | 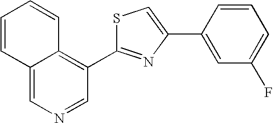 | | | 307.3 | 0.60 (50% EtOAc/hex) | N |
| 599 | 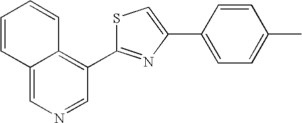 | | | 303.3 | 0.50 (50% EtOAc/hex) | N |
| 600 | 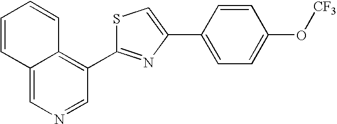 | | | | | O |
| 601 | 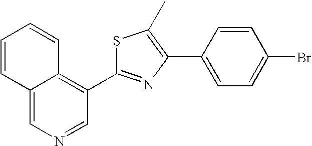 | | | | | O |
| 602 | 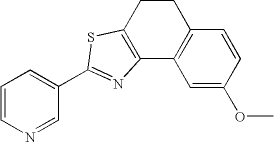 | | | 295 | 0.27 (25% EtOAc/hex) | M |
| 603 | 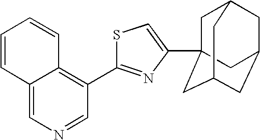 | | 3.68 | 347 | 0.63 (50% EtOAc/hex) | G |
| 604 | 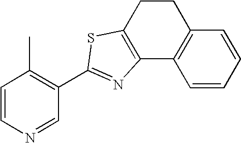 | | 2.49 | 279.2 | 0.32 (40% EtOAc/hex) | O |
| 605 | 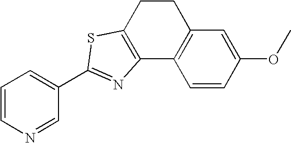 | | 2.73 | 295 | 0.37 (25% EtOAc/hex) | N |
| 606 | 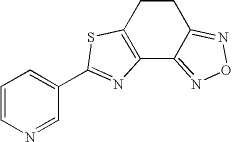 | | 1.91 | 257 | 0.38 (100% EtOAc) | G |
| 607 | 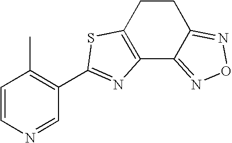 | | 1.65 | 271 | 0.42 (100% EtOAc) | G |
| 608 | 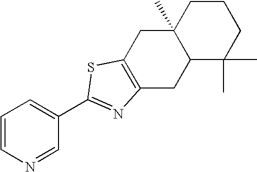 | | 3.38 | 313 | 0.48 (50% EtOAc/hex) | G |
| 609 | 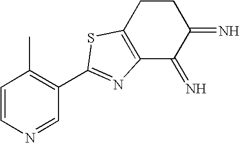 | | 2.29 | 263 | 0.50 50% EtOAc/hex) | G |
| 610 | 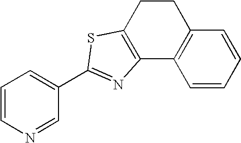 | | 2.81 | 265 | 0.45 (25% EtOAc/hex) | M |
| 611 | 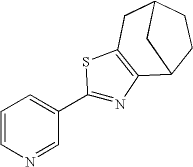 | | 2.31 | 243 | 0.38 (50% EtOAc/hex) | G |
| 612 | 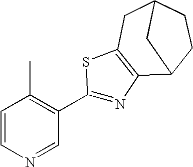 | | 2.10 | 257 | 0.33 (50% EtOAc/hex) | G |
| 613 | 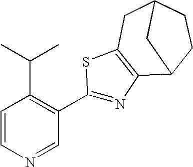 | | 2.63 | 285 | 0.50 (50% EtOAc/hex) | G |
| 614 | 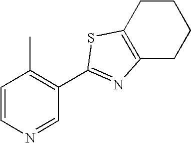 | | 1.80 | 231 | 0.55 (50% EtOAc/hex) | G |
| 615 | 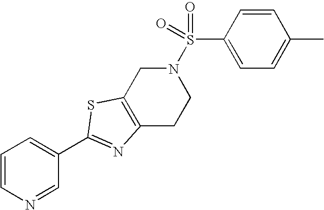 | | 2.44 | 372 | 0.43 (100% EtOAc) | G |
| 616 | 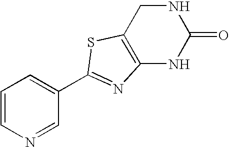 | | 0.64 | 233 | 0.25 (10% MeOH/EtO Ac) | G |
| 617 | 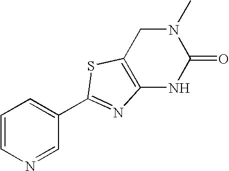 | | 0.80 | 247 | 0.46 (10% MeOH/EtO Ac) | G |
| 618 | 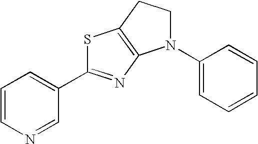 | | 2.80 | 280 | 0.40 (33% EtOAc/hex) | G |
| 619 | 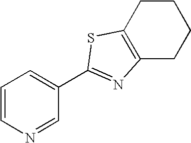 | | 2.02 | 217 | 0.20 (50% EtOAc/hex) | G |
| 620 | 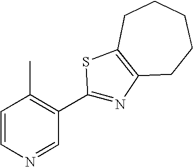 | | 217 | 245 | 0.29 (50% EtOAc/hex) | G |
| 621 | 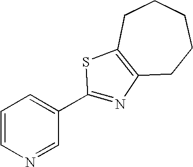 | | 2.36 | 231 | 0.47 (50% EtOAc/hex) | G |
| 622 | 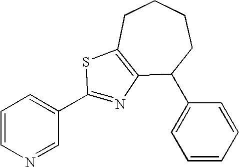 | | 2.93 | 307 | 0.60 (50% EtOAc/hex) | G |
| 623 | 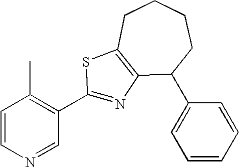 | | 2.64 | 321 | 0.63 (50% EtOAc/hex) | G |
| 624 | 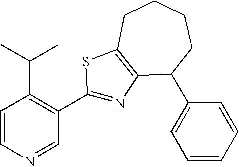 | | 2.98 | 349 | 0.53 (50% EtOAc/hex) | G |
| 625 | 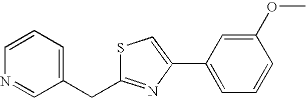 | | | | 0.08 (50% EtOAc/hex) | O |
| 626 | 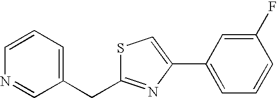 | | | | 0.10 (50% EtOAc/hex) | O |
| 627 | 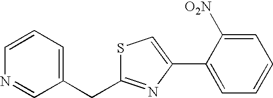 | | | | 0.05 (50% EtOAc/hex) | O |
| 628 | 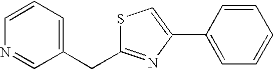 | | | | 0.09 (50% EtOAc/hex) | O |
| 629 | 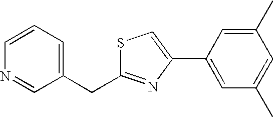 | | | | 0.09 (50% EtOAc/hex) | O |
| 630 | 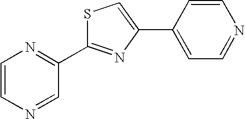 | TFA | | | 0.27 (3% MeOH/CH₂Cl₂) | O |
| 631 | 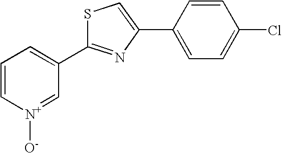 | | | | 0.43 (6% MeOH/CH₂Cl₂) | R |
| 632 | 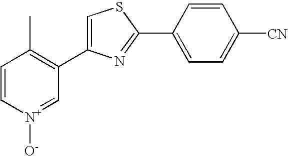 | | 2.89 | 294.33 | 0.11 (10% MeOH/EtO Ac) | R |
| 633 | 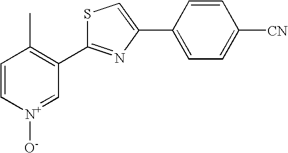 | | 3.18 | 294.33 | 0.13 (10% MeOH/EtO Ac) | R |
| 634 | 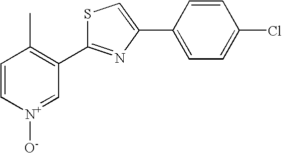 | | 2.54 | 289.2 | 0.43 (6% MeOH/CH₂Cl₂) | R |
| 635 | 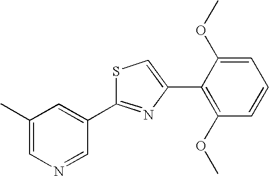 | | | 313 | | N |
| 636 | 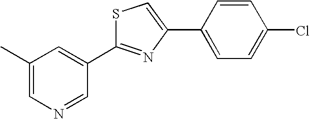 | | | | 0.60 (50% EtOAc/ hex) | N |
| 637 | 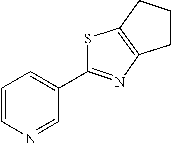 | | 1.76 | 203 | 0.67 (EtOAc) | G |
| 638 | 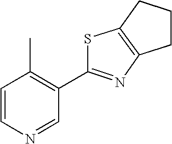 | | 1.51 | 217 | 0.56 (EtOAc) | G |
| 639 | 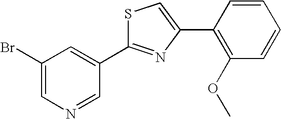 | | | | 0.91 (EtOAc) | O |
| 640 | 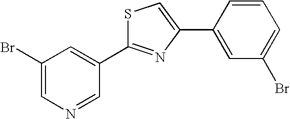 | | | | 0.91 (EtOAc) | O |
| 641 | 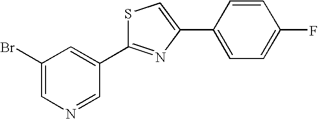 | | | | 0.91 (EtOAc) | O |
| 642 | 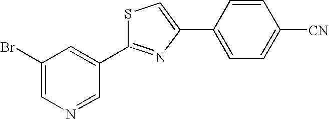 | | | | 0.86 (EtOAc) | O |
| 643 | 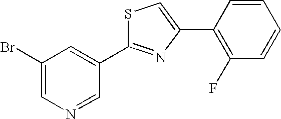 | | | | 0.92 (EtOAc) | O |
| 644 | 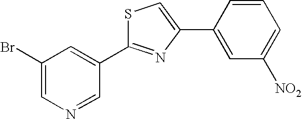 | | | | 0.87 (EtOAc) | O |
| 645 | 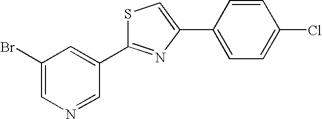 | | | | 0.92 (EtOAc) | O |
| 646 | 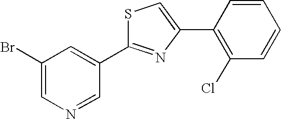 | | | | 0.93 (EtOAc) | O |
| 647 | 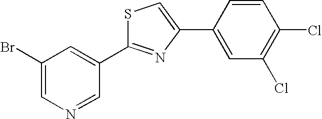 | | | | 0.88 (EtOAc) | O |
| 648 | 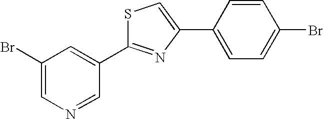 | | | | 0.91 (EtOAc) | O |
| 649 | 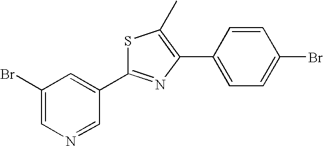 | | | | 0.90 (EtOAc) | O |
| 650 | 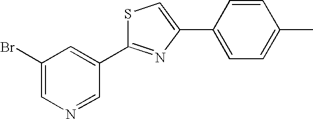 | | | | 0.91 (EtOAc) | O |
| 651 | 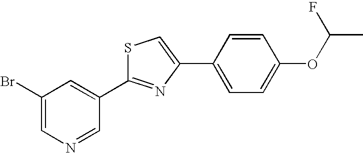 | | | | 0.91 (EtOAc) | O |
| 652 | 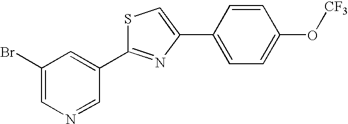 | | | | 0.91 (EtOAc) | O |
| 653 | 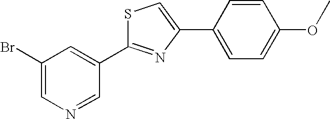 | | | | 0.90 (EtOAc) | O |
| 654 | 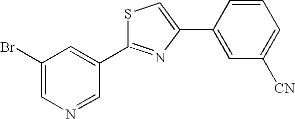 | | | | 0.87 (EtOAc) | O |
| 655 | 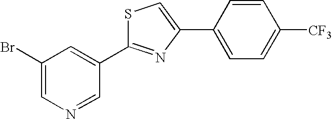 | | | | 0.92 (EtOAc) | O |
| 656 | 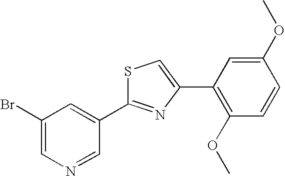 | | | | 0.90 (EtOAc) | O |
| 657 | 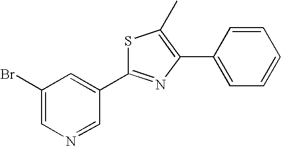 | | | | 0.90 (EtOAc) | O |
| 658 | 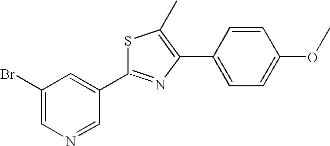 | | | | 0.90 (EtOAc) | O |
| 659 | 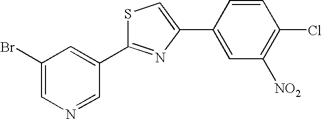 | | | | 0.85 (EtOAc) | O |
| 660 | 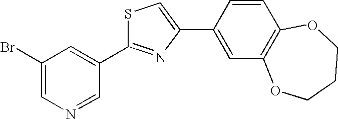 | | | | 0.88 (EtOAc) | O |
| 661 | 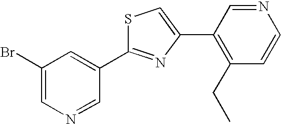 | TFA | | | 0.14 (20% EtOAc/hex) | O |
| 662 | 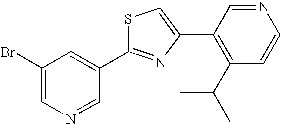 | TFA | | | 0.15 (20% EtOAc/hex) | O |
| 663 | 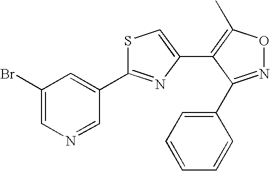 | TFA | | | 0.25 (20% EtOAc/hex) | O |
| Note | | | | | | |
| ^{a}—HPLC—electrospray mass spectra (HPLC ES-MS) were obtained using a Hewlett-Packard 1100 HPLC equipped with a quaternary pump, a variable wavelength detector set at 254 nm, a YMC pro C-18 column (2 x 23 mm, 120A), and a Finnigan LCQ ion trap mass spectrometer with electrospray ionization. Spectra were scanned from 120-1200 amu using a variable ion time according to the number of ions in the source. The eluents were A: 2% | | | | | | |
| # acetonitrile in water with 0.02% TFA and B: 2% water in acetonitrile with 0.018% TFA. Gradient elution from 10% B to 95% over 3.5 minutes at a flowrate of 1.0 mL/min was used with an initial hold of 0.5 minutes and a final hold at 95% B of 0.5 minutes. Total run time was 6.5 minutes. | | | | | | |

Note
^{b}—Molecular ion data obtained via electrospray ionization.
Note
^{c}—The following abbreviation was used: Hex—hexanes
Note
^{d}—NMR spectra data was in agreement with the assigned structure

### General Method Y, as Exemplified by the Preparation of 2-(4-Methyl-3-pyridyl)-4-(3-(diethylamino)phenyl)thiazole

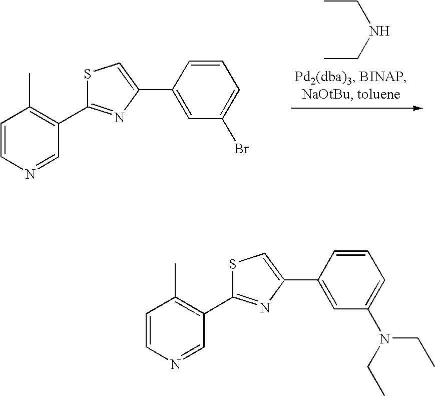

To a solution of 2-(4-methyl-3-pyridyl)-4-(3-bromophenyl)thiazole (0.06 mmol) in toluene (5 mL) was added diethyl amine (0.25 mmol), NaOtBu (0.09 mmol), BINAP (2,2′-bis(diphenylphosphino)-1,1′-binapthyl) (0.0054 mmol), Pd₂(dba)₃ (0.0018 mmol) under argon. The mixture was heated to reflux for 24 h. EtOAc and H₂O were added and the organic and aqueous phase was separated. The organic phase was dried over MgSO₄, filtered and evaporated to dryness. The mixture was purified by Gilson HPLC to afford 4 mg (25%) of the title compound: TLC R_{f} 0.65 (100% EtOAc). The ¹H NMR and MS were consistent with the assigned structure

### Determination of the Activity of the Compounds of the Invention

C17,20 Lyase inhibitory activity of compounds can be determined using, e.g., the biochemical or the cellular assays set forth in the Examples. A person of skill in the art will recognize that variants of these assays can also be used.

The compounds of the invention can also be tested in animal models, e.g., animal models of prostate or breast cancer.

Each of the compounds of the invention was subjected to a biochemical assay and a cellular assay for determining its C17,20 lyase inhibitory activity.

### Human and Murine C17,20 Lyase Biochemical Assays

Recombinant human C17,20 lyase (hLyase) was expressed in (Sf9) cells, and hLyase enriched microsomes were prepared from cultures as described in the following reference: Baculovirus Expression of Bovine P₄₅₀ in Sf9 Cells and Comparison with Expression in Yeast, Mammalian Cells, and E. Coli. Barnes H. J.; Jenkins, C. M.; Waterman, M. R., Archives of Biochemistry and Biophysics (1994) 315(2)489-494. Recombinant murine C17,20 lyase (mLyase) was prepared in a similar manner. hLyase and mLyase preparations were titrated using assay conditions to determine protein concentrations to be used for assays. Both mLyase and hLyase assays were run in an identical manner except that cytochrome b5 was omitted in the murine assays.

Test compounds were diluted 1:4, serially in six steps, with 100% DMSO starting from 800 μM going to 51.2 nM reserving the first 2 columns for the generation of a standard curve. Each of these compound solutions in 100% DMSO was further diluted twenty fold in H₂O to obtain compound concentrations ranging from 40 μM to 2.56 nM in 5% DMSO. Dehydroepiandrosterone (DHEA) standards were serially diluted in 100% DMSO from 400 μM down to 120 nM in half-log dilutions. Each dilution was further diluted twenty fold in H₂O to obtain 20 μM to 6 nM solutions in 5% DMSO using the first 2 columns. Five μl of these 5% DMSO dilutions were used in the assay.

Clear-bottomed opaque 96 well assay plates were loaded with 50 μL of assay buffer (50 mM Na₃PO₄, pH 7.5) and 5 μL of the diluted compounds were added to the wells. Thirty μL of substrate solution (7 mM NADPH (Sigma N1630), 3.35 μM 17-OH-pregnenolone (Steraloids Q4710), 3.35 μg/mL human cytochrome b₅ (Panvera P2252) in 50 mM sodium phosphate pH 7.5 buffer) was added to all wells. Reactions were initiated with the addition of 10 μL hLyase or mLyase in assay buffer.

Enzymatic reactions were allowed to run for 2 h at rt with gentle agitation. Reactions were terminated with the addition of 50 μM (final concentration) YM116, a potent C17,20 lyase inhibitor. The concentration of DHEA generated by hLyase was determined by radioimmunoassay (RIA) as described below.

0.08 μCi ³H-DHEA (1.6 μCi/mL) (NEN (NET814)) in scintillation proximity assay (SPA) buffer (100 mM Tris-HCl, pH 7.5, 50 mM NaCl, 0.5% BSA (Sigma A9647), 0.2% Tween 20) was added to each well. Fifty μL DHEA rabbit antiserum with anti-rabbit SPA beads in SPA buffer was added to all wells. Anti DHEA rabbit antiserum was obtained from Endocrine Sciences (D7-421) (1 mL H₂O to the vial) and anti-Rabbit SPA Beads were obtained from Amersham (RPNQ 0016) (6 mL SPA buffer to the bottle). Mixtures were allowed to equilibrate with gentle agitation for 1 h followed by an overnight equilibration with no agitation. ³H-DHEA bound to the SPA beads was determined by scintillation counting.

The concentration of DHEA generated in each reaction was calculated from raw data (CPM) and the standard curve. The lyase inhibitory activity of each compound was determined as the concentration of DHEA generated in the presence of test compounds, expressed as a percent inhibition compared to the DHEA concentration generated in the absence of test compounds (1−(nM DHEA formed in the presence of test compound/nM DHEA formed in the absence of test compounds)×100).

### Human C17,20 Cellular Assay

Human 293 lyase cells were prepared as described above for the Sf9 cells [Baculovirus Expression of Bovine Cytochrome P₄₅₀ in Sf9 Cells and Comparison with Expression in Yeast, Mammalian Cells, and E. Coli. Barnes, H. J.; Jenkins, C. M.; Waterman, M. R. Archives of Biochemistiy and Biophysics (1994) 315 (2) 489-494]. The cells were cultured in Dulbecco's Modified Eagle's Medium (DMEM)/10% FBS/1% S/P/1% L-Glu/0.8 mg/mLG418/HEPES.

On day one, human 293 lyase cells were plated at 10,000 cells/well/100 μL in columns 2-12 of a 96-well tissue culture plate (Falcon 3075), and allowed to attach overnight (each mother plate needs two cell plates).

On day two, 100 μL H₂O was added to all the wells of a daughter plate (one mother plate one daughter plate Costar 3365). DHEA standard was diluted with RPMI (4.5 μL of 500 μM into 3 mL RPMI, then 1:3 serial dilutions). The media from columns 2-12 of the cell plate was removed and replaced with 100 μL RPMI without phenol red. Diluted DHEA added to column 1 of the cell plate. 50 μL of 100% DMSO was added to columns 1 and 2 of the mother plate. 5 μL of compound was transferred from mother plate to daughter plate, then from the daughter plate to a cell plate using a robot. The cell plate was incubated for 10 min at rt. 15 μL of 10 mM 17-OH-pregnenolone (Steraloids (Q4710) (10 mM stock in 100% DMSO)) was diluted in 30 mL RPMI to obtain a solution of 5 μM 17-OH-pregnenolone. 10 μL of this solution was added to all the wells of the cell plate, except that column received only DMSO. The plate was then incubated for one h at 37° C.

The amount of DHEA produced was determined as follows. 90 μL media was removed from each well of the cell plate and placed into an SPA assay plate (Wallac Isoplate #1450). 50 μL of ³H-DHEA (1.6 μCi/mL, New England Nuclear (Catalog #NET814)) was added to each well of the SPA assay plate. 50 μL of anti-DHEA/anti-rabbit SPA beads (20 μl/mL AB with 10 mg/mL SPA beads) were then added to each well of the plate. The plate was incubated overnight, and the radioactivity counted as described above. The first two columns of the plate were reserved for a standard curve of DHEA and the no compound controls.

The raw data (CPM) was converted to a concentration of DHEA formed (nM) by use of the standard curve. The lyase inhibitory activity of the compounds was determined as the amount of DHEA formed in the presence of compound compared to the amount formed in the absence of compound in the form of a percent inhibition (1−(nM DHEA formed with compound/nM DHEA formed without compound)×100).

A test compound was considered to be active if the IC₅₀ in the human C17,20 biochemical assay or in the human C17,20 cellular assay was less than 10 μM. All the compounds tested have IC₅₀ in the human C17,20 biochemical assay or the human C17,20 cellular assay of less than 10 μM.

### Comparative Testing

The inhibitory activity of 2-[4-methyl-3-pyridyl)-4-(2,4-dichlorophenyl)thiazole was compared to that of 2-(3-pyridyl)-4-(2,4-dichlorophenyl)thiazole, described in EP 411,718. Against C17,20 human lyase, 4-methyl substituted pyridines have been consistently more active than 4-unsubstituted pyridines. In the present case, 2-[4-methyl-3-pyridyl)-4-(2,4-dichlorophenyl)thiazole has an inhibitory IC₅₀ of 15 nM, whereas 2-(3-pyridyl)-4-(2,4-dichlorophenyl)thiazole has an inhibitory IC₅₀ of 406 nM. Surprisingly and unexpectedly, when both compounds were tested against powdery mildew, a fungal species identified in EP 411,718, 2-(3-pyridyl)-4-(2,4-dichlorophenyl)thiazole showed 80% inhibition whereas 2-[4-methyl-3-pyridyl)-4-(2,4dichlorophenyl)thiazole was devoid of activity. *Statistically insignificant

TABLE V
Comparitive Test Data

| | | | % Inhibition |
|---|---|---|---|
| | Human | Mouse | against |
| | Lyase | Lyase | Powdery |
| Structure | IC50 | IC50 | Mildew |
| 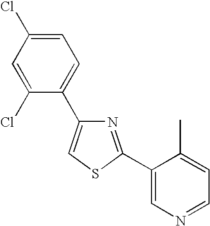 | 1.51E−08 | 3.97E−08 | 10%* |
| 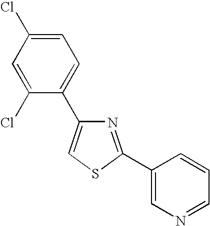 | 4.06E−07 | 1.40E−06 | 80% |

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A compound of the formula (I) 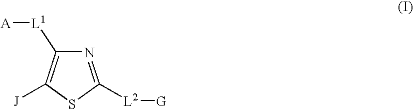
wherein
L¹ represents
a chemical bond;
carbonyl;
—(CH₂)ₐ— wherein
a is 1, 2, or 3;
—CH₂O—;
—OCH₂—;
—O—;
—N(R¹)— wherein
R¹ represents H or C₁₋₄ alkyl;
—NHC(O)—; 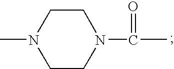
or
—CH₂NHC(O)—;
L² represents
a chemical bond;
—(CH₂)ₐ—;
—CH₂O—;
—N(R¹)—; or
—NH(CH₂)ₐ—;
J represents
H;
C₁₋₄ alkyl; or
halogen; and
1) when L¹ is a chemical bond, A represents 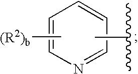
wherein
b is 0, 1, or 2; and
R² is selected from
C₁₋₆ alkyl;
C₁₋₄ haloalkyl;
OR¹;
C₃₋₆ cycloalkyl;
halogen;
phenyl optionally substituted by halogen;
NO₂; 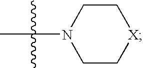
wherein
X represents CH₂, O, S, or N(R¹);
—N(R³)₂ ; wherein
R³ represents H, C₁₋₄ alkyl, C₄₋₆ cycloalkyl, or phenyl optionally substituted by halogen;
—(CH₂)ₐN(R¹)(R⁴) wherein
R⁴ represents —(CH₂)ₐOR¹ or —(CH₂)ₐN(R¹)₂; and
—(CH₂)ₐR⁵; wherein
R⁵ represents 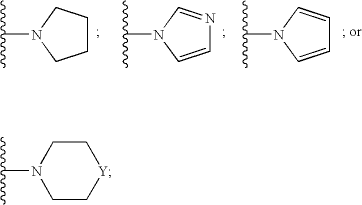
wherein
Y represents N(R¹), O, S, or 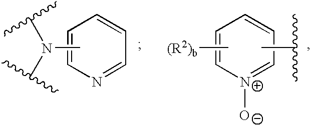
provided that G is other than a pyridyl or an N-oxide-containing group; 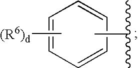
wherein
d is 0, 1, or 2;
R⁶ is selected from
C₁₋₆ alkyl;
C₁₋₄ haloalkyl;
OR⁷; wherein
R⁷ represents H, C₁₋₄ alkyl, C₁₋₄ haloalkyl, phenyl, benzyl, or pyridyl optionally substituted by C₁₋₃ haloalkyl;
halogen;
NO₂;
CN;
CO₂R¹;
C₁₋₄ acyl;
phenyl optionally substituted by halogen;
benzyl;
N(R₁)²; 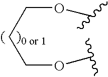
wherein the O atoms are bonded to the phenyl ring at adjacent carbons; 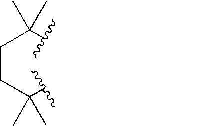
wherein the terminal carbons are bonded to the phenyl ring at adjacent carbons; 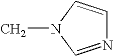
optionally substituted by halogen; 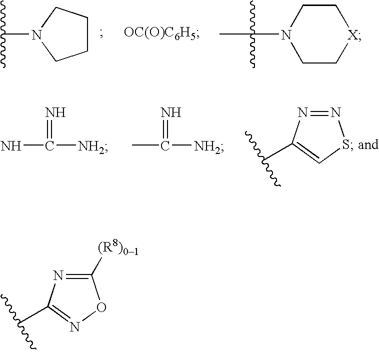
wherein R⁸ represents C₁₋₄ alkyl or phenyl optionally substituted by halogen; 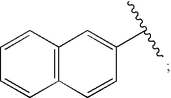
C₃₋₈ cycloalkyl;
C₅₋₆ cycloalkenyl;
adamantyl;
norbomyl; 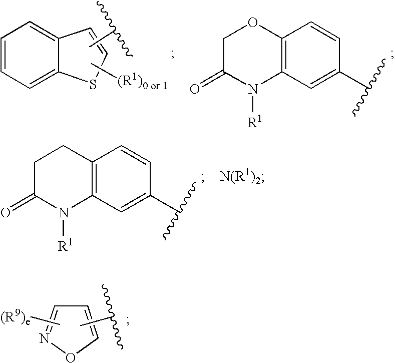
wherein
e is 0, 1, or 2;
R⁹ represents C₁₋₄ alkyl or phenyl optionally substituted by halogen; 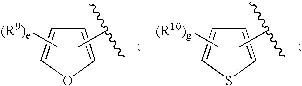
wherein
g is 0, 1, or 2; and
R₁₀ represents CN, NO₂, or halogen;
2) when L² is a bond, G represents 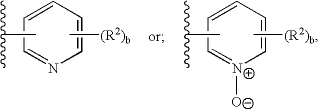
provided that A is other than a pyridyl or an N-oxide-containing group; 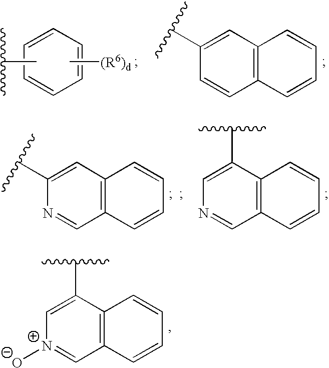
provided that A is other than a pyridyl or an N-oxide-containing group; 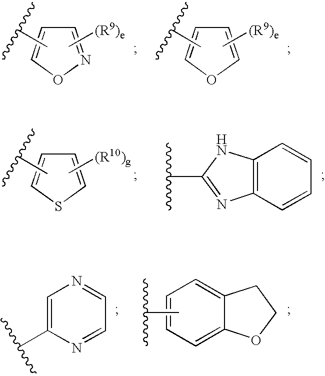
a diazole selected from 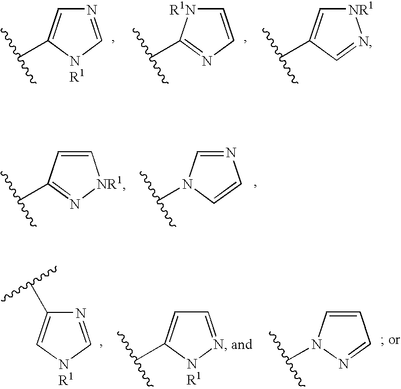
a triazole;
3) when L¹ is carbonyl, A represents 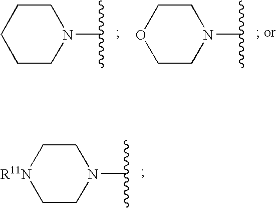
wherein
R¹¹ represents H, C₁₋₄ alkyl, or phenyl optionally substituted by halogen;
4) when L¹ is —(CH₂)ₐ—, A represents 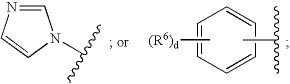
5) when L² is —(CH₂)ₐ—, G represents 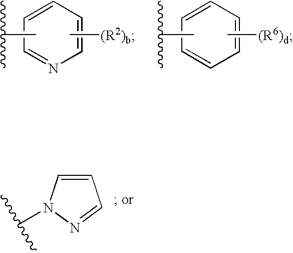
a triazole;
6) when L¹ is —CH₂O, —OCH₂— or O, A represents 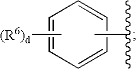
C₁₋₄ alkyl;
C₃₋₈ cycloalkyl; or
C₆₋₇ bicycloalkyl;
7) when L² is —CH₂O—, G represents 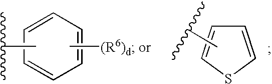
8) when L¹ is —N(R¹)—, A represents 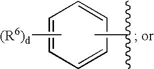
or
C₅₋₆ cycloalkyl;
9) when L² is —N(R¹)— or —NH(CH₂)ₐ—, G represents
C₁₋₆ alkyl;
C₃₋₆ cycloalkyl;
N(R¹)₂; 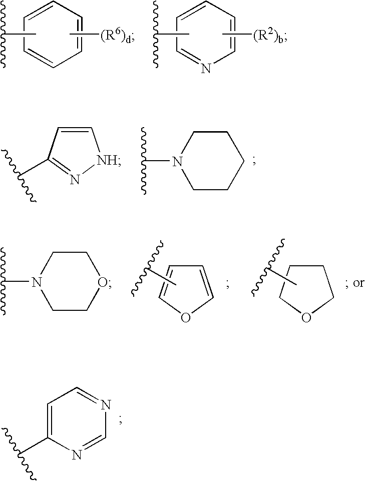
10) when L¹ is —NHC(O)—, 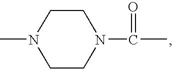
or —CH₂NHC(O)—, A represents 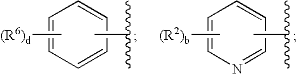
C₅₋₆ cycloalkyl;
C₇₋₈ bicycloalkyl; 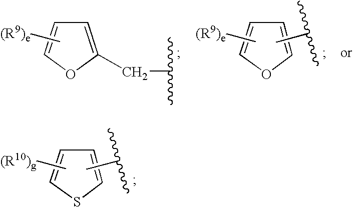
11) one of A and G is a 3-pyridyl moiety of formula (II) or (IIA), or a 4-isoquinolinyl moiety of formula (IIB) or (IIC) 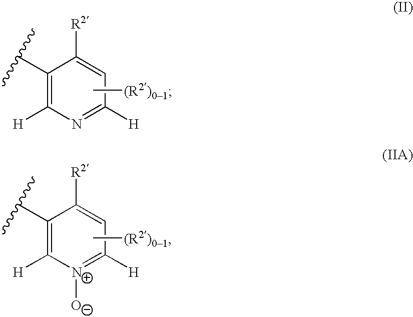
provided that the other of A and G is other than a pyridyl or an N-oxide-containing group; 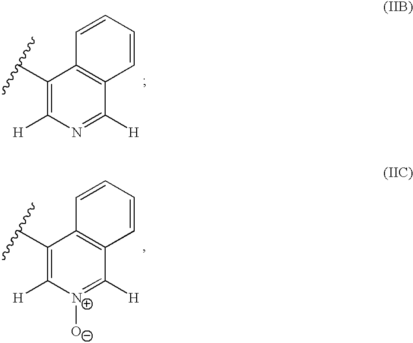
provided that the other of A and G is other than a pyridyl or an N-oxide-containing group;
which is joined to the thiazole ring via a chemical bond L¹ or L² respectively; and the other of A and G is as defined above; and
furthermore, when the other of A and G is joined to the thiazole ring via linker L¹ or L² respectively where L¹ or L² is not a chemical bond, then R^{2′} of formulae (II) and (IIA) is R²; but when each of A and G is joined to the thiazole ring via a chemical bond L¹ and L² respectively, then R^{2′} of formulae (II) and (IIA) is selected from the group consisting of
C₂₋₆ alkyl, provided that when said 3-pyridyl moiety of formula (II) constitutes A, then G is other than phenyl substituted with an amide or sulfonamide group; and when said 3-pyridyl moiety of formula (II) constitutes G, then A is other than phenyl substituted with F;
C₂₋₄ haloalkyl;
C₄₋₆ alkoxy,
C₃₋₆ cycloalkyl;
phenyl optionally substituted by halogen; 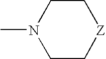
wherein
Z represents CH₂, S, or N(R¹)
—N(R^{3′})₂ wherein
R3′ represents H, C₃₋₄ alkyl, C₄₋₆ cycloalkyl, or phenyl optionally substituted with halogen;
—(CH₂)ₐN(R¹)(R⁴);
—(CH₂)ₐR⁵;
12) alternatively, A-L¹ and J may be joined and together with the carbon atoms to which they are connected form a ring moiety selected from the group consisting of 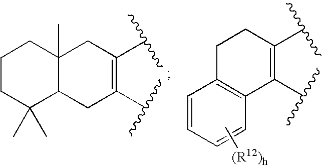
wherein
h is 0, 1, or 2; and
R¹² represents C₁₋₄ alkyl or C₁₋₄ alkoxy, 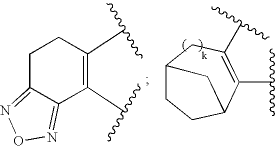
wherein
k is 0 or 1; or 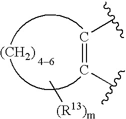
wherein
m is 0, 1, or 2;
R¹³ represents C₁₋₄ alkyl or phenyl;
said ring moiety being joined to the thiazole at the positions indicated by the truncated valences shown in the partial structures above to form a fused ring thiazole; and for these fused ring thiazoles, L² is a bond and G is a 3-pyridyl moiety of formula (III) or (IIIA) 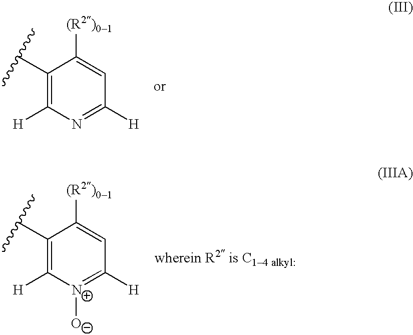
or a pharmaceutically acceptable salt thereof.

2. A compound according to claim 1
wherein
L¹ represents
a chemical bond;
carbonyl;
—(CH₂)ₐ—
—OCH₂—;
L² represents
a chemical bond;
—(CH₂)ₐ—; or
—N(R¹)—;
J represents
H; or
C₁₋₄ alkyl;
1) when L¹ is a chemical bond, A represents 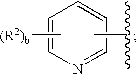
wherein
R² is selected from
C₁₋₆ alkyl;
C₁₋₄ haloalkyl;
C₃₋₆ cycloalkyl;
halogen;
phenyl optionally substituted by halogen; and
—(CH₂)ₐR⁵; 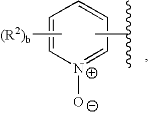
provided that G is other than a pyridyl or an N-oxide-containing group; 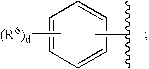
wherein
R⁶ is selected from
C₁₋₆ alkyl;
C₁₋₄ haloalkyl;
OR⁷; wherein
R⁷ represents C₁₋₄ alkyl or C₁₋₄ haloalkyl;
halogen;
NO₂;
CN;
CO₂R¹;
C₁₋₄ acyl; 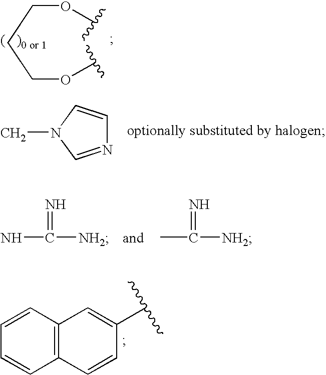
C₃₋₈ cycloalkyl;
C₅₋₆ cycloalkenyl;
adamantyl;
norbornyl; 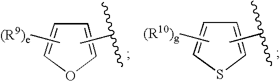
2) when L² is a bond, G represents 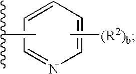
wherein
R² is selected from
C₁₋₆ alkyl;
C₁₋₄ haloalkyl;
C₃₋₆ cycloalkyl;
halogen;
phenyl optionally substituted by halogen; and
—(CH₂)ₐR⁵; 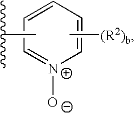
provided that A is other than a pyridyl or an N-oxide-containing group; 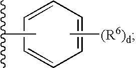
wherein
R⁶is selected from
C₁₋₆ alkyl;
C₁₋₄ haloalkyl;
OR⁷;
halogen;
NO₂;
CN;
CO₂R¹;
C₁₋₄ acyl; 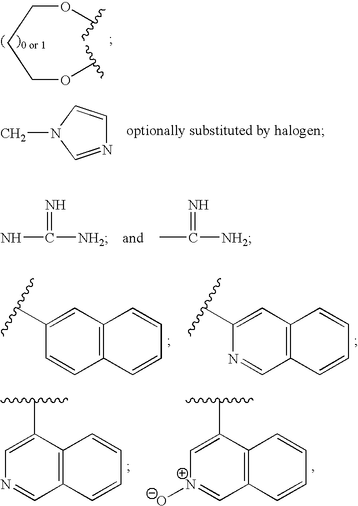
provided that A is other than a pyridyl or an N-oxide-containing group; 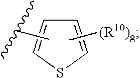
a diazole selected from 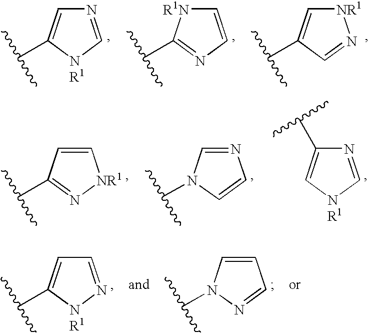
a triazole;
and
when each of A and G is joined to the thiazole ring via a chemical bond L¹ and L² respectively, then R^{2′} of formulae (II) and (IIA) is selected from the group consisting of
C₂₋₆ alkyl, provided that when said 3-pyridyl moiety of formula (II) constitutes A, then G is other than phenyl substituted with an amide or sulfonamide group; and when said 3-pyridyl moiety of formula (II) constitutes G, then A is other than phenyl substituted with F;
C₃₋₆ cycloalkyl;
phenyl optionally substituted by halogen; 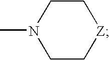
and
—(CH₂)ₐR⁵; and
12) A-L¹ and J may be joined and together with the carbon atoms to which they are connected form a ring moiety selected from the group consisting of

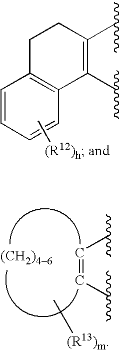

3. A compound according to claim 1
wherein
L¹ represents
a chemical bond;
—(CH₂)ₐ—
—OCH₂—;
L² represents
a chemical bond;
—(CH₂)ₐ—; or
—N(R¹)—;
J represents H;
1) when L¹ is a chemical bond, A represents 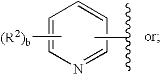
wherein
R² is selected from
C₁₋₆ alkyl;
C₁₋₄ haloalkyl;
C₃₋₆ cycloalkyl; and
phenyl optionally substituted by halogen; 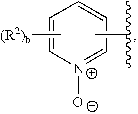
provided that G is other than a pyridyl or an N-oxide-containing group; 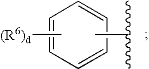
wherein
R⁶ is selected from
C₁₋₆ alkyl;
C₁₋₄ haloalkyl;
OR⁷; wherein
R⁷ represents C₁₋₄ alkyl or C₁₋₄ haloalkyl;
halogen;
NO₂;
CN;
CO₂R¹; and 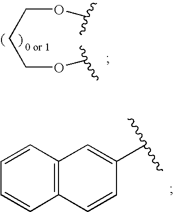
C₃₋₈ cycloalkyl;
C₅₋₆ cycloalkenyl;
adamantyl; or 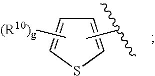
2) when L² is a bond, G represents 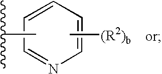
wherein
R² is selected from
C₁₋₆ alkyl;
C₁₋₄ haloalkyl;
C₃₋₆ cycloalkyl; and
phenyl optionally substituted by halogen; 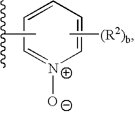
provided that A is other than a pyridyl or an N-oxide-containing group; 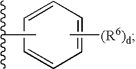
wherein
R⁶ is selected from
C₁₋₆ alkyl;
C₁₋₄ haloalkyl;
OR⁷;
halogen;
NO₂;
CN;
CO₂R¹; and 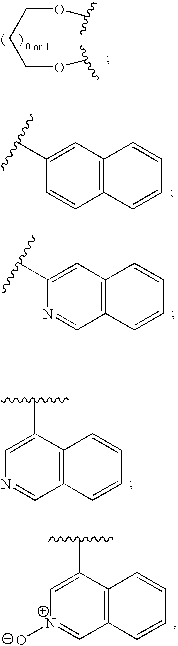
provided that A is other than a pyridyl or an N-oxide-containing group; or 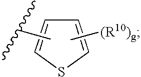
and
when each of A and G is joined to the thiazole ring via a chemical bond L¹ and L² respectively, then R^{2′} of formulae (II) and (IIA) is selected from the group consisting of
C₂₋₆ alkyl, provided that when said 3-pyridyl moiety of formula (II) constitutes A, then G is other than phenyl substituted with an amide or sulfonamide group; and when said 3-pyridyl moiety of formula (II) constitutes G, then A is other than phenyl substituted with F;
C₃₋₆ cycloalkyl; and
phenyl optionally substituted by halogen.

4. A compound according to claim 1
wherein
L¹ represents
a chemical bond;
L² represents
a chemical bond;
J represents H;
1) A represents 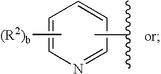
wherein
R² is selected from
C₁₋₆ alkyl;
C₃₋₆ cycloalkyl; and
phenyl optionally substituted by halogen; 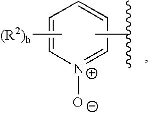
provided that G is other than a pyridyl or an N-oxide-containing group; 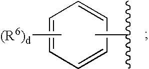
wherein
R⁶ is selected from
C₁₋₆ alkyl;
C₁₋₄ haloalkyl;
OR⁷; wherein
R⁷ represents C₁₋₄ alkyl or C₁₋₄ haloalkyl;
halogen;
NO₂; and
CN;
or 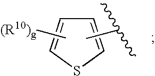
2) G represents 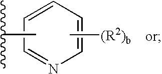
wherein
R² is selected from
C₁₋₆ alkyl;
C₃₋₆ cycloalkyl; and
phenyl optionally substituted by halogen; 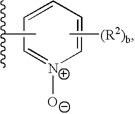
provided that A is other than pyridyl or an N-oxide-containing group; 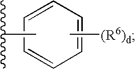
wherein
R⁶ is selected from
C₁₋₆ alkyl;
C₁₋₄ haloalkyl;
OR⁷; wherein
R⁷ represents C₁₋₄ alkyl or C₁₋₄ haloalkyl;
halogen;
NO₂;
CN;
or 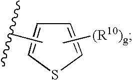
and
when each of A and G is joined to the thiazole ring via a chemical bond L¹ and L² respectively, then R^{2′} of formulae (II) and (IIA) is selected from the group consisting of
C₂₋₆ alkyl, provided that when said 3-pyridyl moiety of formula (II) constitutes A, then G is other than phenyl substituted with an amide or sulfonamide group; and when said 3-pyridyl moiety of formula (II) constitutes G, then A is other than phenyl substituted with F;
and
C₃₋₆ cycloalkyl.

5. A pharmaceutical composition comprising a compound of claim 1 and a pharmaceutically acceptable carrier.

6. A method of inhibiting a lyase enzyme, comprising contacting said lyase enzyme with a compound of claim 1.

7. A method of inhibiting a 17α-hydroxylase-C17,20 lyase, comprising contacting a 17α-hydroxylase-C17,20 lyase with a compound of claim 1.

8. A method for treating a subject having a cancer associated with a 17α-hydroxylase-C17,20 lyase, comprising administering to the subject a therapeutically effective amount of a compound of claim 1.

9. A method for treating prostate cancer in a subject, comprising administering to said subject a therapeutically effective amount of a compound of claim 1, such that the prostate cancer in the subject is treated.

10. A method for treating breast cancer in a subject, comprising administering to said subject a therapeutically effective amount of a compound of claim 1, such that the breast cancer in the subject is treated.

11. The method of any one of claims 8-10, wherein said subject is a primate, equine, canine or feline.

12. The method of any one of claims 8-10, wherein said subject is a human.
